(19) United States
(12) Patent Application Publication
(10) Pub. No.: US 2008/0021024 A1
(43) Pub. Date: Jan. 24, 2008

(54) Metalloprotease inhibitors

(75) Inventors: Irving Sucholeiki, Winchester, MA (US); Christian Gege, Ehingen (DE); Brian M. Gallagher JR., Merrimac, MA (US); Timothy Powers, Boston, MA (US); Hongbo Deng, Southborough, MA (US); Xinyuan Wu, Newton, MA (US); Christoph Steeneck, Dossenheim (DE); Andrew Kiely, Needham, MA (US); Arthur Taveras, Southborough, MA (US)

Correspondence Address: AMGEN INC., MAIL STOP 28-2-C, ONE AMGEN CENTER DRIVE, THOUSAND OAKS, CA, 91320-1799, US

(73) Assignee: Alantos Pharmaceuticals Holding, Inc.

(21) Appl. No.: 11/824,525

(22) Filed: Jun. 29, 2007

Related U.S. Application Data
(60) Provisional application No. 60/817,562, filed on Jun. 29, 2006.

(51) Int. Cl. C07D 239/24; A61K 31/497; A61K 31/505; A61K 31/506; A61K 31/536; A61P 17/00; C12N 9/99; C07D 265/36; A61P 19/00; A61P 25/00; A61P 3/00; A61P 9/00

(52) U.S. Cl. 514/230.5; 435/184; 514/256; 514/269; 544/105; 544/319; 544/333; 544/335

(57) ABSTRACT

The present invention relates to amide containing aromatic MMP inhibiting compounds with a mono-amide heteroaromatic group, of formulas I and II:

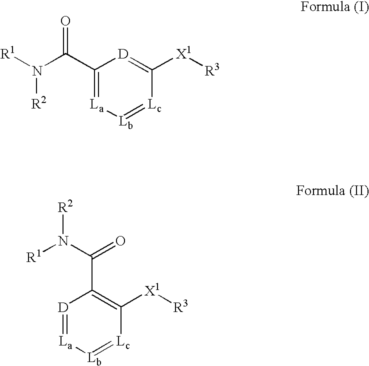

### CROSS REFERENCE TO RELATED APPLICATIONS

This application claims the benefit of U.S. Provisional Application No. 60/817,562, filed Jun. 29, 2006, the contents of which are hereby incorporated by reference.

### FIELD OF THE INVENTION

The present invention relates generally to metalloprotease inhibiting compounds, and more particularly to pyrimidinyl MMP-13 inhibiting compounds.

### BACKGROUND OF THE INVENTION

Matrix metalloproteinases (MMPs) are a family of structurally related zinc-containing enzymes that have been reported to mediate the breakdown of connective tissue in normal physiological processes such as embryonic development, reproduction, and tissue remodeling. Over-expression of MMPs or an imbalance between MMPs has been suggested as factors in inflammatory, malignant and degenerative disease processes characterized by the breakdown of extracellular matrix or connective tissues. MMPs are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The ADAMTSs are a group of proteases that are encoded in 19 ADAMTS genes in humans. The ADAMTSs are extracellular, multidomain enzymes whose functions include collagen processing, cleavage of the matrix proteoglycans, inhibition of angiogenesis and blood coagulation homoeostasis (Biochem. J. 2005, 386, 15-27; Arthritis Res. Ther. 2005, 7, 160-169; Curr. Med. Chem. Anti-Inflammatory Anti-Allergy Agents 2005, 4, 251-264).

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, II, III) and gelatins, proteoglycans, cytokines and other components of ECM (extracellular matrix).

The activation of the MMPs involves the removal of a propeptide portion, which features an unpaired cysteine residue bound to the catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective MMP inhibiting compounds is compounded by several factors, including choice of selective versus broad-spectrum MMP inhibiting activity and rendering such compounds bioavailable via an oral route of administration.

A series of MMP-13 inhibiting compounds containing a bis-amide functional group in combination with a pyridine ring is disclosed in WO 02/064568, while WO 03/049738 discloses that certain bis-amide compounds containing a pyridine and pyrimidine ring and terminally substituted with phenyl rings exhibit selective inhibition of MMP-13 enzymes. However, there are very few amide containing aromatic compounds exhibiting potent and/or selective MMP-13 inhibition.

### SUMMARY OF THE INVENTION

The present invention relates to new classes of amide containing aromatic pharmaceutical agents. In particular, the present invention provides a new class of MMP-13 inhibiting compounds containing an aromatic, particularly a pyrimidinyl, group in combination with an amide and an aryl or hetaryl moiety that exhibit potent MMP-13 inhibiting activity and are highly selective toward MMP-13 compared to currently known MMP inhibitors.

The present invention provides several new classes of amide containing aromatic metalloprotease compounds of the following general formulas: 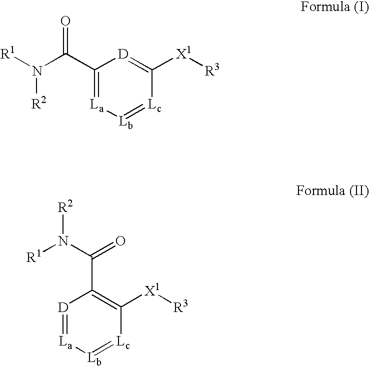
where all variables in the preceding Formulas (I) and (II) are as defined hereinbelow.

The aromatic metalloprotease inhibiting compounds of the present invention may be used in the treatment of metalloprotease mediated diseases, such as rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, chronic wound healing, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, liver fibrosis, lupus, malaria, meningitis, multi-organ dysfunction, necrotizing enterocolitis, osteoporosis, periodontitis, chronic periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

In particular, the amide containing aromatic metalloprotease inhibiting compounds of the present invention may be used in the treatment of MMP-13 mediated osteoarthritis and may be used for other MMP-13 mediated symptoms, inflammatory, malignant and degenerative diseases characterized by excessive extracellular matrix degradation and/or remodelling, such as cancer, and chronic inflammatory diseases such as arthritis, rheumatoid arthritis, osteoarthritis atherosclerosis, abdominal aortic aneurysm, inflammation, multiple sclerosis, and chronic obstructive pulmonary disease, and pain, such as inflammatory pain, bone pain and joint pain.

The present invention also provides amide containing aromatic metalloprotease inhibiting compounds that are useful as active ingredients in pharmaceutical compositions for treatment or prevention of metalloprotease—especially MMP-13—mediated diseases. The present invention also contemplates use of such compounds in pharmaceutical compositions for oral or parenteral administration, comprising one or more of the amide containing aromatic metalloprotease inhibiting compounds disclosed herein.

The present invention further provides methods of inhibiting metalloproteases, by administering formulations, including, but not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular formulations, comprising the amide containing aromatic metalloprotease inhibiting compounds by standard methods known in medical practice, for the treatment of diseases or symptoms arising from or associated with metalloprotease, especially MMP-13, including prophylactic and therapeutic treatment. Although the most suitable route in any given case will depend on the nature and severity of the conditions being treated and on the nature of the active ingredient. The compounds from this invention are conveniently presented in unit dosage form and prepared by any of the methods well-known in the art of pharmacy.

The amide containing aromatic metalloprotease inhibiting compounds of the present invention may be used in combination with a disease modifying antirheumatic drug (such as, for example, methotrexate, azathioptrine, luflunomide, penicillamine, gold salts, mycophenolate, mofetil, cyclophosphamide and the like), a nonsteroidal anti-inflammatory drug (such as, for example, piroxicam, ketoprofen, naproxen, indomethacin, ibuprofen and the like), a COX-2 selective inhibitor (such as, for example, rofecoxib, celecoxib, valdecoxib and the like), a COX-1 inhibitor (such as, for example, piroxicam, tenoxicam and the like), an immunosuppressive (such as, for example, methotrexate, cyclosporin, leflunimide, tacrolimus, rapamycin, sulfasalazine, azathioprine and the like), a steroid (such as, for example, betamethasone, cortisone, prednisone, dexamethasone, fluticasone, mometasone, prednisolone, methylprednisolone, triamcinolone, budesonide, beclomethasone and the like), a biological response modifier (such as, for example, inflixmab, adalimumab, entanercept, ankinra and the like), a viscosupplement (such as, for example, hyaluronates and the like), a pain reducing drug (such as, for example, acetaminophen, aspirin, salicylic acid, codeine, oxymorphone, fentanyl, oxycodone, lidocaine and the like) or other anti-inflammatory agents or therapeutics useful for the treatment of chemokines mediated diseases.

### DETAILED DESCRIPTION OF THE INVENTION

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl(NH₂—CO—), substituted carbamoyl(R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “heteroalkyl” and which may be used interchangeably with the term “alkyl” denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl(NH₂—CO—), substituted carbamoyl(R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl(NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, containing one ring with 3 to 9 carbons. Exemplary unsubstituted such groups include, but are not limited to, cyclopropyl, cyclobutyl, cyclopentyl, cyclohexyl, cycloheptyl, cyclooctyl, cyclodecyl, and cyclododecyl. Exemplary substituents include, but are not limited to, one or more alkyl groups as described above, or one or more groups described above as alkyl substituents.

The term “bicycloalkyl”, as used herein alone or as part of another group, denotes optionally substituted, saturated cyclic bridged hydrocarbon ring systems, desirably containing 2 or 3 rings and 3 to 9 carbons per ring. Exemplary unsubstituted such groups include, but are not limited to, adamantyl, bicyclo[2.2.2]octane, bicyclo[2.2.1]heptane and cubane. Exemplary substituents include, but are not limited to, one or more alkyl groups as described above, or one or more groups described above as alkyl substituents.

The term “spiroalkyl”, as used herein alone or as part of another group, denotes optionally substituted, saturated hydrocarbon ring systems, wherein two rings are bridged via one carbon atom and 3 to 9 carbons per ring. Exemplary unsubstituted such groups include, but are not limited to, spiro[3,5]nonane, spiro[4,5]decane or spiro[2,5]octane. Exemplary substituents include, but are not limited to, one or more alkyl groups as described above, or one or more groups described above as alkyl substituents.

The term “spiroheteroalkyl”, as used herein alone or as part of another group, denotes optionally substituted, saturated hydrocarbon ring systems, wherein two rings are bridged via one carbon atom and 3 to 9 carbons per ring. At least one carbon atom is replaced by a heteroatom independently selected from N, O and S. The nitrogen and sulfur heteroatoms may optionally be oxidized. Exemplary unsubstituted such groups include, but are not limited to, 1,3-diaza-spiro[4,5]decane-2,4-dione. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from N, O and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl; benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

Further examples of heterocycles include, but not are not limited to, “heterobicycloalkyl” groups such as 7-oxa-bicyclo[2.2.1]heptane, 7-aza-bicyclo[2.2.1]heptane, and 1-aza-bicyclo[2.2.2]octane.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl.

“Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more substituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The phrase “fused” means, that the group, mentioned before “fused” is connected via two adjacent atoms to the ring system mentioned after “fused” to form a bicyclic system. For example, “heterocycloalkyl fused aryl” includes, but is not limited to, 2,3-dihydro-benzo[1,4]dioxine, 4H-benzo[1,4]oxazin-3-one, 3H-Benzooxazol-2-one and 3,4-dihydro-2H-benzo[f][1,4]oxazepin-5-one.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocycloalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The phrase “bicyclic fused ring system wherein at least one ring is partially saturated” denotes an 8- to 13-membered fused bicyclic ring group in which at least one of the rings is non-aromatic. The ring group has carbon atoms and optionally 1-4 heteroatoms independently selected from N, O and S. Illustrative examples include, but are not limited to, indanyl, tetrahydronaphthyl, tetrahydroquinolyl and benzocycloheptyl.

The phrase “tricyclic fused ring system wherein at least one ring is partially saturated” denotes a 9- to 18-membered fused tricyclic ring group in which at least one of the rings is non-aromatic. The ring group has carbon atoms and optionally 1-7 heteroatoms independently selected from N, O and S. Illustrative examples include, but are not limited to, fluorene, 10,11-dihydro-5H-dibenzo[a,d]cycloheptene and 2,2a,7,7a-tetrahydro-1H-cyclobuta[a]indene.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Examples therefore may be, but are not limited to, sodium, potassium, choline, lysine, arginine or N-methyl-glucamine salts, and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Pa., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The phrase “pharmaceutically acceptable carrier” denotes media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to poly(D,L)-lactide-co-glycolic acid copolymer (PLGA), poly(L-lactic acid) (PILA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions of the invention may also be formulated as suspensions including a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. The amount of solubility enhancer employed will depend on the amount of the compound of the present invention in the composition.

The term “formulation” denotes a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about −10-80° C., desirably about 0° C.

The term “polymorph” denotes a form of a chemical compound in a particular crystalline arrangement. Certain polymorphs may exhibit enhanced thermodynamic stability and may be more suitable than other polymorphic forms for inclusion in pharmaceutical formulations.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures.

The term “racemic mixture” denotes a mixture that is about 50% of one enantiomer and about 50% of the corresponding enantiomer relative to all chiral centers in the molecule. Thus, the invention encompasses all enantiomerically-pure, enantiomerically-enriched, and racemic mixtures of compounds of Formulas (I) and (II).

Enantiomeric and stereoisomeric mixtures of compounds of the invention can be resolved into their component enantiomers or stereoisomers by well-known methods. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N.Y.). Enantiomers and stereoisomers can also be obtained from stereomerically- or enantiomerically-pure intermediates, reagents, and catalysts by well-known asymmetric synthetic methods.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:
C₁-C₄ alkyl;
C₂-C₄ alkenyl;
C₂-C₄ alkynyl;
CF₃;
halo;
OH;
O—(C₁-C₄ alkyl);
OCH₂F;
OCHF₂;
OCF₃;
ONO₂;
OC(O)—(C₁-C₄ alkyl);
OC(O)—(C₁-C₄ alkyl);
OC(O)NH—(C₁-C₄ alkyl);
OC(O)N(C₁-C₄ alkyl)₂;
OC(S)NH—(C₁-C₄ alkyl);
OC(S)N(C₁-C₄ alkyl)₂;
SH;
S—(C₁-C₄ alkyl);
S(O)—(C₁-C₄ alkyl);
S(O)₂—(C₁-C₄ alkyl);
SC(O)—(C₁-C₄ alkyl);
SC(O)O—(C₁-C₄ alkyl);
NH₂;
N(H)—(C₁-C₄ alkyl);
N(C₁-C₄ alkyl)₂;
N(H)C(O)—(C₁-C₄ alkyl);
N(CH₃)C(O)—(C₁-C₄ alkyl);
N(H)C(O)—CF₃;
N(CH₃)C(O)—CF₃;
N(H)C(S)—(C₁-C₄ alkyl);
N(CH₃)C(S)—(C₁-C₄ alkyl);
N(H)S(O)₂—(C₁-C₄ alkyl);
N(H)C(O)NH₂;
N(H)C(O)NH—(C₁-C₄ alkyl);
N(CH₃)C(O)NH—(C₁-C₄ alkyl);
N(H)C(O)N(C₁-C₄ alkyl)₂;
N(CH₃)C(O)N(C₁-C₄ alkyl)₂;
N(H)S(O)₂NH₂);
N(H)S(O)₂NH—(C₁-C₄ alkyl);
N(CH₃)S(O)₂NH—(C₁-C₄ alkyl);
N(H)S(O)₂N(C₁-C₄ alkyl)₂;
N(CH₃)S(O)₂N(C₁-C₄ alkyl)₂;
N(H)C(O)O—(C₁-C₄ alkyl);
N(CH₃)C(O)O—(C₁-C₄ alkyl);
N(H)S(O)₂O—(C₁-C₄ alkyl);
N(CH₃)S(O)₂O—(C₁-C₄ alkyl);
N(CH₃)C(S)NH—(C₁-C₄ alkyl);
N(CH₃)C(S)N(C₁-C₄ alkyl)₂;
N(CH₃)C(S)O—(C₁-C₄ alkyl);
N(H)C(S)NH₂;
NO₂;
CO₂H;
CO₂—(C₁-C₄ alkyl);
C(O)N(H)OH;
C(O)N(CH₃)OH:
C(O)N(CH₃)OH;
C(O)N(CH₃)O—(C₁-C₄ alkyl);
C(O)N(H)—(C₁-C₄ alkyl);
C(O)N(C₁-C₄ alkyl)₂;
C(S)N(H)—(C₁-C₄ alkyl);
C(S)N(C₁-C₄alkyl)₂;
C(NH)N(H)—(C₁-C₄ alkyl);
C(NH)N(C₁-C₄ alkyl)₂;
C(NCH₃)N(H)—(C₁-C₄ alkyl);
C(NCH₃)N(C₁-C₄ alkyl)₂;
C(O)—(C₁-C₄ alkyl);
C(NH)—(C₁-C₄ alkyl);
C(NCH₃)—(C₁-C₄ alkyl);
C(NOH)—(C₁-C₄ alkyl);
C(NOCH₃)—(C₁-C₄ alkyl);
CN;
CHO;
CH₂OH;
CH₂O—(C₁-C₄ alkyl);
CH₂NH₂;
CH₂N(H)—(C₁-C₄ alkyl);
CH₂N(C₁-C₄ alkyl)₂;
aryl;
heteroaryl;
cycloalkyl;
heterocyclyl; and
keto.

In some cases, a ring substituent may be shown as being connected to the ring by a bond extending from the center of the ring. The number of such substituents present on a ring is indicated in subscript by a number. Moreover, the substituent may be present on any available ring atom, the available ring atom being any ring atom which bears a hydrogen which the ring substituent may replace. For illustrative purposes, if variable R^{X} were defined as being: 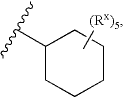
this would indicate that R^{X} is a cyclohexyl ring bearing five R^{X} substituents. The R^{X} substituents may be bonded to any available ring atom. For example, among the configurations encompassed by this are configurations such as:

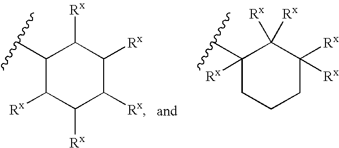

These configurations are illustrative and are not meant to limit the scope of the invention in any way.

In one embodiment of the present invention, the amide containing aromatic metalloprotease compounds may be represented by the general Formula (I): 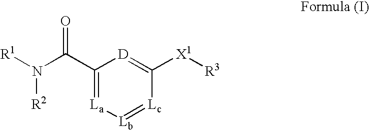
wherein:

R¹ in each occurrence is independently selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

wherein optionally two hydrogen atoms on the same atom of the R¹ group are replaced with ═O, ═S or ═NR¹⁰;

R² in each occurrence is independently selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰ (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹¹, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

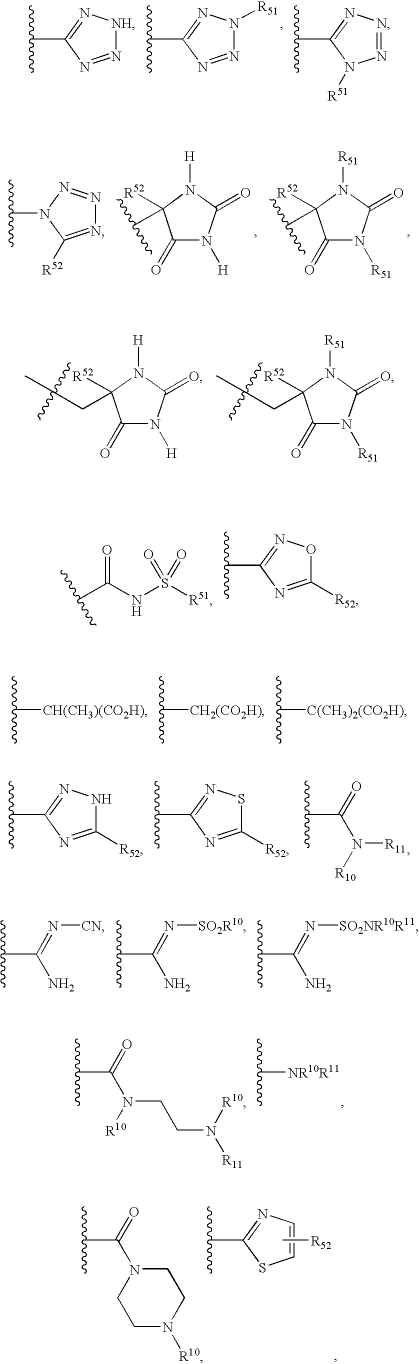

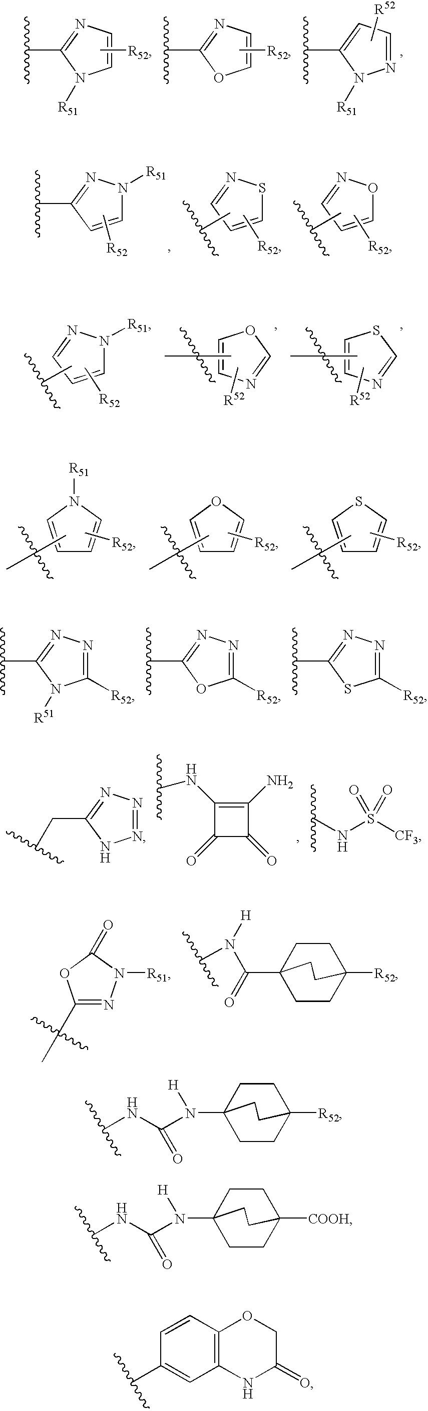

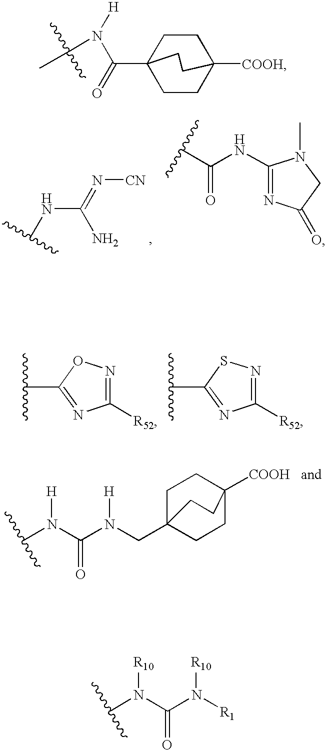

R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)NR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NRt″-CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from the group consisting of hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from the group consisting of hydrogen, alkyl, arylalkyl, cycloalkylalkyl, heteroarylalkyl, heterocycloalkyl and halo, wherein alkyl, arylalkyl, cycloalkylalkyl, heteroarylalkyl and heterocycloalkyl are optionally substituted one or more times.

R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 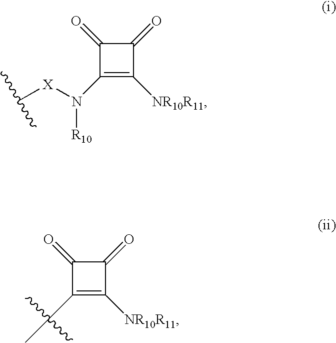
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicylic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from the group consisting of alkyl and (C₀-C₆)-alkyl-aryl, wherein alkyl and aryl are optionally substituted;

R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkcyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

D is a member selected from the group consisting of CR²² and N;

Lₐ, L_{b}, and L_{c} are independently selected from CR⁹ and N with the proviso that Lₐ, L_{b}, and L_{c}, cannot all simultaneously be N;

X¹ is selected from a bond, NR¹⁰, CH₂, CHR²⁰, CR²⁰R²¹, SO₂, SO, S, PO₂, O, C═S, C═O, C═NR¹, C═N—SO₂R¹⁰, C═N—CN, C═N—CONR¹⁰R¹¹, C═N—COR¹⁰, C═N—OR¹⁰, NR¹⁰C═O, NR¹⁰SO₂ and SO₂NR¹⁰;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In another embodiment, compounds of Formula (I) may be selected from the Group I(a):

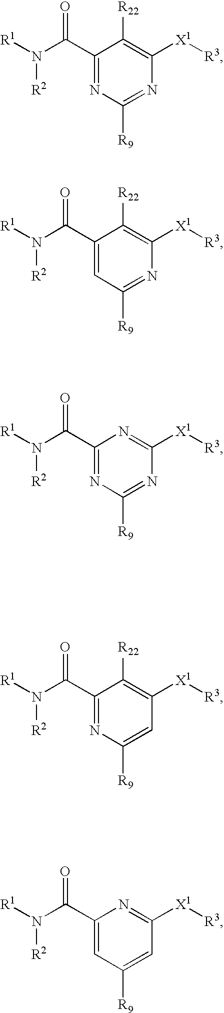

In still another embodiment, compounds of Formula (I) may be selected firom:

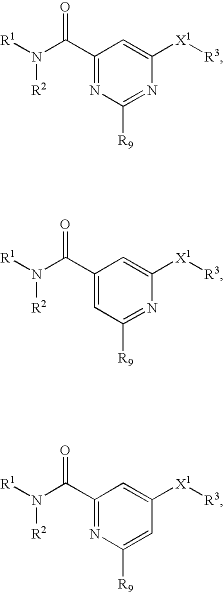

In yet another embodiment, compounds of Formula (I) may be selected from:

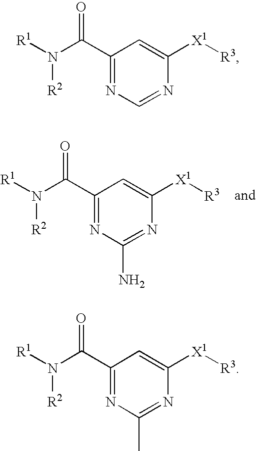

In another embodiment, compounds of Formula (I) may be selected from:

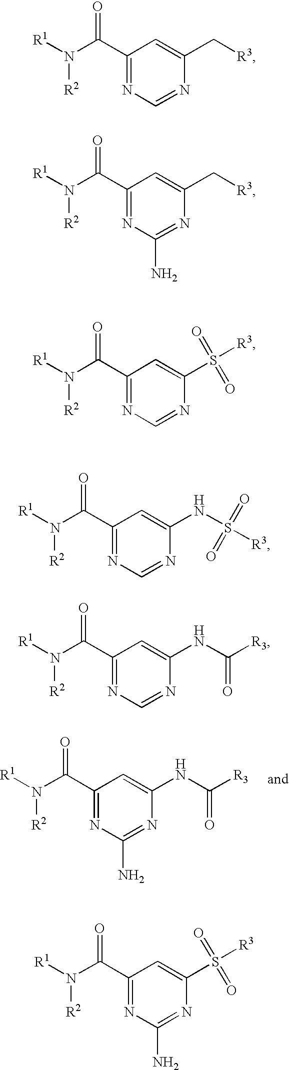

In some embodiments R³ of the compounds of Formula (I) may be selected from Substituent Group 1:

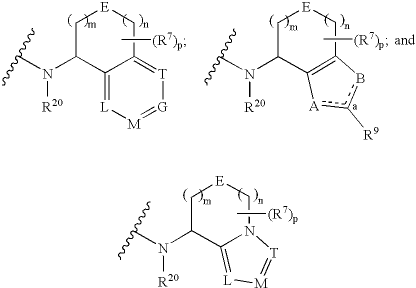

wherein:

R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁷ is independently selected from the group consisting of hydrogen, alkyl, cycloalkyl, halo, R⁴ and NR¹⁰R¹¹ or optionally two R⁷ groups together at the same carbon atom form ═O, ═S or ═NR¹⁰;

A and B are independently selected from the group consisting of CR⁹, CR⁹R¹⁰, NR¹⁰, N, O and S;

E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

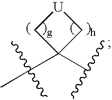

G, L, M and T are independently selected from the group consisting of CR⁹ and N;

U is selected from the group consisting of C(R⁵R¹⁰), NR⁵, O, S, S═O and S(═O)₂;

W¹ is selected from the group consisting of O, NR⁵, S, S═O, S(═O)₂, N(R¹⁰)(C═O), N(R¹⁰)S(═O)₂ and S(═O)₂N(R¹⁰);

g and h are independently selected from 0-2;

m and n are independently selected from 0-3, provided that:
(1) when E is present, m and n are not both 3;
(2) when E is —CH₂—W¹—, m and n are not 3; and
(3) when E is a bond, m and n are not 0; and

p is selected from 0-6;

wherein the dotted line represents a double bond between one of: carbon “a” and A, or carbon “a” and B.

In yet a further embodiment, R³ of Formula (I) may be selected from Substituent Group 3:

hydrogen, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

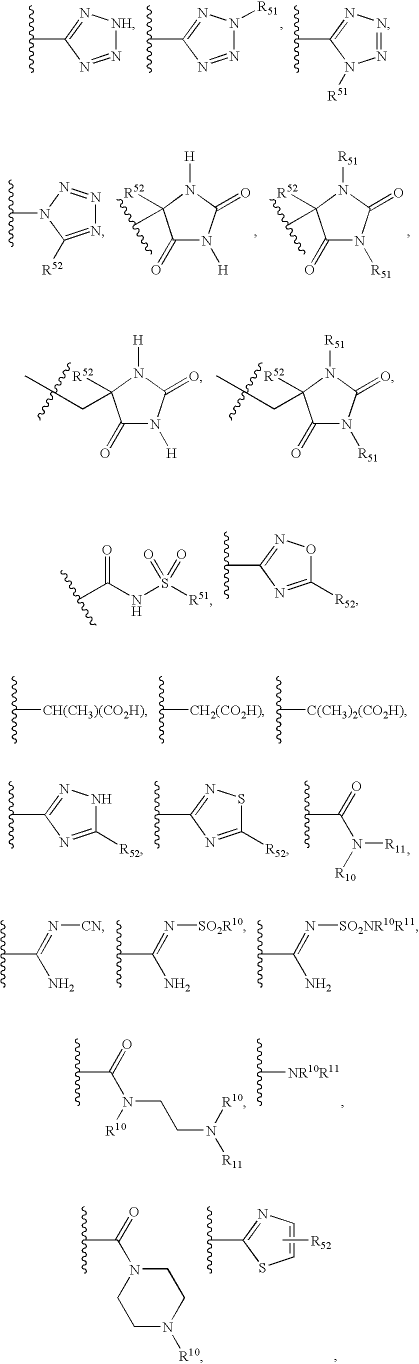

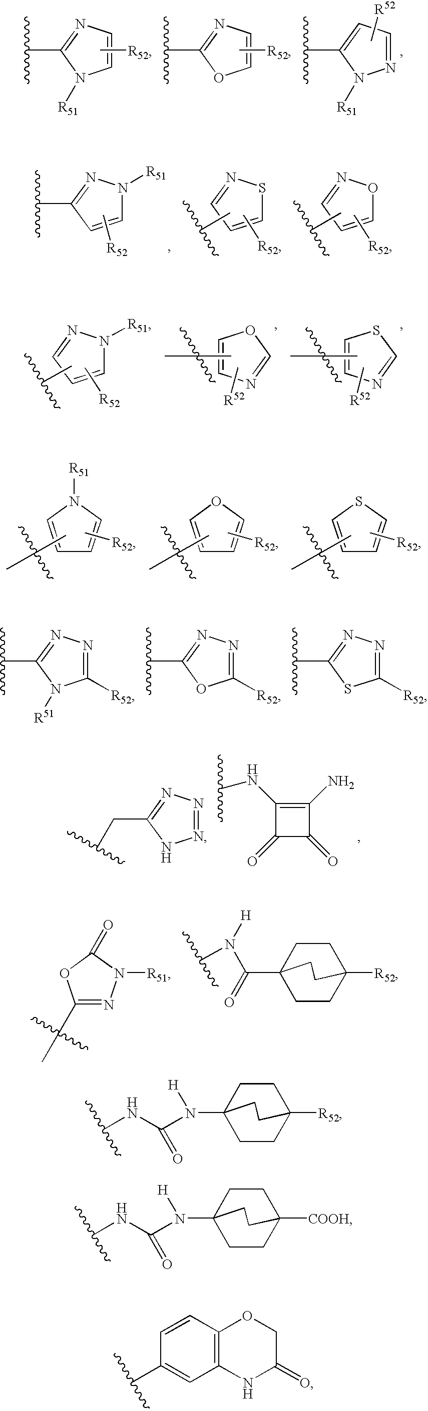

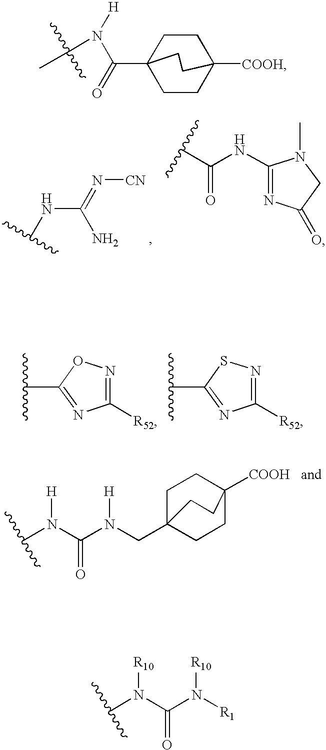

In some embodiments, R³ of Formula (I) may be selected from Substituent Group I(2):

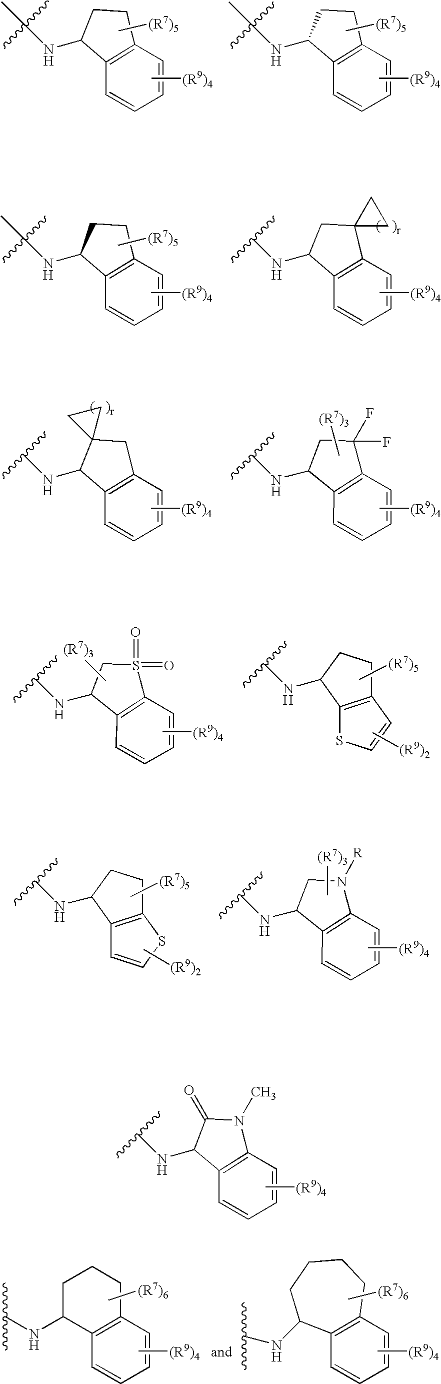

wherein:

R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and

r is selected from 1-4.

For example, in some embodiments, R³ of the compounds of Group I(a) may be selected form Substituent Group 2, as defined hereinabove:

In yet a further embodiment, R³ of Formula (I) may be selected from Substituent Group 3:

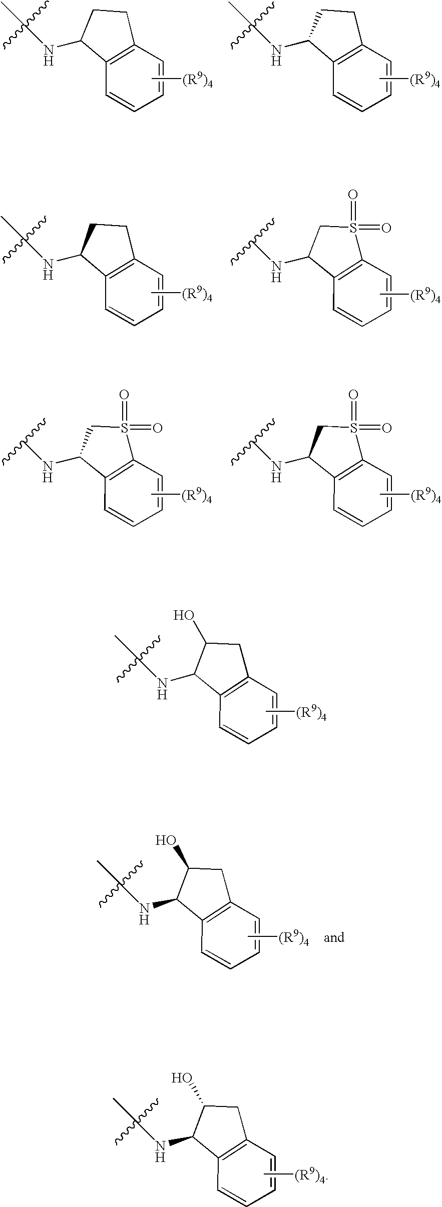

For example, in some embodiments, R³ of the structures of Group I(a) may be selected from Substituent Group 3 as defined hereinabove.

In another embodiment, R⁹ may be selected from Substituent Group 4:

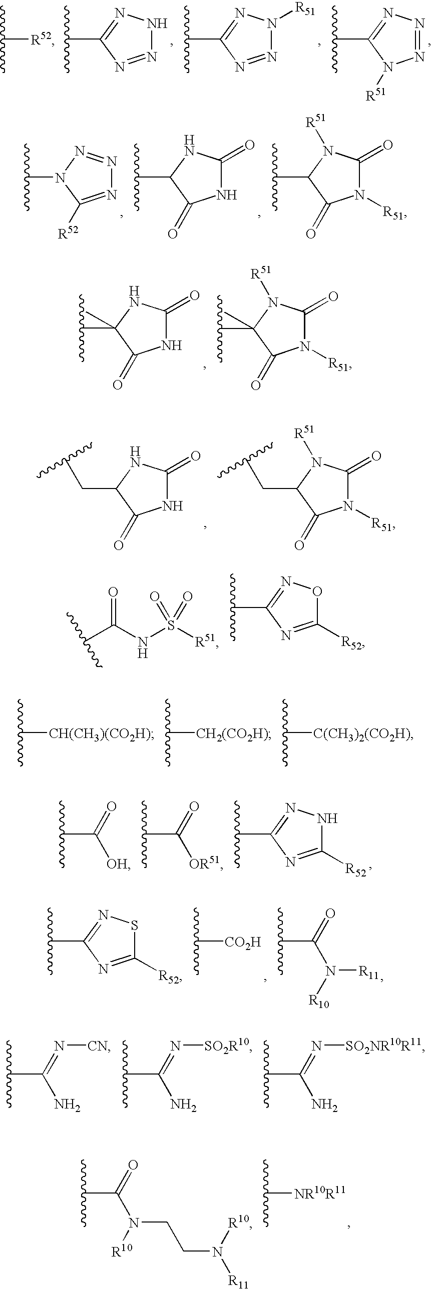

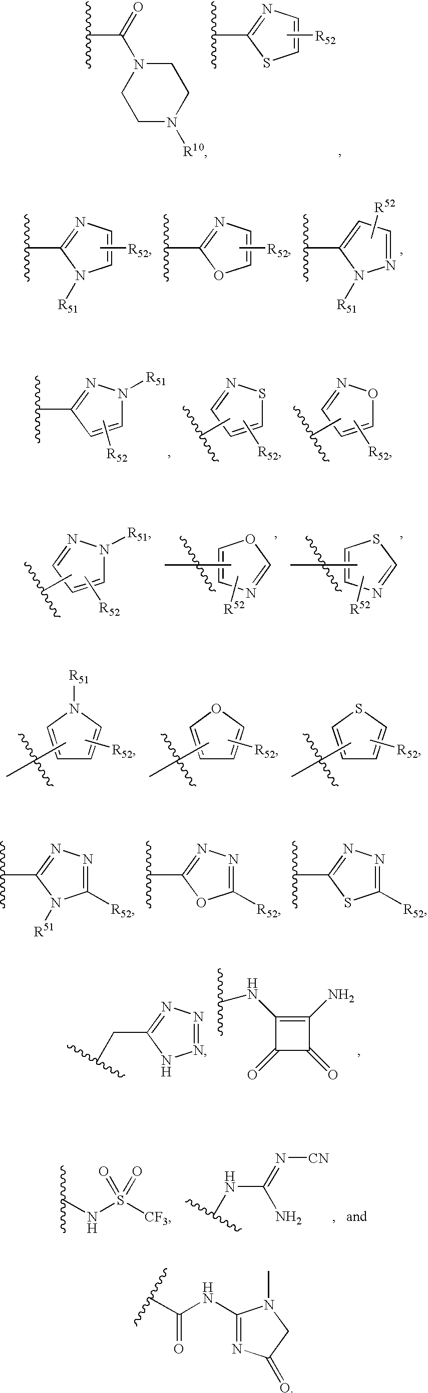

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times.

For example, in some embodiments, R⁹ of Substituent Group 3 may be selected from Substituent Group 4 as defined hereinabove.

In yet a further embodiment, R³ of the structures of Formula (I) may be Substituent Group 16:

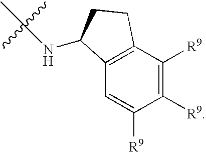

For example, in some embodiments, R³ of the structures of Group I(a) may be selected from Substituent Group 16 as defined hereinabove.

In some embodiments, R³ of the compounds of Formula (I) may be selected from: R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl.

In still a further embodiment, R³ of Formula (I) may be selected from Substituent Group 5: 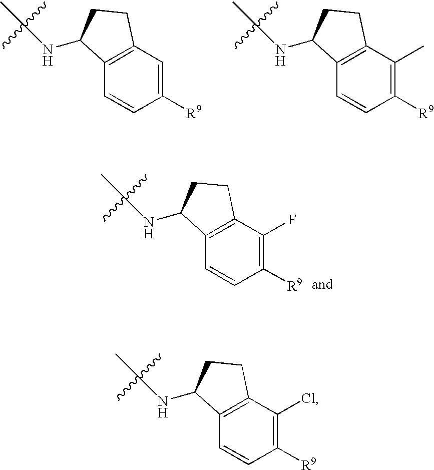
wherein:

R⁹ is selected from the group consisting of hydrogen, fluoro, halo, CN, alkyl, CO₂H,

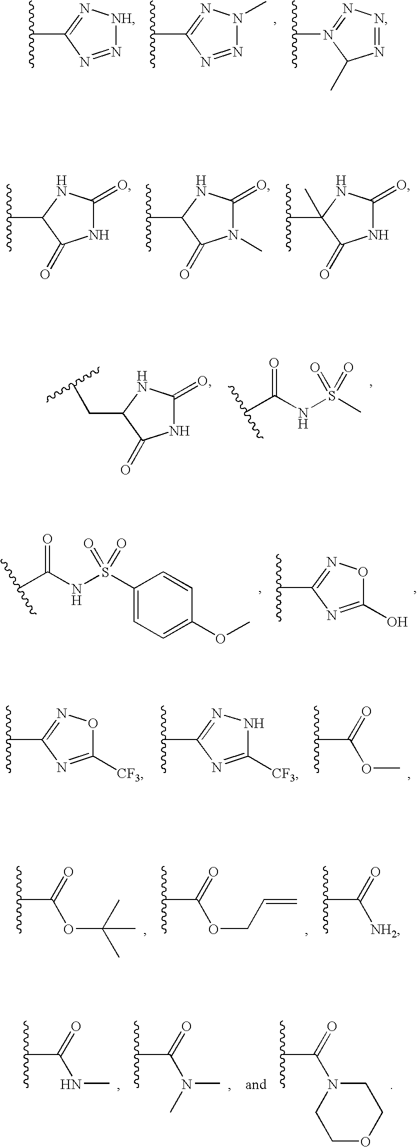

For example, in some embodiments, R³ of the structures of Group I(a) may be selected from Substituent Group 5 as defined hereinabove.

In another embodiment, R¹ of Formula (I) may be selected from Substituent Group 6:

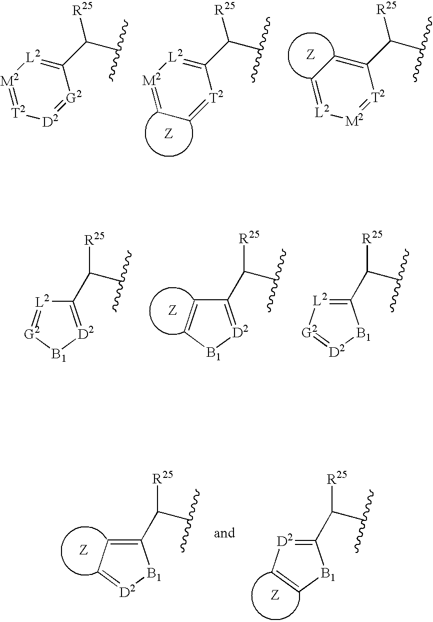

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

B₁ is selected from the group consisting of NR¹⁰, O, SO₂, SO and S;

D², G³, L², M² and T² are independently selected from the group consisting of CR¹⁸ and N; and

Z is a 5- to 8-membered ring selected from the group consisting of cycloalkyl, heterocycloalkyl, aryl and heteroaryl, wherein cycloalkyl, heterocycloalkyl, aryl and heteroaryl are optionally substituted one or more times.

For example, in another embodiment, R¹ of the structures of Group I(a) may be selected from Substituent Group 6 as defined hereinabove.

In yet another embodiment, R¹ of the structures of Group I(a) may be selected from Substituent Group 7:

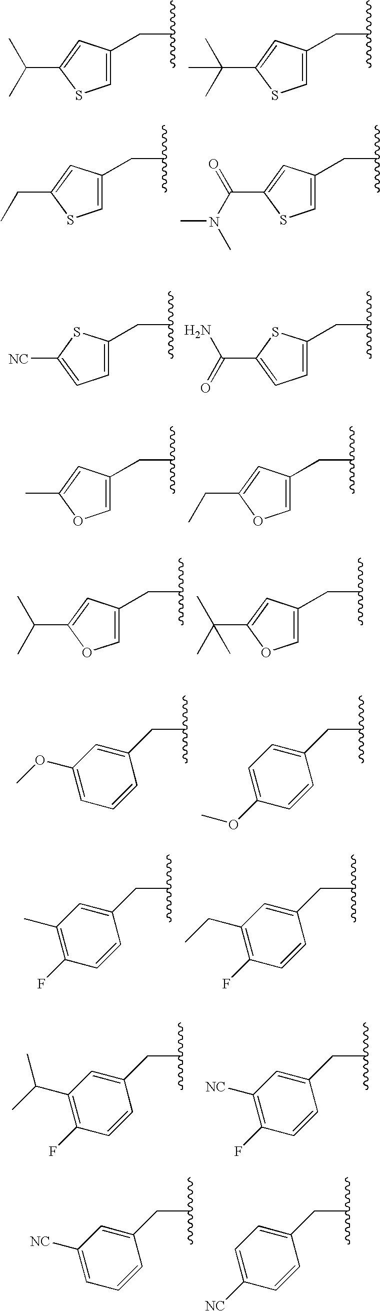

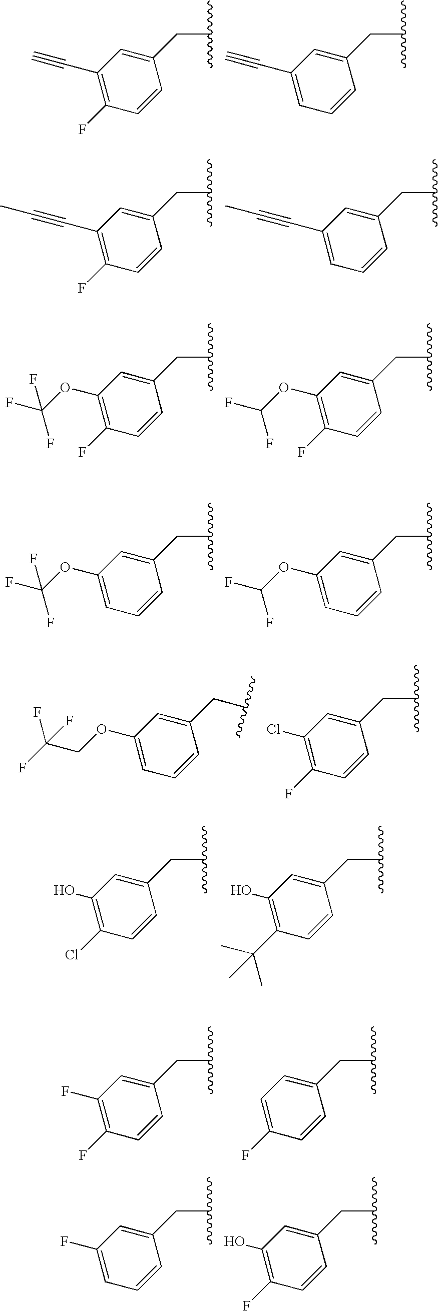

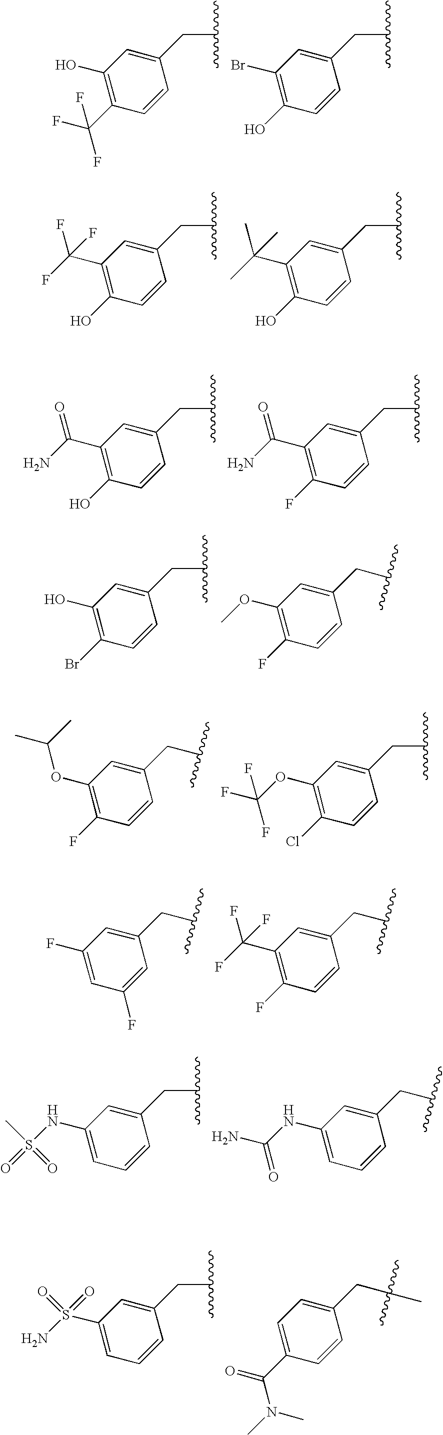

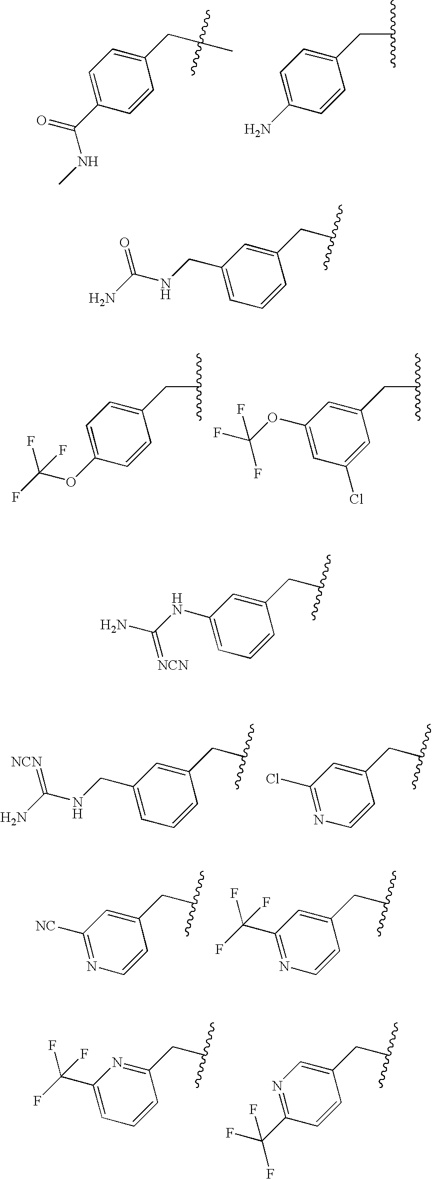

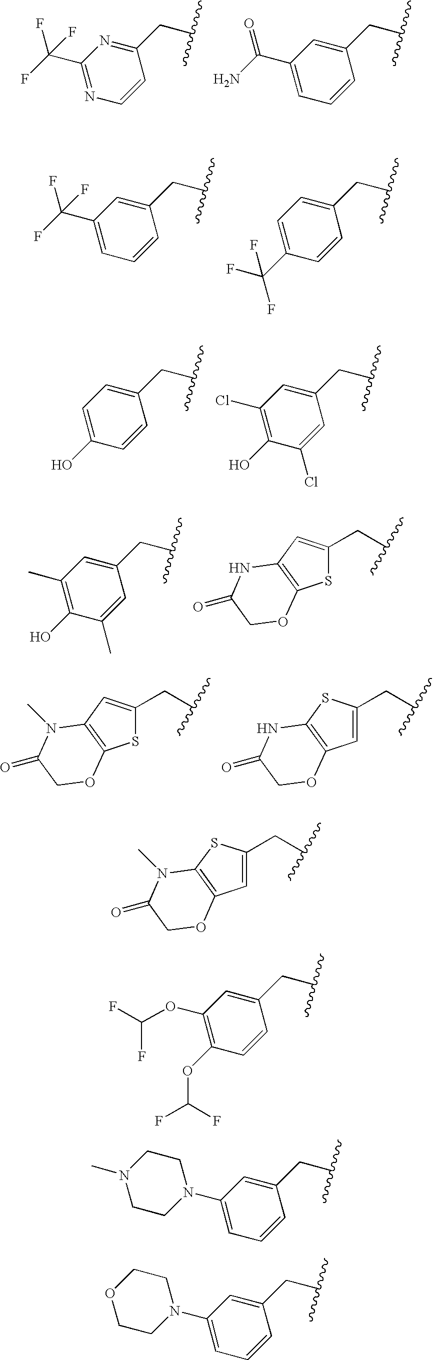

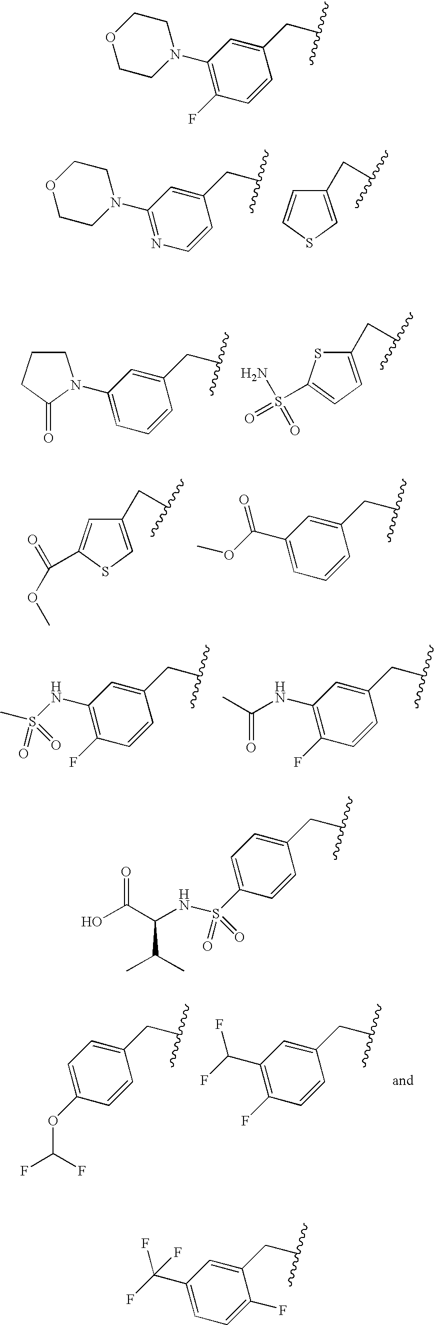

For example, in some embodiments, R¹ of the structures of Group I(a) may be selected from Substituent Group 7 as defined hereinabove.

In still another embodiment, R¹ of Formula (I) may be selected from Substituent Group 8:

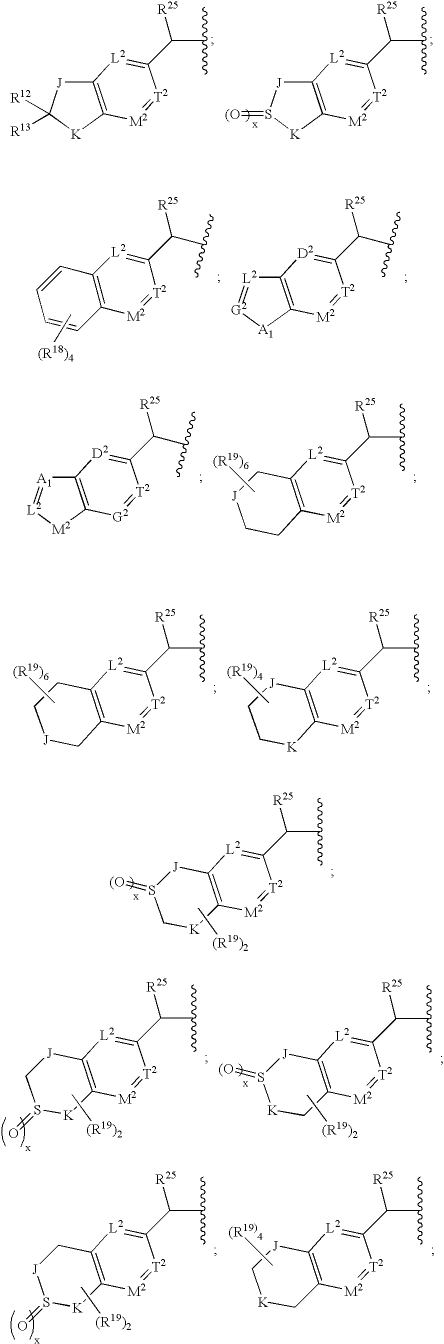

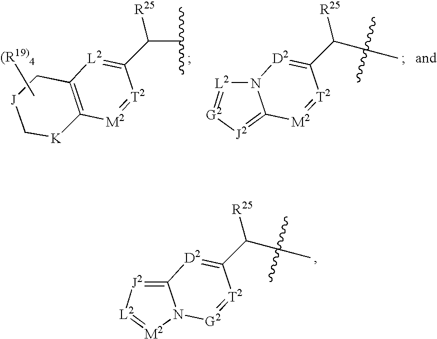

wherein:

R¹² and R¹³ are independently selected from the group consisting of hydrogen, alkyl and halo, wherein alkyl is optionally substituted one or more times, or optionally R¹² and R¹³ together form ═O, ═S or ═NR¹⁰;

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

J and K are independently selected from the group consisting of CR¹⁰R¹⁸, NR¹⁰, O and S(O)ₓ;

A₁ is selected from the group consisting of NR¹⁰, O and S; and

D², G², J², L², M² and T² are independently selected from the group consisting of CR¹⁸ and N.

For example, some embodiments, R¹ of the structures of Group I(a) may be selected from Substituent Group 8 as defined hereinabove.

In a further embodiment, R¹ of Formula (I) may be selected from Substituent Group 9:

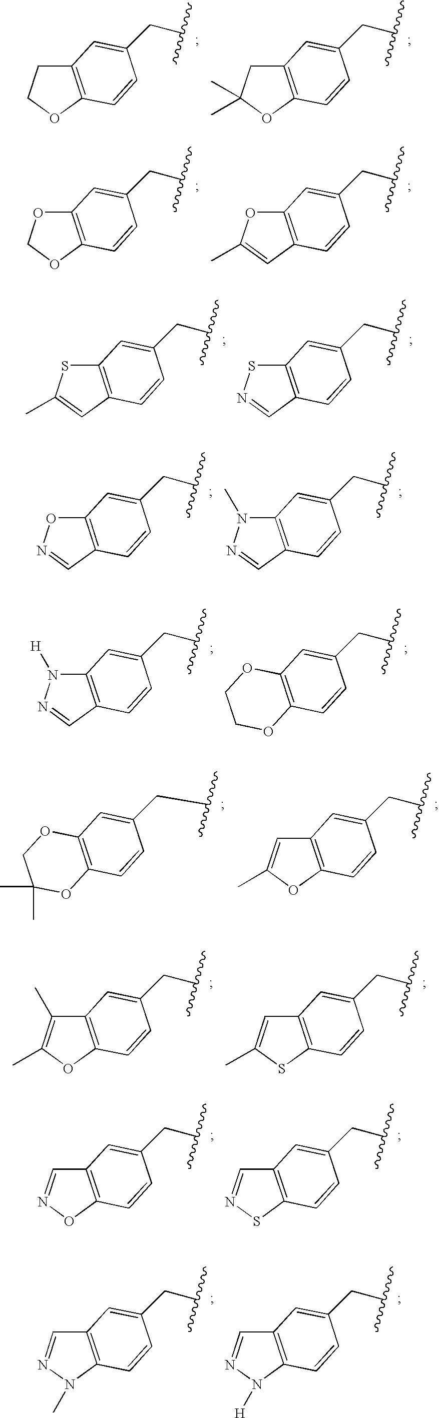

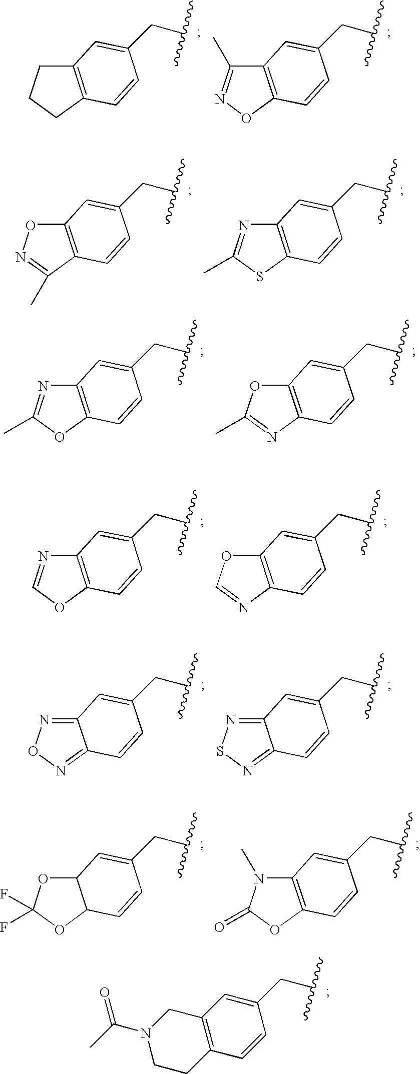

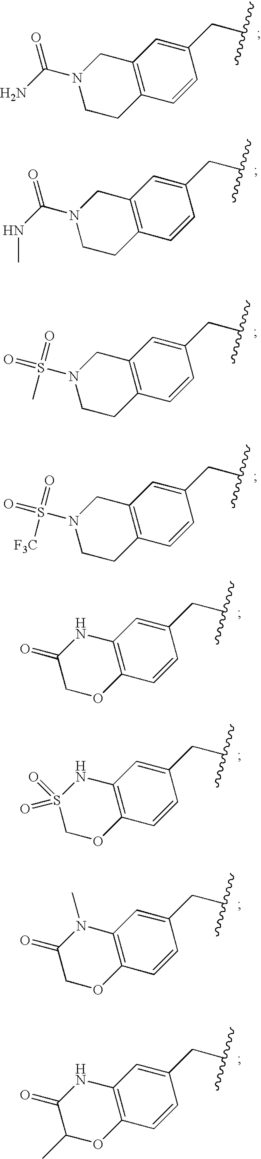

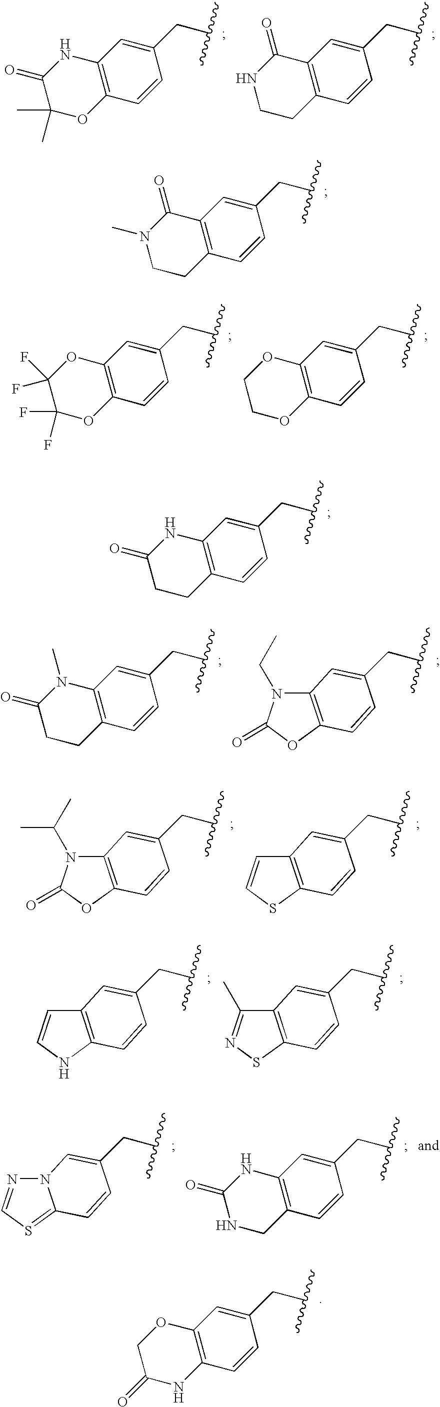

For example, in some embodiments, R¹ of the structures of Group I(a) may be selected from Substituent Group 9 as defined hereinabove.

In yet a further embodiment, R¹ of Formula (I) may be selected from Substituent Group 10:

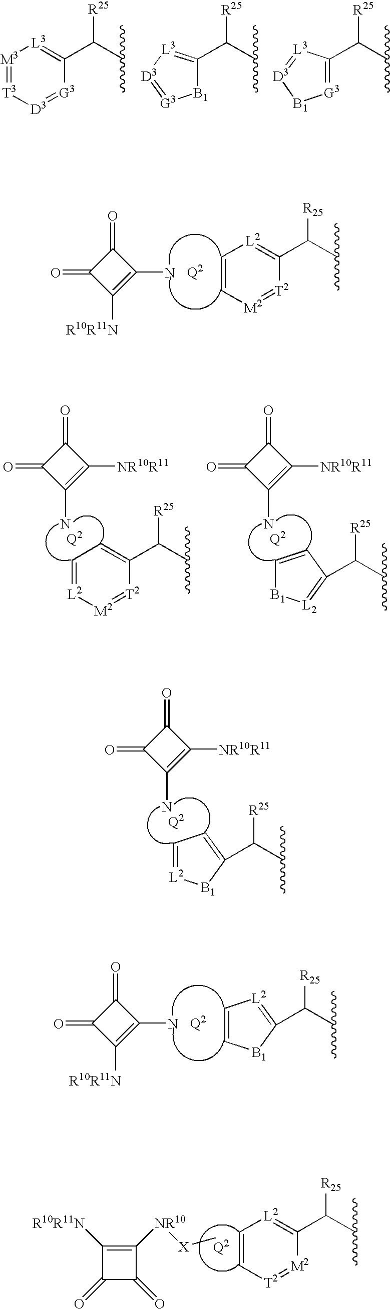

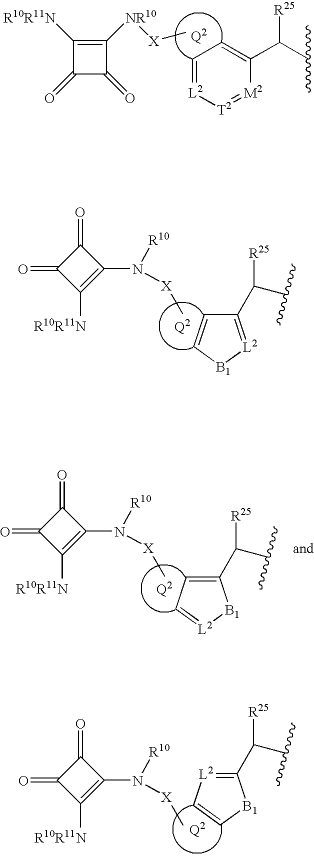

wherein:

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;

E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

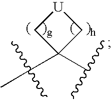

L², M², and T² are independently selected from the group consisting of CR¹⁸ and N;

D³, G³, L³, M³, and T³ are independently selected from N, CR¹⁸, (i) and (ii)

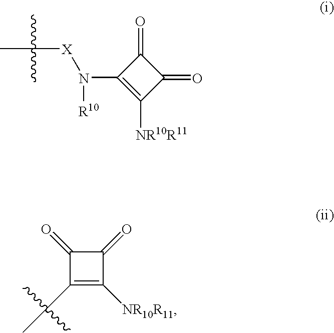

with the proviso that one of L³, M³, T³, D³, and G³ is (i) or (ii);

B₁ is selected from the group consisting of NR¹⁰, O, SO₂, SO and S;

Q² is a 5- to 8-membered ring selected from the group consisting of cycloalkyl, heterocycloalkyl, aryl, and heteroaryl, which is optionally substituted one or more times with R¹⁹;

U is selected from the group consisting of C(R⁵R¹⁰), NR⁵, O, S, S═O and S(═O)₂;

W¹ is selected from the group consisting of O, NR⁵, S, S═O, S(═O)₂, N(R¹⁰)(C═O), N(R¹⁰)S(═O)₂ and S(═O)₂N(R¹⁰);

X is selected from the group consisting of a bond and (CR¹⁰R¹¹)_{w}E(CR¹⁰R¹¹)_{w};

g and h are independently selected from 0-2; and

w is independently selected from 0-4.

The compound wherein R¹ is selected from the group consisting of:

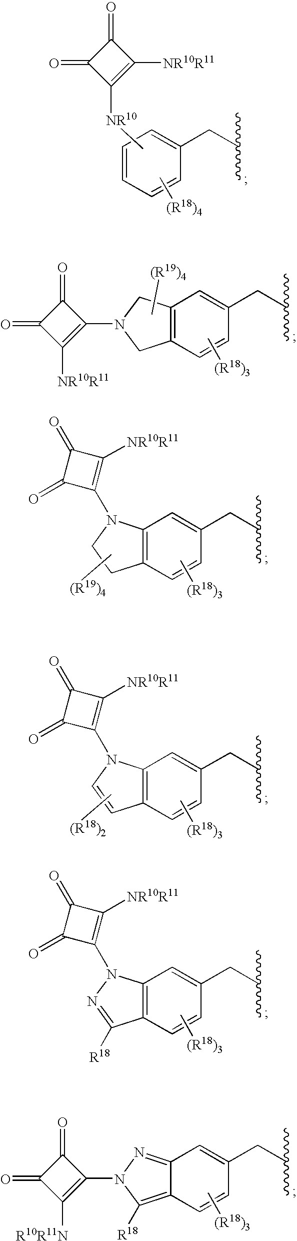

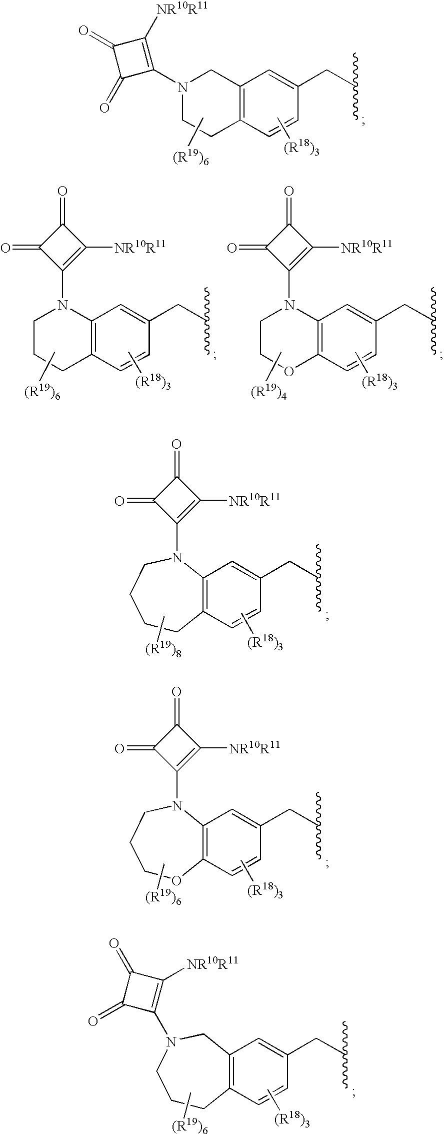

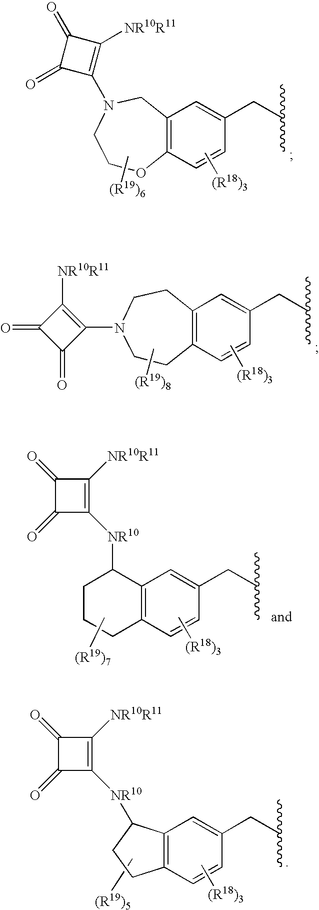

For example, in some embodiments, R¹ of the structures of Group I(a) may be selected from Substituent Group 10 as defined herinabove.

In still a further embodiment, R¹ of Formula (I) may be selected from Substituent Group 11:

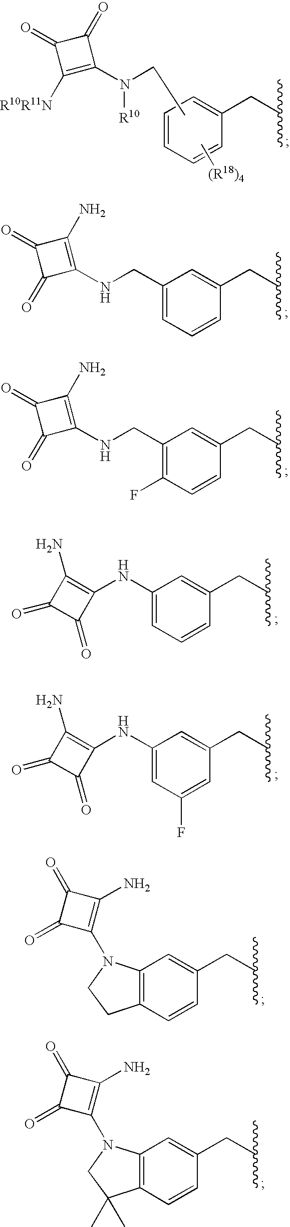

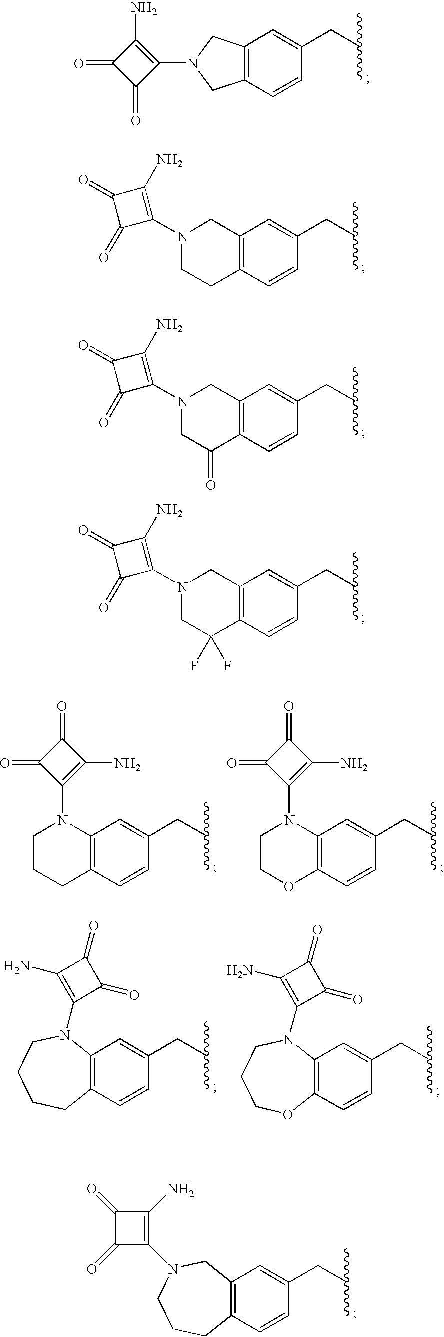

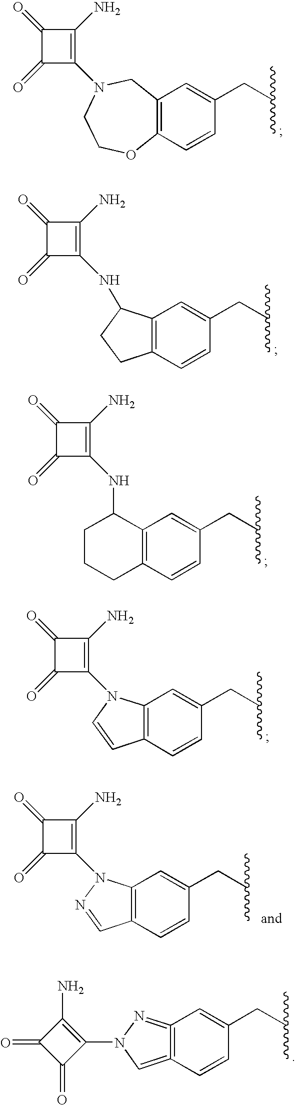

For example, in some embodiments, R¹ of the structures of Group I(a) may be selected from Substituent Group 11 as defined hereinabove.

In some embodiments, the compounds of Formula (I) may be defined wherein:

X¹ is a bond, and

R³ is selected from the group consisting of

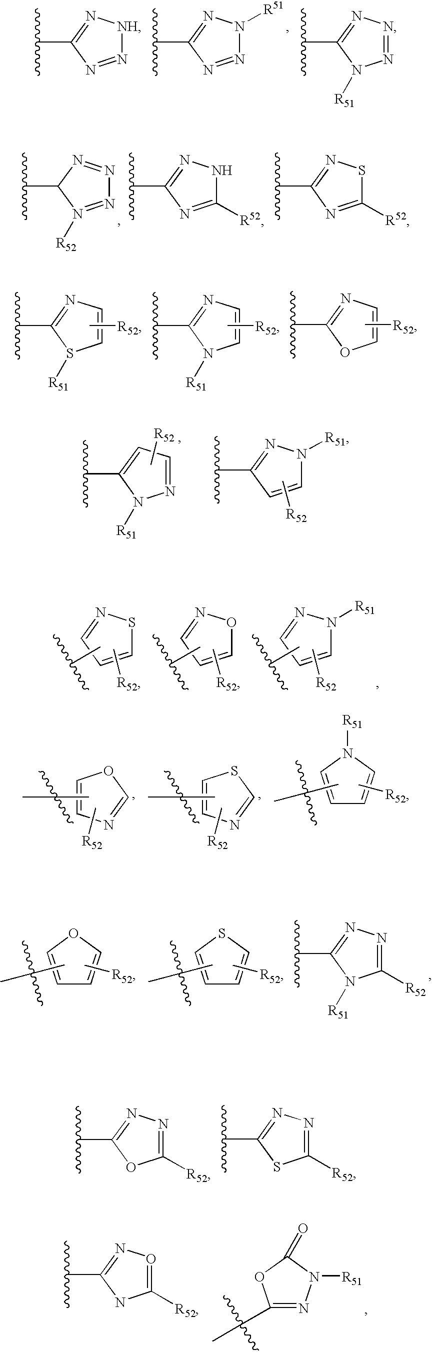

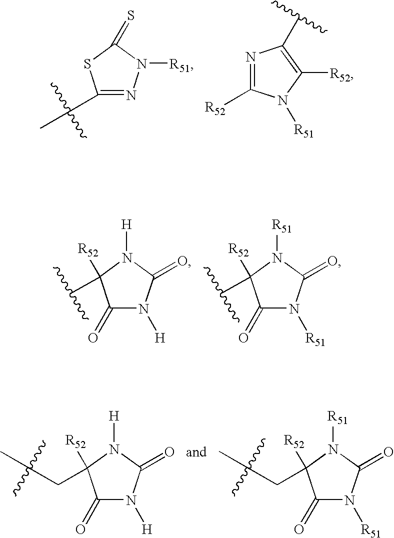

In some embodiments, the compounds of Formula (I) may be selected from:

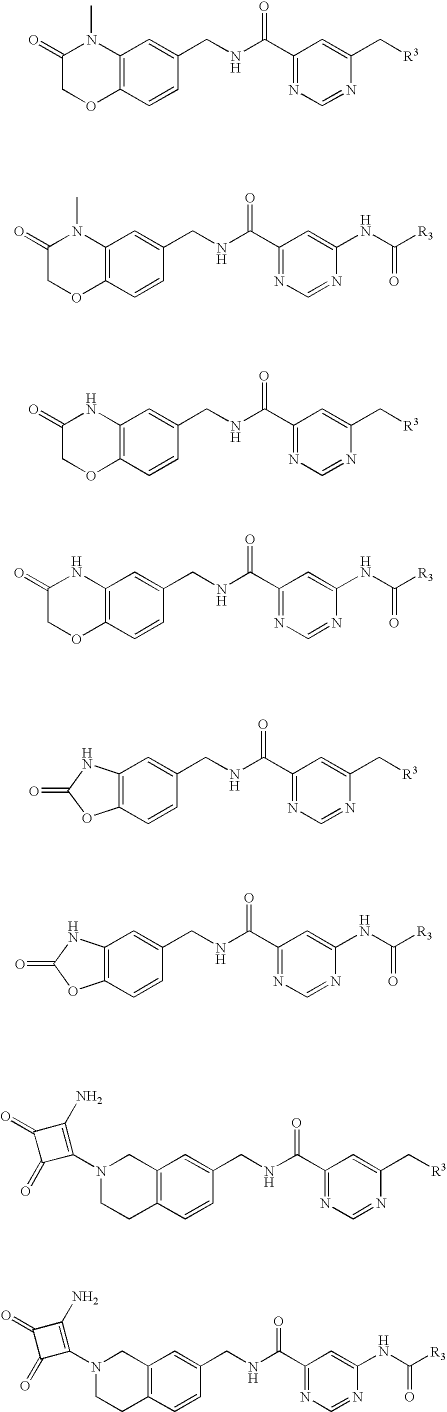

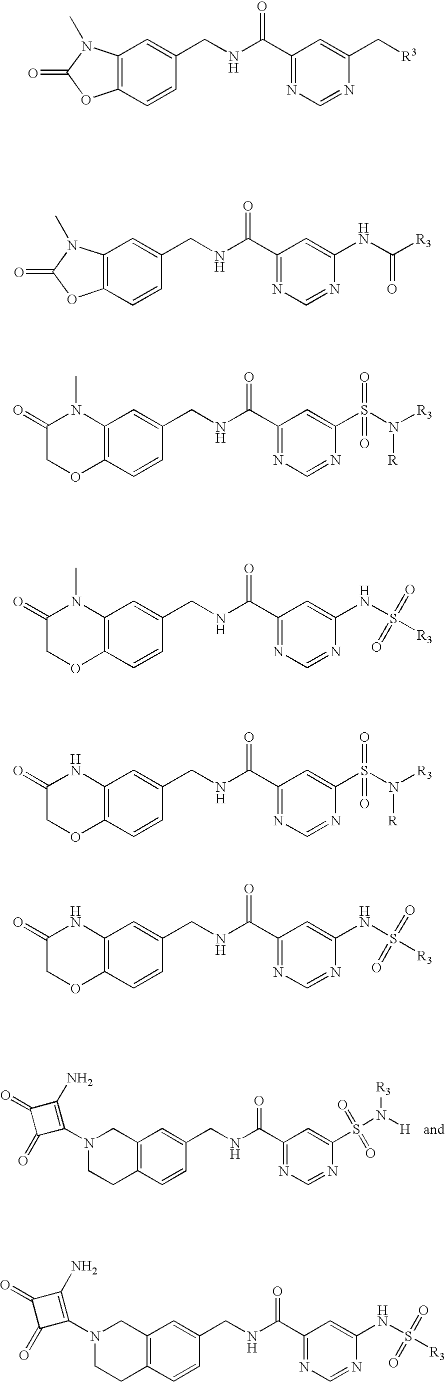

In yet another embodiment, the amide containing aromatic metalloprotease compounds may be represented by the Formula (II):

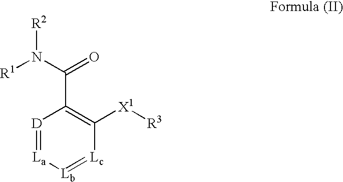

and N-oxides, pharmaceutically acceptable salts, prodrugs, formulation, polymorphs, racemic mixtures and stereoisomers thereof,

wherein:

R¹ in each occurrence is independently selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

wherein optionally two hydrogen atoms on the same atom of the R¹ group are replaced with ═O, ═S or ═NR¹⁰;

R² in each occurrence is independently selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

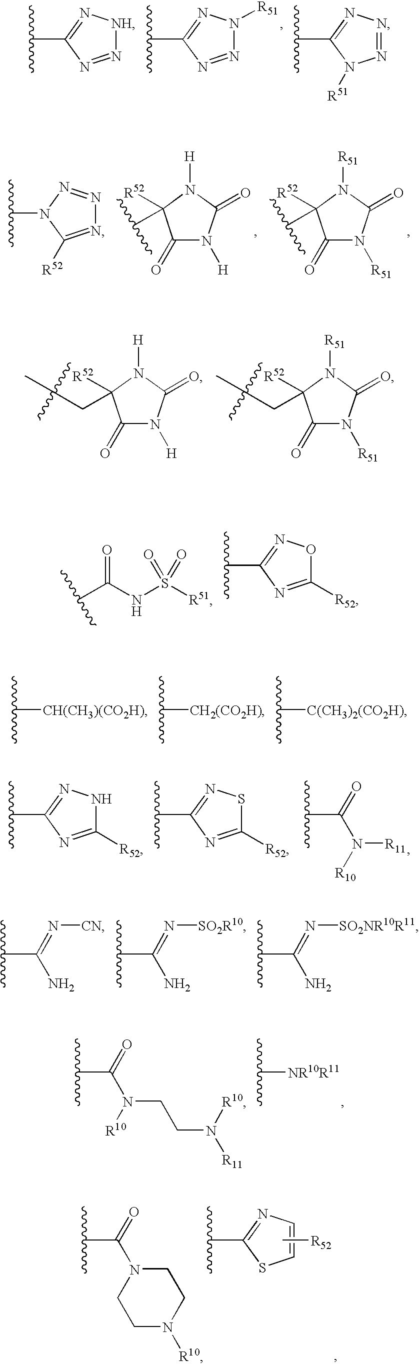

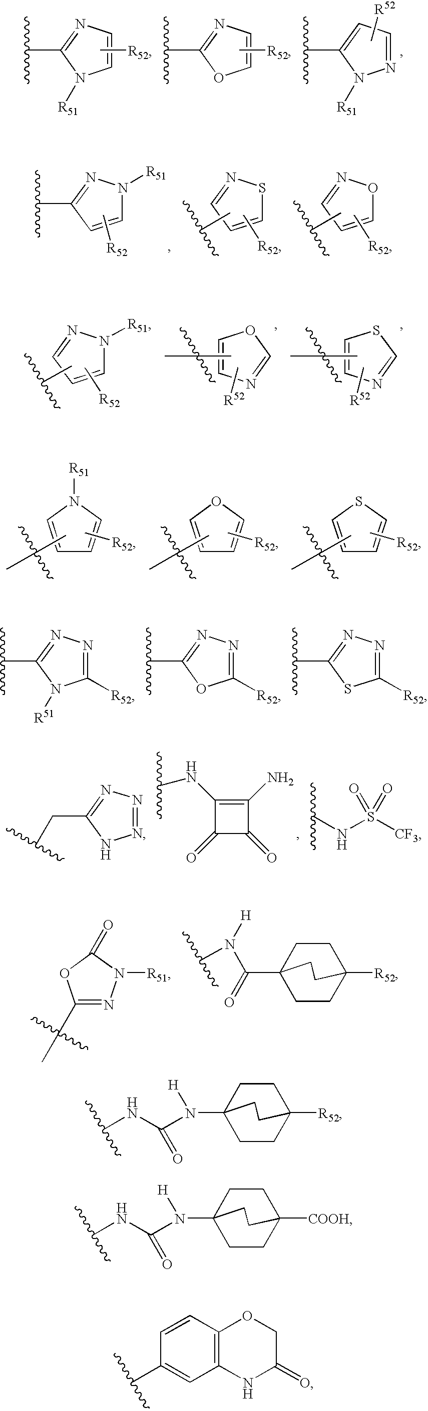

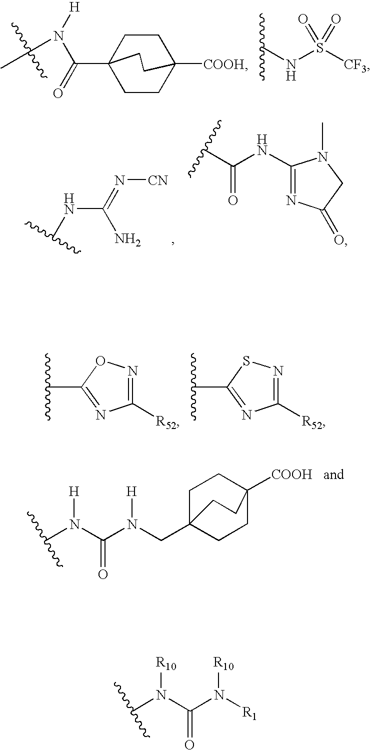

R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from the group consisting of hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from the group consisting of hydrogen, alkyl, arylalkyl, cycloalkylalkyl, heteroarylalkyl, heterocycloalkyl and halo, wherein alkyl, arylalkyl, cycloalkylalkyl, heteroarylalkyl and heterocycloalkyl are optionally substituted one or more times.

R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 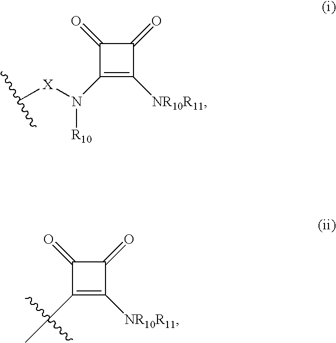
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicylic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from the group consisting of alkyl and (C₀-C₆)-alkyl-aryl, wherein alkyl and aryl are optionally substituted;

R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

D is a member selected from the group consisting of CR²² and N;

Lₐ, L_{b}, and L_{c} are independently selected from CR⁹ and N with the proviso that Lₐ, L_{b}, and L_{c} cannot all simultaneously be N;

X¹ is selected from a bond, NR¹⁰, CH₂, CHR²⁰, CR²⁰R²¹, SO₂, SO, S, PO₂, O, C═S, C═O, C═NR¹, C═N—SO₂R¹⁰, C═N—CN, C═N—CONR¹⁰R¹¹, C═N—COR¹⁰, C═N—OR¹⁰, NR¹⁰C═O, NR¹⁰SO₂ and SO₂NR¹⁰;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In another embodiment, compounds of Formula (II) may be selected from the Group I(a):

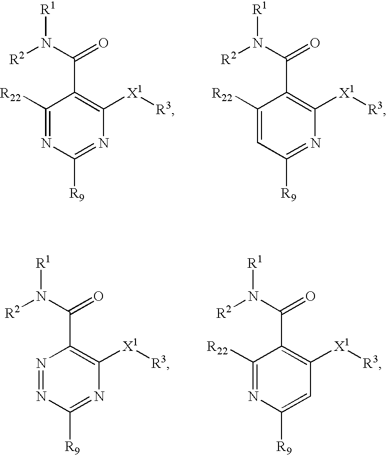

In still another embodiment, compounds of Formula (II) may be selected from:

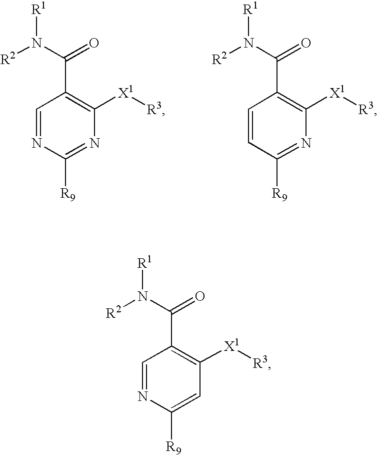

In yet another embodiment, compounds of Formula (II) may be selected from:

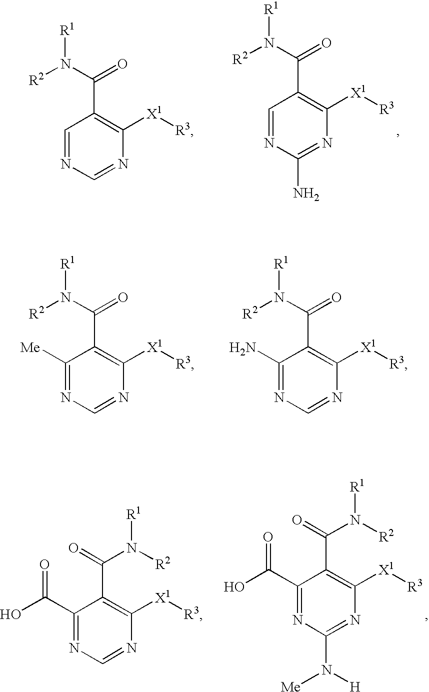

In another embodiment, compounds of Formula (II) may be selected from:

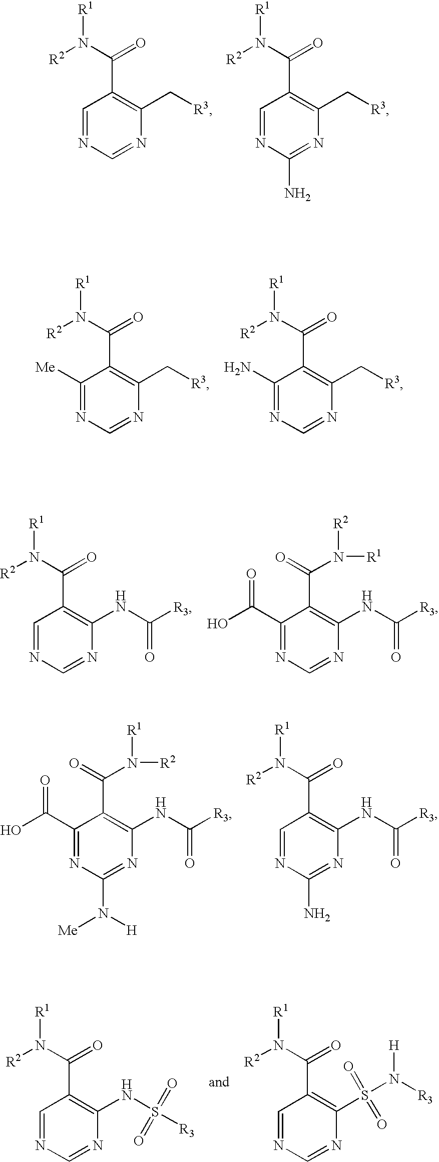

In some embodiments R³ of the compounds of Formula (II) may be selected from Substituent Group 1:

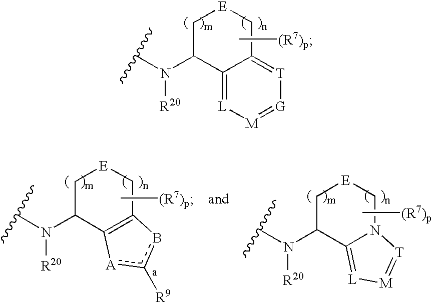

wherein:

R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁷ is independently selected from the group consisting of hydrogen, alkyl, cycloalkyl, halo, R⁴ and NR¹⁰R¹¹ or optionally two R⁷ groups together at the same carbon atom form ═O, ═S or ═NR¹⁰;

A and B are independently selected from the group consisting of CR⁹, CR⁹R¹⁰, NR¹⁰, N, O and S;

E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

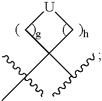

G, L, M and T are independently selected from the group consisting of CR⁹ and N;

U is selected from the group consisting of C(R⁵R¹⁰), NR⁵, O, S, S═O and S(═O)₂;

W¹ is selected from the group consisting of O, NR⁵, S, S═O, S(═O)₂, N(R¹⁰)(C═O), N(R¹⁰)S(═O)₂ and S(═O)₂N(R¹⁰);

g and h are independently selected from 0-2;

m and n are independently selected from 0-3, provided that:
(1) when E is present, m and n are not both 3;
(2) when E is —CH₂—W¹—, m and n are not 3; and
(3) when E is a bond, m and n are not 0; and

p is selected from 0-6;

wherein the dotted line represents a double bond between one of: carbon “a” and A, or carbon “a” and B.

In yet a further embodiment, R³ of Formula (II) may be selected from Substituent Group 3:

hydrogen, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, POR²¹SR¹⁰, SR²¹, CH₂R²⁰, CHR²⁰R²¹, OR¹⁰, OR²¹, NR¹⁰NR⁹, R⁵²,

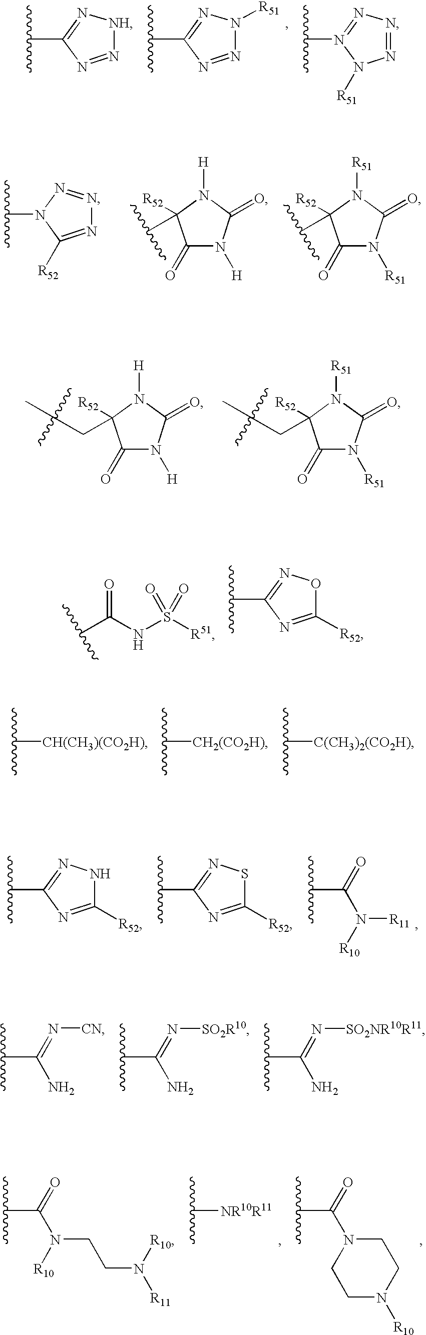

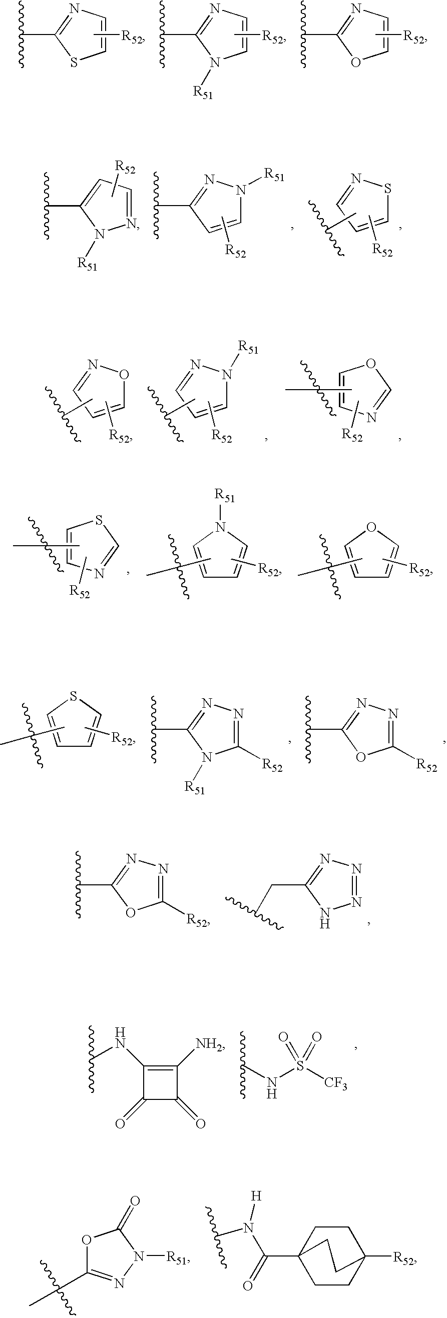

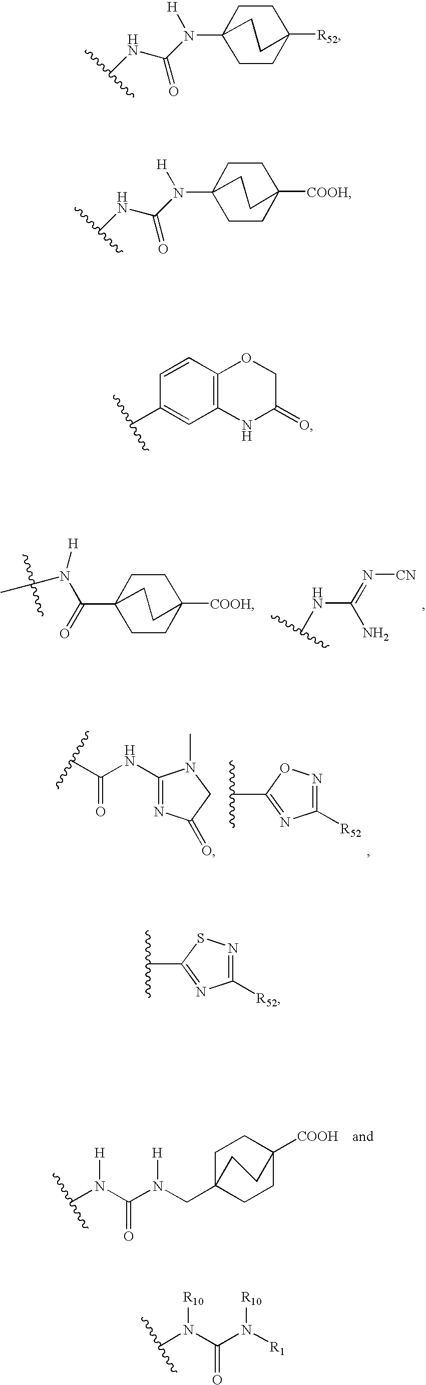

In some embodiments, R³ of the compounds of Formula (II) may be selected from: R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰ (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NROR¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl.

In some embodiments, R³ of Formula (II) may be selected from Substituent Group I(2):

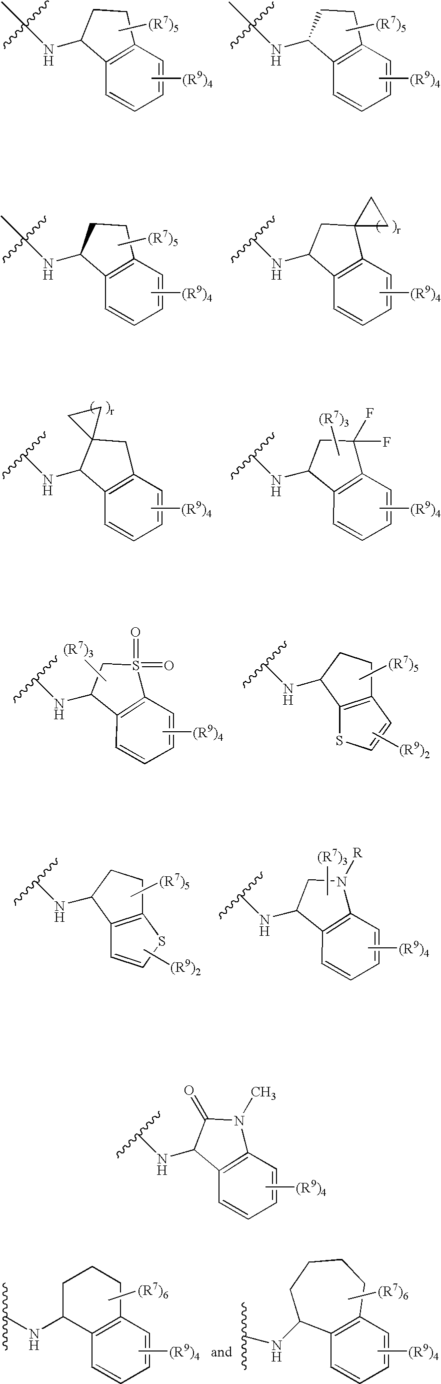

wherein:

R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and

r is selected from 1-4.

For example, in some embodiments, R³ of the compounds of Group I(a) may be selected from Substituent Group 2, as defined hereinabove:

In yet a further embodiment, R³ of Formula (II) may be selected from Substituent Group 3:

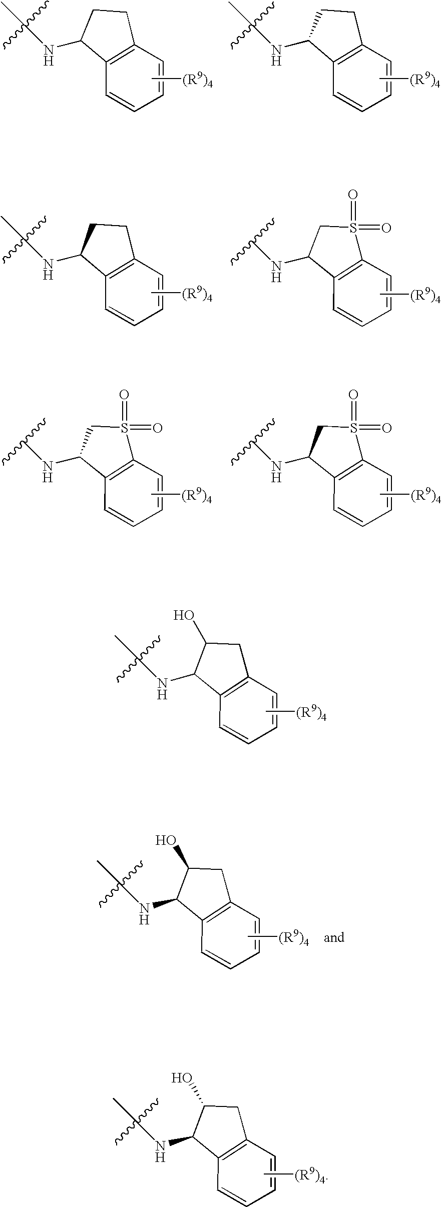

For example, in some embodiments, R³ of the structures of Group I(a) may be selected from Substituent Group 3 as defined hereinabove.

In another embodiment, R⁹ may be selected from Substituent Group 4:

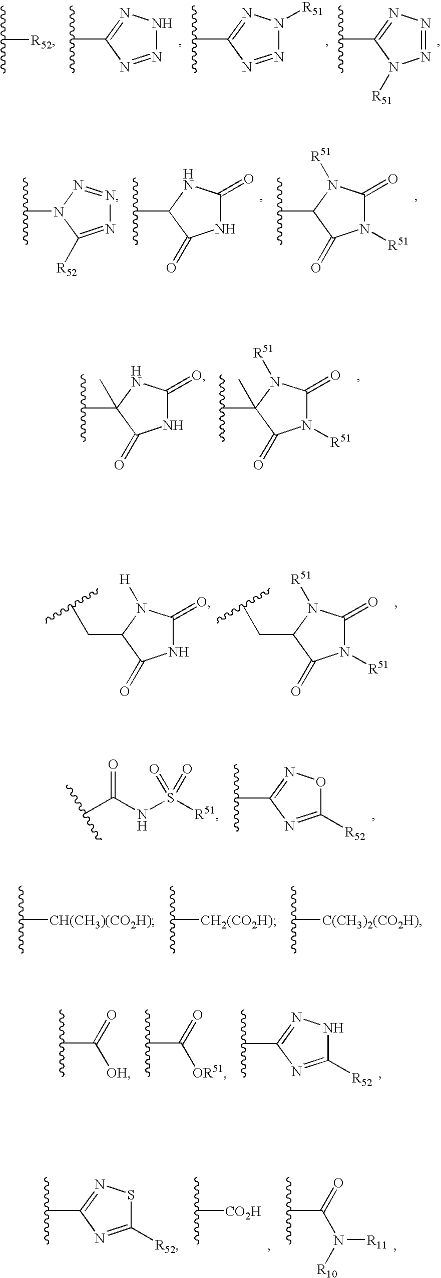

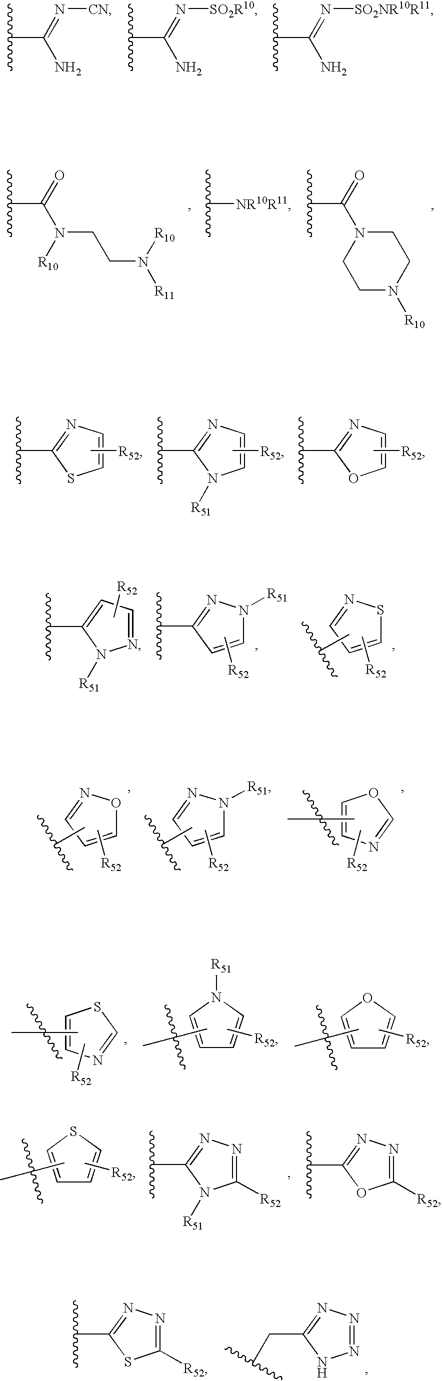

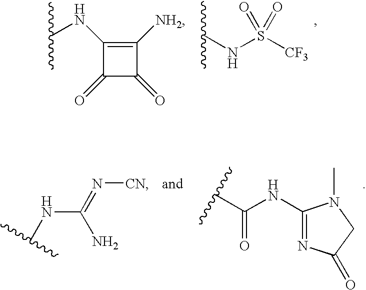

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times.

For example, in some embodiments, R⁹ of Substituent Group 3 may be selected from Substituent Group 4 as defined hereinabove.

In yet a further embodiment, R³ of the structures of Formula (II) may be Substituent Group 16:

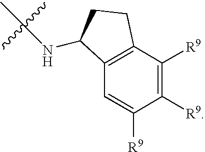

For example, in some embodiments, R³ of the structures of Group I(a) may be selected from Substituent Group 16 as defined hereinabove.

In still a further embodiment, R³ of Formula (II) may be selected from Substituent Group 5: 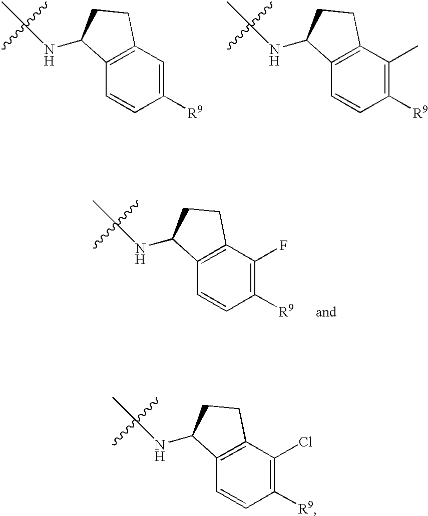
wherein:

R⁹ is selected from the group consisting of hydrogen, fluoro, halo, CN, alkyl, CO₂H,

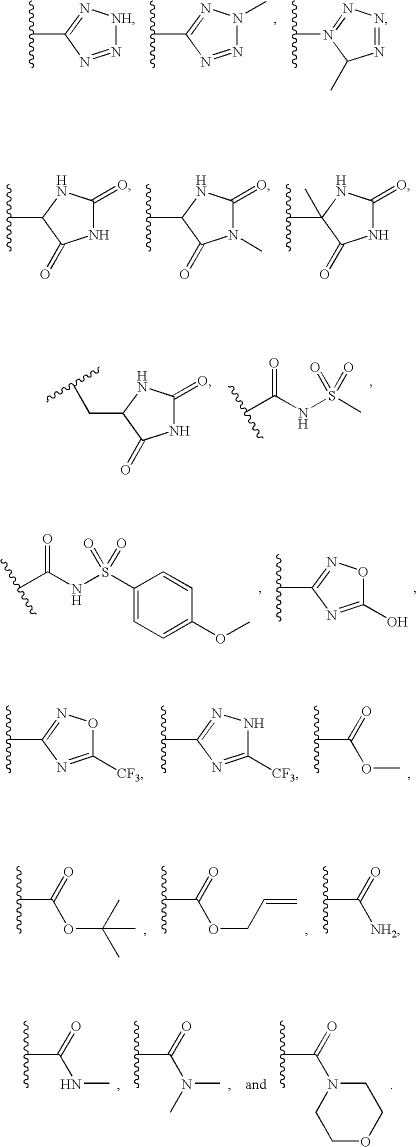

For example, in some embodiments, R³ of the structures of Group I(a) may be selected from Substituent Group 5 as defined hereinabove.

In another embodiment, R¹ of Formula (II) may be selected from Substituent Group 6:

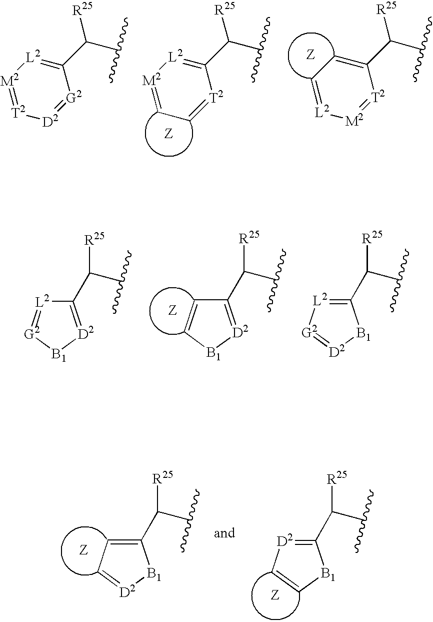

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

B₁ is selected from the group consisting of NR¹⁰, O and S;

D², G², L², M² and T² are independently selected from the group consisting of CR¹⁸ and N; and

Z is a 5- to 8-membered ring selected from the group consisting of cycloalkyl, heterocycloalkyl, aryl and heteroaryl, wherein cycloalkyl, heterocycloalkyl, aryl and heteroaryl are optionally substituted one or more times.

For example, in another embodiment, R¹ of the structures of Group I(a) may be selected from Substituent Group 6 as defined hereinabove.

In yet another embodiment, R¹ of the structures of Group I(a) may be selected from Substituent Group 7:

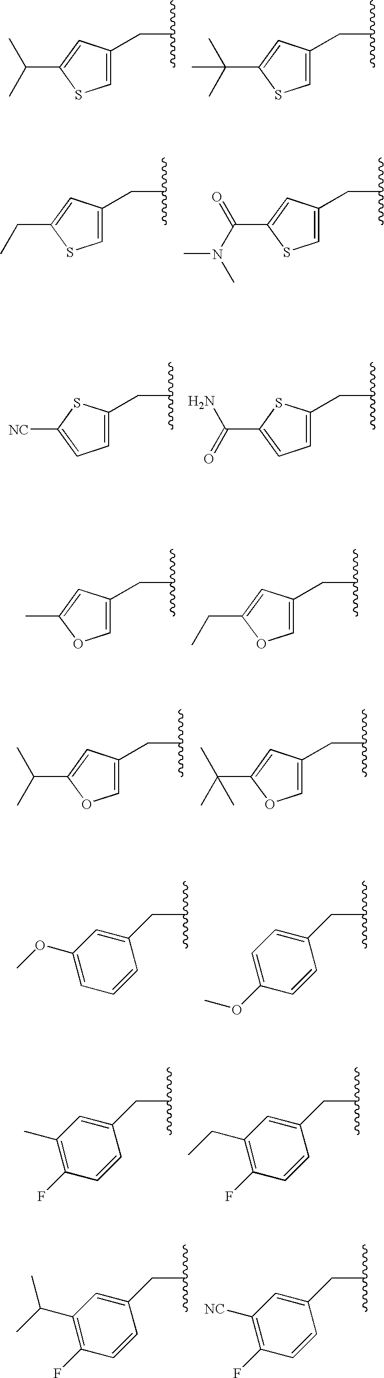

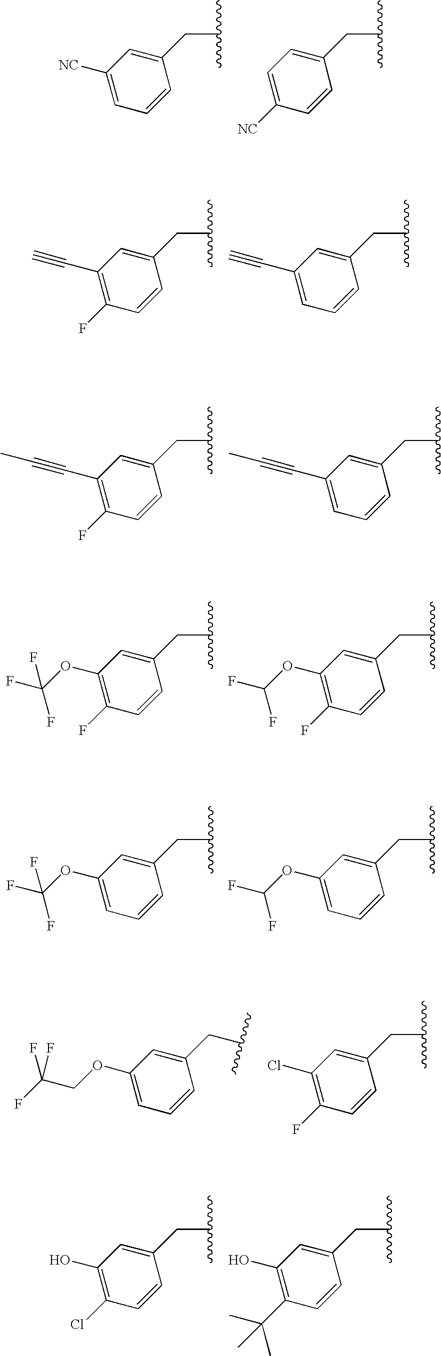

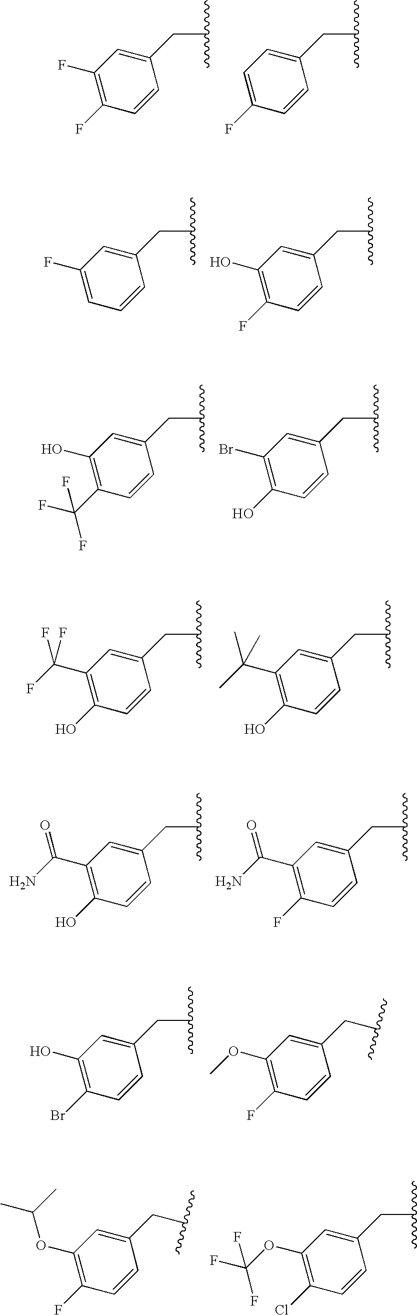

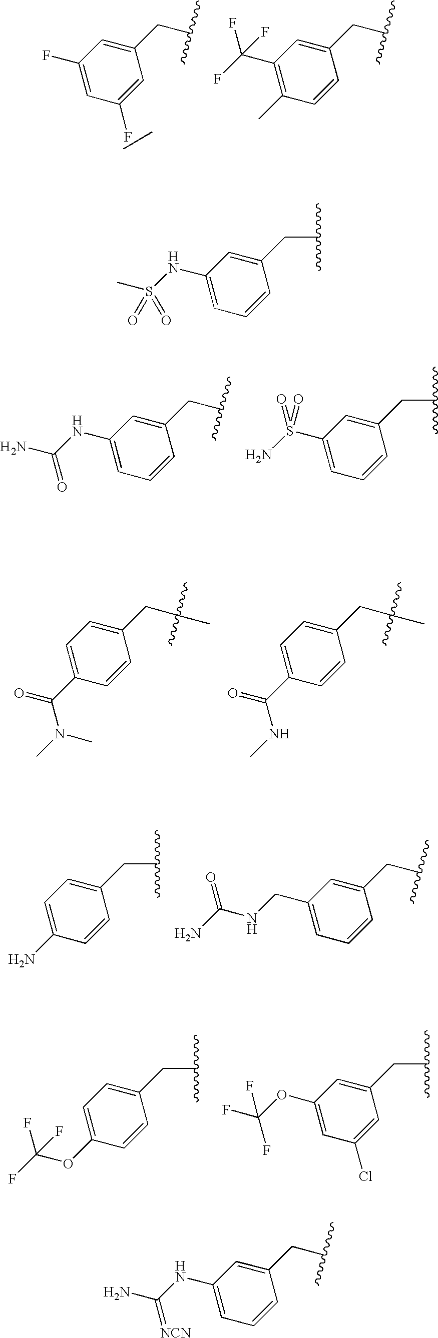

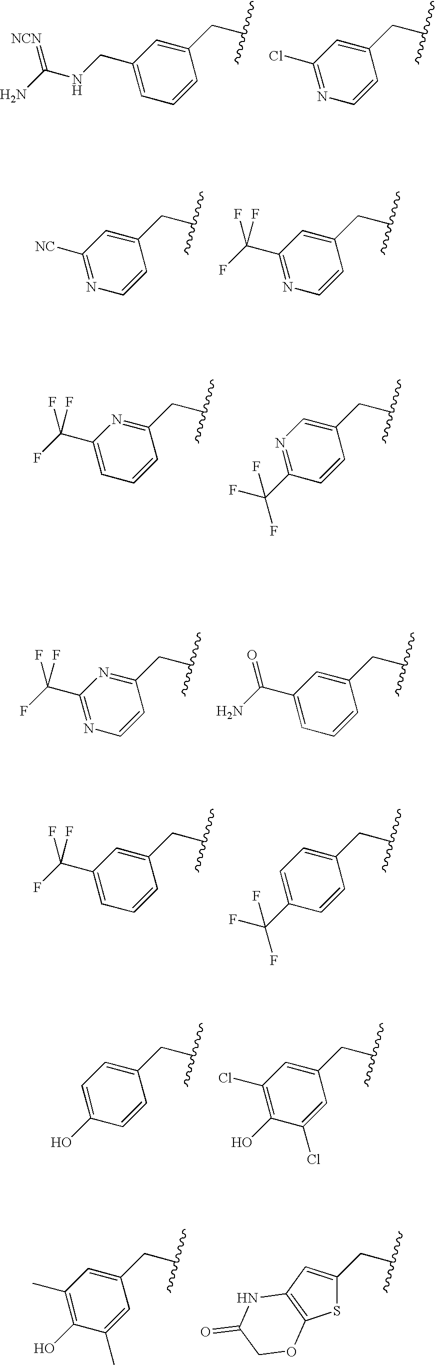

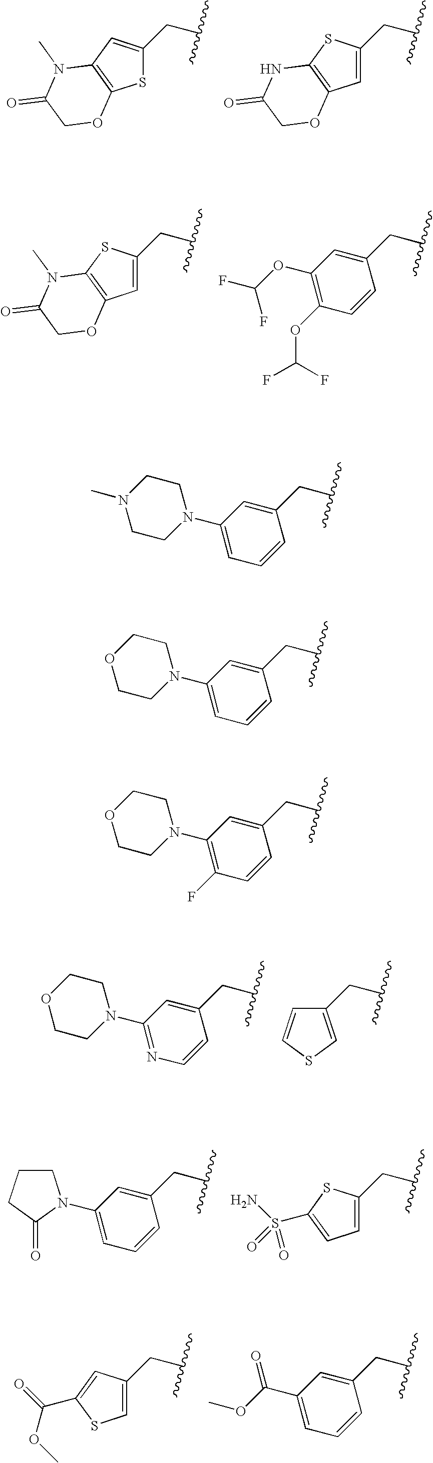

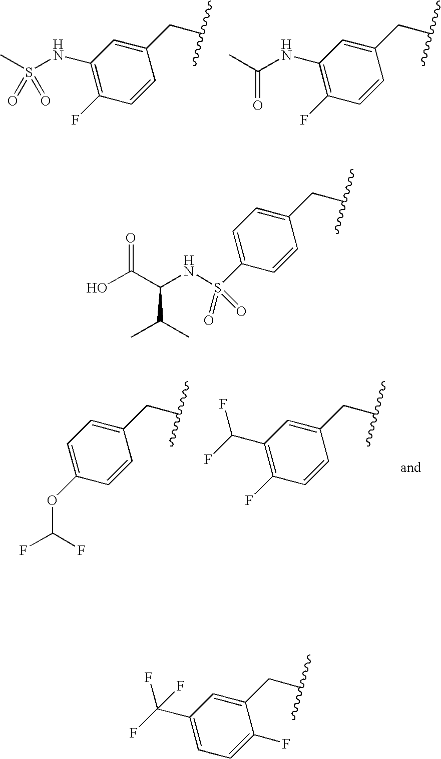

For example, in some embodiments, R¹ of the structures of Group I(a) may be selected from Substituent Group 7 as defined hereinabove.

In still another embodiment, R¹ of Formula (II) may be selected from Substituent Group 8:

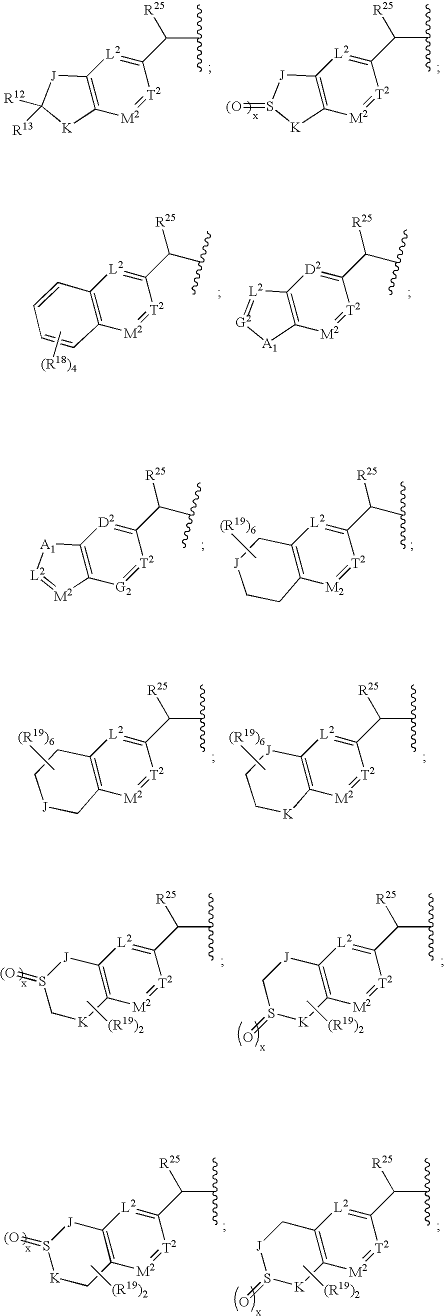

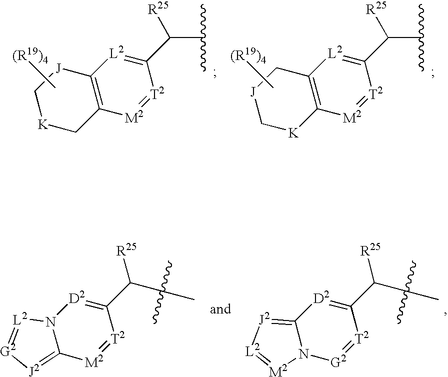

wherein:

R¹² and R¹³ are independently selected from the group consisting of hydrogen, alkyl and halo, wherein alkyl is optionally substituted one or more times, or optionally R¹² and R¹³ together form ═O, ═S or ═NR¹⁰;

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

J and K are independently selected from the group consisting of CR¹⁰R¹⁸, NR¹⁰, O and S(O)ₓ;

A₁ is selected from the group consisting of NR¹⁰, O, SO₂, SO and S; and

D2, G², J², L², M² and T² are independently selected from the group consisting of CR¹⁸ and N.

For example, some embodiments, R¹ of the structures of Group I(a) may be selected from Substituent Group 8 as defined hereinabove.

In a further embodiment, R¹ of Formula (II) may be selected from Substituent Group 9:

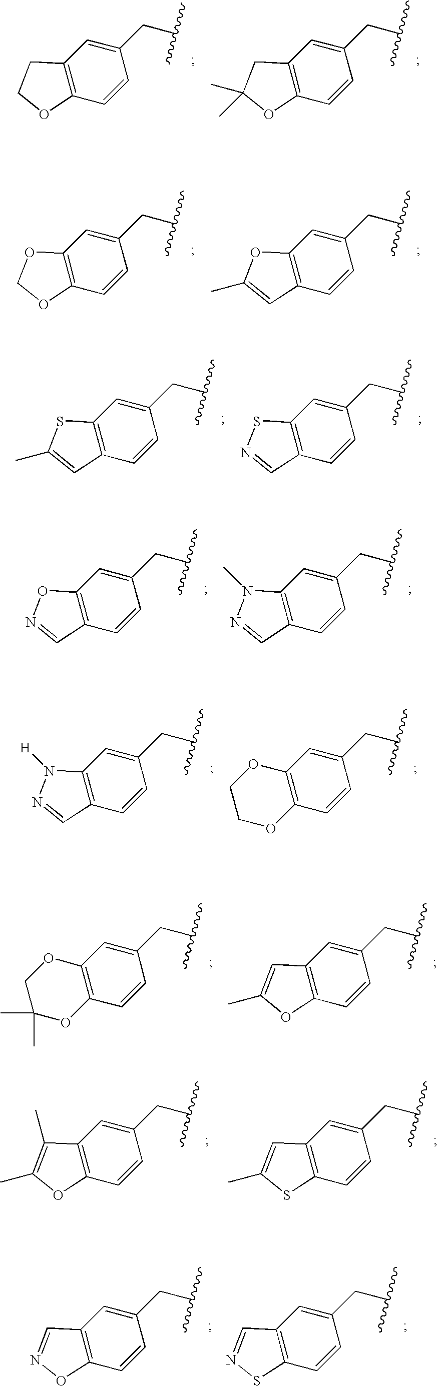

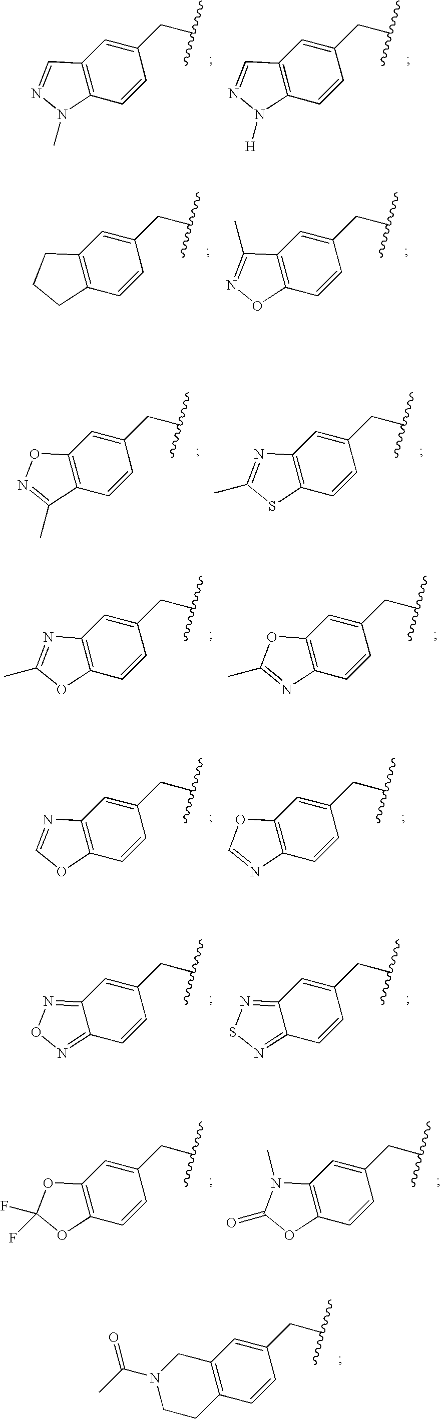

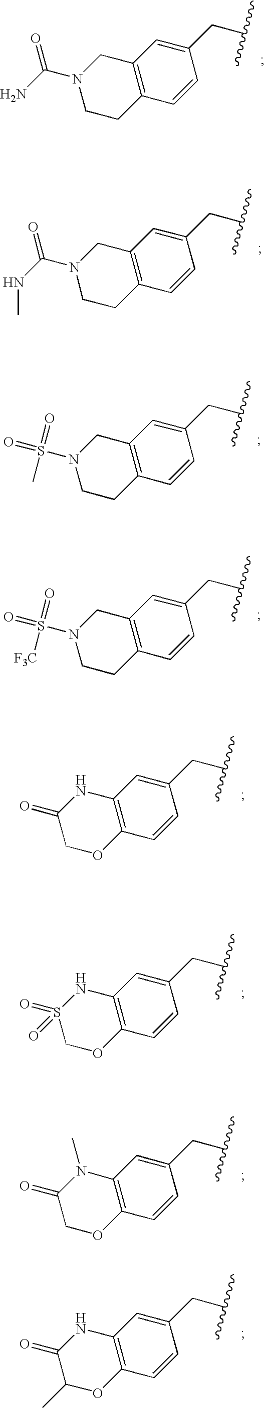

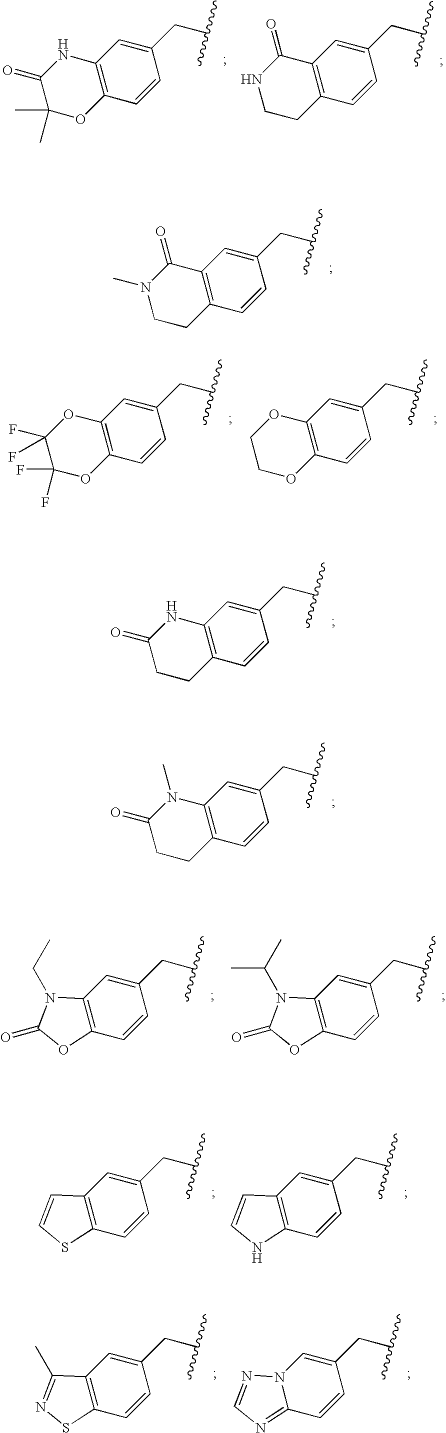

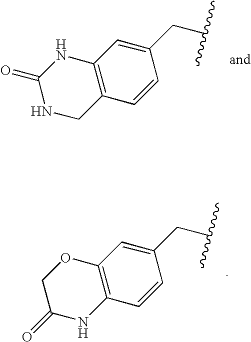

For example, in some embodiments, R¹ of the structures of Group I(a) may be selected from Substituent Group 9 as defined hereinabove.

In yet a further embodiment, R¹ of Formula (II) may be selected from Substituent Group 10:

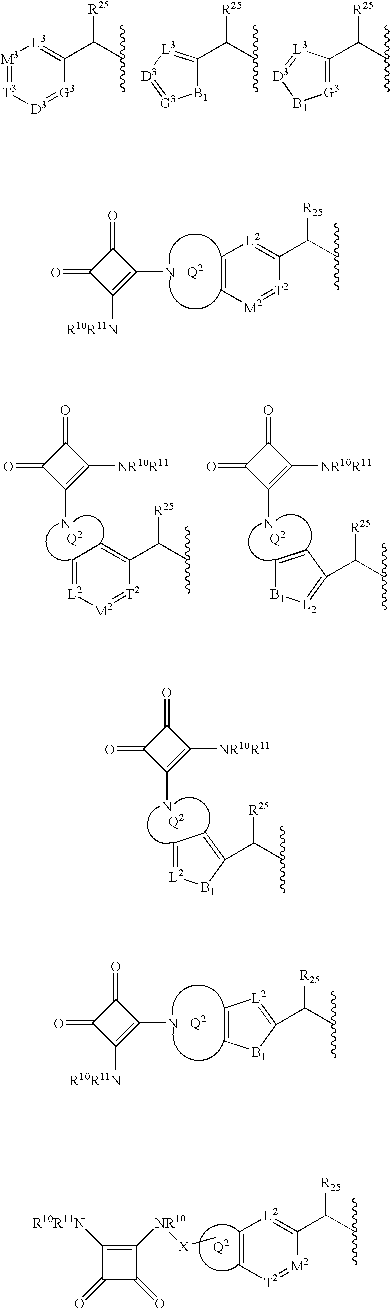

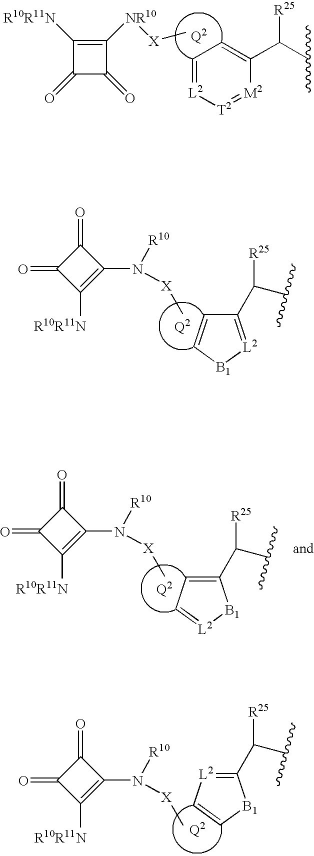

wherein:

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R¹⁸ is independently selected from the group consisting of hydrogen, alkyl, haloalkyl, cycloalkyl, heterocycloalkyl, alkynyl, aryl, heteroaryl, OH, halo, CN, C(O)NROR¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;

E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

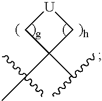

L², M², and T² are independently selected from the group consisting of CR¹⁸ and N;

D³, G³, L³, M³, and T³ are independently selected from N, CR¹⁸, (i) and (ii)

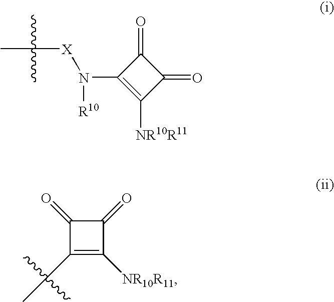

with the proviso that one of L³, M³, T³, D³, and G³ is (i) or (ii);

B₁ is selected from the group consisting of NR¹⁰, O and S; and

Q² is a 5- to 8-membered ring selected from the group consisting of cycloalkyl, heterocycloalkyl, aryl, and heteroaryl, which is optionally substituted one or more times with R¹⁹;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2; and
w is independently selected from 0-4.

The compound wherein R¹ is selected from the group consisting of:

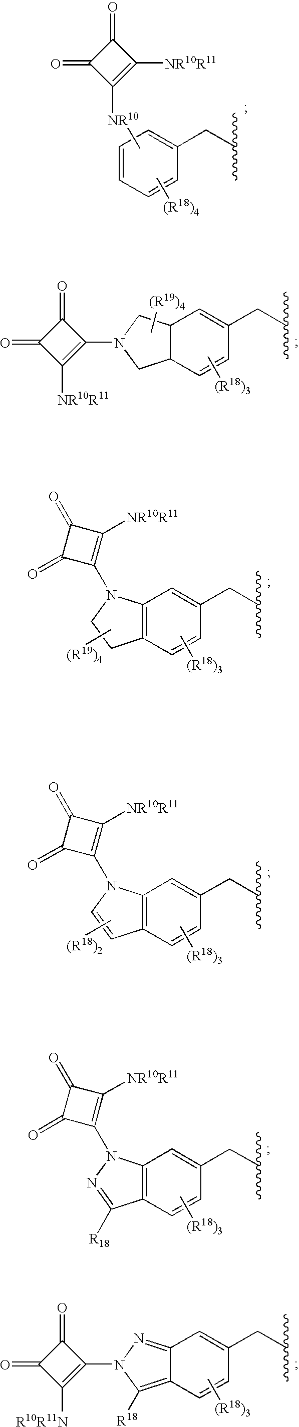

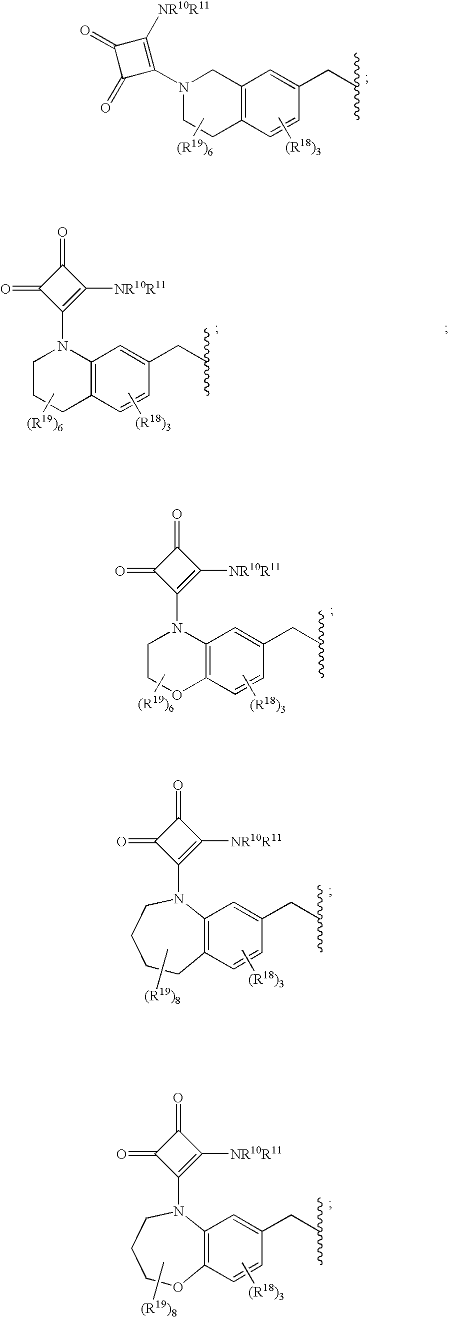

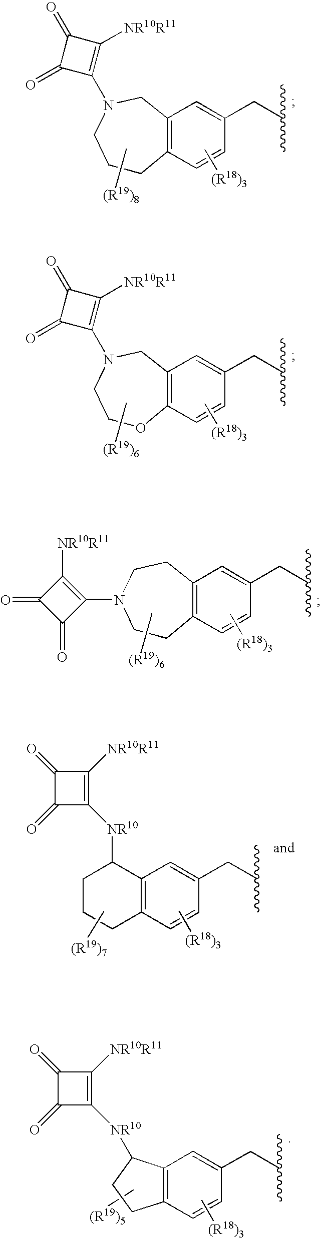

For example, in some embodiments, R¹ of the structures of Group I(a) may be selected from Substituent Group 10 as defined herinabove.

In still a further embodiment, R¹ of Formula (II) may be selected from Substituent Group 11:

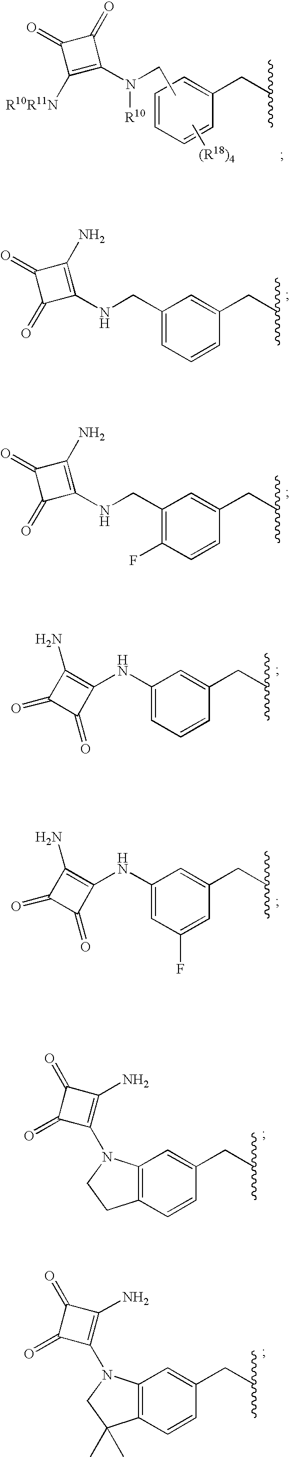

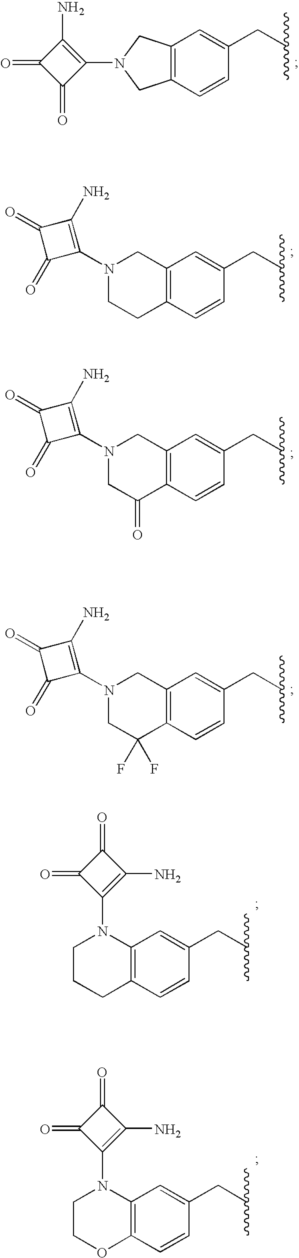

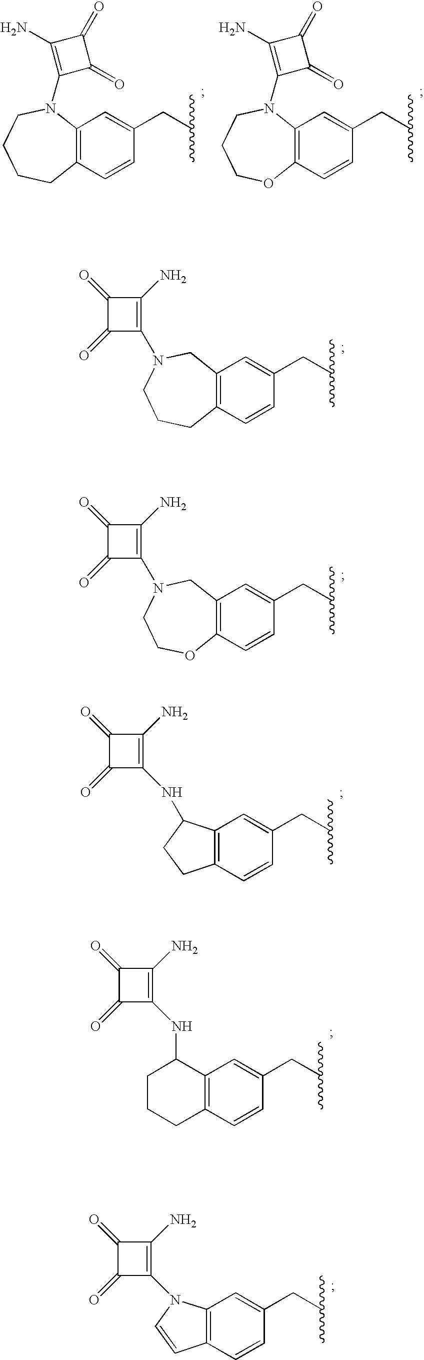

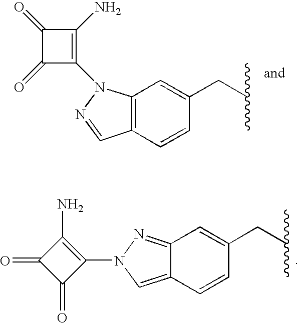

For example, in some embodiments, R¹ of the structures of Group I(a) may be selected from Substituent Group 11 as defined hereinabove.

In some embodiments, the compounds of Formula (II) may be defined wherein:

X¹ is a bond, and

R³ is selected from the group consisting of

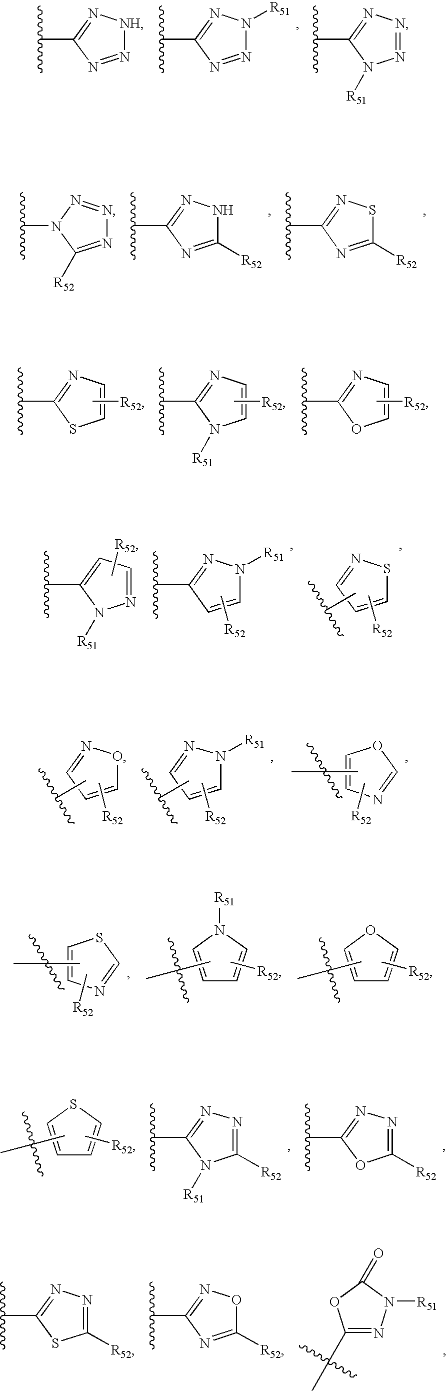

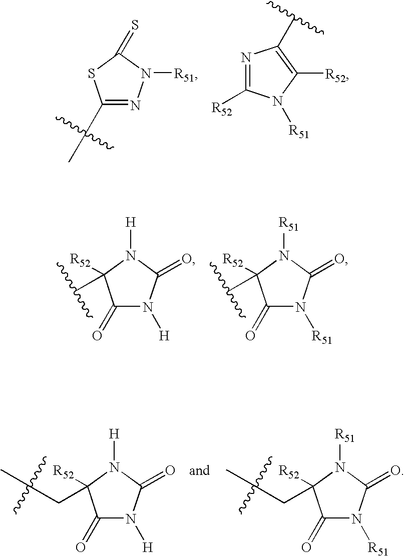

In some embodiments, the compounds of Formula (II) are selected from:

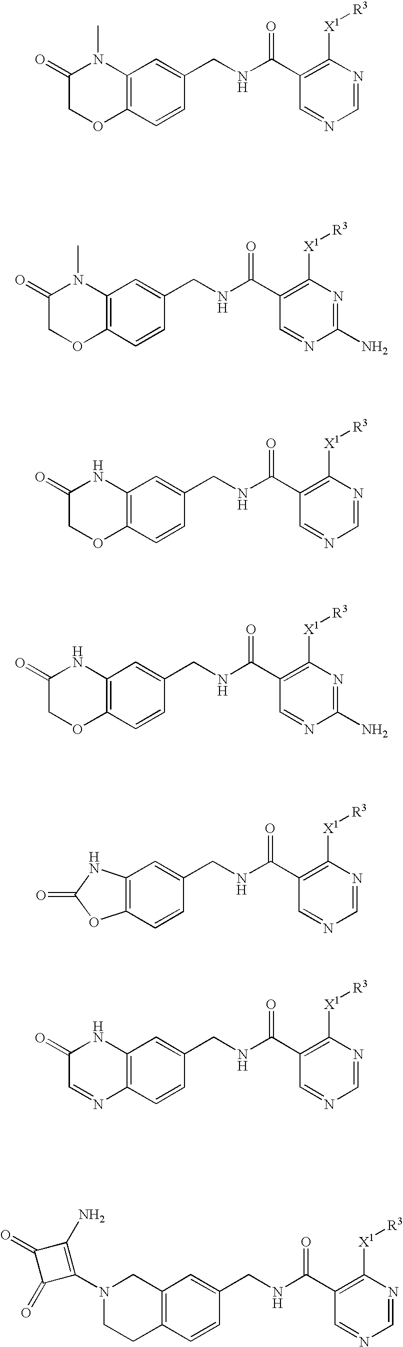

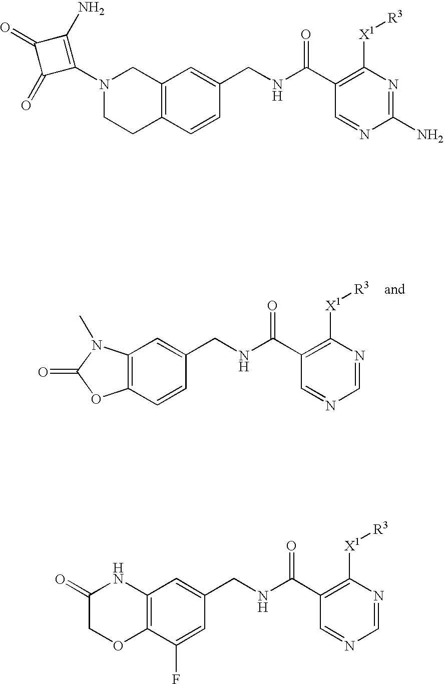

In some embodiments, the compounds described herein are selected from: 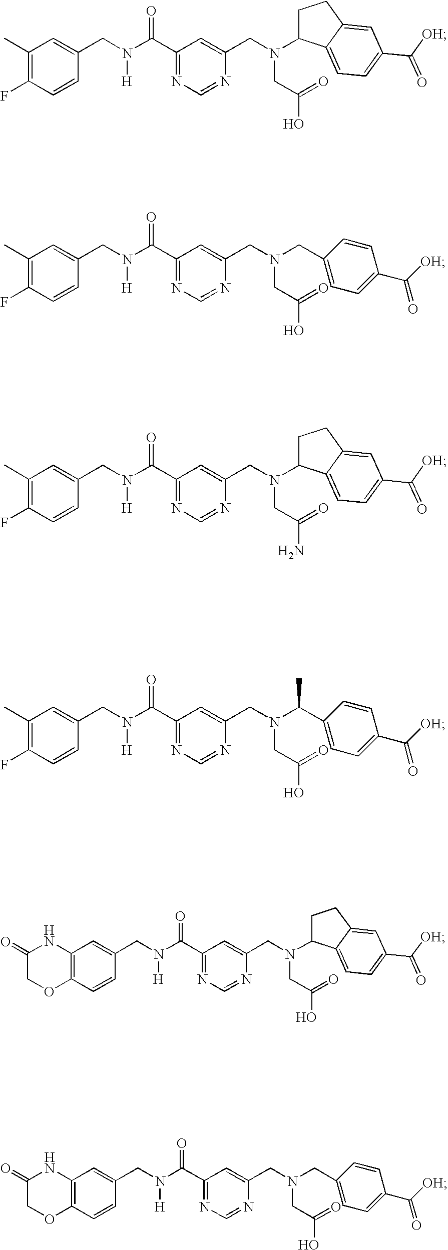 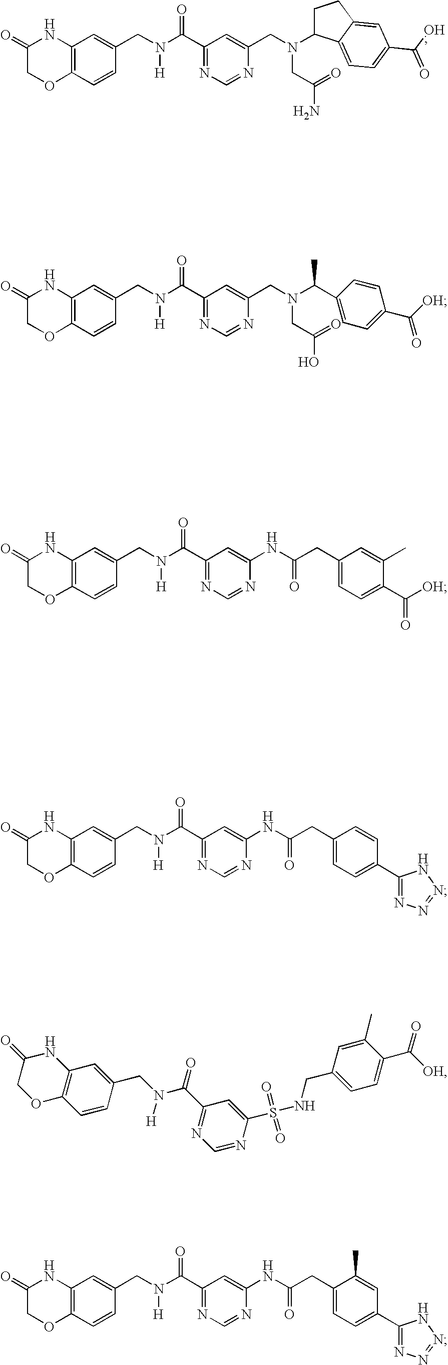 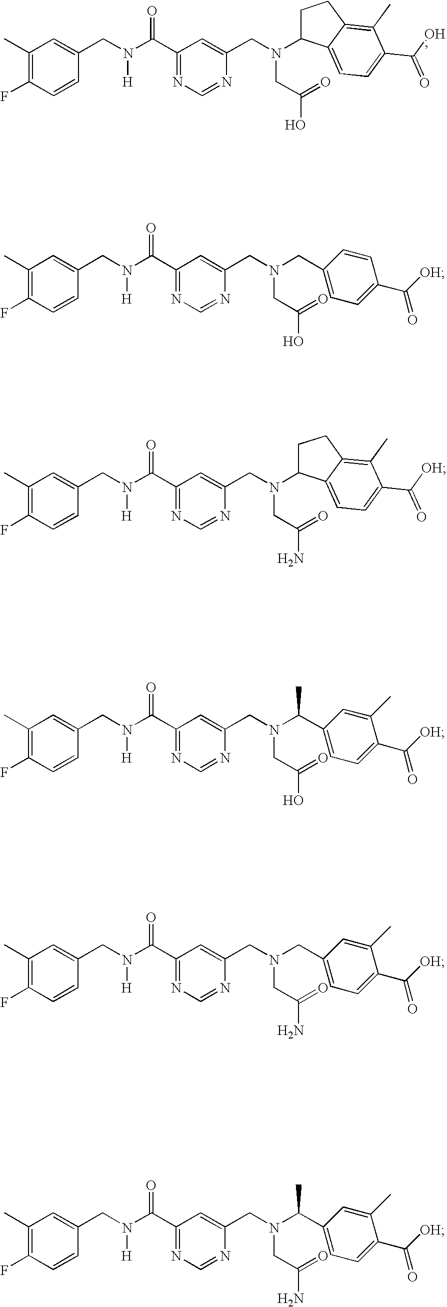 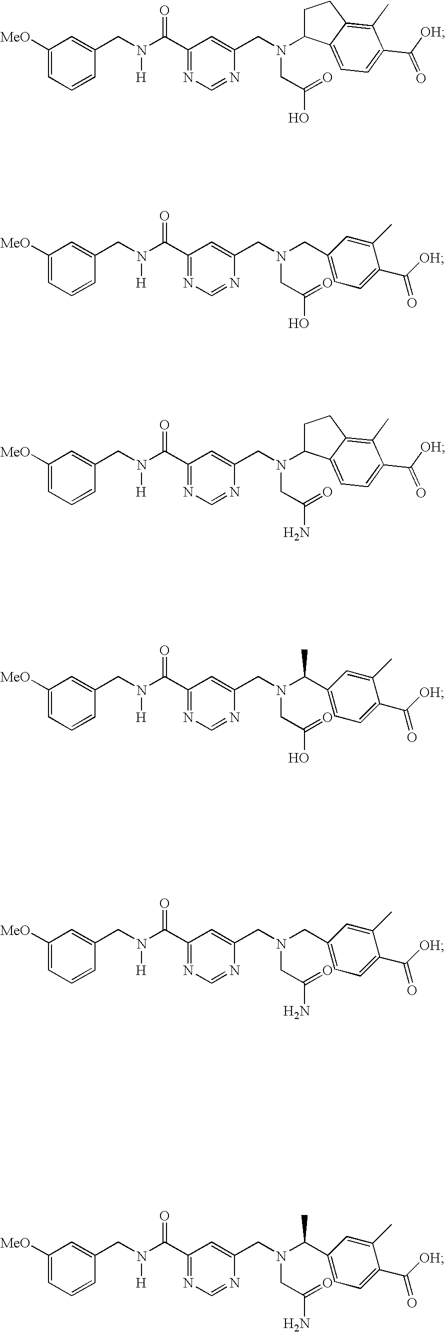 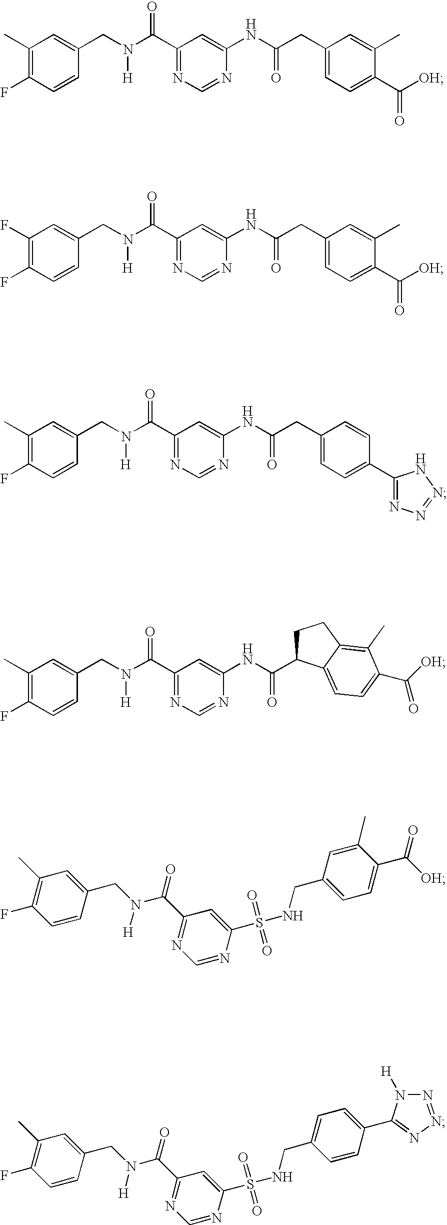 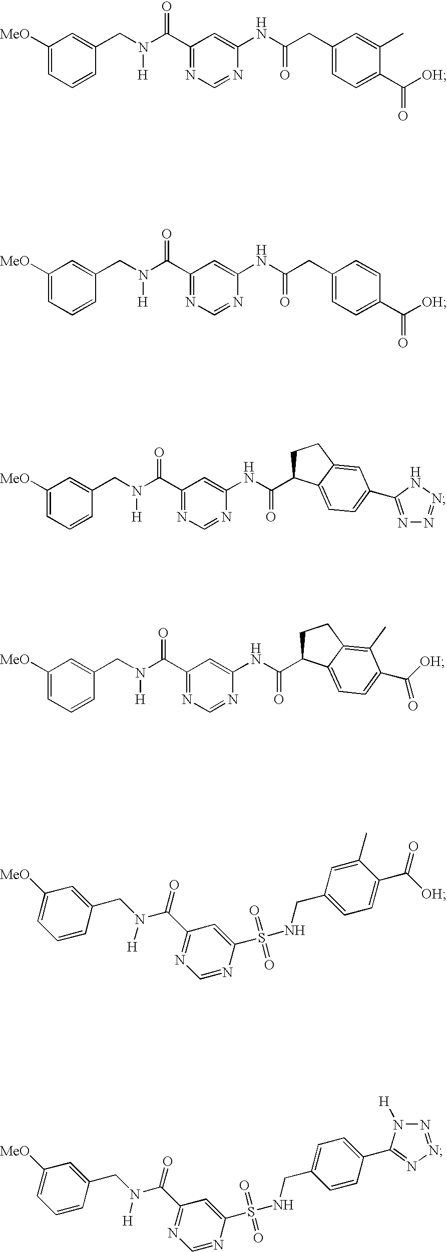 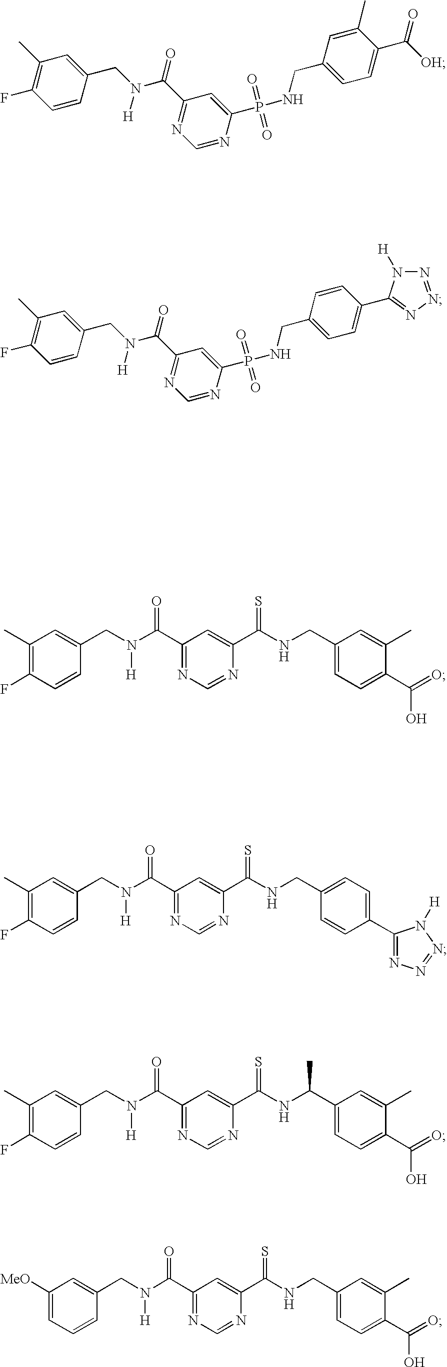 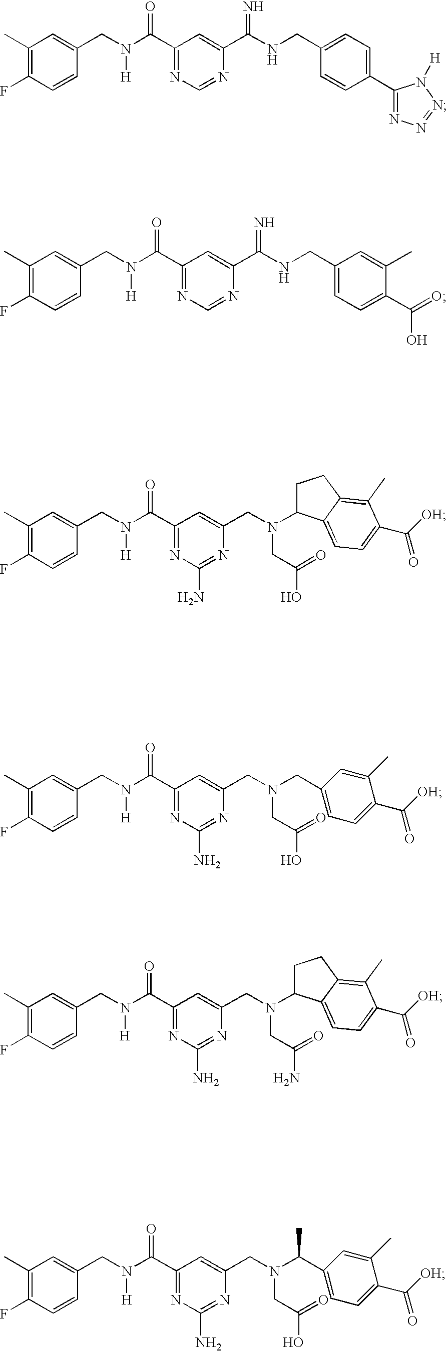 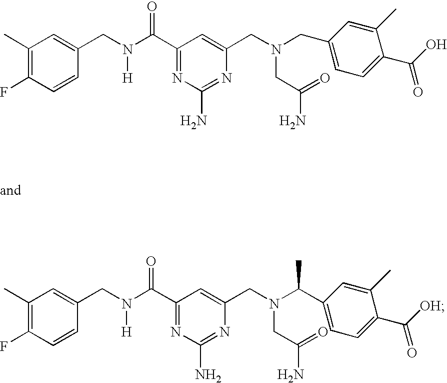
or a pharmaceutically acceptable salt thereof.

In some embodiments, the compounds described herein are selected from: 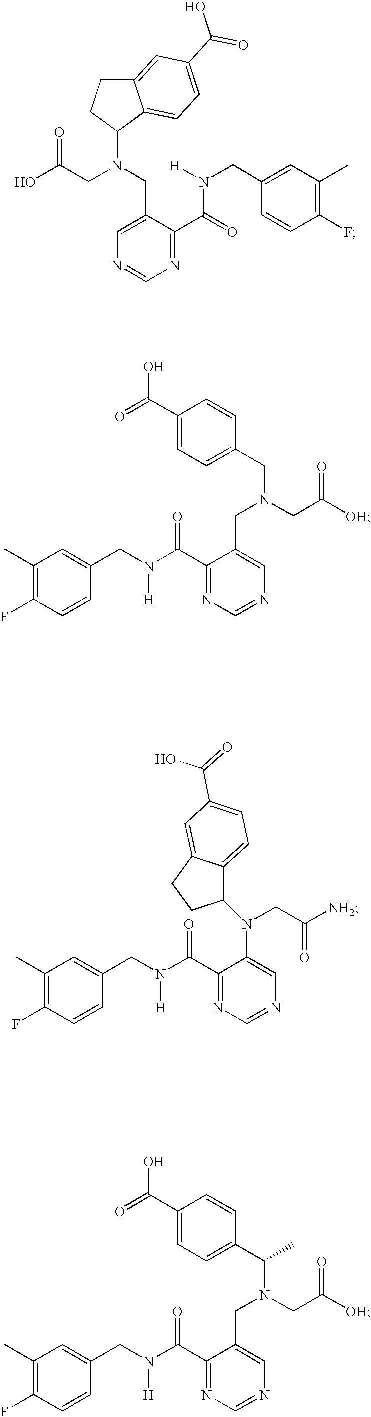 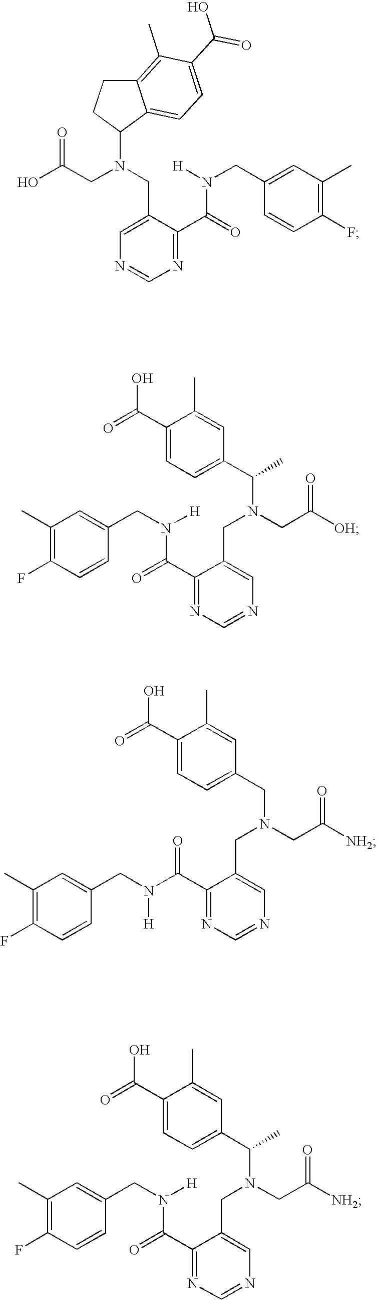 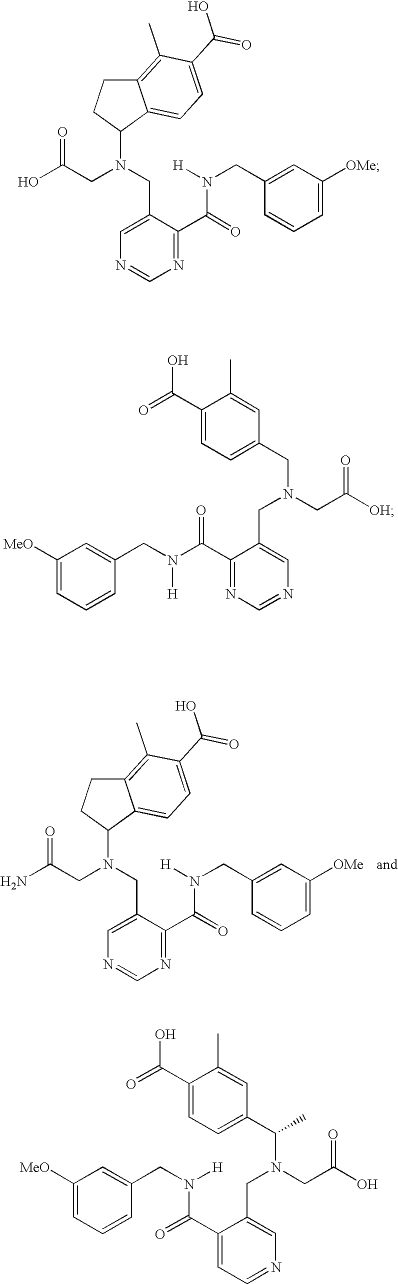
or a pharmaceutically acceptable salt thereof.

In some embodiments, the compounds described herein are selected from: 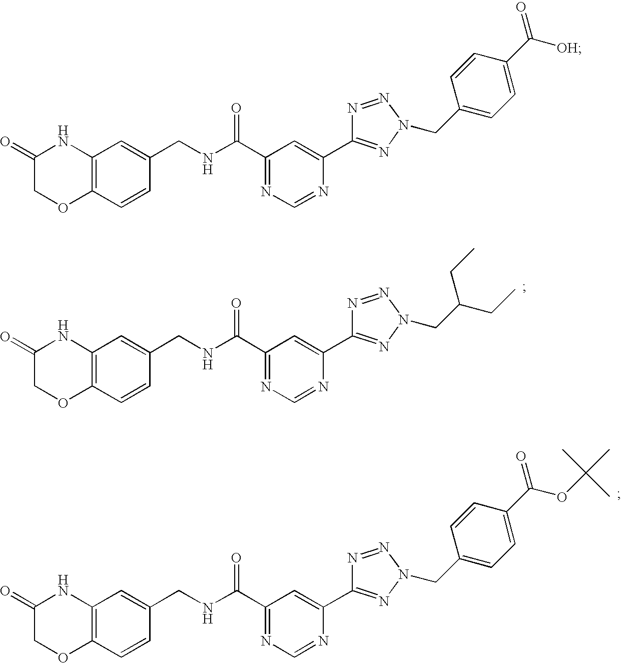 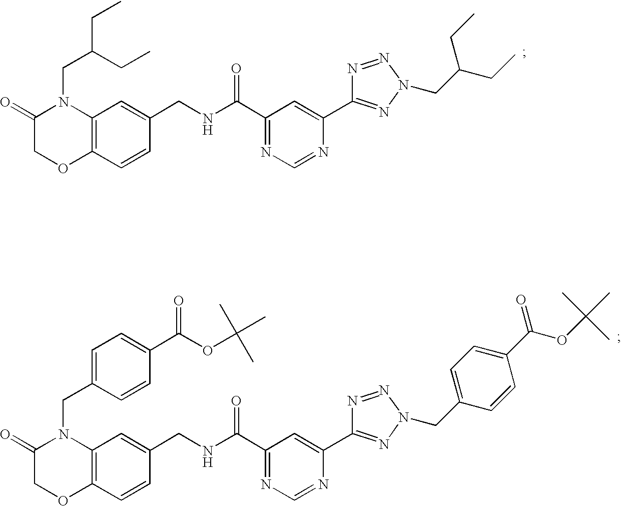 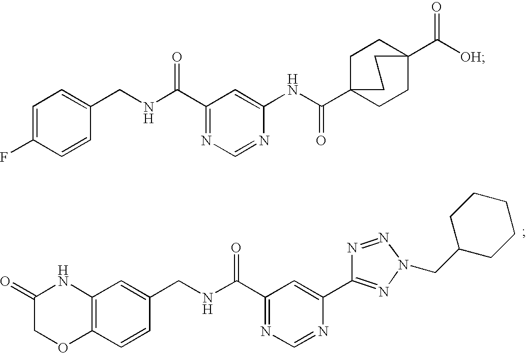 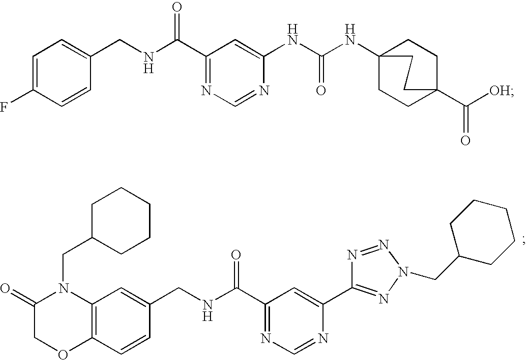 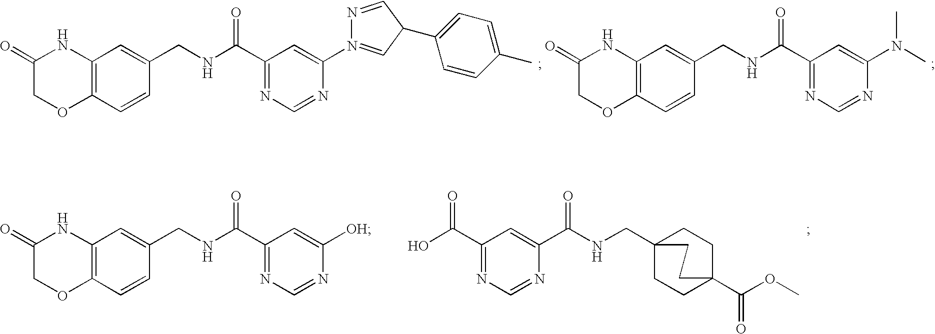 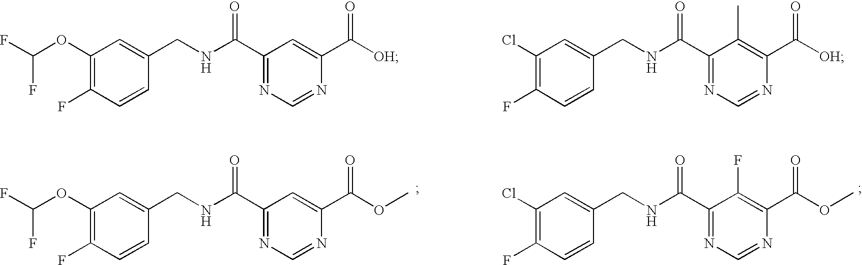 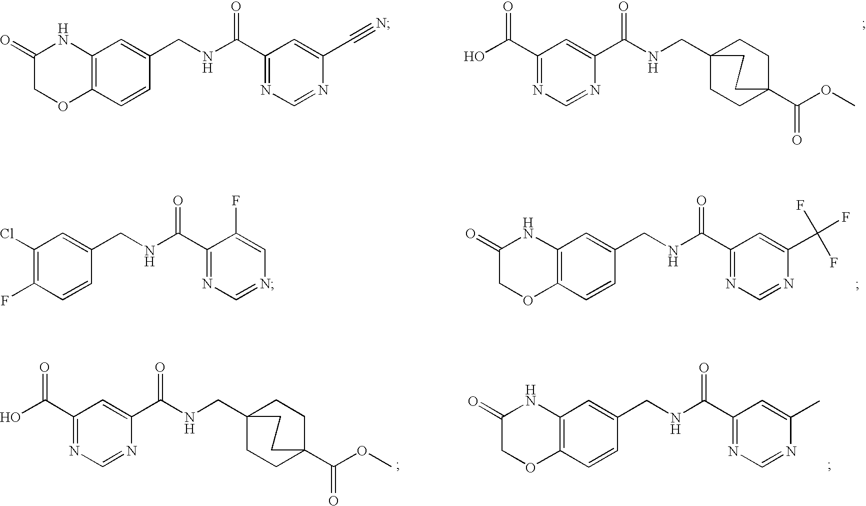 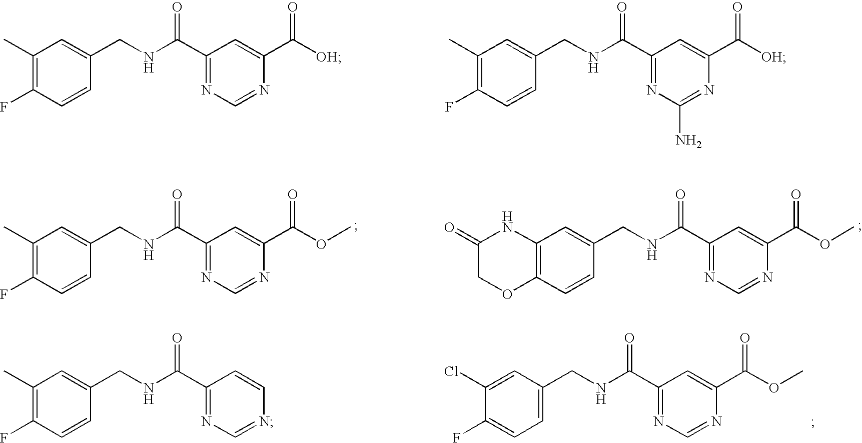 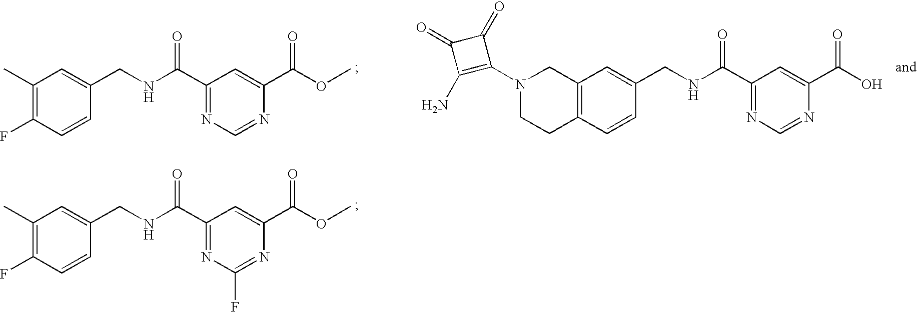
or a pharmaceutically acceptable salt thereof.

In one embodiment, the compound of Formula (I) has the following structure: 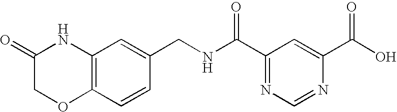
or a pharmaceutically acceptable salt thereof.

In another embodiment, the compound of Formula (I) has the following structure: 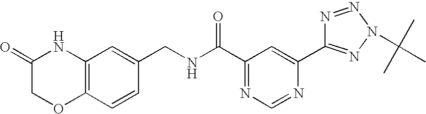
or a pharmaceutically acceptable salt thereof.

In another embodiment, the compound of Formula (I) has the following structure: 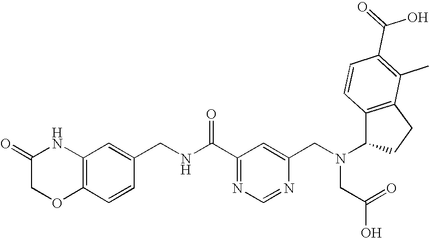
or a pharmaceutically acceptable salt thereof.

In another embodiment, the compound of Formula (I) has the following structure: 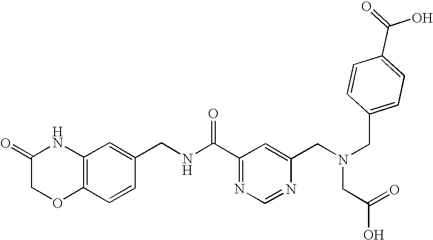
or a pharmaceutically acceptable salt thereof.

In some embodiments, the present invention provides pharmaceutical compositions including at least one compound, as described herein, selected from:

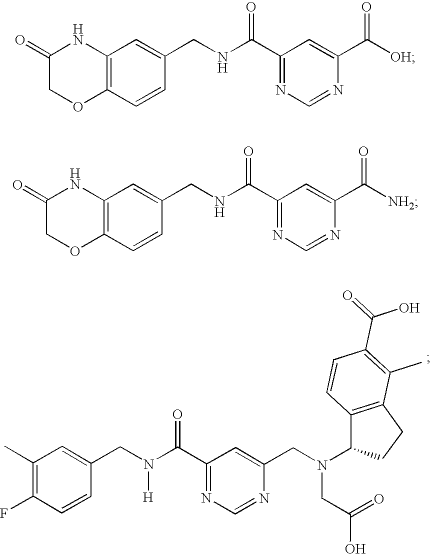

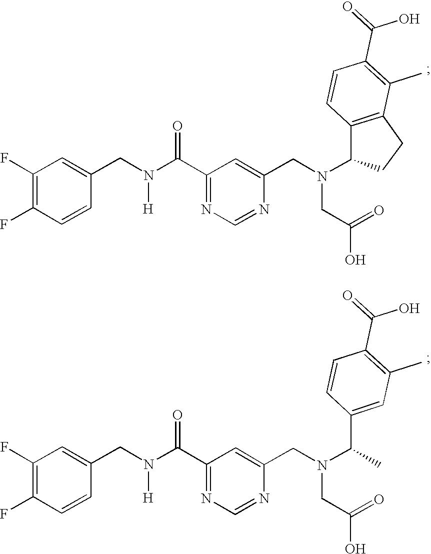

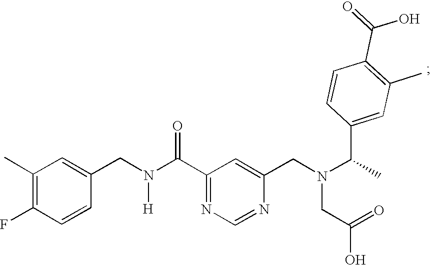

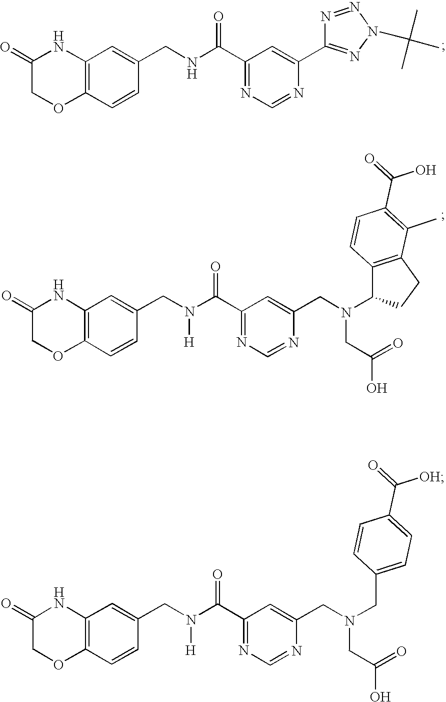

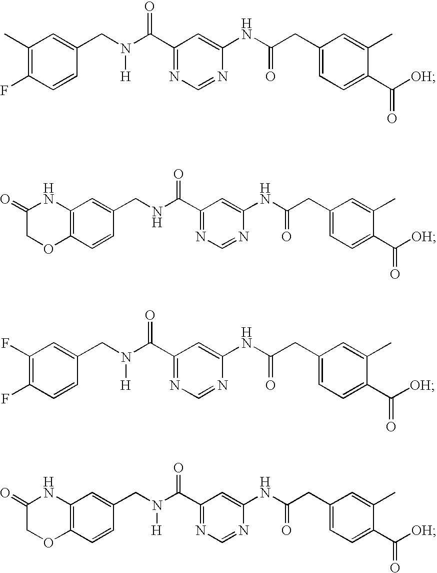

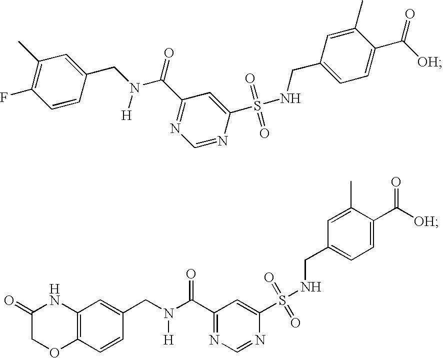

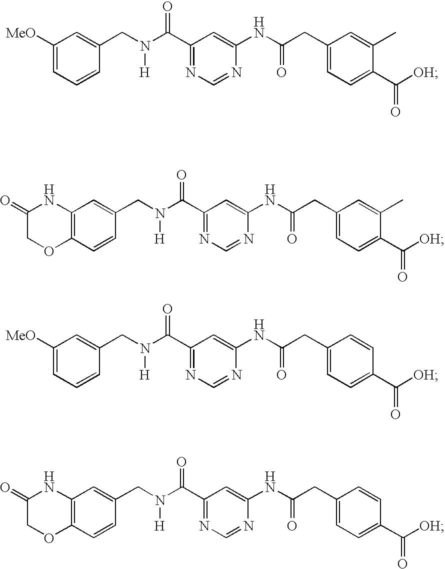

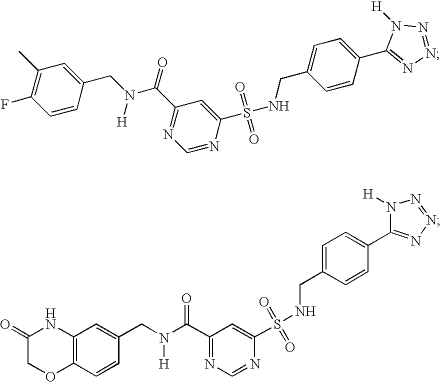

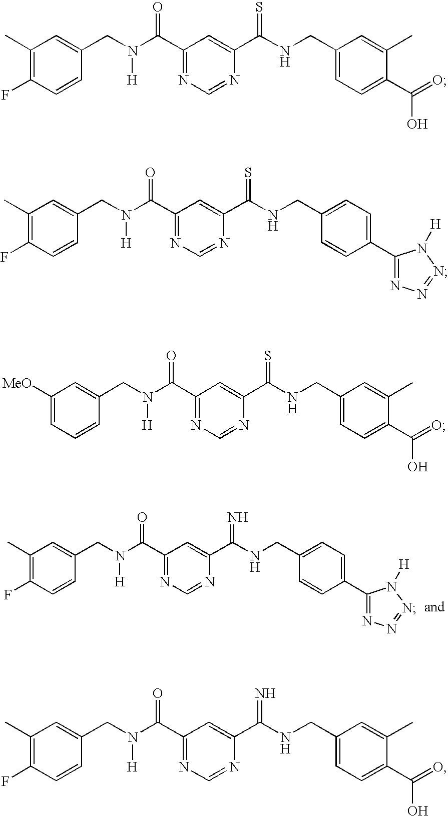

N-oxides, pharmaceutically acceptable salts, prodrugs, formulations, polymorphs, tautomers, racemic mixtures and stereoisomers thereof.

It is contemplated that the compounds of the present invention represented by the Formulas described above include all diastereomers and enantiomers, as well as racemic mixtures. Racemic mixtures may by separated by chiral salt resolution or by chiral column HPLC chromatography.

The present invention also is directed to pharmaceutical compositions including any of the MMP-13 inhibiting compounds of the present invention described above. In accordance therewith, some embodiments of the present invention provide a pharmaceutical composition which may include an effective amount of a MMP-13 inhibiting compound of the present invention and a pharmaceutically acceptable carrier.

The present invention also is directed to methods of inhibiting MMP-13 and methods of treating diseases or symptoms mediated by an MMP-13 enzyme. Such methods include administering a MMP-13 inhibiting compound of the present invention, such as a compound of Formula (I), as defined above, or an N-oxide, pharmaceutically acceptable salt or stereoisomer thereof. Examples of diseases or symptoms mediated by an MMP-13 enzyme include, but are not limited to, rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, viral infection, stroke, atherosclerosis, cardiovascular disease, reperfusion injury, traumna, chemical exposure or oxidative damage to tissues, pain, inflammatory pain, bone pain and joint pain.

In some embodiments of the present invention, the mono-amide MMP-13 inhibiting compounds defined above are used in the manufacture of a medicament for the treatment of a disease mediated by an MMP-13 enzyme.

In some embodiments, the MMP-13 inhibiting compounds defined above may be used in combination with a drug, agent or therapeutic such as, but not limited to: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

Examples of disease modifying antirheumatic drugs include, but are not limited to, methotrexate, azathioptrineluflunomide, penicillamine, gold salts, mycophenolate, mofetil and cyclophosphamide.

In some embodiments, the MMP-13 inhibiting compounds defined above may be used in combination with a biological response modifier (such as, for example, inflixmab, adalimumab, entanercept, ankinra and the like), a viscosupplement (such as, for example, hyaluronates and the like), a pain reducing drug (such as, for example, acetaminophen, aspirin, salicylic acid, codeine, oxymorphone, fentanyl, oxycodone, lidocaine and the like) or other anti-inflammatory agents or therapeutics useful for the treatment of chemokines mediated diseases.

Examples of nonsteroidal anitinflammatory drugs include, but are not limited to, piroxicam, ketoprofen, naproxen, indomethacin, and ibuprofen.

Examples of COX-2 selective inhibitors include, but are not limited to, rofecoxib, celecoxib, and valdecoxib.

An example of a COX-1 inhibitor includes, but is not limited to, piroxicam and tenoxicam.

Examples of immunosuppressives include, but are not limited to, methotrexate, cyclosporin, leflunimide, tacrolimus, rapamycin and sulfasalazine.

Examples of steroids include, but are not limited to, p-methasone, prednisolone and dexamethasone, betamethasone, cortisone, fluticasone, mometasone, methylprednisolone, triamcinolone, budesonide and beclomethasone.

Examples of biological response modifiers include, but are not limited to, anti-TNF antibodies, TNF-α antagonists, IL-1 antagonists, anti-CD40, anti-CD28, IL-10 and anti-adhesion molecules (such as, for example, inflixmab, adalimumab, entanercept, ankinra and the like), a viscosupplement (such as, for example, hyaluronates and the like), a pain reducing drug (such as, for example, acetaminophen, aspirin, salicylic acid, codeine, oxymorphone, fentanyl, oxycodone, lidocaine and the like) or other anti-inflammatory agents or therapeutics useful for the treatment of chemokines mediated diseases.

In accordance with another embodiment of the present invention, a pharmaceutical composition may include an effective amount of a compound of the present invention, a pharmaceutically acceptable carrier and a drug, agent or therapeutic selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

In some embodiments of the present invention, the compounds of Formula I are synthesized by the general method shown in Scheme 1.

The MMP-13 inhibiting activity of the MMP-13 inhibiting compounds of the present invention may be measured using any suitable assay known in the art. A standard in vitro assay for MMP-13 inhibiting activity is described in Example 3000.

The MMP-13 inhibiting compounds of the invention have an MMP-13 inhibition activity (IC₅₀ MMP-13) ranging from about 1 nM to about 20 nM, and typically, from about 8 nM to about 2 μM. MMP-13 inhibiting compounds of the invention desirably have an MMP inhibition activity ranging from about 1 nM to about 20 nM. Table 1 lists typical examples MMP-13 inhibiting compounds of the invention that have an MMP-13 activity lower than about 1 μM.

TABLE 1
Summary of MMP-13 Activity for Compounds of Formula I

| Structure | IC₅₀ | |
|---|---|---|
| 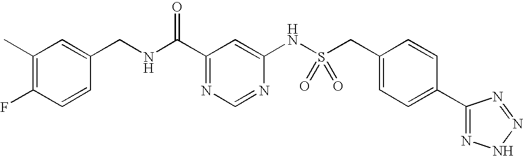 | <1000 | nM |
| 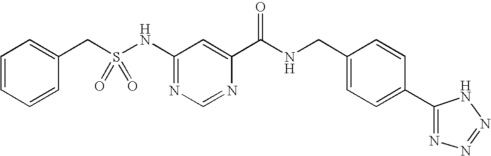 | >5 | nM |
| 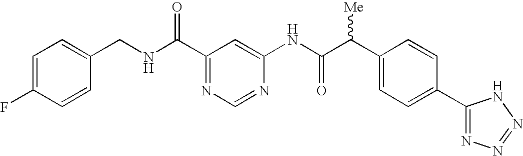 | >5 | nM |
| 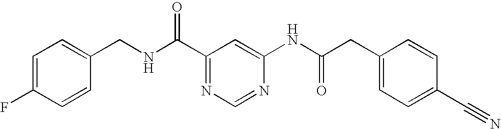 | <1000 | nM |
| 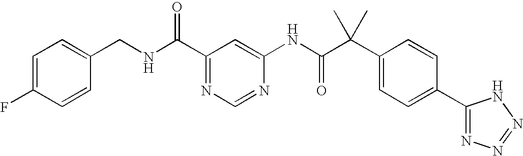 | <1000 | nM |
| 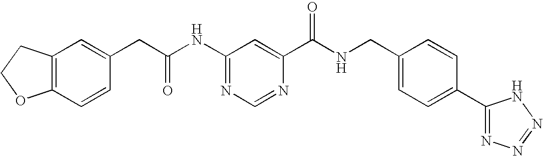 | <1000 | nM |
| 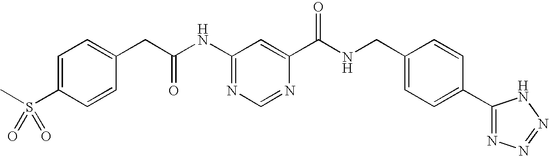 | >5 | nM |
| 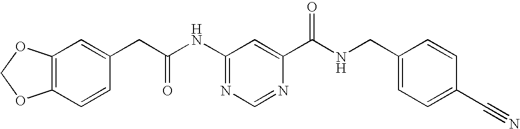 | <1000 | nM |

The synthesis of MMP-13 inhibiting compounds of the invention and their biological activity assay are described in the following examples which are not intended to be limiting in any way.

### EXAMPLES AND METHODS

All reagents and solvents were obtained from commercial sources and used without further purification. Proton (¹H) spectra were recorded on a 250 MHz NMR spectrometer in deuterated solvents. Flash chromatography was performed using Merck silica gel, grade 60, 70-230 mesh using suitable organic solvents as indicated in specific examples. Thin layer chromatography (TLC) was carried out on silica gel plates with UV detection.

### Preparative Example 1

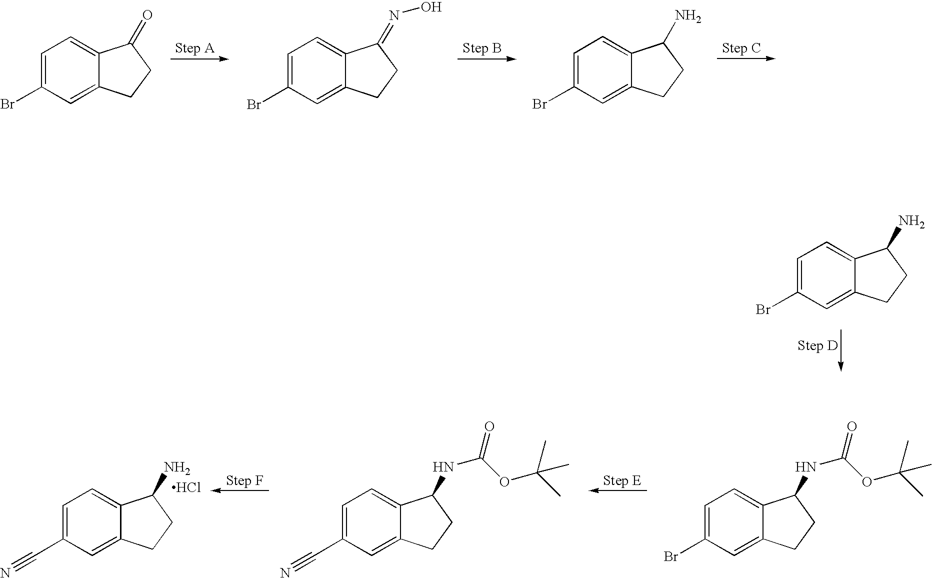 Step A

A mixture of commercially available 5-bromo-indan-1-one (1.76 g), hydroxylamine hydrochloride (636 mg) and sodium acetate (751 mg) in methanol (40 mL) was allowed to stir for 16 h at room temperature. Water (100 mL) was added and the resulting precipitate was filtered and washed with water (3×20 mL) to afford the title compound (1.88 g; >99%) as a colourless solid. [MH]+=226/228.

Step B

To a solution of the title compound from Step A above (1.88 g) in diethyl ether (20 mL) at −78° C. under an atmosphere of argon was slowly added a 1M solution of lithium aluminum hydride in diethyl ether (42.4 mL). The mixture was heated to reflux (40° C.) and allowed to stir for 5 h. The mixture was cooled to 0° C. and water (1.6 mL), 15% aqueous sodium hydroxide (1.6 mL) and water (4.8 mL) were carefully and sequentially added. The resulting mixture was filtered through Celite® and the filtrate was concentrated to give the title compound (1.65 g; 94%) as a clear oil. [MH]⁺=212/214.

Step C

To a boiling solution of the title compound from Step B above (1.13 g) in methanol (2.3 mL) was added a hot solution of commercially available N-acetyl-L-leucine (924 mg) in methanol (3 mL). The solution was allowed to cool to room temperature, which afforded a white precipitate. The solid was separated from the supernatant and washed with methanol (2 mL). The solid was recrystallized two times from methanol. To the resulting solid were added 10% aqueous sodium hydroxide (20 mL) and diethyl ether (20 mL). Once the solid was dissolved, the organic layer was separated and the aqueous layer was washed with diethyl ether. The combined organic layers were dried (MgSO₄), filtered and concentrated to give the title compound (99 mg; 18%) as a clear oil. [MH]⁺=212/214.

Step D

To a solution of the title compound from Step C above (300 mg), di-tert-butyl dicarbonate (370 mg) and triethylamine (237 μL) in tetrahydrofurane (10 mL) was allowed to stir for 16 h at room temperature. The solution was concentrated and the remaining residue was purified by chromatography (silica, hexanes/ethyl acetate) to give the title compound (460 mg; >99%) as a clear oil. [(M-isobutene)H]⁺=256/258, [MNa]⁺=334/336.

Step E

A mixture of the title compound from Step D above (460 mg), tetrakis triphenylphosphinepalladium (89 mg), zinc cyanide (200 mg) in N,N-dimethylformamide (5 mL) under an atmosphere of argon in a sealed vial was allowed to stir for 18 h at 110° C. The mixture was allowed to cool to room temperature before diethyl ether (20 mL) and water (20 mL) were added. The separated aqueous layer was washed with diethyl ether (4×10 mL). The combined organic layers were washed with water (3×10 mL) and brine (10 mL), dried (MgSO₄), filtered and concentrated. The resulting residue was purified by chromatography (silica, hexanes/ethyl acetate) to afford the title compound (170 mg; 47%) as a clear oil. [MH]⁺=259, [MNa]⁺=281.

Step F

To the title compound from Step E above (170 mg) was added a 4M solution of hydrochloric acid in dioxane (2 mL). The resulting solution was allowed to stir for 3 h at room temperature at which time a precipitate had formed. The mixture was concentrated to give the title compound (128 mg; >99%). [M-Cl]⁺=159.

### Preparative Example 2

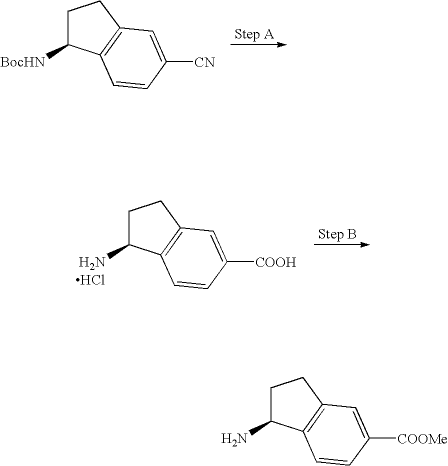 Step A

The title compound from the Preparative Example 1, Step E above (1.0 g) was suspended in 6N hydrochloric acid (50 mL) and heated to 110-112° C. for 20 h upon which the solution became homogeneous. The solvent was removed under reduce pressure to give the intermediate. [M-Cl]⁺=178.

Step B

The intermediate from Step A above was dissolved in anhydrous MeOH (150 mL) and saturated with anhydrous hydrogen chloride gas. The reaction mixture was then heated to reflux for 20 h. After cooling to room temperature, the solvent was removed under reduced pressure to give an oil. The oil was taken up in dichloromethane and washed with saturated NaHCO₃. The organic phase was separated and dried over MgSO₄, filtered and concentrated to give the title compound (0.66 g, 89% over two steps) as an oil which slowly crystallized into a light brown solid.

### Preparative Example 3

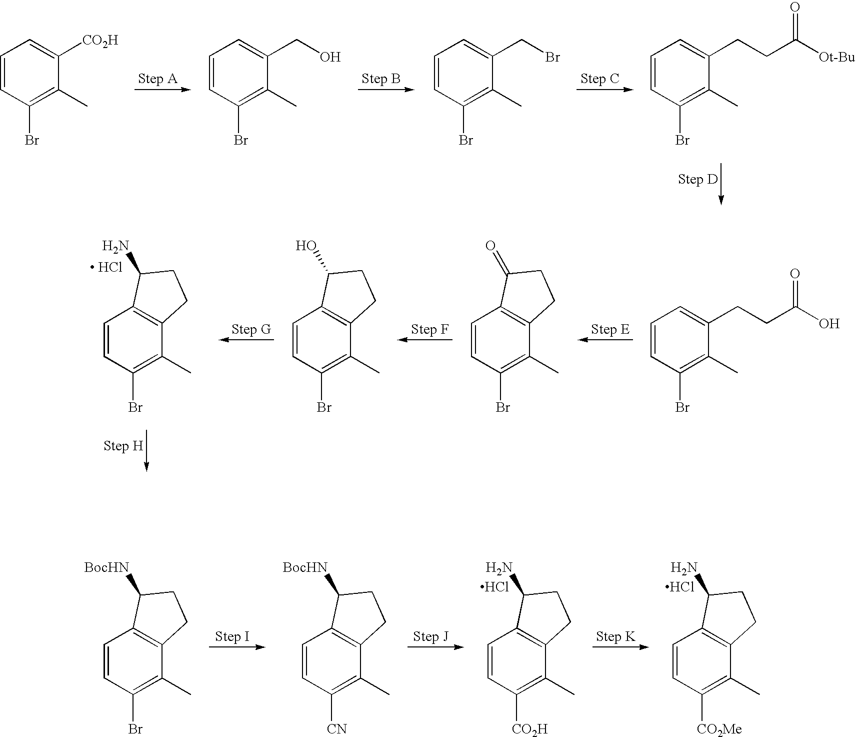 Step A

3-Bromo-2-methyl-benzoic acid (20.0 g) was dissolved in anhydrous THF (200 mL) under nitrogen and the reaction vessel was cooled to 0° C. in an ice bath. To this cooled solution was added BH₃-THF complex (1M in THF, 140 mL) dropwise over a 3 h period. Once gas evolution had subsided, the reaction mixture was warmed to room temperature and stirred for an additional 12 h. The mixture was then poured into 1N hydrochloric acid (500 mL) cooled with ice and then extracted with Et₂O (3×150 mL). The organic extracts were combined, dried over anhydrous MgSO₄, filtered, and then concentrated to afford the intermediate (18.1 g; 97%) as a colourless solid. ¹H-NMR (CDCl₃) δ=2.40 (s, 3H), 4.70 (s, 2H), 7.10 (t, 1H), 7.30 (d, 1H), 7.50 (d, 1H).

Step B

The intermediate from Step A above (18.1 g) was dissolved in anhydrous CH₂Cl₂ (150 mL) under nitrogen and the reaction vessel was cooled to 0° C. in an ice bath. To this cooled solution was added PBr₃ (5.52 mL) over a 10 min period. Once the addition was complete, the reaction mixture was warmed to room temperature and stirred for an additional 12 h. The mixture was cooled in an ice bath and quenched by the dropwise addition of MeOH (20 mL). The organic phase was washed with saturated NaHCO₃ (2×150 mL), dried over anhydrous MgSO₄, filtered, and then concentrated to afford the intermediate (23.8 g; 97%) as viscous oil. ¹H-NMR (CDCl₃) δ=2.50 (s, 3H), 4.50 (s, 2H), 7.00 (t, H), 7.25 (d, 1H), 7.50 (d, 1H).

Step C

t-Butyl acetate (12.7 mL) was dissolved in anhydrous THF (200 mL) under nitrogen and the reaction vessel was cooled to −78° C. in a dry ice/acetone bath. To this cooled solution was added dropwise lithium diispropylamide (1.5M in cyclohexane, 63.0 mL) and the mixture was allowed to stir for an additional 1 h upon which a solution of intermediate from Step B above (23.8 g) was added in THF (30 mL). Once the addition was complete, the reaction mixture was gradually warmed to room temperature over a 12 h period. The mixture was concentrated and the remaining viscous oil was dissolved in Et₂O (300 mL), washed with 0.5N hydrochloric acid (2×100 mL), dried over anhydrous MgSO₄, filtered, and then concentrated to afford the intermediate (21.5 g; 80%) as a pale-yellow viscous oil. ¹H-NMR (CDCl₃) δ=1.50 (s, 9H), 2.40 (s, 3H), 2.50 (t, 2H), 3.00 (t, 2H), 7.00 (t, 1H), 7.25 (d, 1H), 7.50 (d, 1H).

Step D

The intermediate from Step C above (21.5 g) was combined with polyphosphoric acid (250 g) and placed in a 140° C. oil bath for 10 min while mixing the thick slurry occasionally with a spatula. To this mixture was then added ice water (1 L) and the mixture was stirred for 2 h. The mixture was then filtered and the solid was washed with H₂O (2×100 mL) and dried to afford the intermediate (16.7 g; 96%). ¹H-NMR (CDCl₃) δ=2.40 (s, 3H), 2.65 (t, 2H), 3.00 (t, 2H), 7.00 (t, 1H), 7.20 (d, 1H), 7.50 (d, 1H).

Step E

The intermediate from Step D above (11.6 g) was dissolved in anhydrous CH₂Cl₂ (100 mL) under nitrogen and the reaction vessel was cooled to 0° C. in an ice bath. To this mixture was added dropwise oxalyl chloride (12.0 mL) and the mixture was stirred for 3 h after which the mixture was concentrated under reduced pressure. The remaining dark residue was dissolved in anhydrous CH₂Cl₂ (300 mL) and to this mixture was added AlCl₃ (6.40 g). Once the addition was complete, the mixture was refluxed for 4 h upon which the mixture was poured into ice water (500 mL) and extracted with CH₂Cl₂ (2×11 mL). The combined extracts were combined, dried over anhydrous MgSO₄, filtered, and then concentrated to afford the intermediate (10.6 g; 98%) as a light brown solid. ¹H-NMR (CDCl₃) δ=2.40 (s, 9H), 2.70 (t, 2H), 3.05 (t, 2H), 7.50 (d, 1H), 7.65 (d, 1H).

Step F

To a cooled solution of (S)-2-methyl-CBS-oxazaborolidine (1M in toluene, 8.6 mL) and borane-methyl sulfide complex (1M in CH₂Cl₂, 43.0 mL) at −20° C. (internal temperature) in CH₂Cl₂ (200 mL) was added a solution of intermediate from Step E above (9.66 g, in 70 mL CH₂Cl₂), over a 10 h period via a syringe pump. After the addition was complete, the mixture was then quenched by the addition of MeOH (100 mL) at −20° C., warmed to room temperature and concentrated. The crude mixture was purified by flash chromatography (10% to 30% Et₂O/CH₂Cl₂ gradient) to afford the intermediate (8.7 g; 90%) as a colourless solid. ¹H-NMR (CDCl₃) δ=2.00 (m, 1H), 2.35 (s, 3H), 2.50 (m, 1H), 2.90 (m, 1H), 3.10 (m, 1H), 5.25 (m, 1H), 7.20 (d, 1H), 7.50 (d, 1H).

Step G

To a −78° C. cooled solution of intermediate from step F above (8.7 g) in CH₂Cl₂ (200 mL) under nitrogen was added triethylamine (15.9 mL) followed by methanesulfonyl chloride (4.5 mL). This mixture was stirred for 90 min and then NH₃ (˜150 mL) was condensed into the mixture using a dry ice/acetone cold finger at a rate of ˜3 mL/minute. After stirring at −78° C. for an additional 2 h, the mixture was gradually warmed to room temperature allowing the NH₃ to evaporate from the reaction mixture. 1N NaOH (200 mL) was added and the aqueous layer was extracted with CH₂Cl₂ (2×100 mL). The combined extracts were dried over anhydrous MgSO₄, filtered, and then concentrated to afford crude material as a light brown oil. This oil was dissolved in Et₂O (200 mL) and hydrogen chloride (4M in dioxane, 10 mL) was added and the precipitate was collected and dried to give the intermediate (9.0 g; 90%). [M-NH₃Cl]⁺=209/211.

Step H

The intermediate from Step G above (5.2 g) was mixed in dry CH₂Cl₂ (50 mL) and cooled to 0° C. and to this cooled solution was added di-tert-butyl dicarbonate (5.0 g) followed by Et₃N (9.67 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid. ¹H-NMR (CDCl₃, free base) δ=1.80 (m, 1H), 2.30 (s, 3H), 2.60 (m, 1H), 2.80 (m, 1H), 2.90 (m, 1H), 4.30 (t, 1H), 7.00 (d, 1H), 7.40 (m, H).

Step I

The intermediate from Step H above (7.2 g), zinc(II) cyanide (5.2 g) and Pd(PPh₃)₄ (2.6 g) were combined under nitrogen and anhydrous DMF (80 mL) was added. The yellow mixture was heated to 100° C. for 18 h and then concentrated under reduced pressure to afford crude material which was purified by flash chromatography (20% CH₂Cl₂/EtOAc) to give the intermediate (4.5 g; 75%) as an off-white solid. ¹H-NMR (CDCl₃) δ=1.50 (s, 3H), 1.90 (m, 1H), 2.40 (s, 3H), 2.70 (m, 1H), 2.80 (m, H), 2.95 (m, 1H), 4.75 (m, 1H), 5.15 (m, 1H), 7.20 (d, 1H), 7.50 (d, 1H).

Step J

The intermediate from Step I above (1.0 g) was suspended in 6N hydrochloric acid (20 mL) and heated to 100° C. for 12 h upon which the solution become homogeneous. The solvent was removed under reduce pressure to give the intermediate (834 mg; quantitative) as a colourless solid. [M-NH₃Cl]⁺=175.

Step K

The intermediate from Step J above (1.0 g) was dissolved in anhydrous MeOH (20 mL) and cooled to 0° C. and anhydrous hydrogen chloride was bubbled through this solution for 2-3 min. The reaction mixture was then heated to reflux for 12 h. After cooling to room temperature, the solvent was removed under reduced pressure to give the title compound (880 mg; quantitative) as a colourless solid. [M-NH₃Cl]⁺=189.

### Preparative Example 4

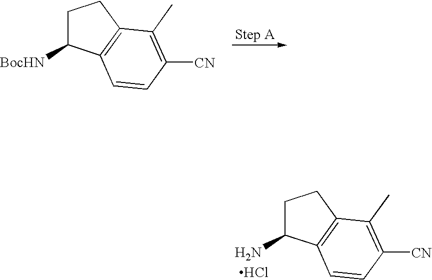 Step A

To the intermediate from the Preparative Example 3, Step I above (108 mg) was added a solution of hydrogen chloride (4M in dioxane, 2 mL) and the resulting solution was allowed to stir at 22° C. for 6 h at which time a precipitate had formed. The mixture was concentrated to give the title compound (83 mg, >99%) as a colourless powder. [M-NH₃Cl]⁺=156.

### Preparative Example 5

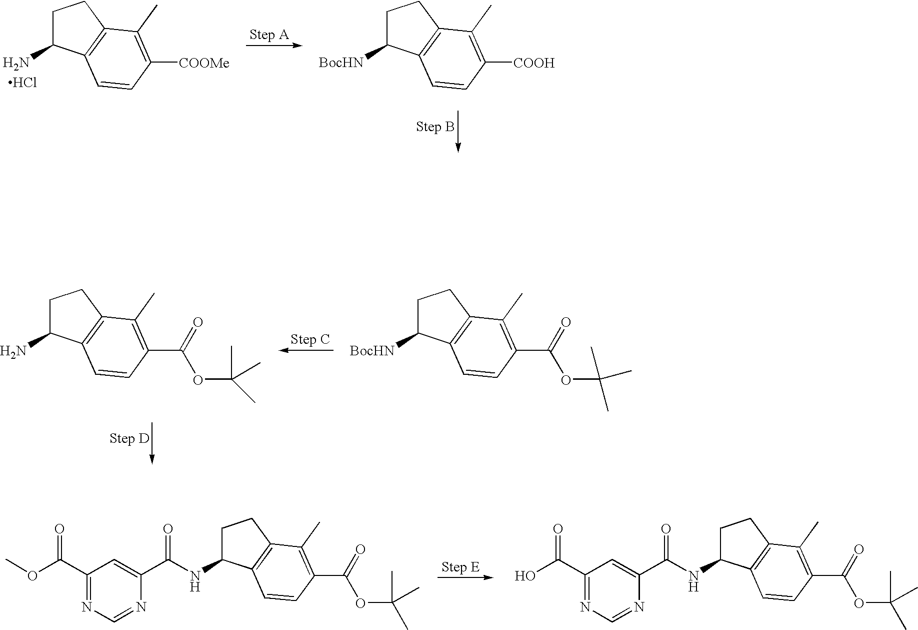 Step A

The intermediate from Preparative Example 3, Step K (1.5 g) was mixed in dry CH₂Cl₂ (50 mL) and cooled to 0° C. and to this cooled solution was added di-tert-butyl dicarbonate (1.6 g) followed by Et₃N (1 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid which was dissolved in tedrahydrofurane (60 mL). To the mixture was added a 1M aqueous LiOH solution (60 mL) and the mixture was stirred at 50° C. for 2 h. The mixture was concentrated to dryness and redissolved in water, acidified to pH=5 with hydrochloric acid and extracted with ethyl acetate. The organic layer was dried (MgSO₄) and concentrated to afford the intermediate as colourless solid (1.87 g). [MNa]⁺=314.

Step B

To a solution of the title compound from Step A above (1.87 g) in dry toluene (15 mL) was added Di-tert-butoxymethyl dimethylamine (6.2 mL) at 80° C. At this temperature the mixture was stirred for 3 h. After cooling to room temperature the mixture was concentrated and purified by column chromatography (silica, dichloromethane) to afford the intermediate (820 mg; 38%) as a colourless solid. [MNa]⁺=370.

Step C

To a solution of the title compound from Step B above (820 mg) in tert-butyl acetate (40 mL) was added sulfuric acid (0.65 mL) at room temperature. The mixture was stirred for 5 h and concentrated to dryness. The residue was dissolved ethyl acetate and washed with a saturated solution of sodium hydrogen carbonate and brine. After drying (MgSO₄) the intermediate (640 mg; 99%) was obtained as a colourless solid. [M-NH₂]⁺=231.

Step D

To a solution of the title compound from Step C above (360 mg) in dry dimethylformamide (5 mL) was added bromotrispyrrolidinophosphonium hexafluorophosphate (1.1 g), the intermediate from the Preparative Example 2117, Step A (310 mg) and N-methylmorpholine (0.5 mL). The mixture was stirred at room temperature overnight and concentrated to dryness. The residue was dissolved in water and extracted with ethyl acetate. After drying (MgSO₄) the solution was concentrated and purified by chromatography (silica, cyclohexene/ethyl acetate) to afford the title compound as a colourless solid (285 mg; 48%). [MNa]⁺=434.

Step E

The title compound from Step D above (285 mg) was dissolved in a 0.5M solution of sodium hydroxide in dry methanol (1.5 mL). The reaction mixture was stirred at room temperature for 2 h and then concentrated to afford a beige solid. This material was dissolved in water (6.2 mL) and treated with a 1M aqueous solution of hydrochloric acid (2 mL). The resulting suspension was diluted with water and extracted with ethyl acetate. After drying (MgSO₄) the solution was concentrated to afford the title compound (282 mg; quantitative) as a colourless solid. [MNa]⁺=420.

### Preparative Example 6

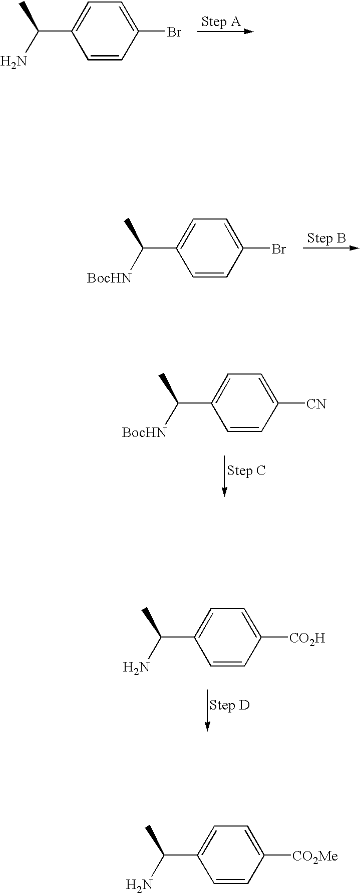 Step A

Commercially obtained (S)-(-)-1-(4-bromophenyl)ethylamine (2.0 g, 10.1 mmol) was dissolved in 50 mL dry tetrahydrofuran (THF) and cooled to 0° C. and to this cooled solution was added di-t-butyl dicarbonate (2.0 g, 9.1 mmol) dissolved in 3.0 mL of metheylene chloride (CH₂Cl₂) followed by Et₃N (2.8 mL, 20.1 mmol). The solution was allowed to warm to room temperature. After stirring for 3 hours, the mixture was concentrated and re-dissolved in 100 mL methylene chloride (CH₂Cl₂). This solution was washed with 1N HCl (2×50 mL) and saturated NaHCO₃ (1×50 mL). The CH₂Cl₂ layer was dried over anhydrous MgSO₄, filtered, and concentrated to afford 2.5 g of the desired boc product in 92% yield as a white solid.

¹H-NMR δ (CDCl₃) 1.35 (br. s, 12H), 4.72 (br. s, 2H), 7.17 (d, 2H), 7.43 (d, 2H).

Step B

The Boc amine product (4.0 g, 13.3 mmol), ZnCN₂ (3.0 g, 24.4 mmol), and Pd[PPh₃]₄ (1.5 g, 1.3 mmol) were combined under nitrogen and anhydrous dimethylformamide (25 mL) was added. The yellow mixture was heated to 100° C. for 18 h and then concentrated under reduced pressure to afford crude cyano product which was purified by flash chromatography (20% hexane/CH₂Cl₂) to give 2.0 g of the desired cyano compound as an oil in 60% yield.

¹H-NMR δ (CDCl₃) 0.89-1.62 (br. m, 12H), 4.81 (br. s, 2H), 7.42 (d, 2H), 7.65 (d, 2H).

MH⁺=247

Step C

The cyano compound (2.0 g, 8.1 mmol) was suspended in 6N HCl (50 mL) and heated to 100-105° C. for 20 hours upon which the solution becomes homogeneous. The solvent was removed under reduce pressure to give 1.8 g of the free acid as the hydrochloride salt in quantitative yield as a white solid.

Step D

The hydrochloride salt of the free acid (1.0 g, 4.9 mmol) was dissolved in anhydrous MeOH (150 mL) saturated with anhydrous HCl gas. The reaction mixture was then heated to reflux for 20 hours. After cooling to room temperature, the solvent was removed under reduced pressure to give a solid. The solid was taken up in methylene chloride (CH₂Cl₂) and washed with saturated NaHCO₃. The organic was separated and dried over MgSO₄, filtered and concentrated to give 0.31 g of the free base of the desired methyl ester in 35% yield as an oil which slowly crystallized into a light brown solid.

MH⁺=180

### Preparative Example 7

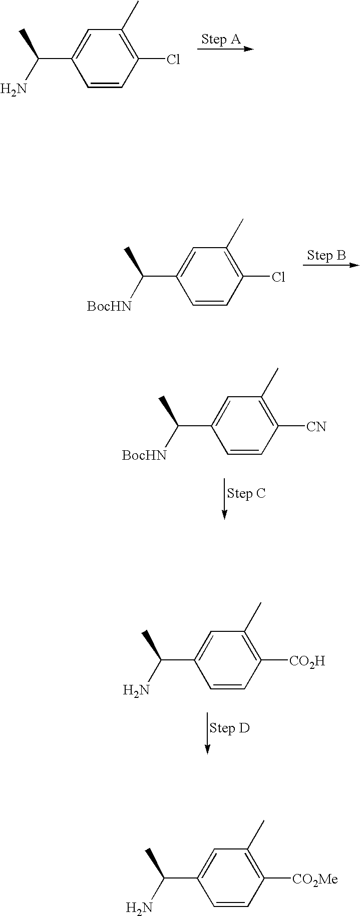 Step A

Commercially available (S)-1-(4-chloro-3-methylophenyl)ethylamine (1.5 mmol) was dissolved in 10 mL dry tetrahydrofuran (THF) and cooled to 0° C. and to this cooled solution was added di-t-butyl dicarbonate (1.5 mmol) dissolved in 1.0 mL of metheylene chloride (CH₂Cl₂) followed by Et₃N (2.8 mL, 5 mmol). The solution was allowed to warm to room temperature. After stirring for 3 hours, the mixture was concentrated and re-dissolved in 100 mL methylene chloride (CH₂Cl₂). This solution was washed with 1N HCl (2×50 mL) and saturated NaHCO₃ (1×50 mL). The CH₂Cl₂ layer was dried over anhydrous MgSO₄, filtered, and concentrated to afford the desired Boc amine compound.

Step B

The desired Boc amine compound (1 mmol), ZnCN₂ (2 mmol), and Pd[PPh₃]₄ (0.1 mmol) were combined under nitrogen and anhydrous dimethylformamide (6 mL) was added. The yellow mixture was heated to 100° C. for 18 h and then concentrated under reduced pressure to afford crude cyano compound which was purified by flash chromatography (20% hexane/CH₂Cl₂) to give the desired cyano compound.

Step C

If the cyano compound (0.5 mmol) were suspended in 6N HCl (10 mL) and heated to 100-105° C. for 20 and the solvent removed under reduce pressure one would produce the free acd as the hydrochloride salt.

Step D

If the hydrochloride salt of the free acid (0.5 mmol) were dissolved in anhydrous MeOH (50 mL) saturated with anhydrous HCl gas and the reaction mixture heated to reflux for 20 hours and then after cooling to room temperature the volatile solvents were removed under reduced pressure one would produce the resulting methyl ester as the hydrochloride salt. If the salt was then taken up in methylene chloride (CH₂Cl₂) and washed with saturated NaHCO₃ and the organic separated and dried over MgSO₄ then filtered and concentrated one would produce the desired methyl ester as the free base of the methyl ester compound.

### Preparative Example 8

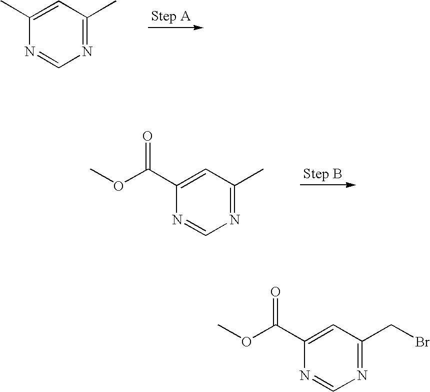 Step A

To a 500 ml round bottom flask was added 400 mL H₂O and KMnO₄ (140 mmoles) and then commercially available 4,6-dimethyl-pyrmidine (35 mmole) and mixture refluxed for 20 hours. The mixture was filtered through celite and then acidified to pH ˜3. The aqueous was then evaporated under reduced pressure to give a solid. To the solid was then added 300 ml of methanol saturated with dry HCl. The mixture was then refluxed for 15 hours. The volatile components of the reaction mixture was then removed under reduced pressure to give an oil. To the oil was then added 150 ml of methylene chloride and organic washed with saturated NaHCO3. The aqueous was removed and then the organic layer was dried over MgSO4, filtered and then the volatile components removed under reduced pressure to give and oil. The oil was purified by column chromatography (SiO₂, 10% either-methylene chloride) to give 6-methyl-pyrimidine-4-carboxylic acid.

Step B

To 6-methyl-pyrimidine-4-carboxylic acid (6.5 mmole) in 25 ml round bottom flask containing a stir bar was added 5 ml of acetic acid and bromine (6.5 mmole) and mixture heated at 75° C. for 5-10 minutes. The volatile components of the reaction mixture was removed under reduced pressure to give an oil. The oil was taken up in 100 ml of methylene chloride and the organic washed with saturated NaHCO3. The organic was separated, dried over MgSO4, filtered and the volatile components removed under reduced pressure to give an oil which was purified by column chromatography (SiO₂, 10% diethyl ether-methylene chloride) to give the desired 6-bromomethyl-pyrimidine-4-carboxylic acid methyle ester.

### Example 1

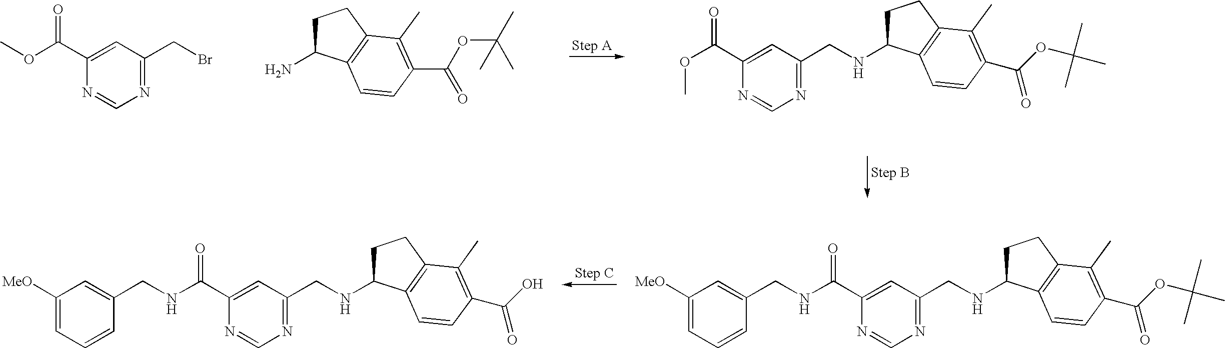 Step A

If to a 5 ml round bottom flask was added 6-bromomethyl-pyrimidine-4-carboxylic acid methyl ester (0.2 mmole) and 1-amino-4-methyl-indan-5-carboxylic acid tert-butyl ester (0.23 mmole) and triethylamine (0.61 mmole) and 0.6 ml of dimethylformamide and mixture were heated at 100° C. for 10 minutes and then if the reaction mixture was concentrated under reduced pressure and the resulting residue purified by column chromatography one would produce the desired 6-[(5-tert-Butoxycarbonyl-4-methyl-indan-1-ylamino)-methyl]-pyrimidine-4-carboxylic acid methyl ester.

Step B

If to a 5 ml thick walled vessel was added 6-[(5-tert-Butoxycarbonyl-4-methyl-indan-1-ylamino)-methyl]-pyrimidine-4-carboxylic acid methyl ester (0.09 mmoles), 3-Methoxy-benzylamine (0.7 mmoles) and 0.5 ml of dimethylformamide and if the reaction mixture was heated via microwaves under closed atmosphere at a temperature of 120° C. for 30 minutes one would produce after purification the desired 1-{[6-(3-Methoxy-benzylcarbamoyl)-pyrimidin-4-ylmethyl]-amino}-4-methyl-indan-5-carboxylic acid tert-butyl ester product.

Step C

If to a 5 ml round bottom flask containing a stir bar was added 1-{[6-(3-Methoxy-benzylcarbamoyl)-pyrimidin-4-ylmethyl]-amino}-4-methyl-indan-5-carboxylic acid tert-butyl ester (0.045 mmoles) and 2 ml of 50% trifluoroacetic acid in methylene chloride and solution stirred for 3 hours one would produce the desired 1-{[6-(3-Methoxy-benzylcarbamoyl)-pyrimidin-4-ylmethyl]-amino)-4-methyl-indan-5-carboxylic acid as the mono trifluoroacetic acid salt.

### Preparative Example 9A

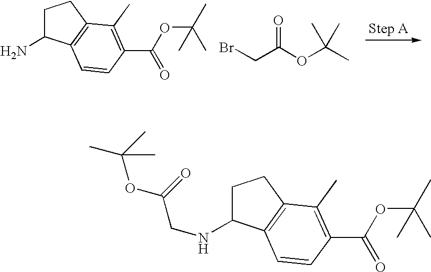 Step A

1-Amino-4-methyl-indan-5-carboxylic acid tert-butyl ester (0.63 mmoles) from Preparative Example 5 (step C) was added to a thick walled vessel containing a stir bar. To the vessel was then added 6 ml of tetrahydrofuran, triethylamine (1.25 mmoles) and Bromo-acetic acid tert-butyl ester (0.63 mmoles) and mixture heated at 80° C. under closed atmosphere for 25 minutes. The volatile components were removed under reduced pressure to give a solid. The solid was purified by column chromatography (SiO2, 20% ether-methylene chloride) to give the desired 1-(tert-Butoxycarbonylmethyl-amino)-4-methyl-indan-5-carboxylic acid tert-butyl ester.

### Example 2

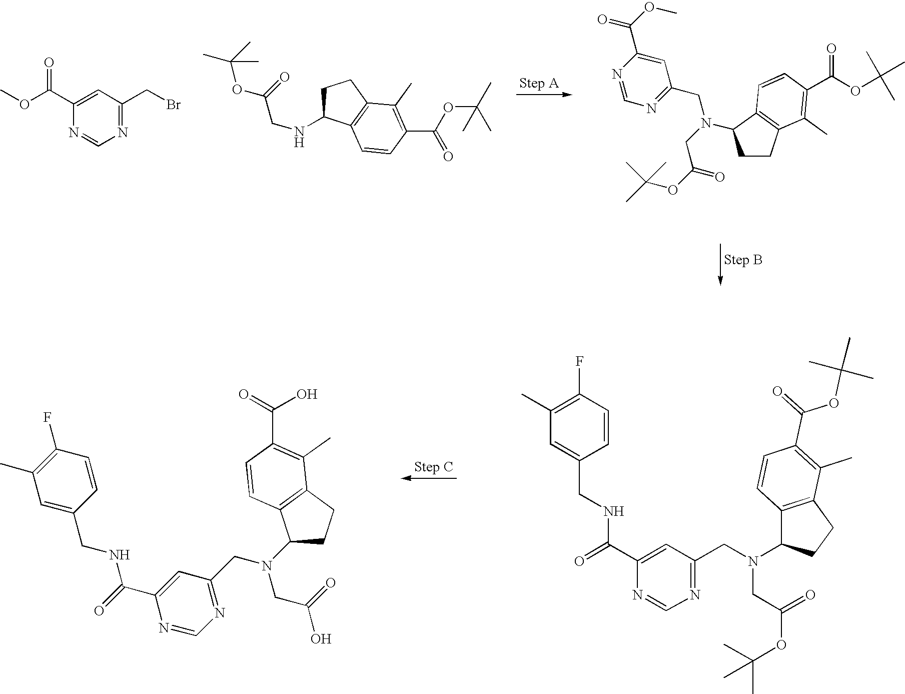 Step A

If to a 5 ml round bottom flask was added 6-Bromomethyl-pyrimidine-4-carboxylic acid methyl ester and 1-(tert-Butoxycarbonylmethyl-amino)-4-methyl-indan-5-carboxylic acid tert-butyl ester from Preparative Example 10 and triethylamine and 0.5 ml of dimethylformamide and mixture heated at 80° C. for 15 minutes and then concentrated under reduced pressure and one would produce the desired 6-{[tert-Butoxycarbonylmethyl-(5-tert-butoxycarbonyl-4-methyl-indan-1-yl)-amino]-methyl}-pyrimidine-4-carboxylic acid methyl ester.

Step B

If to a 5 ml thick walled vessel was added 6-{[tert-Butoxycarbonylmethyl-(5-tert-butoxycarbonyl-4-methyl-indan-1-yl)-amino]-methyl}-pyrimidine-4-carboxylic acid methyl ester and 3-methyl-4-fluoro-benzylamine in 0.5 ml of dimethylformamide and mixture was heated via microwaves under closed atmosphere to a temperature of 80° C. for 30 minutes one would get the desired give 1-{tert-Butoxycarbonylmethyl-[6-(4-fluoro-3-methyl-benzylcarbamoyl)-pyrimidin-4-ylmethyl]-amino}-4-methyl-indan-5-carboxylic acid tert-butyl ester product.

Step C

If to a 5 ml round bottom flask containing a stir bar was added 1-{tert-Butoxycarbonylmethyl-[6-(4-fluoro-3-methyl-benzylcarbamoyl)-pyrimidin-4-ylmethyl]-amino}-4-methyl-indan-5-carboxylic acid tert-butyl ester from step B and 40% trifluoroacetic acid in methylene chloride and solution stirred for 24 then after the resulting oil was triturated with diethyl either one would produce the desired 1-{Carboxymethyl-[6-(4-fluoro-3-methyl-benzylcarbamoyl)-pyrimidin-4-ylmethyl]-amino}-4-methyl-indan-5-carboxylic acid product.

### Example 3

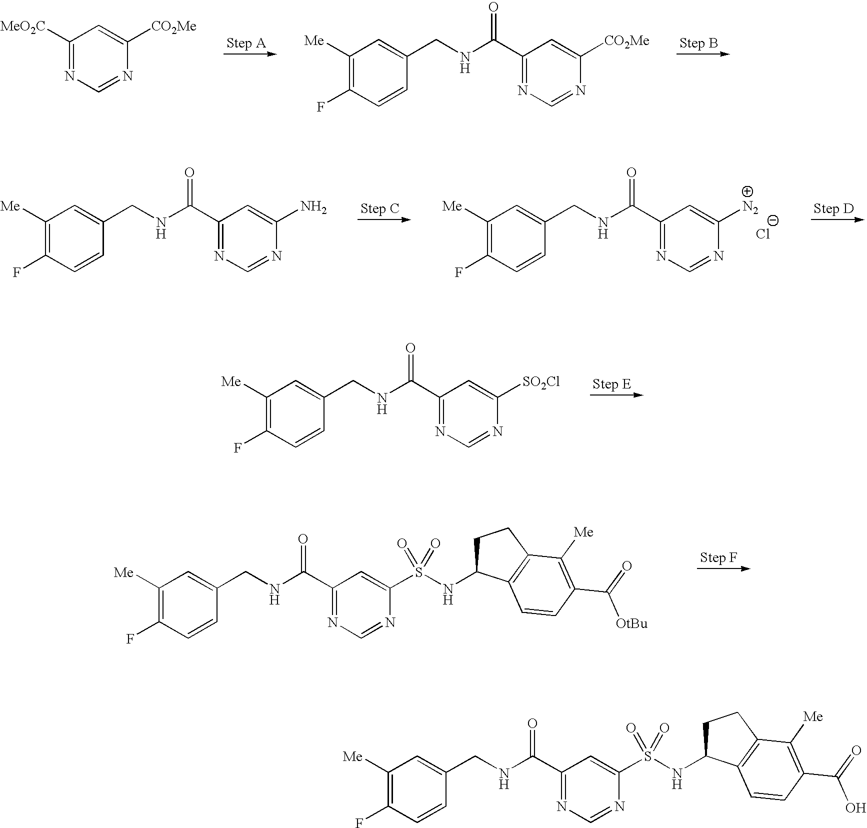 Step A

If one were to dissolve commercially available pyrimidine-4,6-dicarboxylic acid dimethyl ester (1.00 g, 5.10 mmol) and 4-fluoro-3-methylbenzylamine (0.71 g, 5.10 mmol) in DMF (20 mL) and heat to 60° C. overnight, then concentrate and chromatograph the product one would obtain the monoamide.

Step B

If one were to dissolve the monoamide (150 mg, 0.49 mmol) from above in LiOH (0.50 mL of a 1M aqueous solution) and MeOH (2 mL), and stir at room temperature until complete hydrolysis, then quench with HCl (0.50 mL of a 1M aqueous solution), and concentrate one would obtain an acid. If one took the resulting acid, diphenylphosphoryl azide (270 mg, 1.0 mmol), and triethylamine (0.14 mL, 1.0 mmol) in t-butanol (2 mL) and heated to reflux, then concentrated and the resulting intermediate was treated with HCl (4M solution in dioxane) and concentrated one would obtain the amine.

Step C

If the amine (96 mg, 0.37 mmol) was added portionwise to a cooled solution of concentrated aqueous HCl (1 mL) followed by addition of a solution of sodium nitrite (27 mg, 0.39 mmol) the diazonium compound would be obtained.

Step D

If one were to take a solution of the diazonium from Step C and add it to a solution of copper(II) chloride (15 mg, 0.11 mmol) in glacial acetic acid (2 mL) which was saturated with sulfur dioxide, then poured into cold water and the product filtered one would obtain the sulfonyl chloride.

Step E

If one dissoled the sulfonyl chloride from Step D (76 mg, 0.22 mmol) in THF (1 mL) and triethylamine (92 μL, 0.66 mmol) and the appropriate amine (42 g, 0.24 mmol) was added, then concentrated and chromatographed one would obtain the sulfonamide.

Step F

If the product from Step E (90 mg, 0.19 mmol) was dissolved in a 40% TFA/CH₂Cl₂ (1 mL) solution and stirred at room temperature for 1 h, then water (0.2 mL) was added and the reaction was concentrated one would obtain the product.

### Example 4

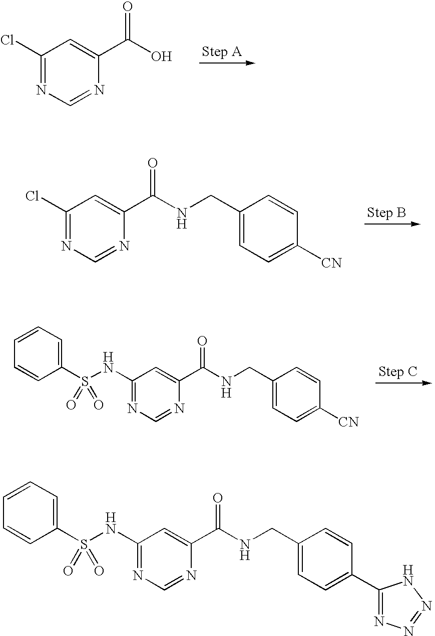 Step A

4-Cyanolbenzylamine (132 mg, 1 mmol), 6-chloro-4-pyrimidine carboxylic acid (158 mg, 1 mmol) were mixed with EDCI (216 mg, 1.1 mg) and HOBt (149 mg, 1.1 mmol) in dichloromethane (5 mL). The reaction was stirred at room temperature overnight. Normal aqueous workup and pourification with ethyl acetate and hexane (gradient) to give product as white solid (225.6 mg). MS (M+H): 272

Step B

A dry round bottom flask was charged with Pd(II) acetate (2.4 mg, 0.02 mmol, 2 mol %), Xantphos (18 mg, 0.03 mmol), pyrimidine chloride (136 mg, 0.5 mmol), phenylsulfonamide (103 mg, 0.6 mmol), cesium cabonate (244 mg, 0.75 mmol), evacuated and backfilled with argon; this evacuation/backfill sequence was repeated one additional time. 1,4-Dioxane (1 mL) were added through the septum. The mixture was refluxed overnight. The reaction mixture was then cooled to room temperature, The aqueous layer was washed with ethyl acetate (8 mL). The combined organic layers were extracted with sodium hydroxide (1 N, 4×3 mL) and were discarded. The product went into the aqueous layer in this case. The combined aqueous layers were acidified with hydrochloric acid (1 N, 3 mL) and extracted with ethyl acetate (5 mL). This ethyl acetate layer was dried over sodium sulfate and concentrated to give the crude product, which was purified by silica gel chromatography (ethyl acetate and hexane, gradient) to give product as white solid (41 mg); MS (M+H): 408.

Step C

To the mixture of cyano diamide (41 mg, 0.1 mmol) and azidotrimethylsilane (27 uL, 0.2 mmol) in toluene (3 mL) was added dibutyltin oxide (2.5 mg, 0.01 mmol). The suspension was heated to reflux overnight, and then concentrated to dryness. The product was washed with methylene chloride (2×1 mL) to give pure product as off white solid (30 mg);

MS (M+H): 451

### Example 5

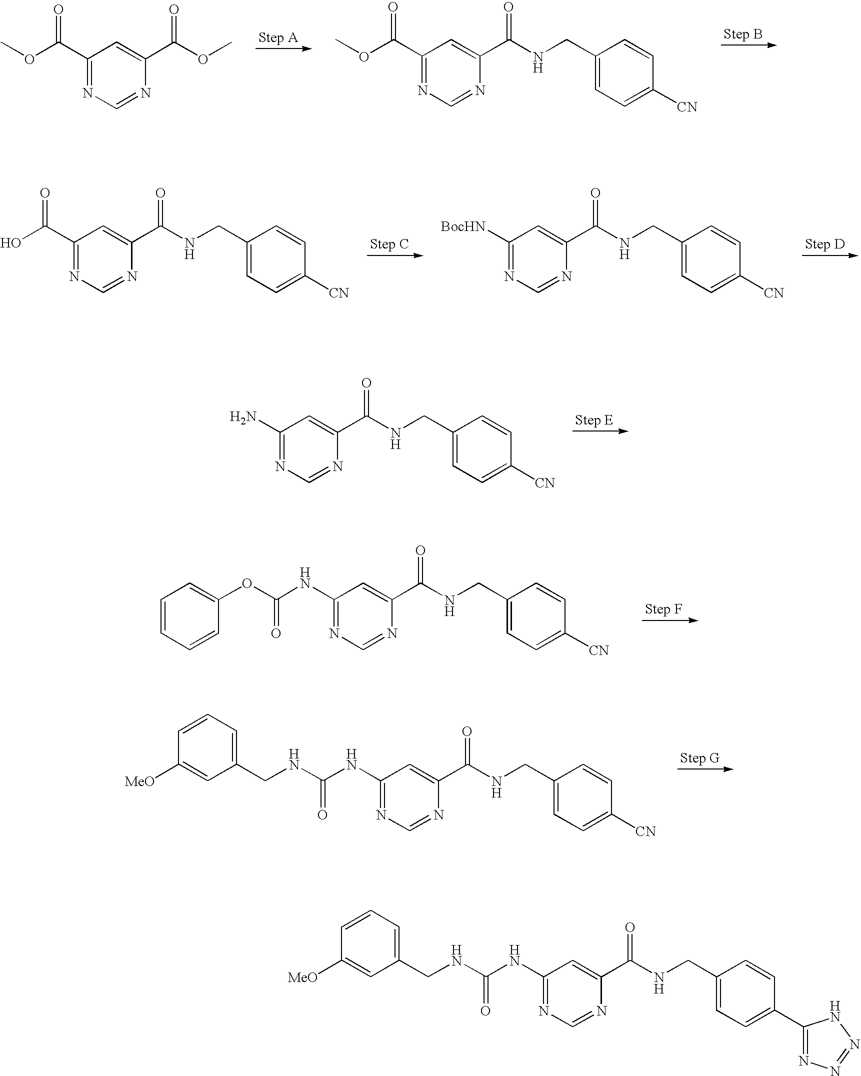 Step A

4-Cyanolbenzylamine (1.1 g, 8.33 mmol), 4,6-pyrimidine dicarboxylic acid methyl ester (1.77 g, 8.33 mmol) were dissolved in N,N-dimethylformamide (20 mL). The reaction was stirred 60° C. overnight and concentrated. The brown solid was purification with ethyl acetate and hexane (gradient) to give product as light brown solid (1.18 g, 48% yield).

Step B

At 0° C., the above ester (1.18 g, 4 mmol) in THF (20 mL) was added aqueous lithium hydroxide (4 mL, 1M). After 1 h, The mixture was neutralized with sodium hydrogen sulfate (2M, 2 mL) and concentrated. The resulting solid was added THF (50 mL), filter the solution through a bed of celite and concentrated again to give white solid (1.06 g), which is pure enough for the next reaction.

Step C

The mixture of above acid (25 mg, 0.1 mmol), diphenylphosphinoazide (43 μL, 0.2 mmol) and triethyl amine (31 μL, 0.22 mmol) in t-butanol (3 mL) was refluxed for 5 h. The solution was concentrated to dryness. The crude was used without further purification

Step D

To the above solid was added hydrogen chloride in dioxane (4 N, 2 mL). After 1 h, the solution was diluted with ether (5 mL), and the resulting solid was collected and rinsed with ether (5 mL). The product was dried in vaaco (28.3 mg, 97% for 2 steps).

Step E & F

To the above 6-aminopyridine (29 mg, 0.1 mmol) in pyridine (1 mL) was added chlorophenylformate (38 μL, 0.3 mmol). The mixture was heated to 100° C. After 2 h, the reaction is cooled down and concentrated to dryness.

The solid was dissolved in DMSO (1 mL) and 3-methoxylbenzylamine (14 mg, 0.1 mmol) was added. The reaction was stirred for another 1 h, concentrated and purified by silica gel chromatography to give product as white solid (28 mg, 67% yield).

Step G

To the mixture of cyano diamide (68.5 mg, 0.163 mmol) and azidotrimethylsilane (90 μL, 0.7 mmol) in toluene (2 mL) was added dibutyltin oxide (8.1 mg, 0.035 mmol). The suspension was heated to reflux overnight, and then concentrated to dryness. The product was washed with methylene chloride (2×1 mL) to give pure product as off white solid (52 mg, 69% yield); MS (M+H): 463.

### Example 6A

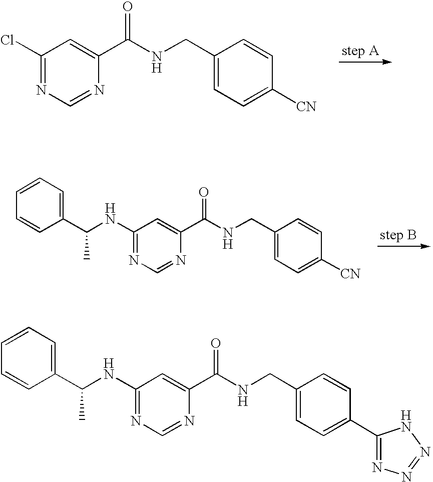 Step A

The mixture of chloropyrimidine (55 mg, 0.2 mmol) and (S)-phenyl ethylamine (0.2 mL) wa heated to 100° C. for 3 h. The reaction was complete. The product was concentrated and purified by silica gel chromatography (Methylene chloride/methanol 20/1) to give product (64 mg, 90% yield). MS (M+H): 358.

Step B

To the mixture of cyano diamide (64 mg, 0.18 mmol) and azidotrimethylsilane (56 μL, 0.4 mmol) in toluene (2 mL) was added dibutyltin oxide (8.1 mg, 0.035 mmol). The suspension was heated to reflux overnight, and then concentrated to dryness. The product was washed with methylene chloride (2×1 mL) to give pure product as off white solid (52 mg, 71% yield); MS (M+H): 401.

### Example 6B

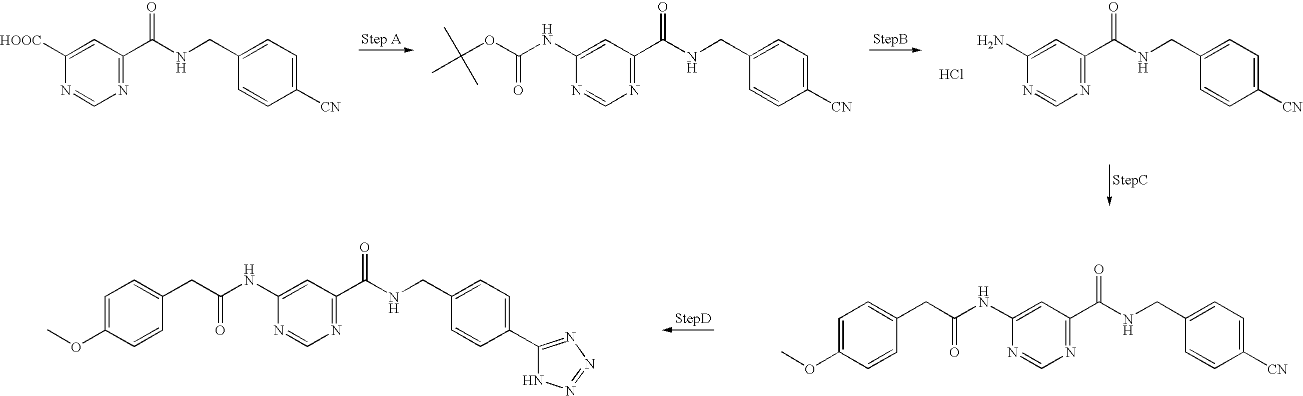 Step A

6-(4-Cyanobenzylcarbamoyl)pyrimidine-4-carboxylic acid (101 mg, 0.36 mmol) and Diphenylphosphoryl azide (DPPA, 197 mg, 0.72 mmol) were dissolved in t-butanol (10 mL) and triethylamine (0.11 mL, 0.78 mmol). The mixture was stirred at 82° C. for 16 h and concentrated under reduced pressure. The residue was purified by silica gel chromatography (methanol/dichloromethane) to afford tert-butyl 6-(4-cyanobenzylcarbamoyl)pyrimidin-4-ylcarbamate as white solid (68.1 mg, 54%). [MH]⁺=354.2.

Step B

Tert-butyl 6-(4-cyanobenzylcarbamoyl)pyrimidin-4-ylcarbamate (220 mg) was added to HCl (4 N in dioxane, 5 mL). The reaction was stirred for 15 h and ether was added. White solid was collected through filtration to afford N-(4-cyanobenzyl)-6-aminopyrimidine-4-carboxamide hydrochloride (177.4 mg, 98%). [MH]⁺=254.1

Step C

To a solution of N-(4-cyanobenzyl)-6-aminopyrimidine-4-carboxamide hydrochloride (17.8 mg, 0.061 mmol) in pyridine (0.5 mL) was added 2-(4-methoxyphenyl)acetyl chloride (114 mg). The mixture was stirred at 60° C. for 15 h and purified by silica gel chromatography (methanol/dichloromethane) to afford title compound as white solid (24 mg, 98%). [MH]⁺=402.1.

Step D

The corresponding carbonitrile from Step C (23 mg), Bu₂SnO (3.2 mg) and TMSN₃ (43 microliters) were added to dioxane (0.5 mL). The mixture was heated up to 100° C. and stirred for 24 h. The solvent was evaporated in vacuo. The residue was purified by silica gel chromatography (methanol/dichloromethane) to afford title compound as solid (23.5 mg, 92%). [MH]⁺=445.2.

### Examples 7-14

Following a similar procedure as that described in Example 6B, Step C, except using the acid chloride indicated in Table 1 below, the following compounds were prepared.

TABLE 1

| | | | Yield |
|---|---|---|---|
| Ex. # | Acid chloride | Product | MS |
| 7 | 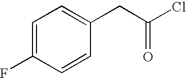 | 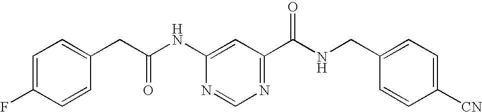 | 82% [MH]⁺ = 390.4 |
| 8 | 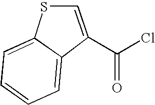 | 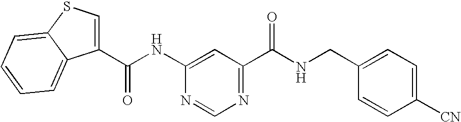 | 92% [MH]⁺ = 414.1 |
| 9 | 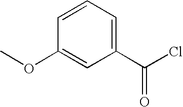 | 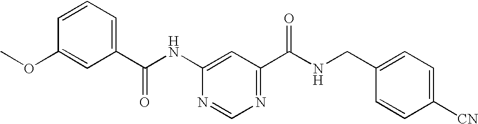 | 42% [MH]⁺ = 388.2 |
| 10 | 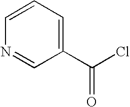 | 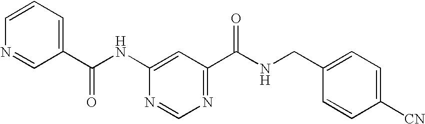 | 73% [MH]⁺ = 359.2 |
| 11 | 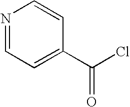 | 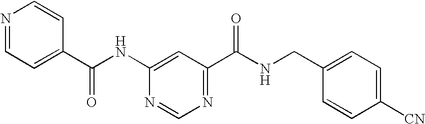 | 90% [MH]⁺ = 359.1 |
| 12 | 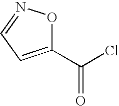 | 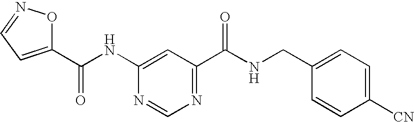 | 30% [MH]⁺ = 349.2 |
| 13 | 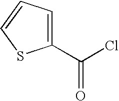 | 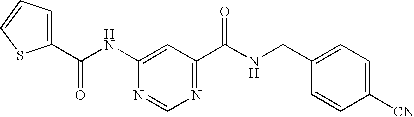 | 84% [MH]⁺ = 364.1 |
| 14 | 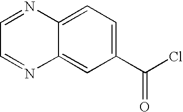 | 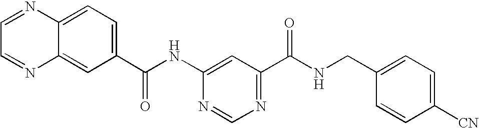 | 80% [MH]⁺ = 410.2 |

### Examples 15-23

Following a similar procedure as that described in Example 6B, Step D, except using the carbonitrile indicated in Table 2 below, the following compounds were prepared.

TABLE 2

| Ex. | | | Yield |
|---|---|---|---|
| # | carbonitrile | Product | MS |
| 15 | 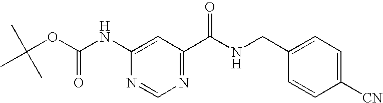 | 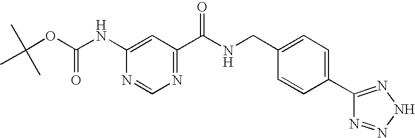 | 65% [MH]⁺ =397.2 |
| 16 | 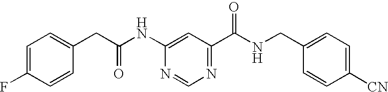 | 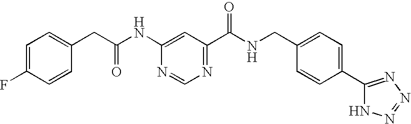 | 57% [MH]⁺ =433.2 |
| 17 | 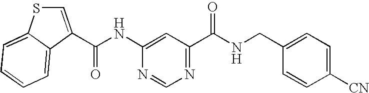 | 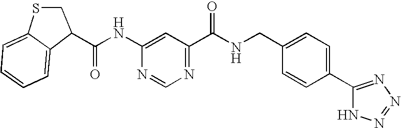 | 72% [MH]⁺ =457.4 |
| 18 | 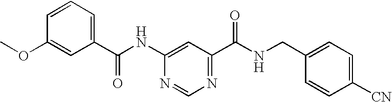 | 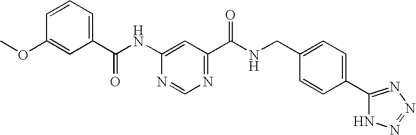 | 78% [MH]⁺ =431.2 |
| 19 | 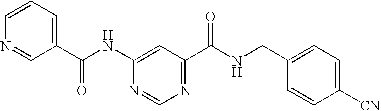 | 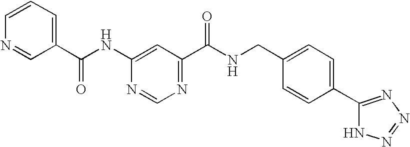 | 85% [MH]⁺ =402.1 |
| 20 | 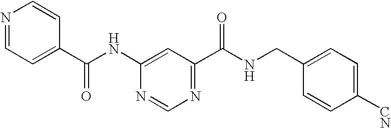 | 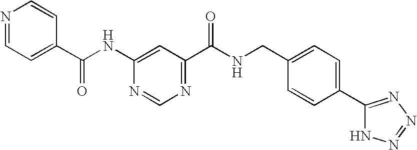 | 85% [MH]⁺ =402.1 |
| 21 | 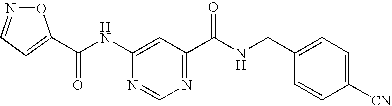 | 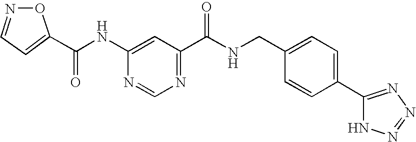 | 80% [MH]⁺ =392.2 |
| 22 | 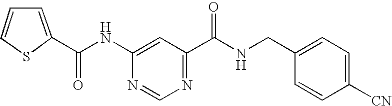 | 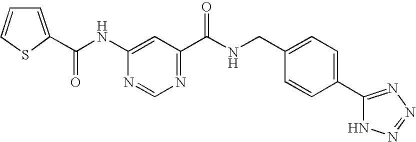 | 92% [MH]⁺ =407.2 |
| 23 | 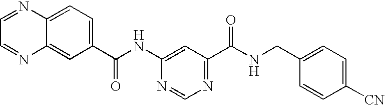 | 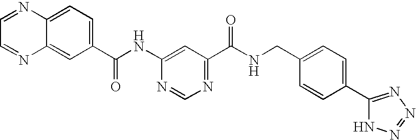 | 53% [MH]⁺ =453.2 |

### Example 24

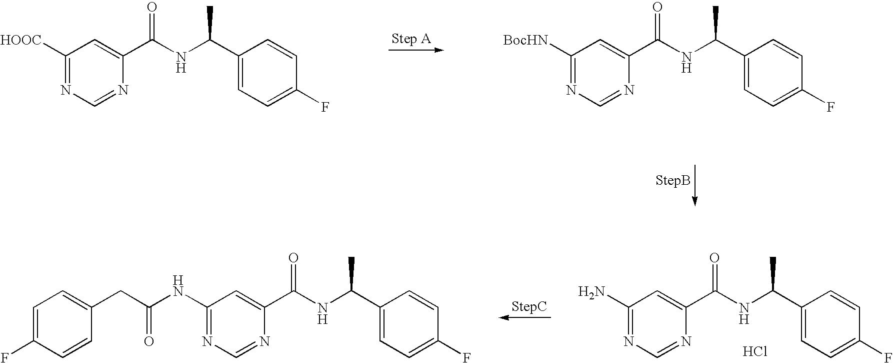 Step A

6-((S)-1-(4-Fluorophenyl)ethylcarbamoyl)pyrimidine-4-carboxylic acid (512 mg, 1.77 mmol) and Diphenylphosphoryl azide (DPPA, 974 mg, 3.54 mmol) were dissolved in t-butanol (20 mL) and triethylamine (0.54 mL, 3.89 mmol). The mixture was stirred at 100° C. for 16 h and concentrated under reduced pressure. The residue was purified by silica gel chromatography (methanol/dichloromethane) to afford tert-butyl 6-((S)-1-(4-fluorophenyl)ethylcarbamoyl)pyrimidin-4-ylcarbamate as white solid (278.4 mg, 44%).

[MH]⁺=361.1.

Step B

tert-Butyl 6-((S)-1-(4-fluorophenyl)ethylcarbamoyl)pyrimidin-4-ylcarbamate (276 mg) was added to HCl (4 N in dioxane, 6 mL). The reaction was stirred for 15 h and ether was added. White solid was collected through filtration to afford 6-amino-N—((S)-1-(4-fluorophenyl)ethyl)pyrimidine-4-carboxamide hydrochloride (220 mg, 97%). [MH]⁺=261.1

Step C

To a solution of 6-amino-N—((S)-1-(4-fluorophenyl)ethyl)pyrimidine-4-carboxamide hydrochloride (139 mg, 0.468 mmol) in pyridine (1 mL) was added 2-(4-fluorophenyl)acetyl chloride (242 mg). The mixture was stirred at 60° C. for 15 h and purified by silica gel chromatography (methanol/dichloromethane) to afford title compound as white solid (102.5 mg, 55%). [MH]⁺=397.2.

### Example 25

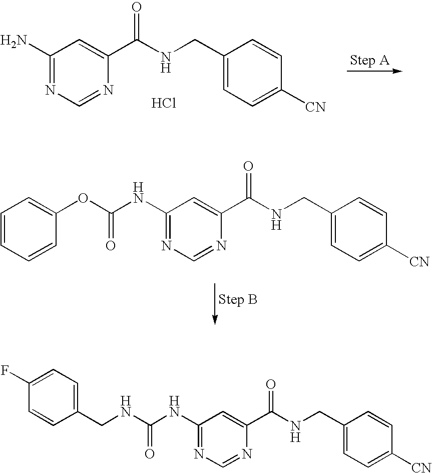 Step A

To a solution of N-(4-cyanobenzyl)-6-aminopyrimidine-4-carboxamide hydrochloride (40.3 mg, 0.139 mmol) in pyridine (0.8 mL) was added phenyl chloroformate (108 mg). The mixture was stirred at 60° C. for 15 h and purified by silica gel chromatography (methanol/dichloromethane) to afford phenyl 6-(4-cyanobenzylcarbamoyl)pyrimidin-4-ylcarbamate as white solid (17.3 mg, 33%). [MH]⁺=374.2.

Step B

To a solution of N phenyl 6-(4-cyanobenzylcarbamoyl)pyrimidin-4-ylcarbamate (17 mg) in DMSO (1 mL) was added 4-florobenzylamine (8.5 mg). The mixture was stirred at room temperature for 1 h and diluted with ethyl acetate. The organic solution was washed with HCl (1 N aq), water, NaOH (1 N aq.) and brine, dried over MgSO4, concentrated and purified by silica gel chromatography (methanol/dichloromethane) to afford title compound as white solid (3.3 mg, 18%). [MH]⁺=405.1.

### Preparative Example 9B

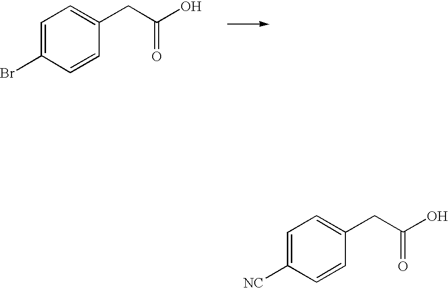

A solution of commercially available 4-bromophenyl-acetic acid (1.5 g), Zn(CN)₂ (492 mg) and Pd(PPh₃)₄ (403 mg) in DMF was stirred at 80° C. for 18 h. The mixture was concentrated and purified by column chromatography (silica, chloroform/MeOH, 95:5) to afford the title compound (470 mg; 42%). [MH]⁺=162.

### Preparative Example 10

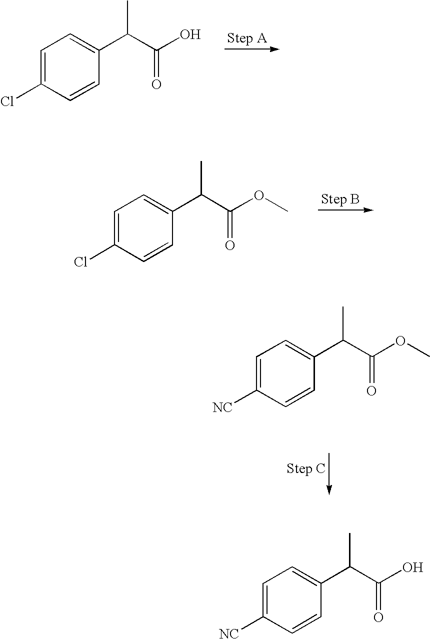 Step A

A solution of commercially available 2-(4-chlorophenyl)-propionic acid (5.5 g) and ion exchange resin IR-120(H⁺) in dry MeOH (200 mL) was stirred under reflux for 24 h, filtered and the solvent was evaporated to dryness, cooled and the formed precipitate was filtered off to afford the title compound (5.84 g; 99%) as a colourless oil. [MH]⁺=199.

Step B

To a solution of the title compound from step A above (2.55 g), Pd₂(dba)₃ (235 mg), dppf (285 mg), Zn(CN)₂ (900 mg) and zinc (100 mg) in dry, degassed DMA (20 mL) was heated under argon at 120° C. overnight. The mixture was evaporated and dissolved in EtOAc, washed with 1N HCl and brine, dried and purified by chromatography (silica, cyclohexane/EtOAc 95:5 to 4:1) to afford the title compound (672 mg; 28%) as a yellow oil.

[MH]⁺=190.

Step C

The title compound from Step B above (672 mg) was dissolved in THF (10 mL) and a solution of lithium hydroxide monohydrate (300 mg) in water (10 mL) was added. The mixture was vigorously stirred for 2¼ h, acidified with 10% citric acid and extracted with EtOAc. The organic layer was dried (MgSO₄) and concentrated to afford the title compound (623 mg; quant.) as bright yellow crystals. [MH]⁺=176.

### Preparative Example 11

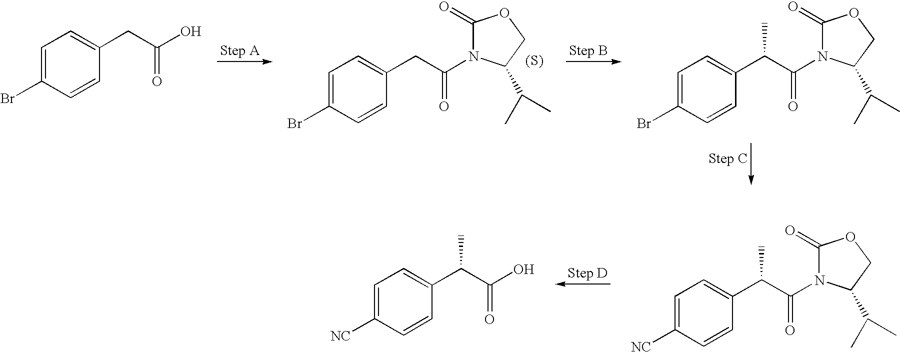 Step A

A solution of commercially available 4-bromophenyl-acetic acid (5.13 g), (4S)-(−)-4-isopropyl-2-oxazolidinone (3.08 g), pivaloyl chloride (3.4 mL) and NEt₃ (7.6 mL) in dry toluene was stirred at 110° C. for 18 h. Then additional 4-bromophenyl-acetic acid (5 g), pivaloyl chloride (3.4 mL) and NEt₃ (10 mL) was added and the mixture was refluxed for additional 24 h. The mixture was diluted with EtOAc, washed with 1N HCl, brine, 2N NaOH, saturated aqueous NH₄Cl solution and brine, dried and purified by column chromatography (silica, cyclohexane/EtOAc 9:1 to 4:1) to afford the title compound (4.04 g; 52%) as colourless needles after crystallization from EtOAc/pentane. [MH]⁺=326/328.

Step B

A solution of the intermediate from step A above (3.50 g) in dry THF was cooled to −70° C. under argon, then LiHMDS (11.6 mL) was added in portions at −70° C. and the solution was allowed to reach 0° C., stirred at 0° C. for ½ h. Then methyl iodide (830 μL) was added and the solution was stirred for 1 h, evaporated and purified by column chromatography (silica, cyclohexane/EtOAc 9:1 to 85:15) to afford the title compound (2.77 g; 76%) as a colourless oil. [MH]⁺=340/342.

Step C

A solution of the intermediate from step B above (2.77 g), Zn(CN)₂ (718 mg) and Pd(PPh₃)₄ (471 mg) in dry, degassed DMF (20 mL) was stirred at 80° C. for 18 h under argon. The mixture was concentrated, diluted with EtOAc, washed with 0.5N HCl and brine, dried and purified by column chromatography (silica, cyclohexane/EtOAc 4:1) to afford the title compound (1.68 g; 72%). [MH]⁺=287.

Step D

The title compound from Step C above (537 mg) was dissolved in THF (30 mL), cooled to −10° C. and a solution of lithium hydroxide monohydrate (79 mg) in water (10 mL) and H₂O₂ (1 mL, 35%) was added. The mixture was vigorously stirred for ¾ h, acidified with 10% citric acid and extracted with EtOAc. Purification by column chromatography (silica, cyclohexane/EtOAc 6:4 to 1:1) afforded the title compound (255 mg; 58%) as a colourless oil. [MH]⁺=176.

### Preparative Example 12

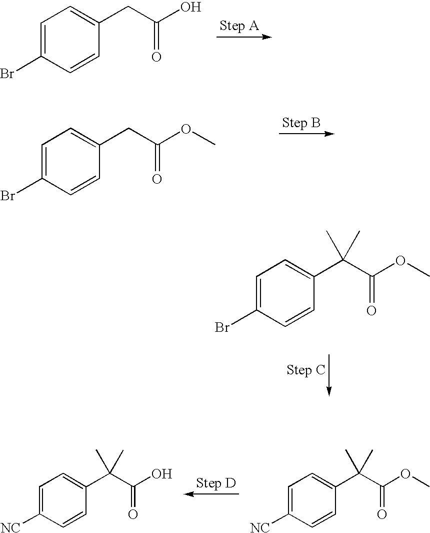 Step A

A solution of commercially available (4-bromophenyl)-acetic acid (4.05 g) and ion exchange resin IR-120(H⁺) in dry MeOH (100 mL) was stirred at 65° C. overnight. After addition of NEt₃ the resin was filterd and the solution evaporated to dryness to afford the title compound (4.46 g; quant.) as a colourless oil. [MH]⁺=229/231.

Step B

To a solution of the intermediate from step A above (4.46 g) in dry DMF (40 mL) was added NaH (1.8 g) in portions and then slowly methyl iodide (47 mL) under cooling. The mixture was stirred overnight, acidified with 6N HCl, diluted with EtOAc, washed with water and brine, dried and purified by column chromatography (silica, cyclohexane/EtOAc 1:0 to 95:5) to afford the title compound (4.05 g; 84%) as a light red coloured liquid. [MH]⁺=257/259.

Step C

A solution of the intermediate from step B above (4.05 g), Zn(CN)₂ (1.3 g) and Pd(PPh₃)₄ (456 mg) in dry, degassed DMF (30 mL) was stirred at 80° C. overnight under argon. The mixture was concentrated, diluted with EtOAc, washed with 0.5N HCl and brine, dried and purified by column chromatography (silica, cyclohexane/EtOAc 9:1 to 4:1) to afford the title compound (3.05 g; 95%) as a clear oil. [MH]⁺=204.

Step D

The title compound from Step C above (3.05 g) was dissolved in THF (90 mL) and a solution of lithium hydroxide monohydrate (1.26 g) in water (30 mL) was added. The mixture was vigorously stirred for 4 h, acidified with 10% citric acid and extracted with EtOAc. The organic layer was dried (MgSO₄) and concentrated to afford the title compound (2.73 g; 96%) as colourless crystals. [MH]⁺=190.

### Preparative Example 13

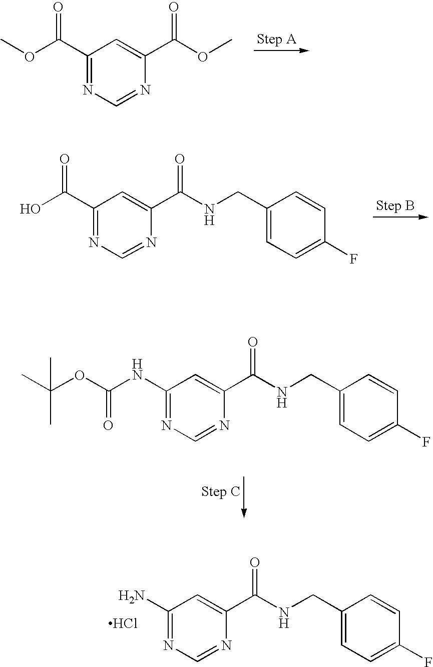 Step A

A solution of commercially available pyrimidine-4,6-dicarboxylic acid dimethyl ester (1.61 g) and 4-fluorobenzylamine (1.23 g) in DMF (30 mL) was stirred at 60° C. for 24 h. The solvent was evaporated to dryness, the residue dissolved in THF/H₂O 1:1 (10 mL) and LiOH H₂O (314 mg) was added. The resulting mixture was stirred at rt for 2 h and H₂O (50 mL) was added. The reaction mixture was extracted with DCM and acidified with concentrated HCl. The formed precipitate was filtered off and washed with H₂O to afford the title compound (1.147 g; 51%). [MH]⁺=276.

Step B

To a solution of the title compound from step A above (1.14 g) in tert.-butanol (40 mL) triethylamine (922 mg) and diphenylphosphoryl azide (2.28 g) were added. The mixture was heated at reflux for 24 h, concentrated and purified by column chromatography (silica, cyclohexane/EtOAc, 7:3) to afford the title compound (1.07 g; 75%). [MH]⁺=347.

Step C

The title compound from step B above (1.07 g) was dissolved in a 4M solution of HCl in dioxane (10 mL) and stirred at room temperature for 2 d. The solvent was evaporated to afford the title compound (680 mg; 100%) as a colourless solid. [MH]⁺=247.

### Preparative Example 14-16

Following a similar procedure as described in Preparative Example 13, except using the amine listed in the table below, the following compound were prepared.

| Prep. Ex. # | Amine | product | yield |
|---|---|---|---|
| 14 | 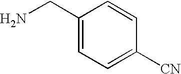 | 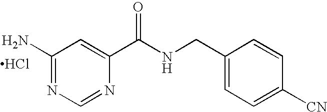 | n.d. [M + H] = 254 |
| 15 | 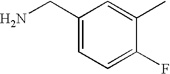 | 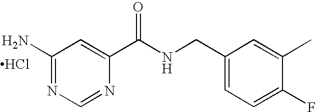 | 26% [M + H] = 261 |
| 16 | 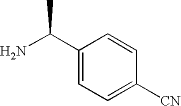 | 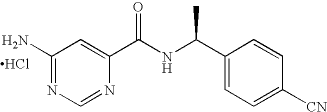 | n.d. [M + H] = 268 |

### Preparative Example 17-29

If one were to follow a similar procedure as described in Preparative Example 13, except using the amine listed in the table below, the following compound would be obtained.

| Prep. Ex. # | amine | product |
|---|---|---|
| 17 | 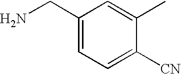 | 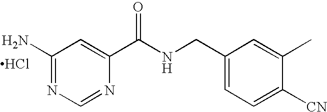 |
| 18 | 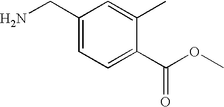 | 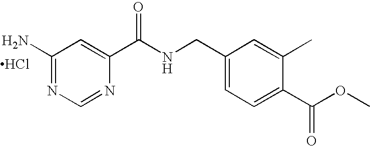 |
| 19 | 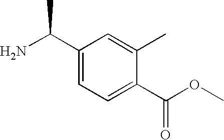 | 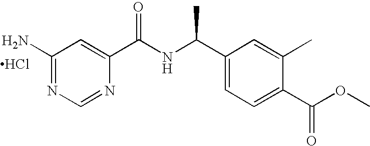 |
| 20 | 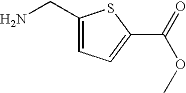 | 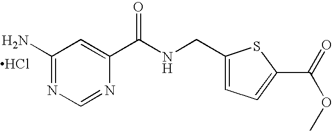 |
| 21 | 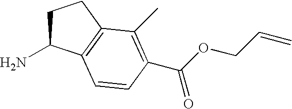 | 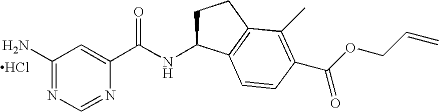 |
| 22 | 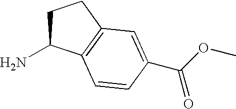 | 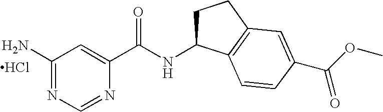 |
| 23 | 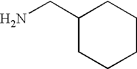 | 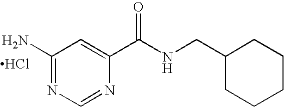 |
| 24 | 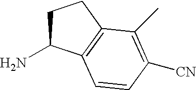 | 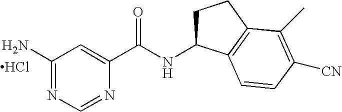 |
| 25 | 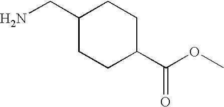 | 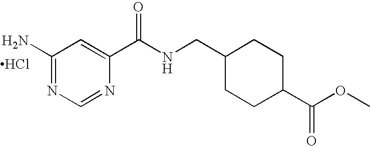 |
| 26 | 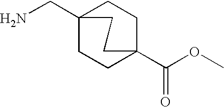 | 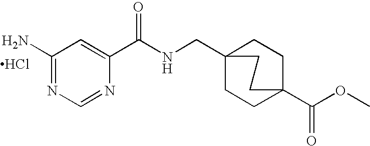 |
| 27 | 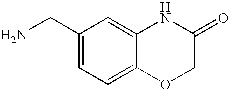 | 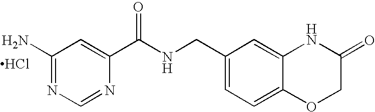 |
| 28 | 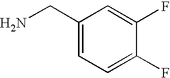 | 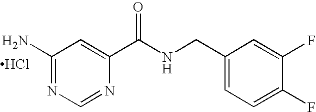 |
| 29 | 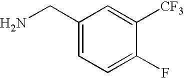 | 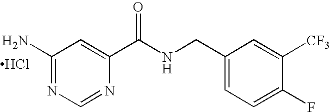 |

### Example 26

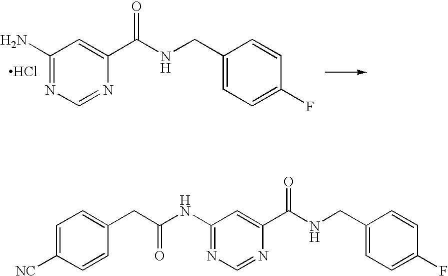

The title compound from Preparative Example 2000 (470 mg) was dissolved in dichloromethane (15 mL). DMF (10 μL) and oxalylchloride (1.27 mL of a 2M solution in dichloromethane) were added and the mixture was stirred at rt for 2.5 h. The mixture was concentrated to afford the crude acid chloride. The title compound from Preparative Example 2100, Step C (170 mg) was added as a solution in pyridine (5 mL). The mixture was stirred at 60° C. for 16 h, concentrated and dissolved in ethyl acetate. The organic layer was washed with saturated ammonium chloride and brine, dried (MgSO₄), concentrated and purified by column chromatography (silica, cyclohexane/EtOAc, 4:6) to afford the title compound (69 mg; 30%). [MH]⁺=390.

### Examples 27-42

Following a similar procedure as described in Example 26, except using the amine and acid listed in the table below, the following compound were prepared.

| Ex. | amine | | |
|---|---|---|---|
| # | acid | product | yield |
| 27 | 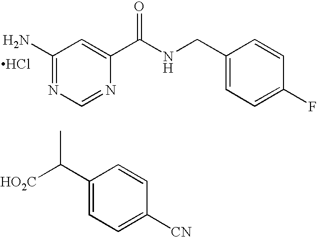 | 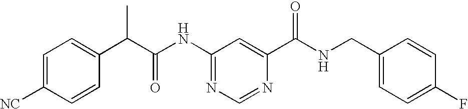 | 88% [MH]⁺ =404 |
| 28 | 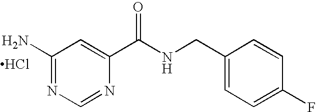 | 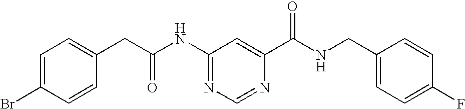 | 81% [MH]⁺ =443/445 |
| | 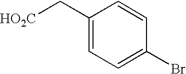 | | |
| 29 | 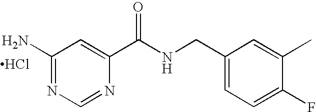 | 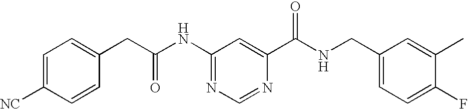 | 55% [MH]⁺ =404 |
| | 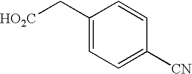 | | |
| 30 | 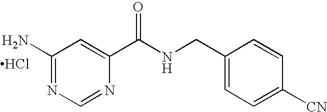 | 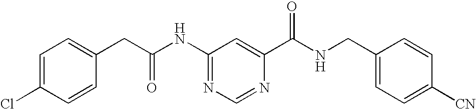 | 72% [MH]⁺ =406 |
| | 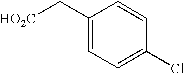 | | |
| 31 | 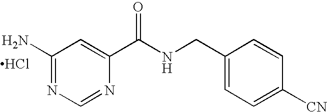 | 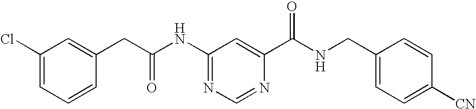 | 96% [MH]⁺ =406 |
| | 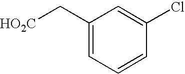 | | |
| 32 | 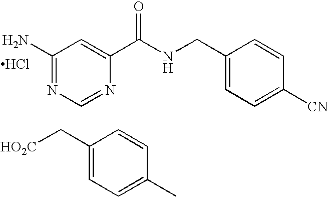 | 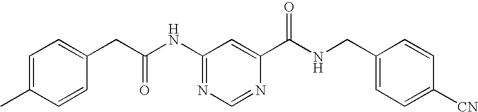 | 76% [MH]⁺ = 386 |
| 33 | 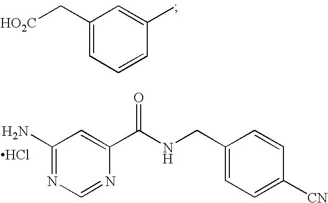 | 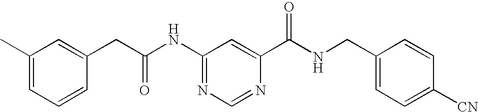 | 82% [MH]⁺ =386 |
| 34 | 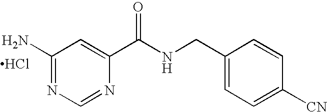 | 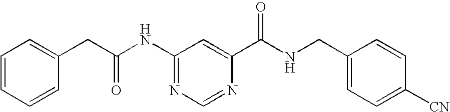 | 69% [MH]⁺ =372 |
| | 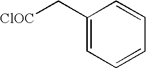 | | |
| 35 | 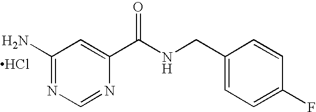 | 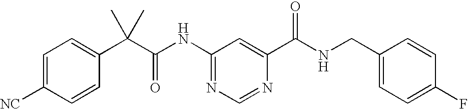 | 45% [MH]⁺ =418 |
| | 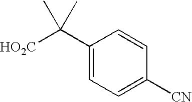 | | |
| 36 | 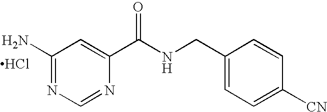 | 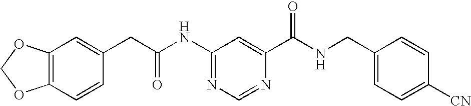 | 57% [MH]⁺ =416 |
| | 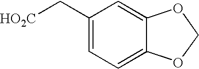 | | |
| 37 | 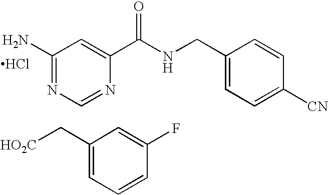 | 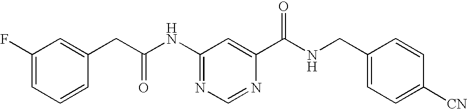 | 48% [MH]⁺ =390 |
| 38 | 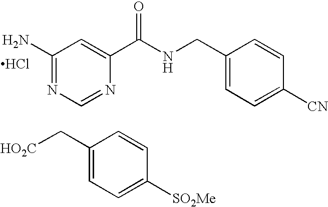 | 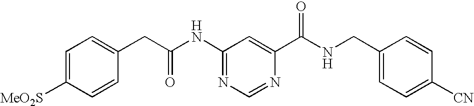 | n.d. [MH]⁺ =450 |
| 39 | 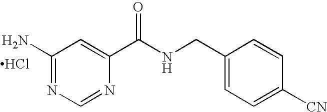 | 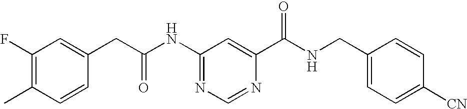 | 59% [MH]⁺ =404 |
| | 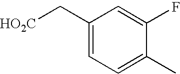 | | |
| 40 | 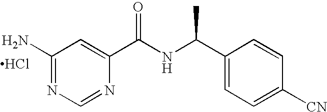 | 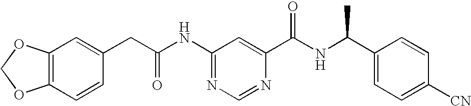 | 56% [MH]⁺ =430 |
| | 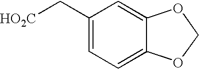 | | |
| 41 | 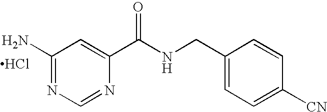 | 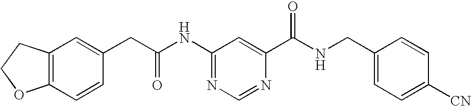 | 95% [MH]⁺ =414 |
| | 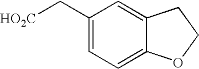 | | |
| 42 | 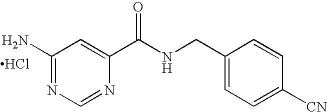 | 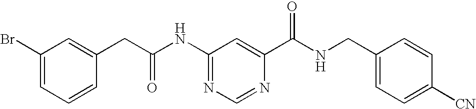 | 68% [MH]⁺ =450/452 |
| | 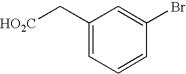 | | |

### Example 43

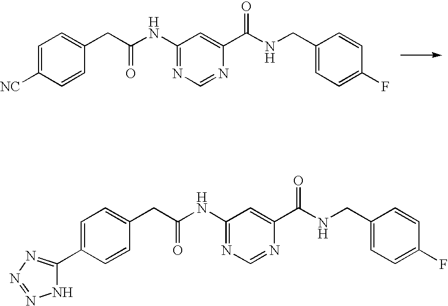

The title compound from Example 26 (60.0 mg), dibutyltinoxide (8.0 mg) and trimethylsilyl azide (266 mg) were dissolved in toluene (5 mL). The mixture was stirred at 100° C. for 24 h, concentrated and purified by column chromatography (silica, chloroform/methanol 8:2) to afford the title compound (34.4 mg; 52%). [MH]⁺=433.

### Examples 44-59

Following a similar procedure as described in Preparative Example 43, except using the nitrile listed in the table below, the following compound were prepared.

| Ex. | | | |
|---|---|---|---|
| # | nitrile | product | yield |
| 44 | 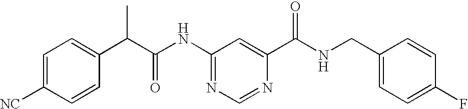 | 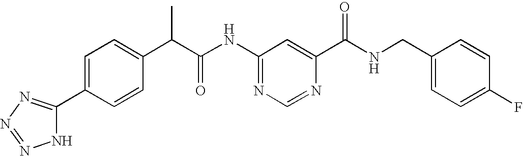 | 46% [MH]⁺ =447 |
| 45 | 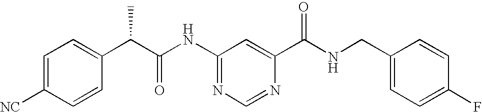 | 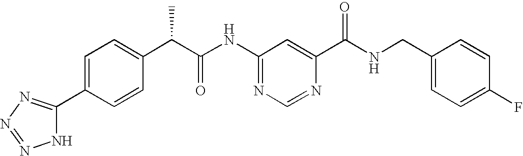 | 86% [MH]⁺ =447 |
| 46 | 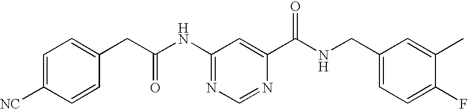 | 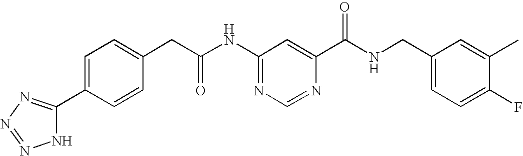 | 59% [MH]⁺ =447 |
| 47 | 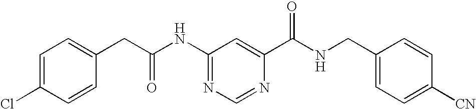 | 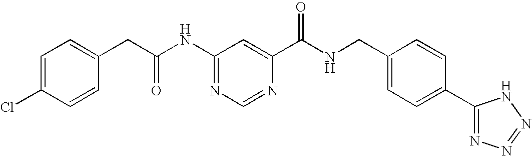 | 59% [MH]⁺ =449 |
| 48 | 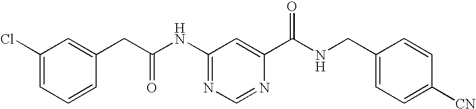 | 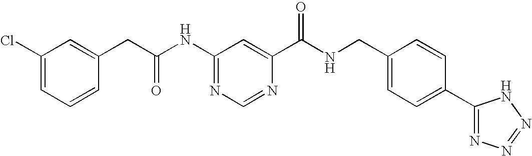 | 73% [MH]⁺ =449 |
| 49 | 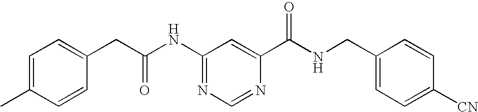 | 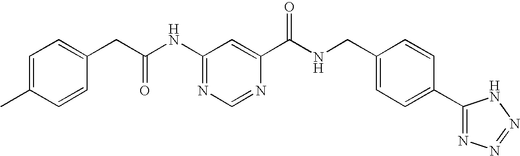 | 61% [MH]⁺ =429 |
| 50 | 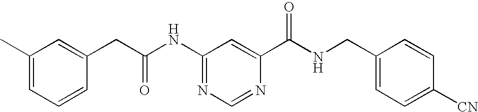 | 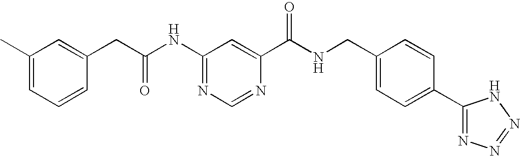 | 69% [MH]⁺ =429 |
| 51 | 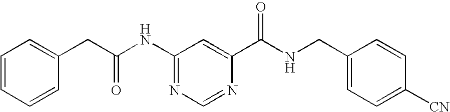 | 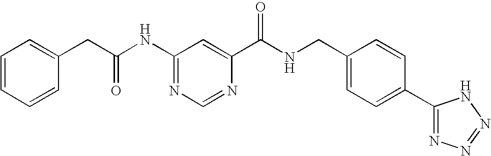 | 76% [MH]⁺ =415 |
| 52 | 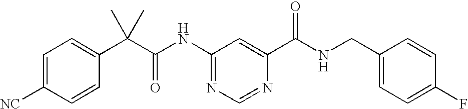 | 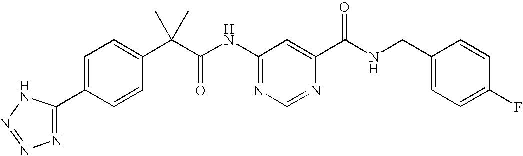 | 63% [MH]⁺ =461 |
| 53 | 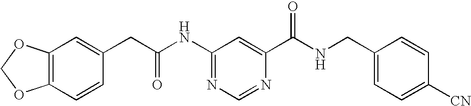 | 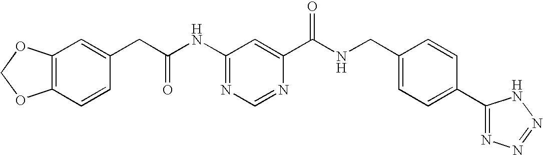 | 41% [MH]⁺ =459 |
| 54 | 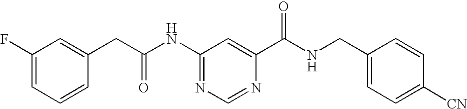 | 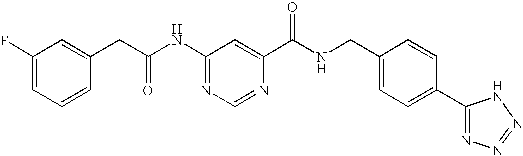 | 69% [MH]⁺ =433 |
| 55 | 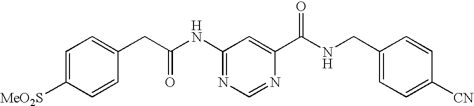 | 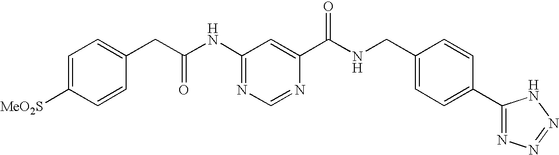 | 13% [MH]⁺ =493 |
| 55 | 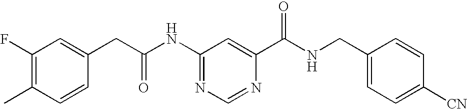 | 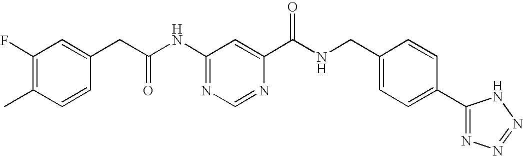 | 48% [MH]⁺ =447 |
| 57 | 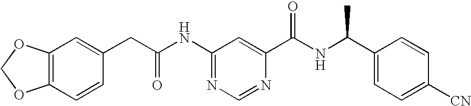 | 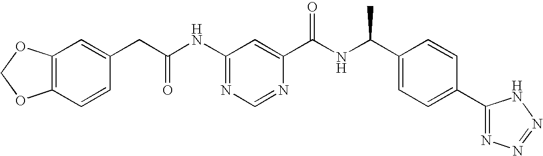 | 59% [MH]⁺ =447 |
| 58 | 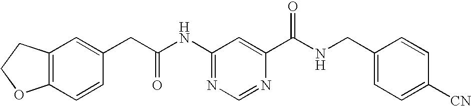 | 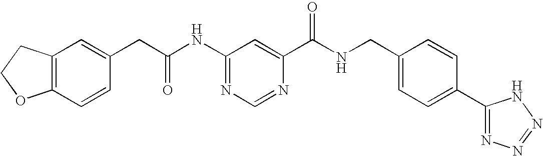 | 41% [MH]⁺ =459 |
| 59 | 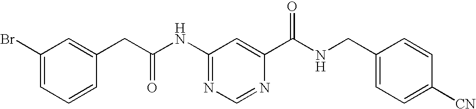 | 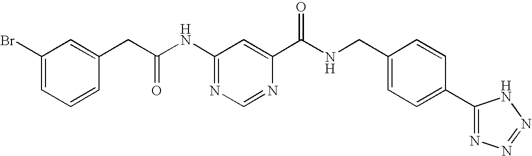 | 82% [MH]⁺ =494 |

### Examples 60-77

If one were to follow a similar procedure as described in Example 26, except using the amine and acid listed in the table below, the following compound would be obtained.

| Ex. | amine | |
|---|---|---|
| # | acid | product |
| 60 | 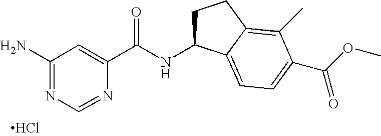 | 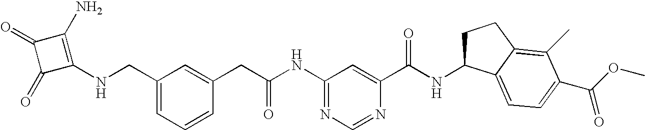 |
| | 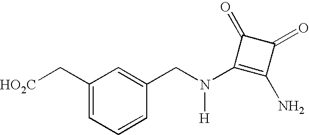 | |
| 61 | 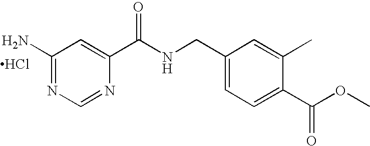 | 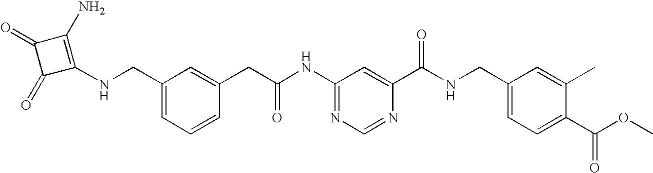 |
| | 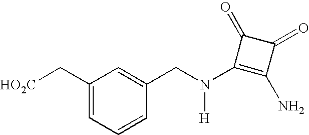 | |
| 62 | 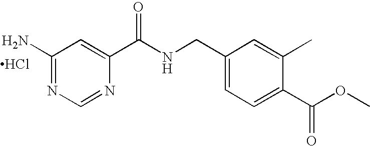 | 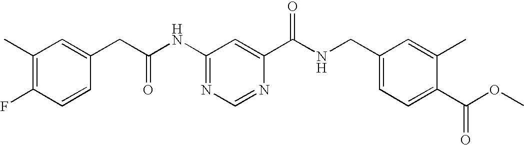 |
| | 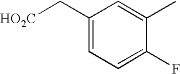 | |
| 63 | 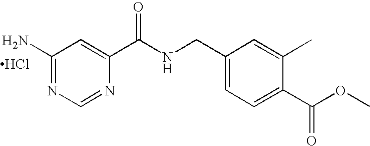 | 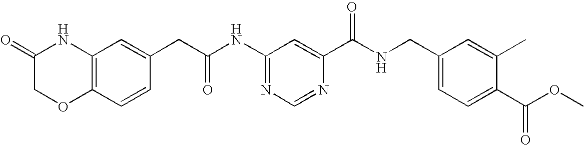 |
| | 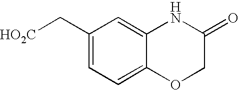 | |
| 64 | 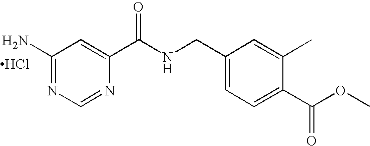 | 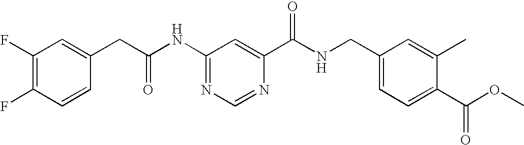 |
| | 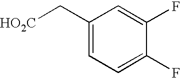 | |
| 65 | 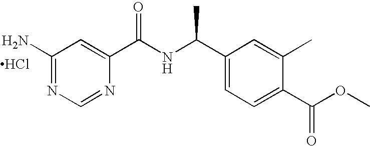 | 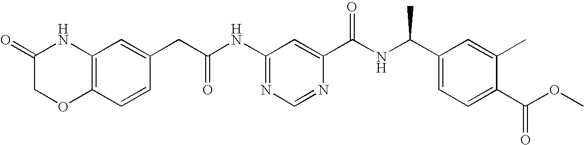 |
| | 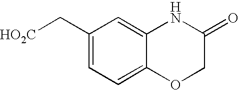 | |
| 66 | 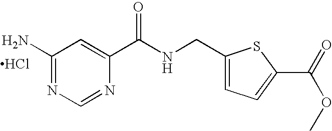 | 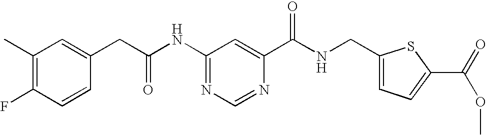 |
| | 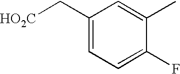 | |
| 66 | 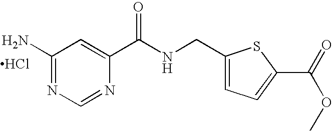 | 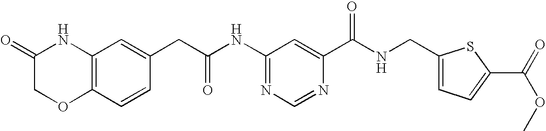 |
| | 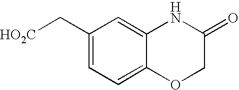 | |
| 67 | 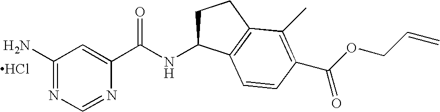 | 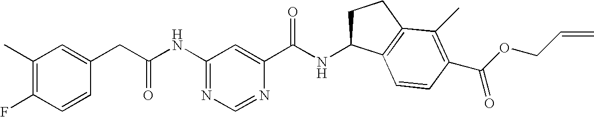 |
| | 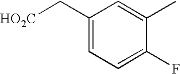 | |
| 68 | 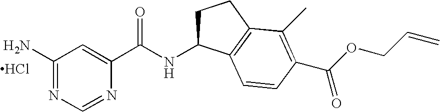 | 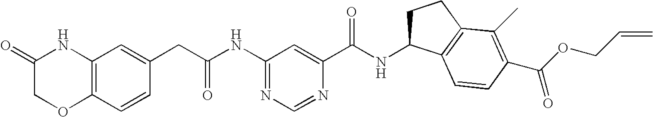 |
| | 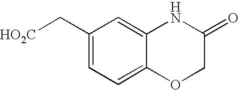 | |
| 69 | 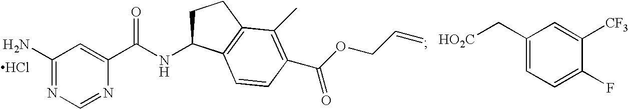 | 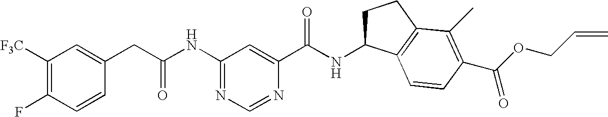 |
| 70 | 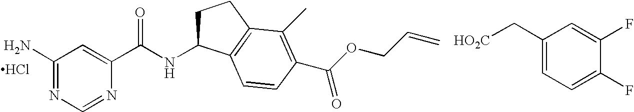 | 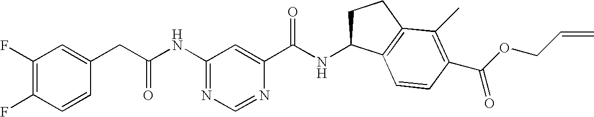 |
| 71 | 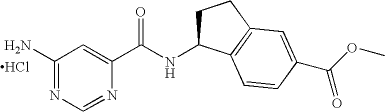 | 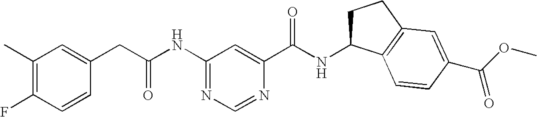 |
| | 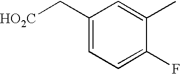 | |
| 72 | 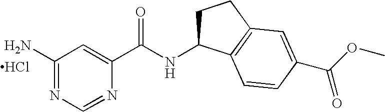 | 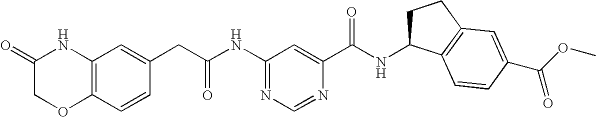 |
| | 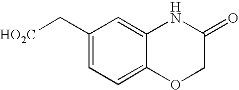 | |
| 73 | 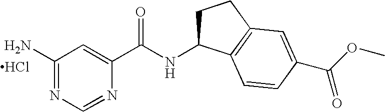 | 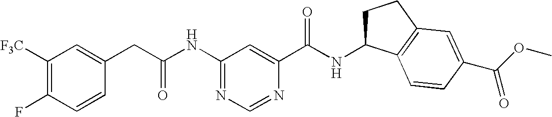 |
| | 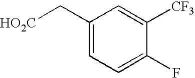 | |
| 74 | 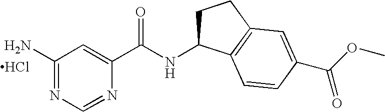 | 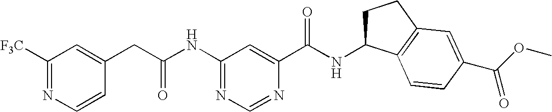 |
| | 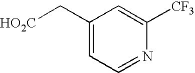 | |
| 75 | 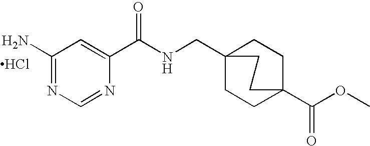 | 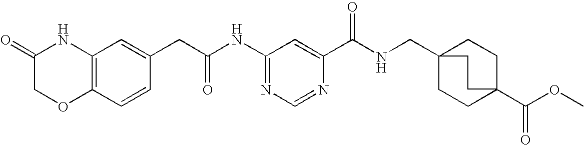 |
| | 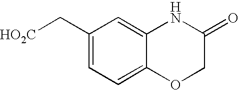 | |
| 76 | 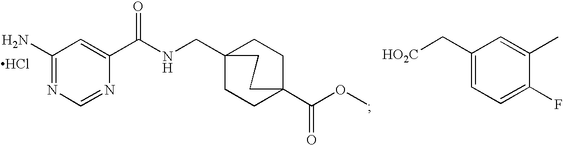 | 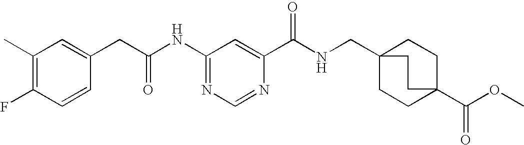 |
| 77 | 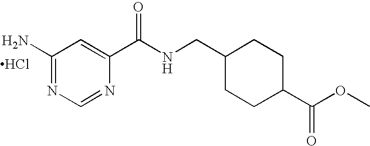 | 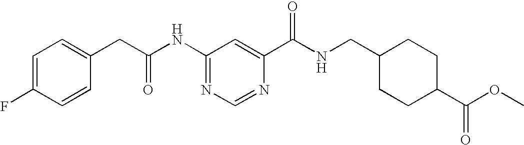 |
| | 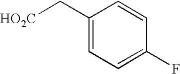 | |

### Examples 2300-2319

If one were to deprotect the esters as described in Greene T. W. and Wuts G. M, Protective groups in organic synthesis, Wiley, New York, 1999, the following compound would be obtained.

| Ex. | |
|---|---|
| # | ester |
| 2300 | 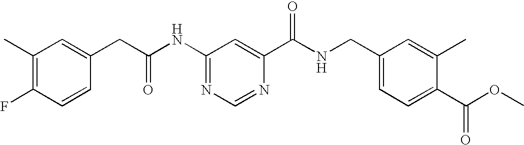 |
| 2301 | 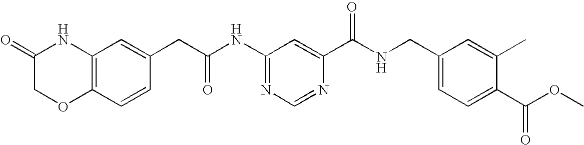 |
| 2303 | 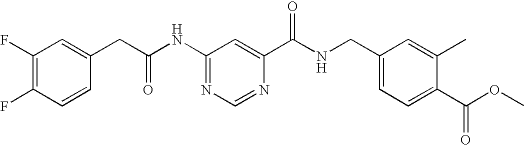 |
| 2304 | 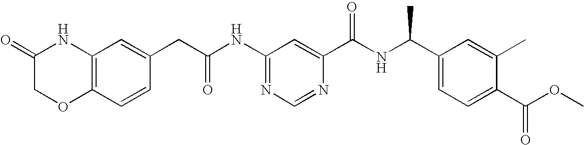 |
| 2305 | 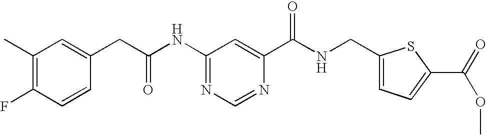 |
| 2306 | 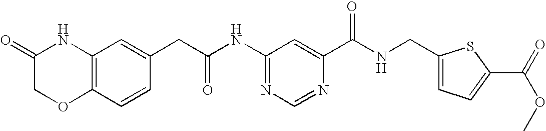 |
| 2307 | 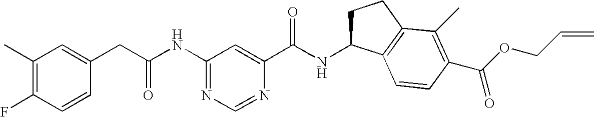 |
| 2308 | 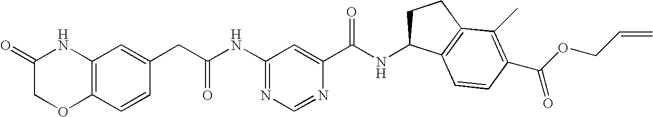 |
| 2309 | 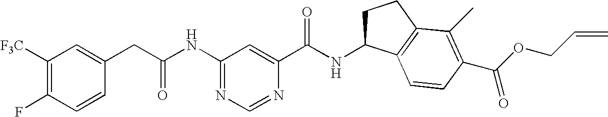 |
| 2310 | 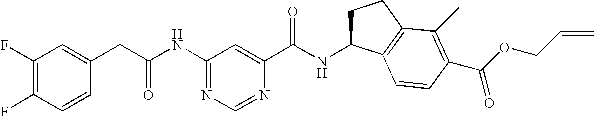 |
| 2311 | 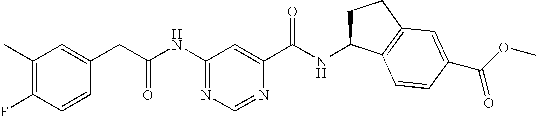 |
| 2312 | 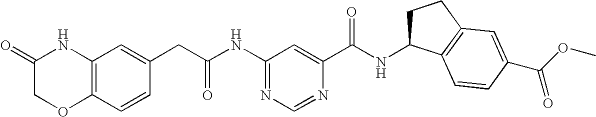 |
| 2313 | 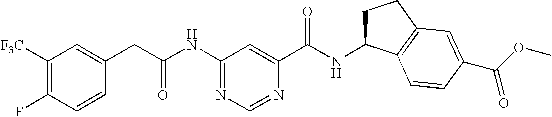 |
| 2314 | 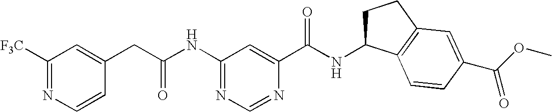 |
| 2315 | 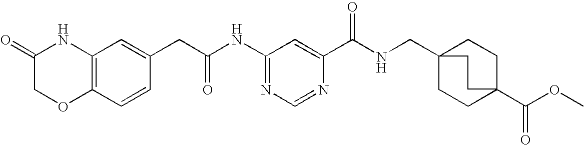 |
| 2316 | 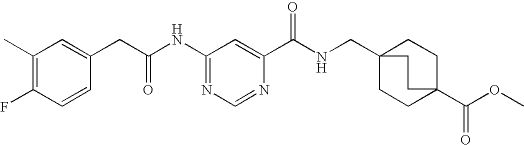 |
| 2317 | 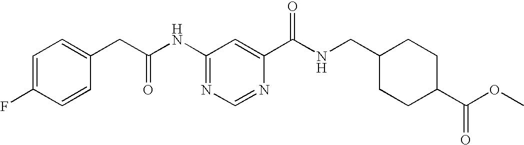 |
| 2318 | 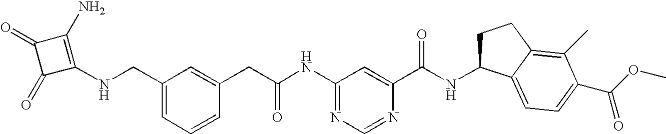 |
| 2319 | 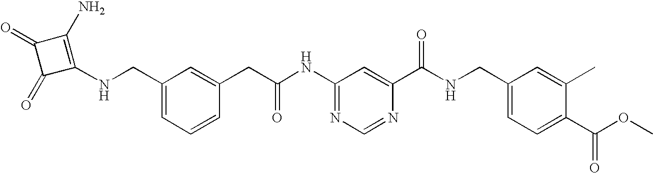 |

| Ex. | |
|---|---|
| # | acid |
| 2300 | 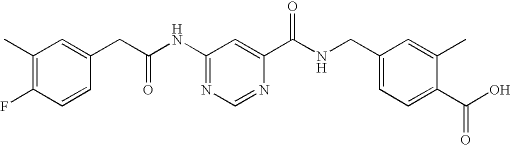 |
| 2301 | 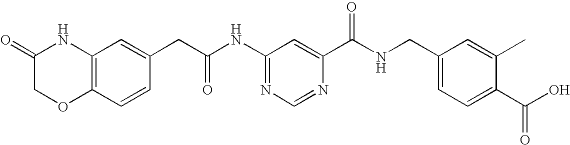 |
| 2303 | 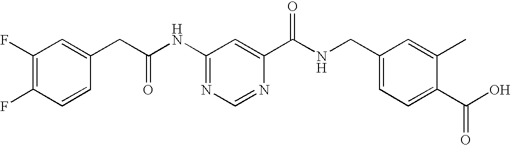 |
| 2304 | 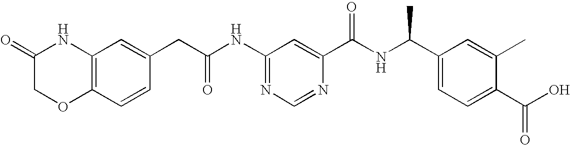 |
| 2305 | 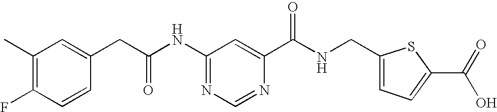 |
| 2306 | 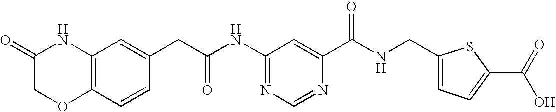 |
| 2307 | 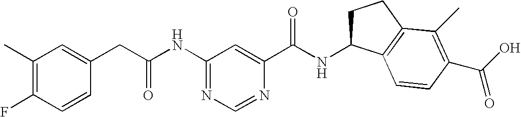 |
| 2308 | 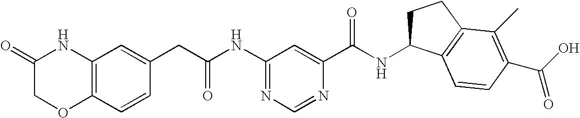 |
| 2309 | 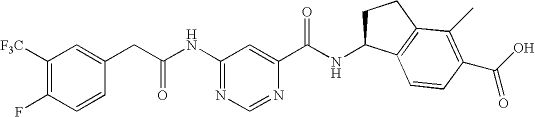 |
| 2310 | 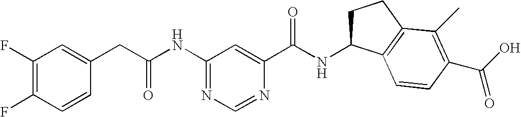 |
| 2311 | 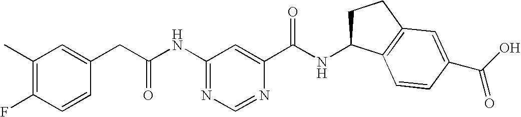 |
| 2312 | 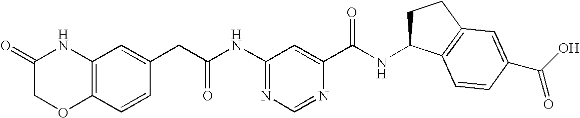 |
| 2313 | 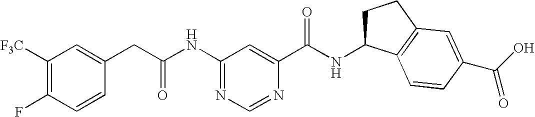 |
| 2314 | 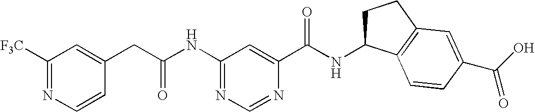 |
| 2315 | 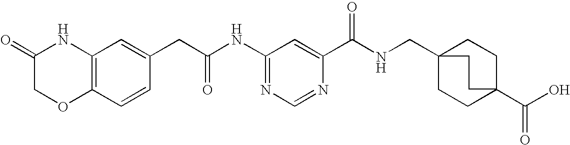 |
| 2316 | 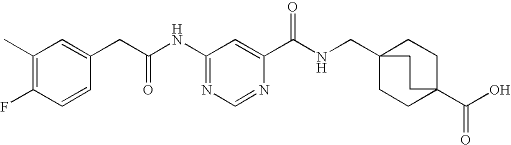 |
| 2317 | 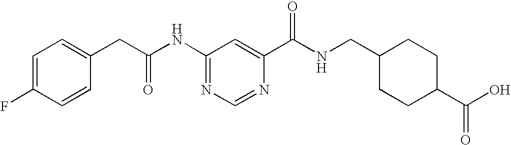 |
| 2318 | 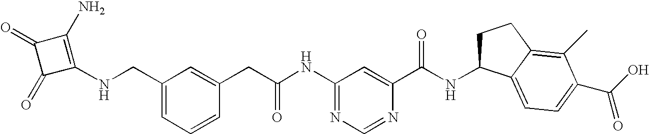 |
| 2319 | 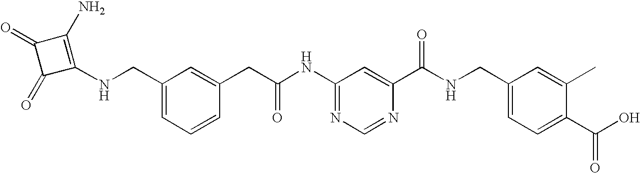 |

### Example 3000

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of catalytic domain of MMP-13 enzyme (produced by Alantos) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of MMP-13 fluorescent substrate (Calbiochem, Cat. No. 444235). The time-dependent increase in fluorescence is measured at the 320 nm excitation and 390 nm emission by automatic plate multireader. The IC₅₀ values are calculated from the initial reaction rates.

### Example 3001

### Assay for Determining MMP-3 Inhibition

The typical assay for MMP-3 activity is carried out in assay buffer comprised of 50 mM MES, pH 6.0, 10 mM CaCl₂ and 0.05% Brij-35. Different concentrations of tested compounds are prepared in assay buffer in 50 μL aliquots. 10 μL of a 100 nM stock solution of the catalytic domain of MMP-3 enzyme (Biomol, Cat. No. SE-109) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of NFF-3 fluorescent substrate (Calbiochem, Cat. No. 480455). The time-dependent increase in fluorescence is measured at the 330 nm excitation and 390 nm emission by an automatic plate multireader. The IC₅₀ values are calculated from the initial reaction rates.

### Example 3002

### Assay for Determining MMP-8 Inhibition

The typical assay for MMP-8 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of activated MMP-8 enzyme (Calbiochem, Cat. No. 444229) is added to the compound solution. The mixture of enzyme and compound in assay buffer is thoroughly mixed and incubated for 10 min at 37° C. Upon the completion of incubation, the assay is started by addition of 40 μL of a 10 μM stock solution of OmniMMP fluorescent substrate (Biomol, Cat. No. P-126). The time-dependent increase in fluorescence is measured at the 320 nm excitation and 390 nm emission by an automatic plate multireader at 37° C. The IC₅₀ values are calculated from the initial reaction rates.

### Example 3003

### Assay for Determining MMP-12 Inhibition

The typical assay for MMP-12 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of the catalytic domain of MMP-12 enzyme (Biomol, Cat. No. SE-138) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### Example 3004

### Assay for Determining Aggrecanase-1 Inhibition

The typical assay for aggrecanase-1 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 75 nM stock solution of aggrecanase-1 (Invitek) is added to the compound solution. The mixture of enzyme and compound in assay buffer is thoroughly mixed. The reaction is started by addition of 40 μL of a 250 nM stock solution of aggrecan-IGD substrate (Invitek) and incubation at 37° C. for exact 15 min. The reaction is stopped by addition of EDTA and the samples are analysed by using aggrecanase ELISA (Invitek, InviLISA, Cat. No. 30510111) according to the protocol of the supplier. Shortly: 100 μL of each proteolytic reaction are incubated in a pre-coated micro plate for 90 min at room temperature. After 3 times washing, antibody-peroxidase conjugate is added for 90 min at room temperature. After 5 times washing, the plate is incubated with TMB solution for 3 min at room temperature. The peroxidase reaction is stopped with sulfurous acid and the absorbance is red at 450 nm. The IC₅₀ values are calculated from the absorbance signal corresponding to residual aggrecanase activity.

What is claimed is:

1. A compound having Formula (I): 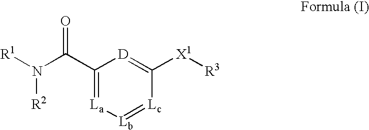
wherein:
R¹ in each occurrence is independently selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
wherein optionally two hydrogen atoms on the same atom of the R¹ group are replaced with ═O, ═S or ═NR¹⁰;
R² in each occurrence is independently selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)^{x}, or NR⁵⁰ and which is optionally substituted one or more times;
R³ is selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵², 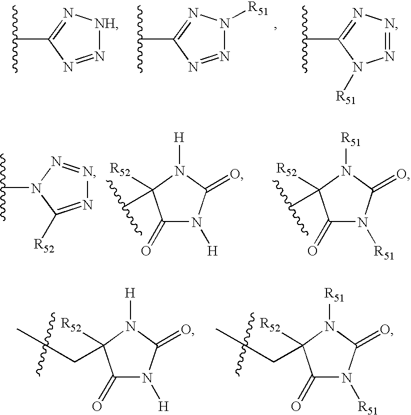 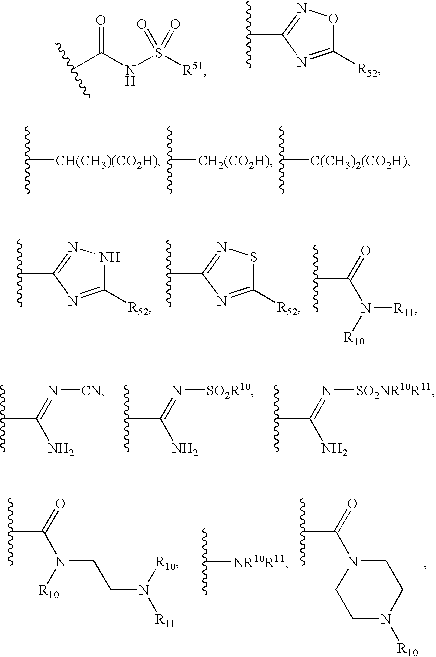 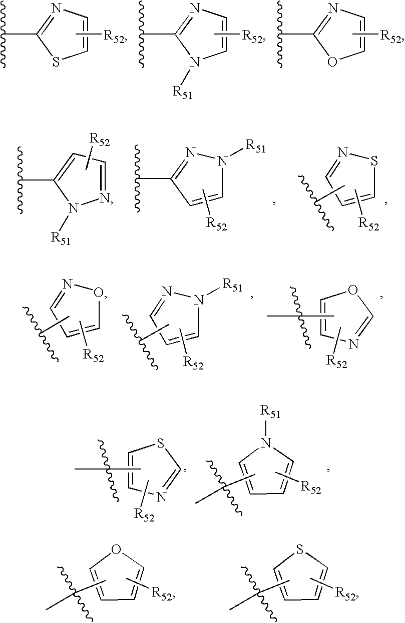 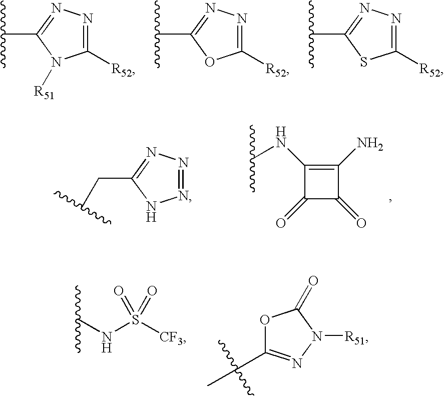 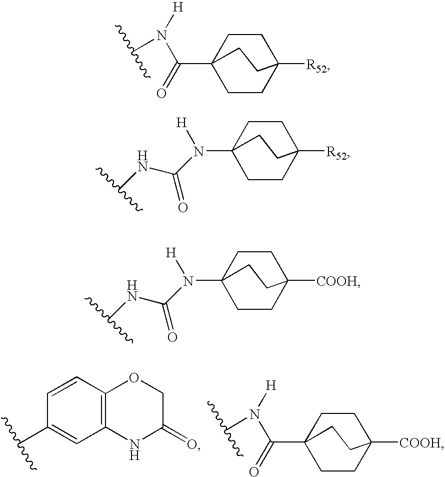 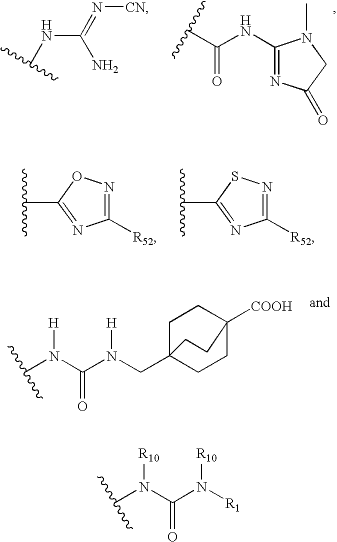
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R¹⁰, (C₀-C₆)-alkyl-S(O)ₓR¹⁰, (C₀-C₆)-alkyl-OC(O)R¹¹, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)ORto, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from the group consisting of hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from the group consisting of hydrogen, alkyl, arylalkyl, cycloalkylalkyl, heteroarylalkyl, heterocycloalkyl and halo, wherein alkyl, arylalkyl, cycloalkylalkyl, heteroarylalkyl and heterocycloalkyl are optionally substituted one or more times.
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 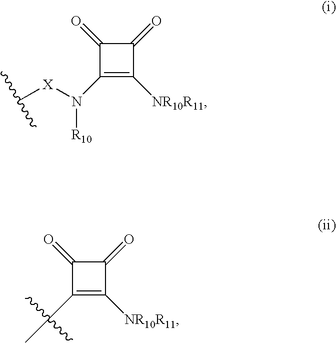
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicylic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
D is a member selected from the group consisting of CR²² and N;
Lₐ L_{b}, and L_{c} are independently selected from CR⁹ and N with the proviso that Lₐ, L_{b}, and L_{c}, cannot all simultaneously be N;
X¹ is selected from the group consisting of a bond, NR¹⁰, CH₂, CHR²⁰, CR²⁰R²¹, SO₂, SO, S, PO₂, O, C═S, C═O, C═NR¹, C═N—SO₂R¹⁰, C═N—CN, C═N—CONR¹⁰R¹¹, C═N—COR¹⁰, C═N—OR¹⁰, NR¹⁰C═O, NR¹⁰SO₂ and SO₂NR¹⁰;
x is selected from 0 to 2;
y is selected from 1 and 2; and
N-oxides, pharmaceutically acceptable salts, prodrugs, formulations, polymorphs, tautomers, racemic mixtures and stereoisomers thereof.

2. The compound of claim 1, selected from the group consisting of:

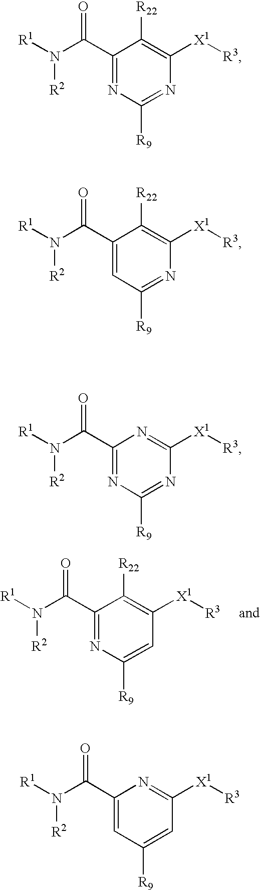

3. The compound of claim 2, selected from the group consisting of:

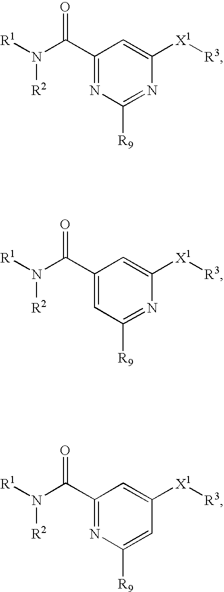

4. The compound of claim 3, selected from the group consisting of:

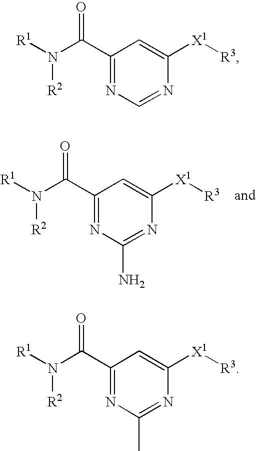

5. The compound of claim 4, selected from the group consisting of:

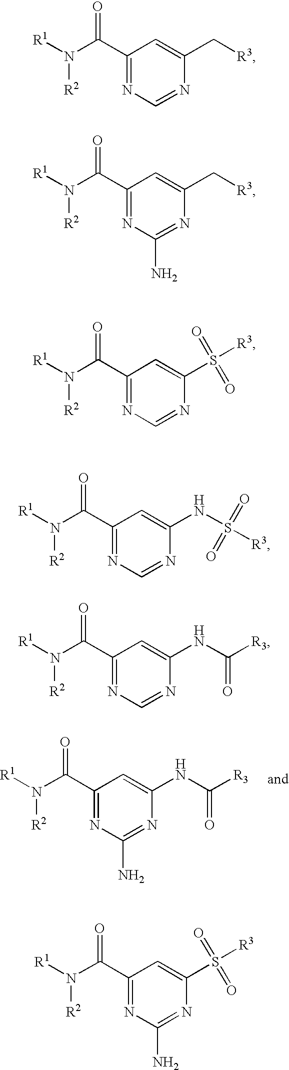

6. The compound of claim 5, wherein R³ is selected from the group consisting of: 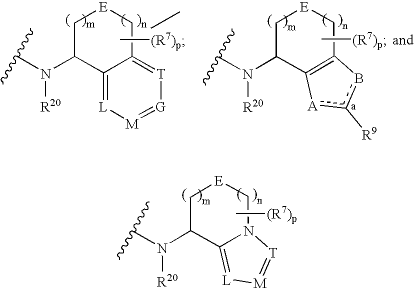
wherein:
R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³³, (C₀-C₆)-alkyl-S(O)ₓNR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹⁰—CN, O—(C₀-C₆)-alkyl-C(O)NR¹⁰R¹¹, S(O), —(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁷ is independently selected from the group consisting of hydrogen, alkyl, cycloalkyl, halo, R⁴ and NR¹⁰R¹¹, or optionally two R⁷ groups together at the same carbon atom form ═O, ═S or ═NR¹⁰;
A and B are independently selected from the group consisting of CR⁹, CR⁹R¹⁰, NR¹⁰, N, O, SO, SO₂ and S;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W₁— and 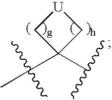
G, L, M and T are independently selected from the group consisting of CR⁹ and N;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
g and h are independently selected from 0-2;
m and n are independently selected from 0-3, provided that:
(1) when E is present, m and n are not both 3;
(2) when E is —CH₂—W¹—, m and n are not 3; and
(3) when E is a bond, m and n are not 0; and
p is selected from 0-6;
wherein the dotted line represents a double bond between one of: carbon “a” and A, or carbon “a” and B.

7. The compound of claim 5, wherein R³ is selected from the group consisting of:
hydrogen, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

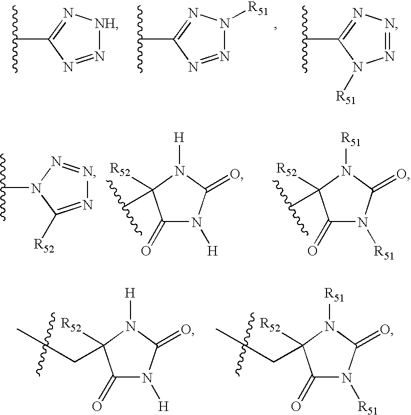

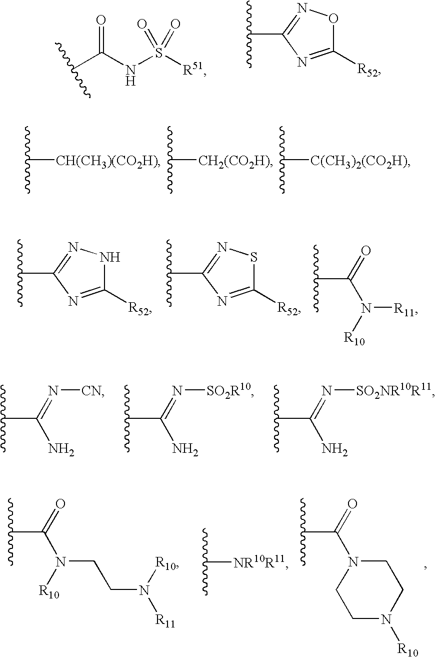

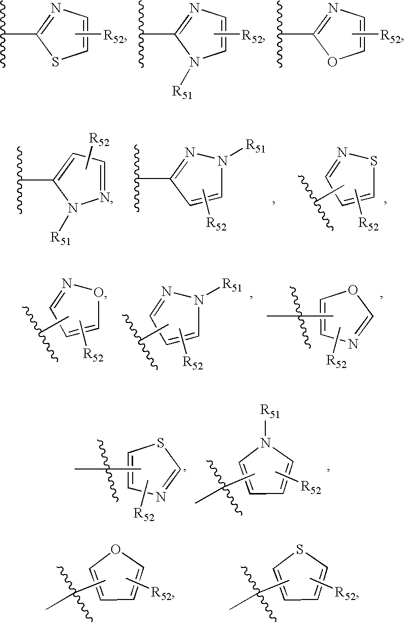

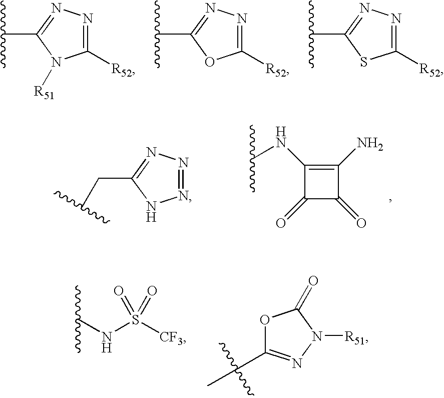

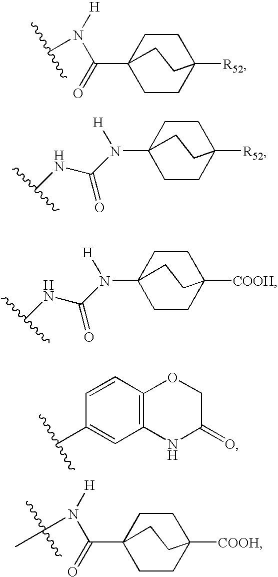

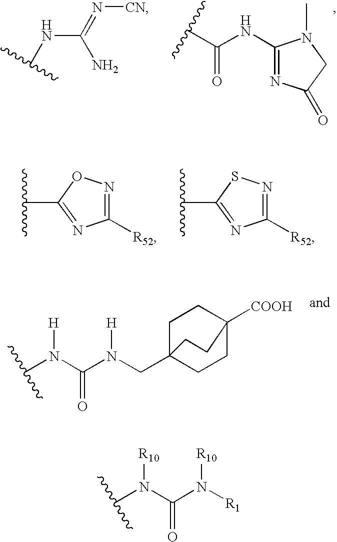

8. The compound according to claim 6, wherein R³ is selected from the group consisting of: 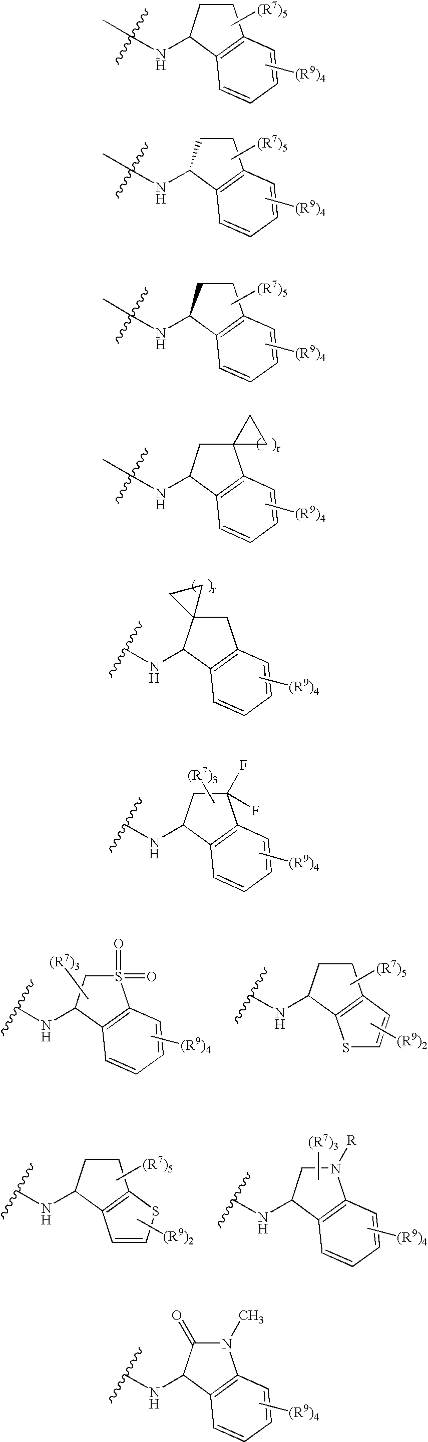 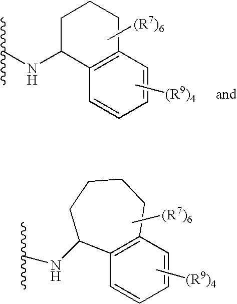
wherein:
R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and
r is selected from 1-4.

9. The compound according to claim 8, wherein R³ selected from the group consisting of:

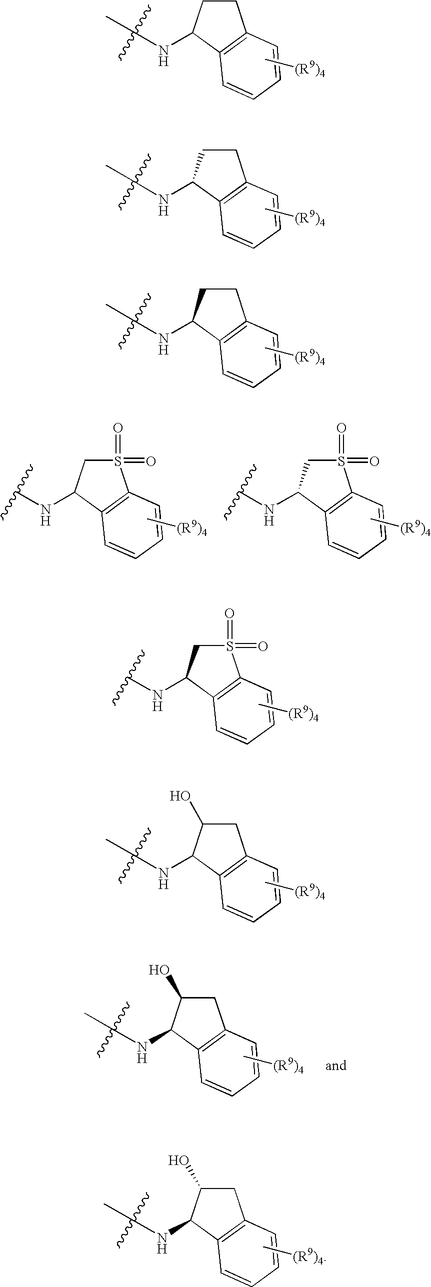

10. The compound according to claim 9, wherein R⁹ is selected from the group consisting of:

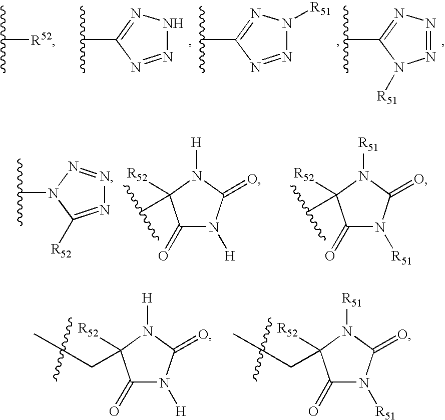

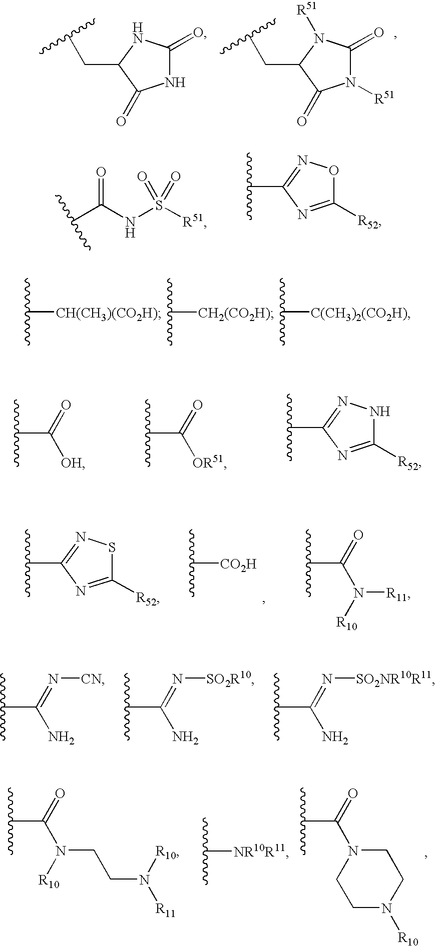

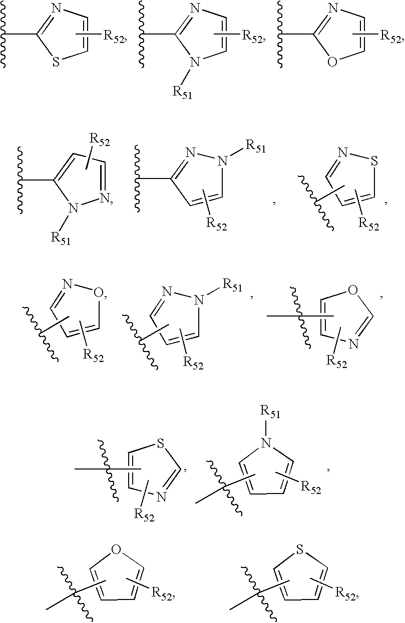

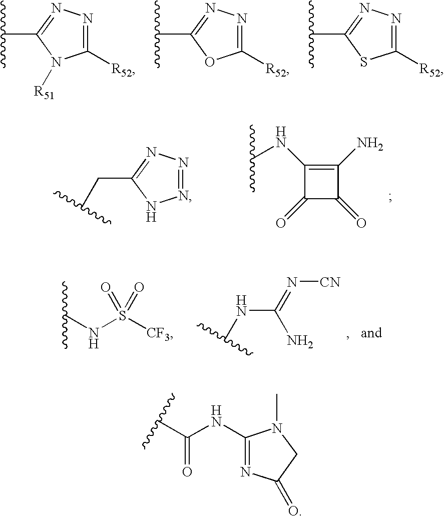

11. The compound according to claim 9, wherein R³ is

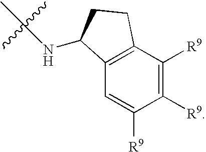

12. The compound according to claim 11, wherein R³ is selected from the group consisting of: 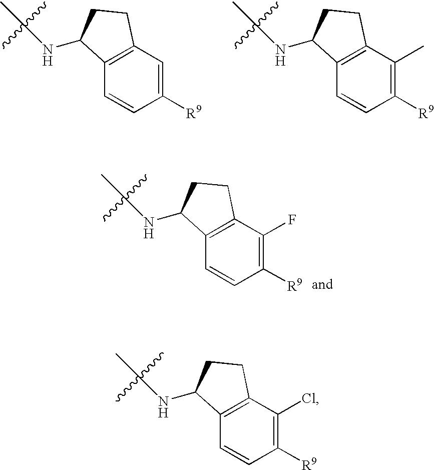
wherein:
R⁹ is selected from the group consisting of hydrogen, fluoro, halo, CN, alkyl, CO₂H,

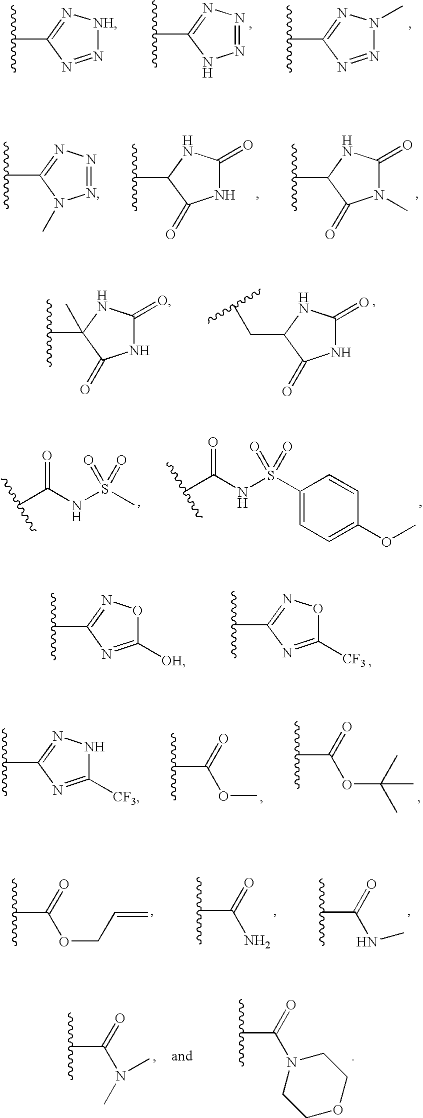

13. The compound according to claim 5, wherein R³ is selected from the group consisting of: R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰ (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl.

14. The compound according to claim 1, wherein R¹ is selected from the group consisting of: 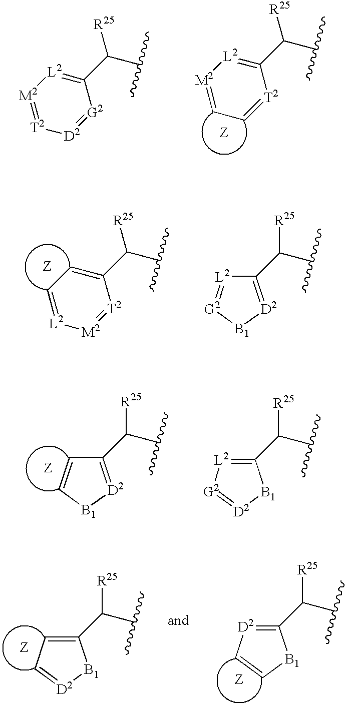
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
B, is selected from the group consisting of NR¹⁰, O, SO, SO₂, and S;
D², G², L², M² and T² are independently selected from the group consisting of CR¹⁸ and N; and
Z is a 5- to 8-membered ring selected from the group consisting of cycloalkyl, heterocycloalkyl, aryl and heteroaryl, wherein cycloalkyl, heterocycloalkyl, aryl and heteroaryl are optionally substituted one or more times.

15. The compound according to claim 14, wherein R¹ is selected from the group consisting of:

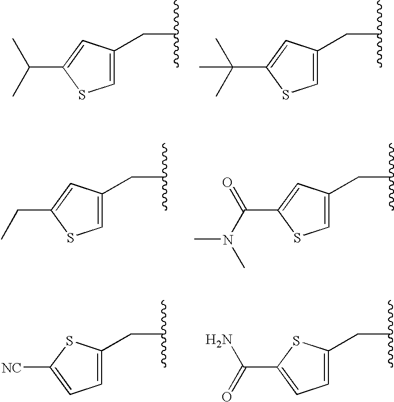

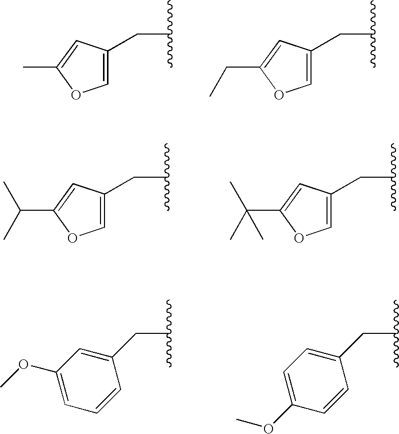

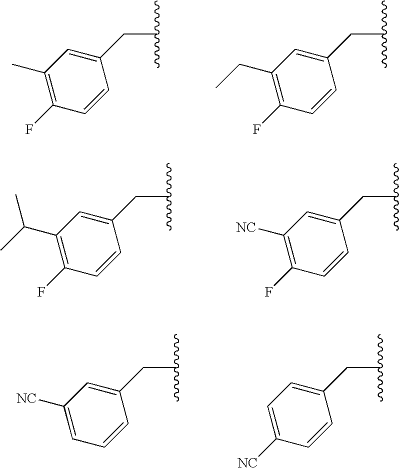

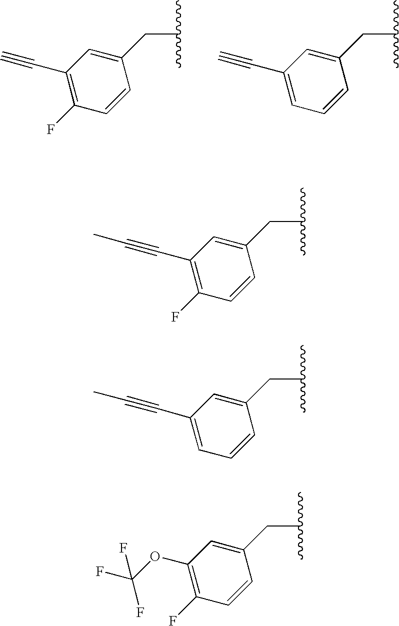

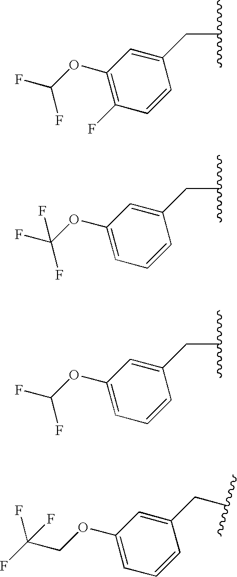

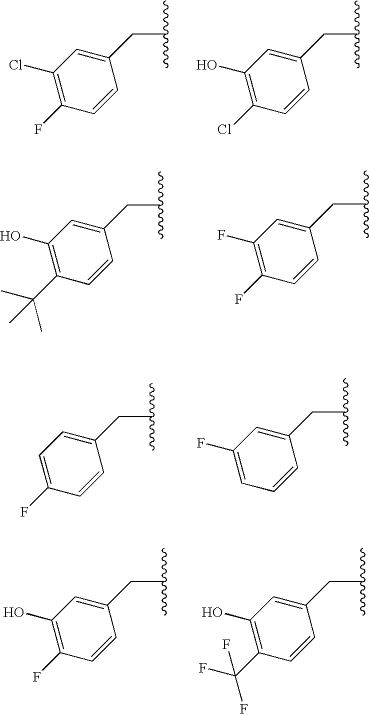

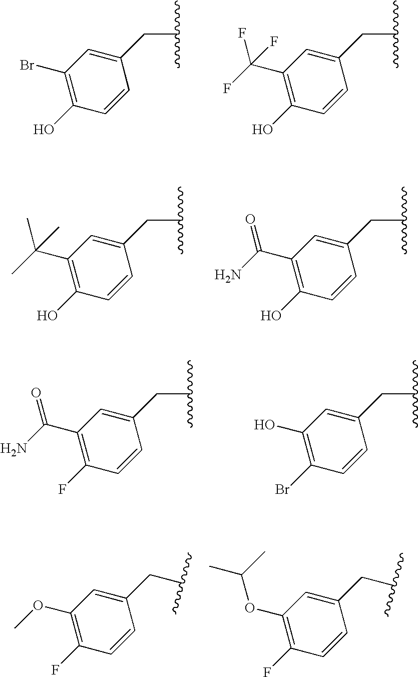

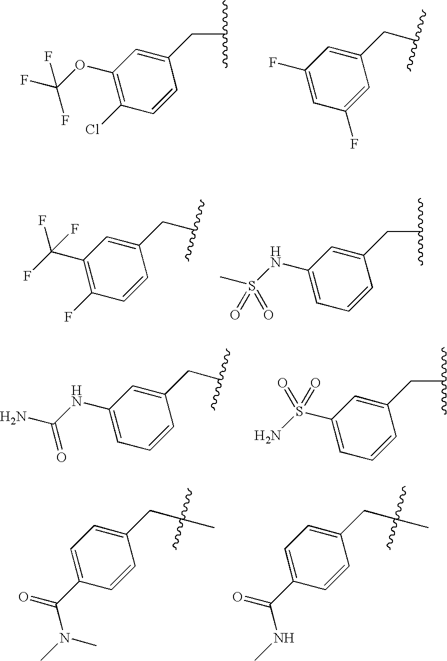

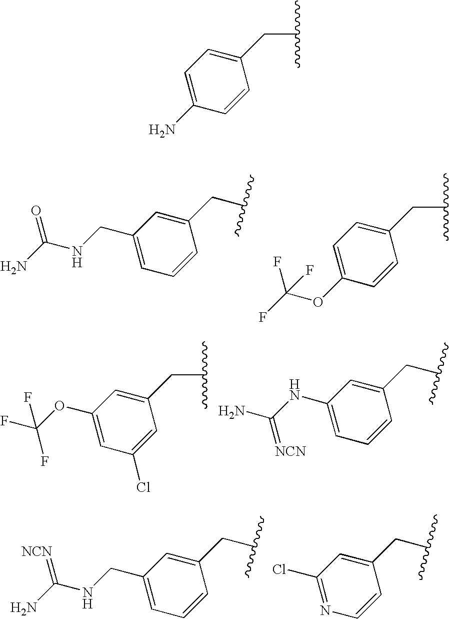

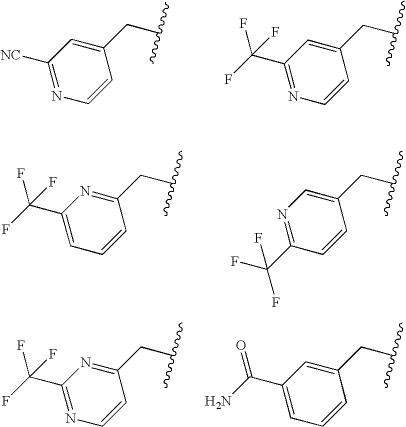

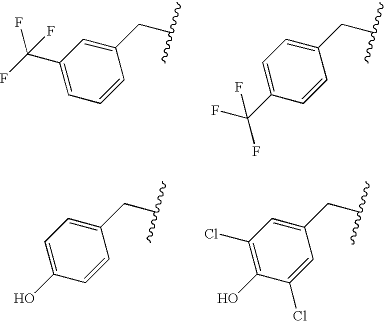

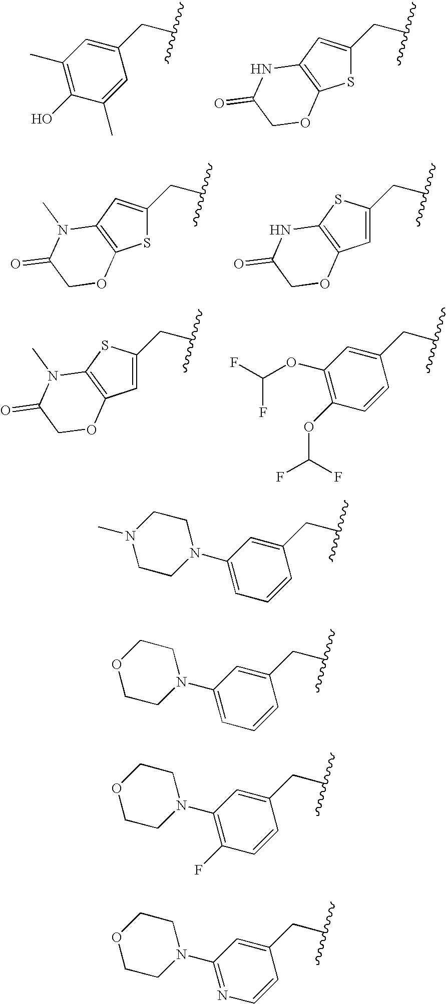

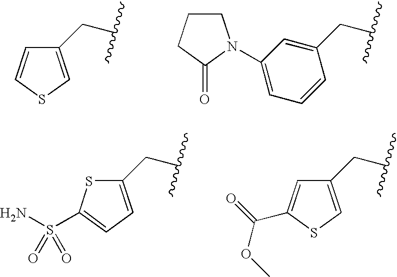

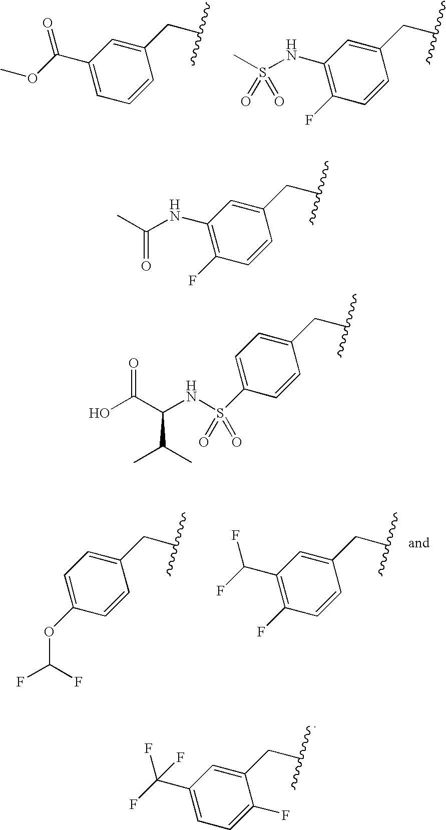

16. The compound according to claim 1, wherein R¹ is selected from the group consisting of: 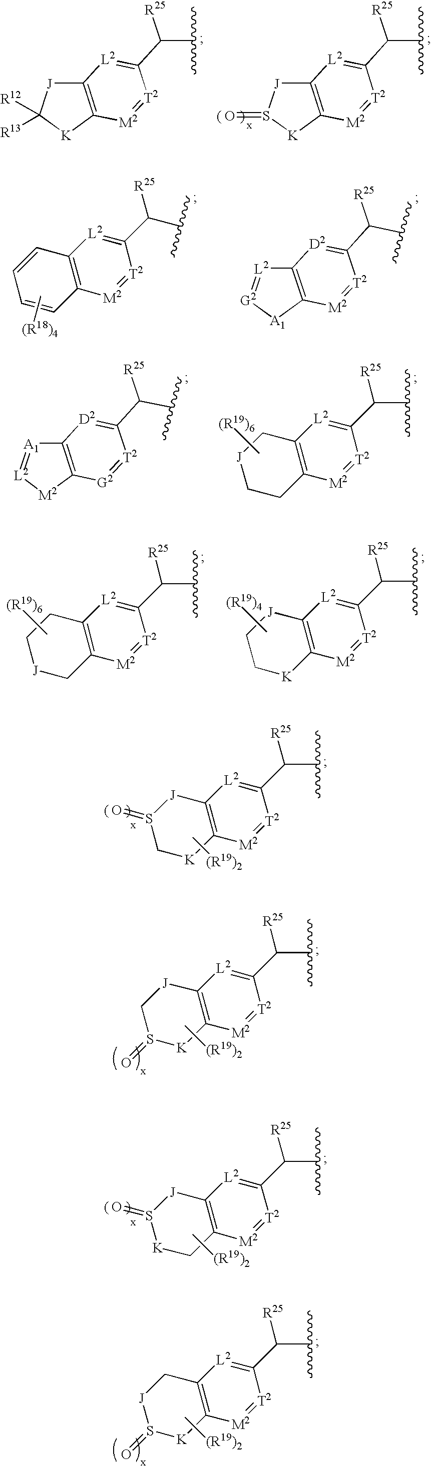 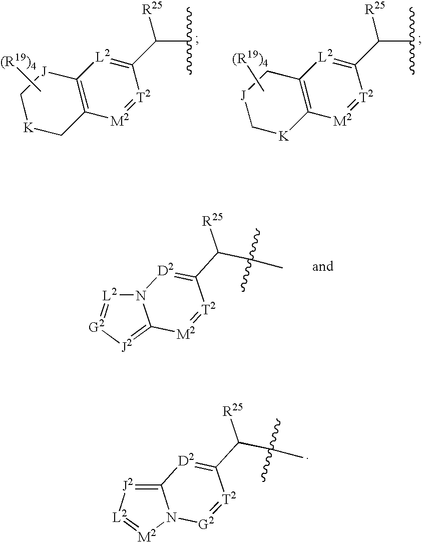
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups taken together with the carbon atom to which they are attached form ═O, ═S or ═NR¹⁰;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O, SO, SO₂, and S; and
D², G², J², L², M² and T² are independently selected from the group consisting of CR¹⁸ and N.

17. The compound according to claim 16, wherein R¹ is selected from the group consisting of:

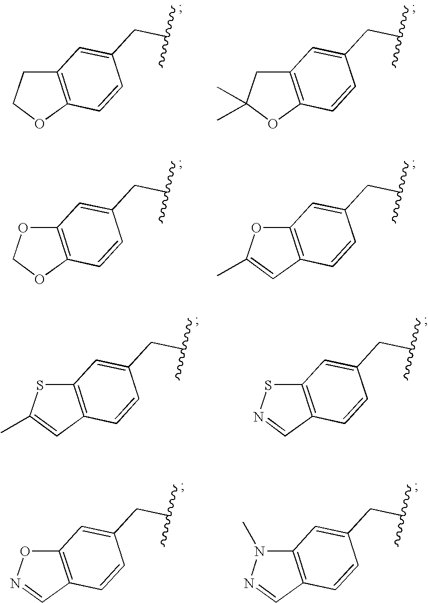

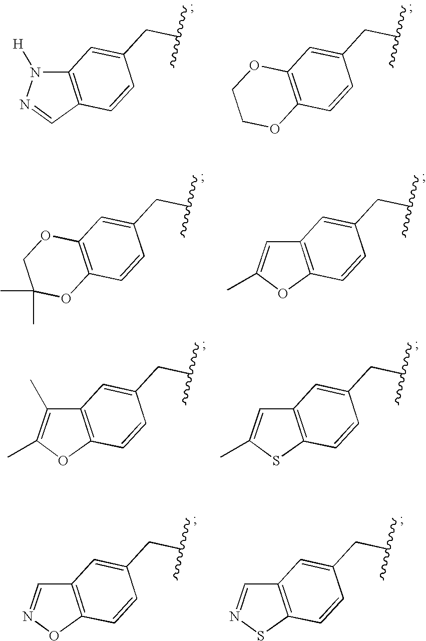

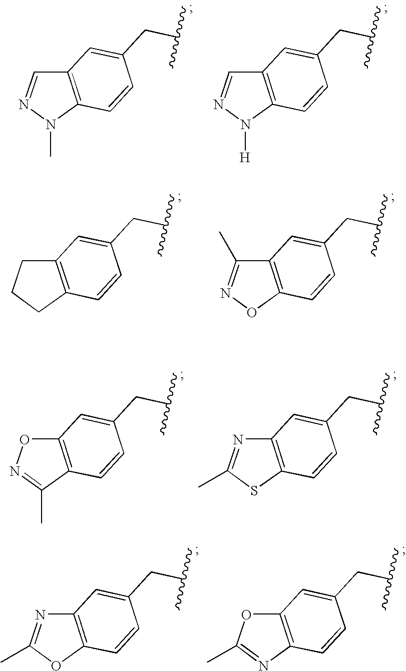

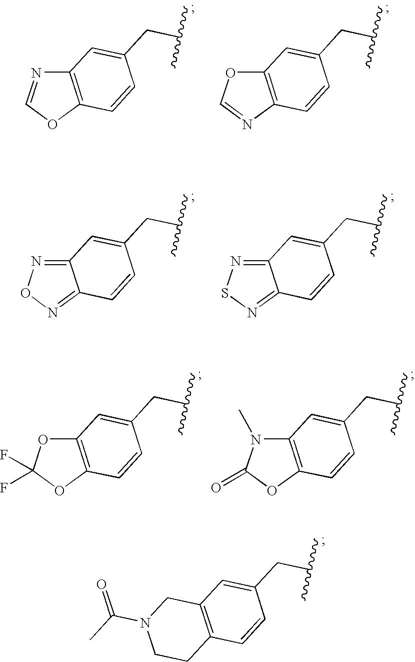

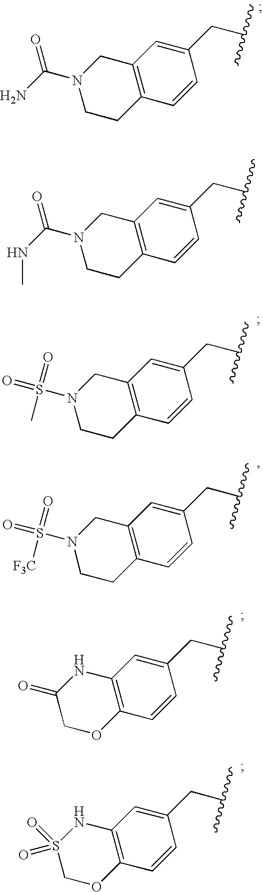

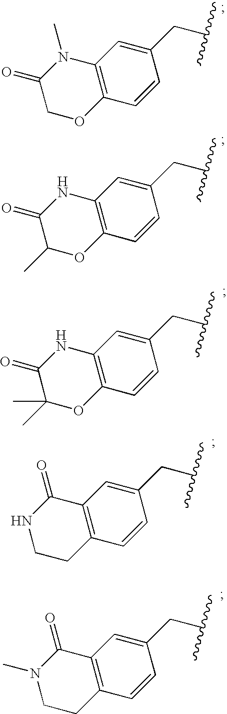

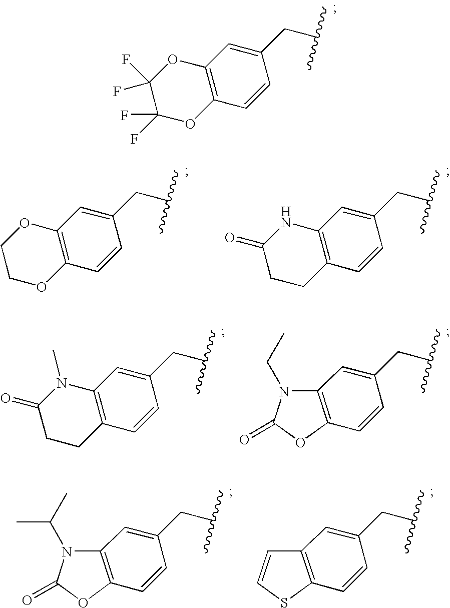

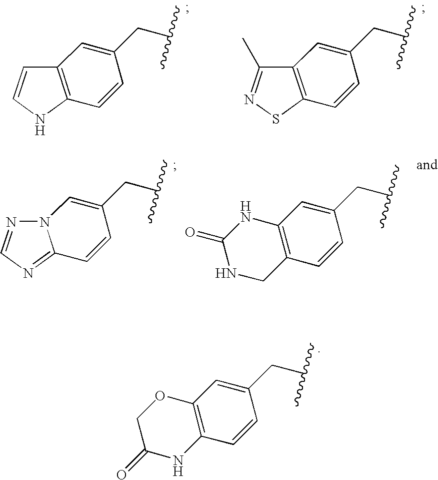

18. The compound according to claim 1, wherein R¹ selected from the group consisting of: 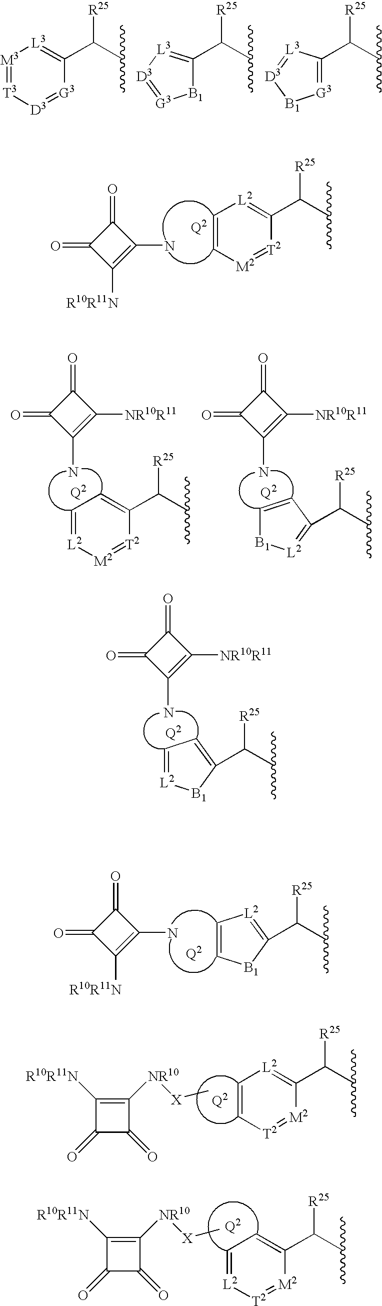 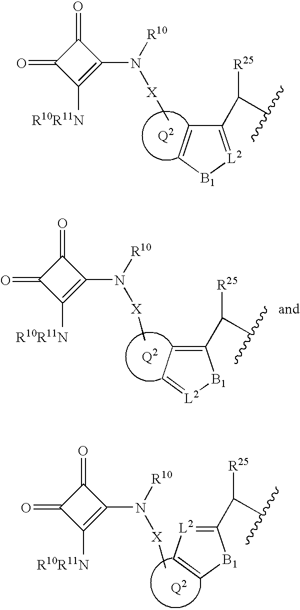
wherein:
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═0, ═S or ═NR¹⁰;
R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 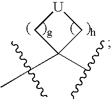
L², M², and T² are independently selected from the group consisting of CR¹⁸ and N;
D³, G³, L³, M³, and T³ are independently selected from N, CR¹⁸, (i) and (ii), 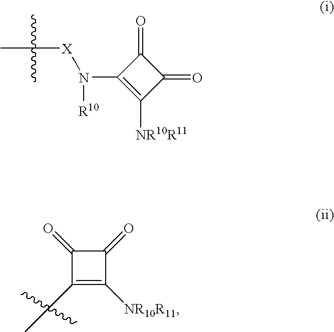
with the proviso that one of L³, M³, T³, D³, and G³ is (i) or (ii);
B, is selected from the group consisting of NR¹⁰, O and S; and
Q² is a 5- to 8-membered ring selected from the group consisting of cycloalkyl, heterocycloalkyl, aryl, and heteroaryl, which is optionally substituted one or more times with R¹⁹;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2; and
w is independently selected from 0-4.

19. The compound according to claim 18, wherein R¹ is selected from the group consisting of:

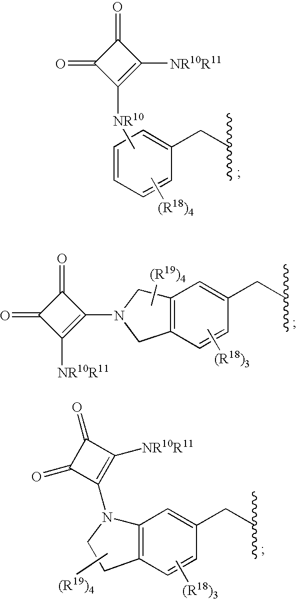

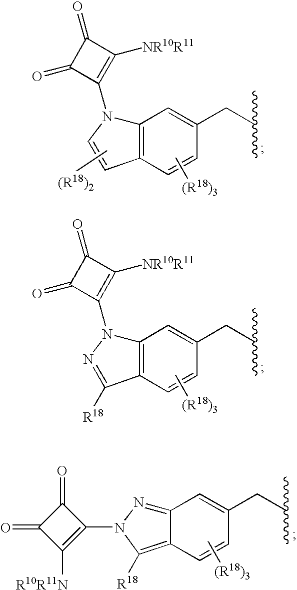

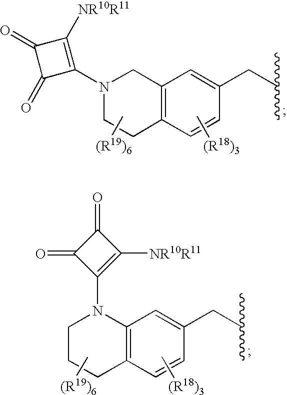

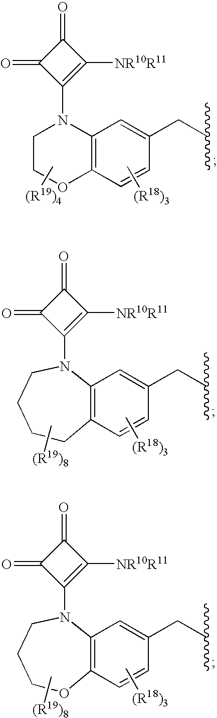

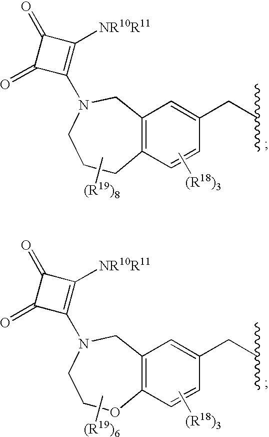

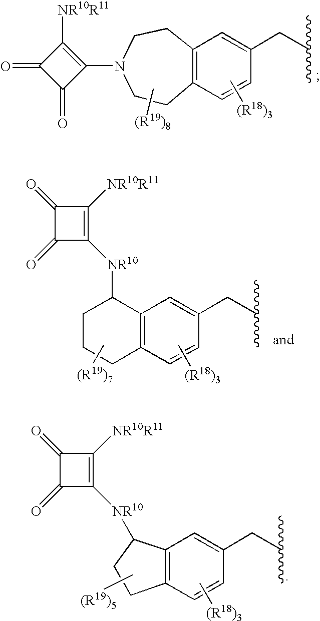

20. The compound according to claim 19, wherein R¹ is selected from the group consisting of:

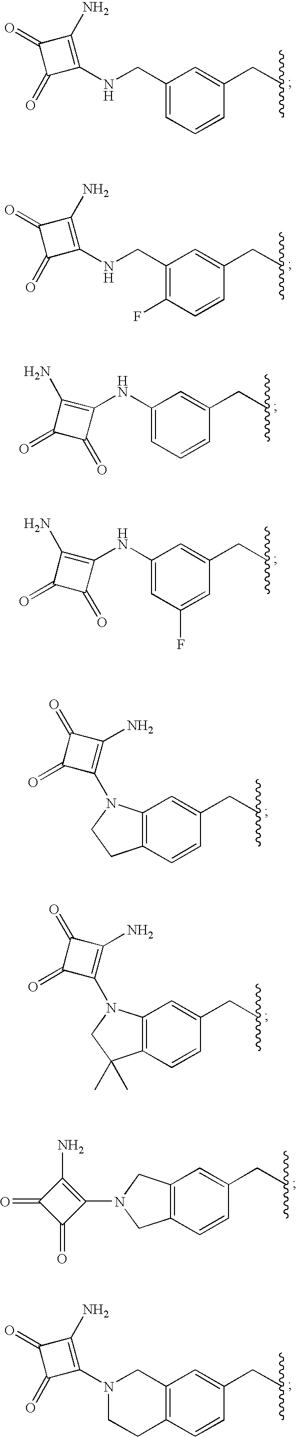

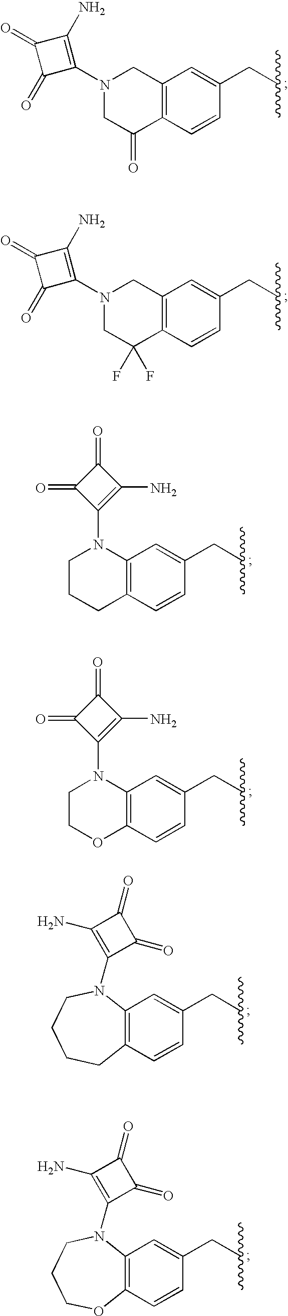

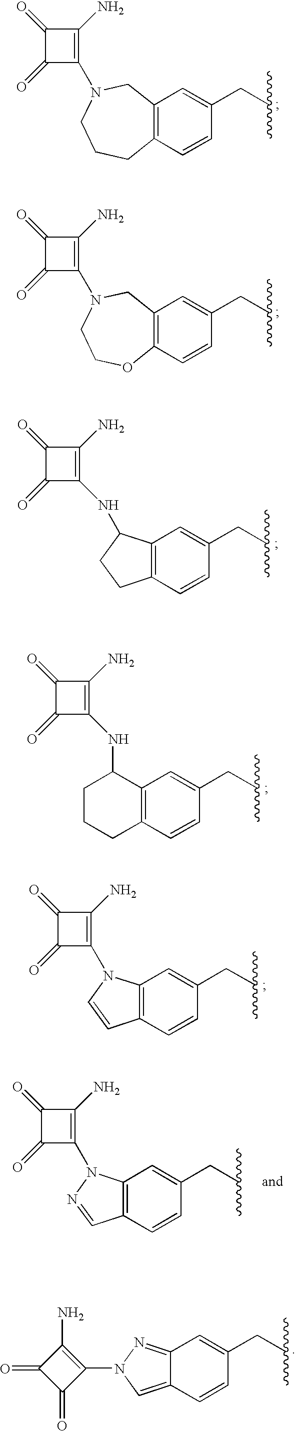

21. The compound of claim 1, wherein
X¹ is a bond, and
R³ is selected from the group consisting of

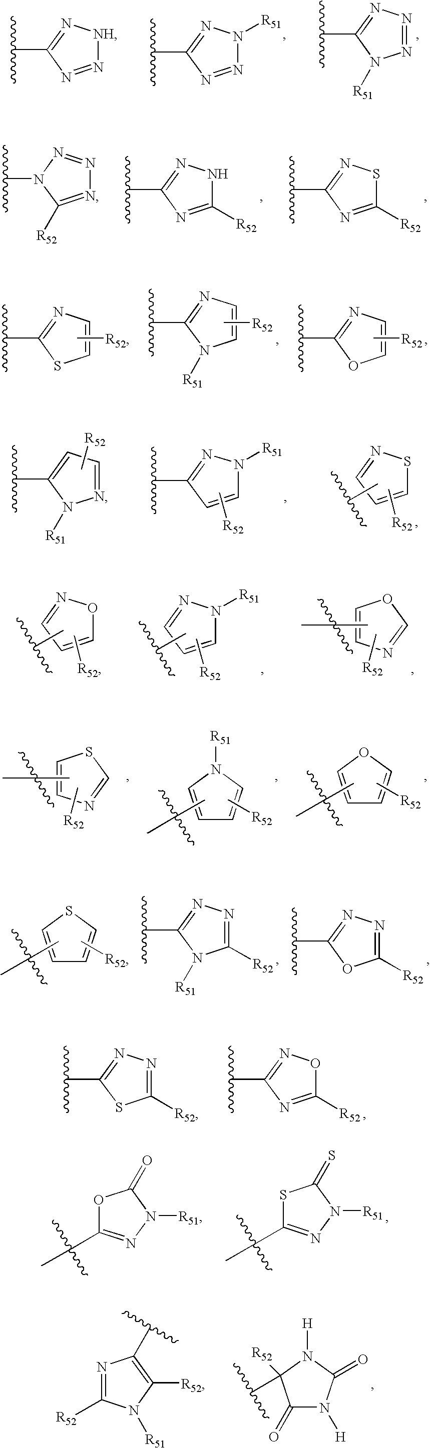

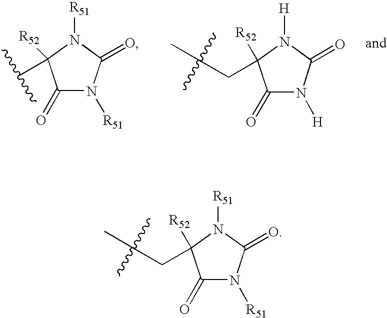

22. The compound of claim 1, wherein said compound is selected from the group consisting of:

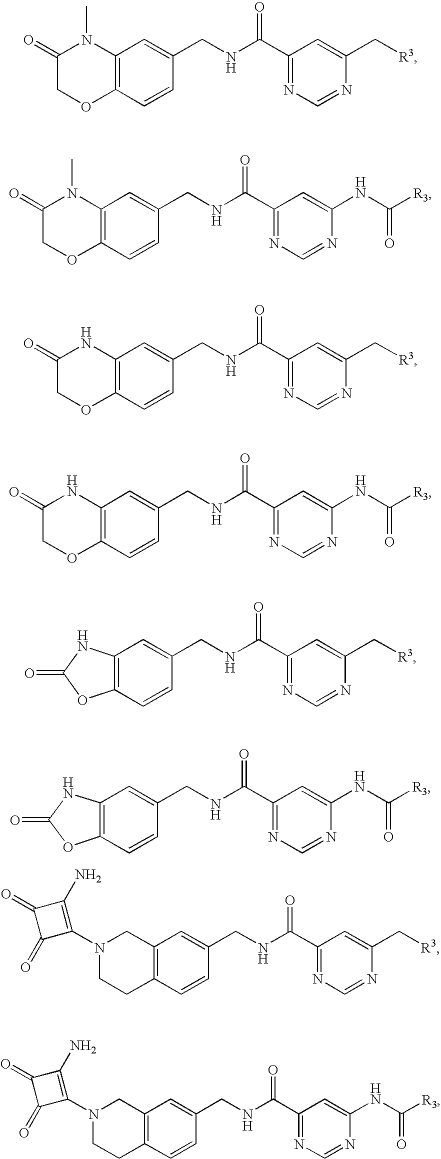

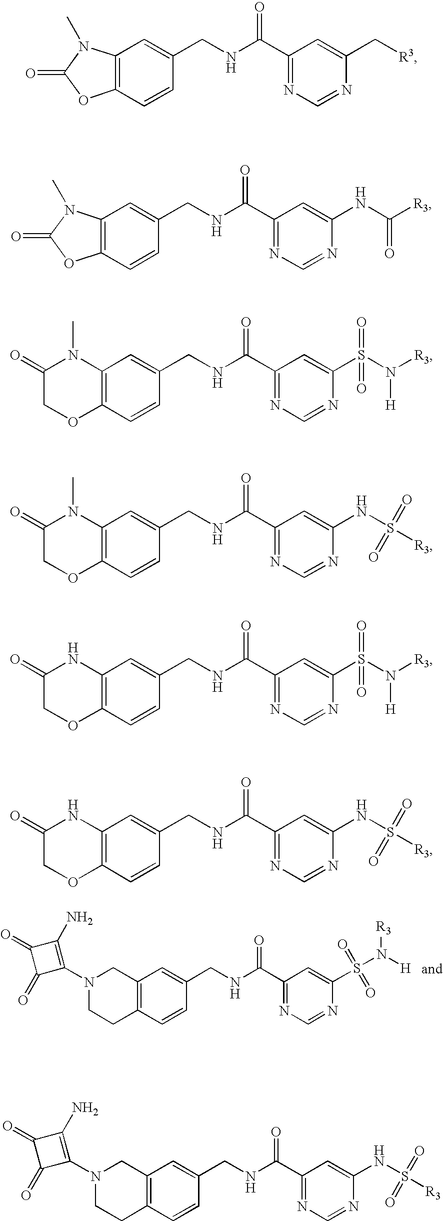

23. A compound having Formula (II): 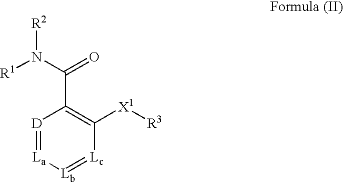
wherein:
R¹ in each occurrence is independently selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
wherein optionally two hydrogen atoms on the same atom of the R¹ group are replaced with ═O, ═S or ═NR¹⁰;
R² in each occurrence is independently selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹¹, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NROR¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NROR¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl, NR²⁰R²¹, NR¹⁰R¹¹, COR¹⁰, COR²¹, COOR¹⁰, COOR²¹, CR²⁰R²¹R¹, SO₂R¹⁰, SO₂R¹⁰, SO₂NR¹⁰R¹¹SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵², 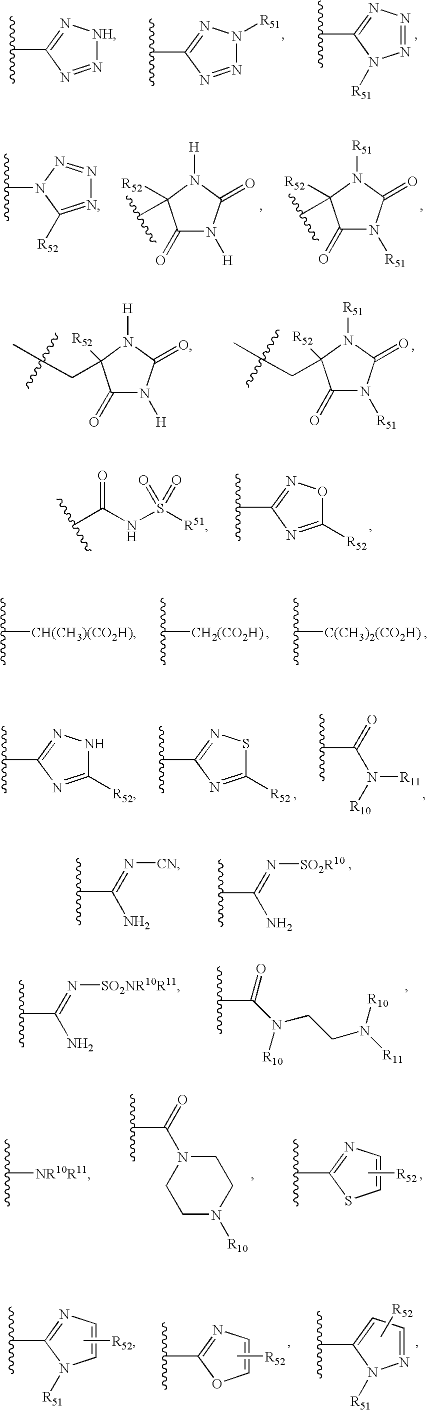 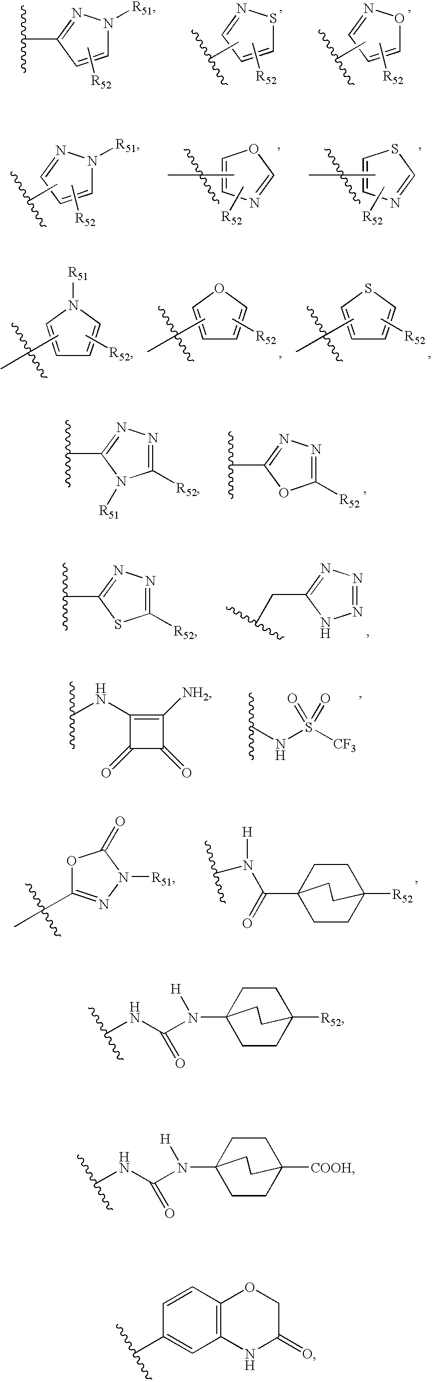 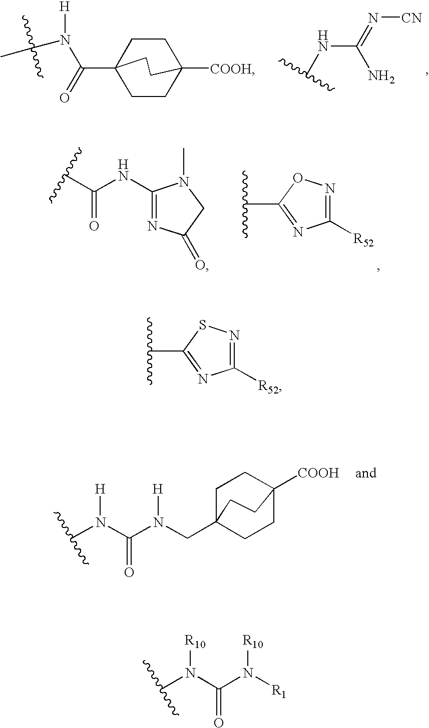
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹¹—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from the group consisting of hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from the group consisting of hydrogen, alkyl, arylalkyl, cycloalkylalkyl, heteroarylalkyl, heterocycloalkyl and halo, wherein alkyl, arylalkyl, cycloalkylalkyl, heteroarylalkyl and heterocycloalkyl are optionally substituted one or more times.
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 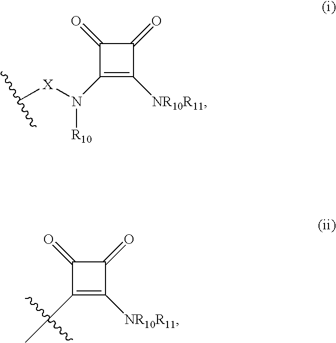
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicylic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
D is a member selected from the group consisting of CR²² and N;
Lₐ, L_{b}, and L_{c}, are independently selected from CR⁹ and N with the proviso that Lₐ, L_{b}, and L_{c}, cannot all simultaneously be N;
X¹ is selected from the group consisting of a bond, NR¹⁰, CH₂, CHR¹⁰, CR²⁰R²¹, SO₂, SO, S, PO₂, O, C═S, C═O, C═NR¹, C═N—SO₂R¹⁰, C═N—CN, C═N—CONR¹⁰R¹¹, C═N—COR¹⁰, C═N—OR¹⁰, NR¹⁰C═O, NR¹⁰SO₂ and SO₂NR¹⁰;
x is selected from 0 to 2;
y is selected from 1 and 2; and
N-oxides, pharmaceutically acceptable salts, prodrugs, formulations, polymorphs, tautomers, racemic mixtures and stereoisomers thereof.

24. The compound of claim 23, selected from the group consisting of:

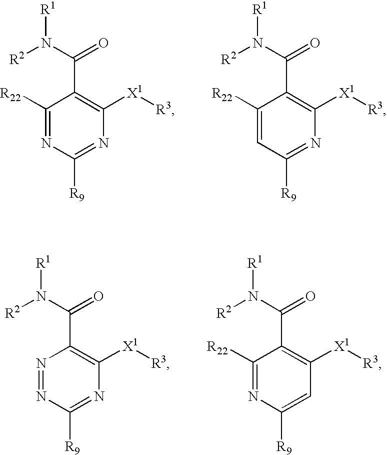

25. The compound of claim 24, selected from the group consisting of:

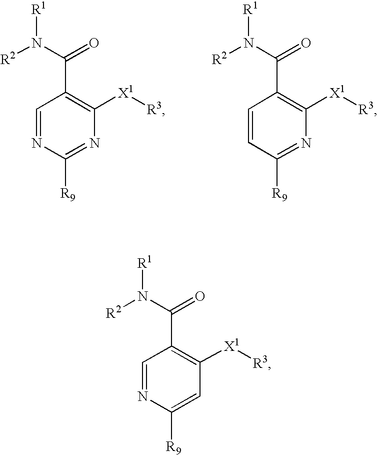

26. The compound of claim 24, selected from the group consisting of:

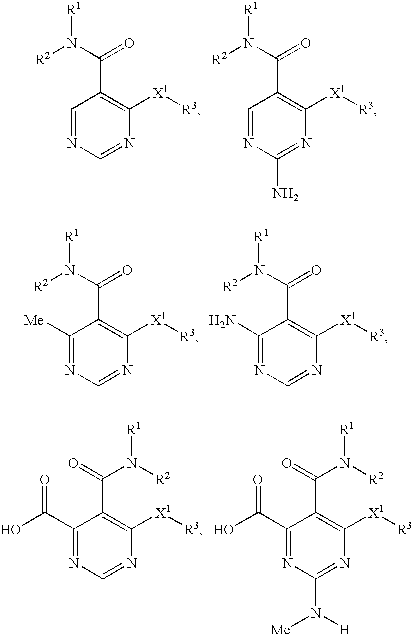

27. The compound of claim 26, selected from the group consisting of:

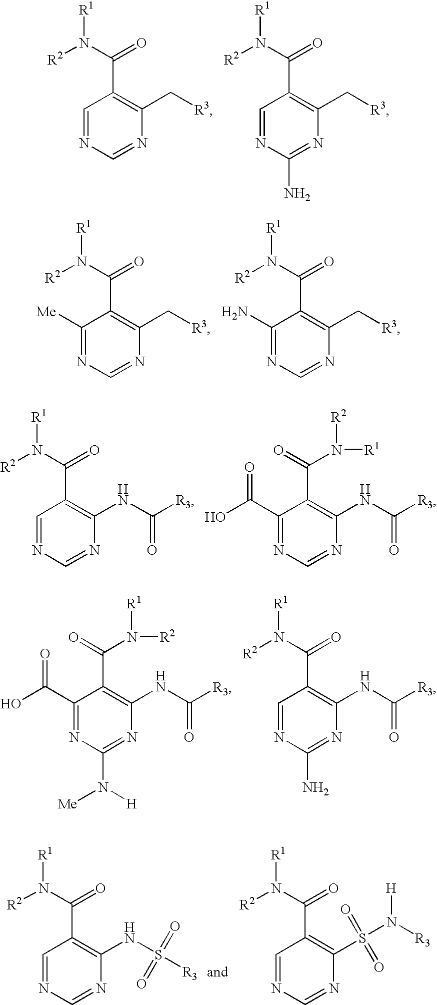

28. The compound of claim 23, wherein R³ is selected from the group consisting of: 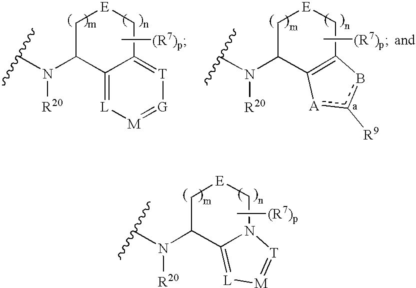
wherein:
R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹¹CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁷ is independently selected from the group consisting of hydrogen, alkyl, cycloalkyl, halo, R⁴ and NR¹⁰R¹¹ or optionally two R⁷ groups together at the same carbon atom form ═O, ═S or ═NR¹⁰;
A and B are independently selected from the group consisting of CR⁹, CR⁹R¹⁰, NR¹⁰, N, O, SO, SO₂ and S;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 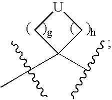
G, L, M and T are independently selected from the group consisting of CR⁹ and N;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹¹)(C═O), N(R¹⁰)S(═O)₂ and S(═O)₂N(R¹⁰);
g and h are independently selected from 0-2;
m and n are independently selected from 0-3, provided that:
(1) when E is present, m and n are not both 3;
(2) when E is —CH₂—W¹—, m and n are not 3; and
(3) when E is a bond, m and n are not 0; and
p is selected from 0-6;
wherein the dotted line represents a double bond between one of: carbon “a” and A, or carbon “a” and B.

29. The compound of claim 27, wherein R³ is selected from the group consisting of:
hydrogen, NR²⁰R²¹, NR¹⁰R¹¹, COR¹⁰, COR²¹, COOR²¹, COOR¹⁰, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

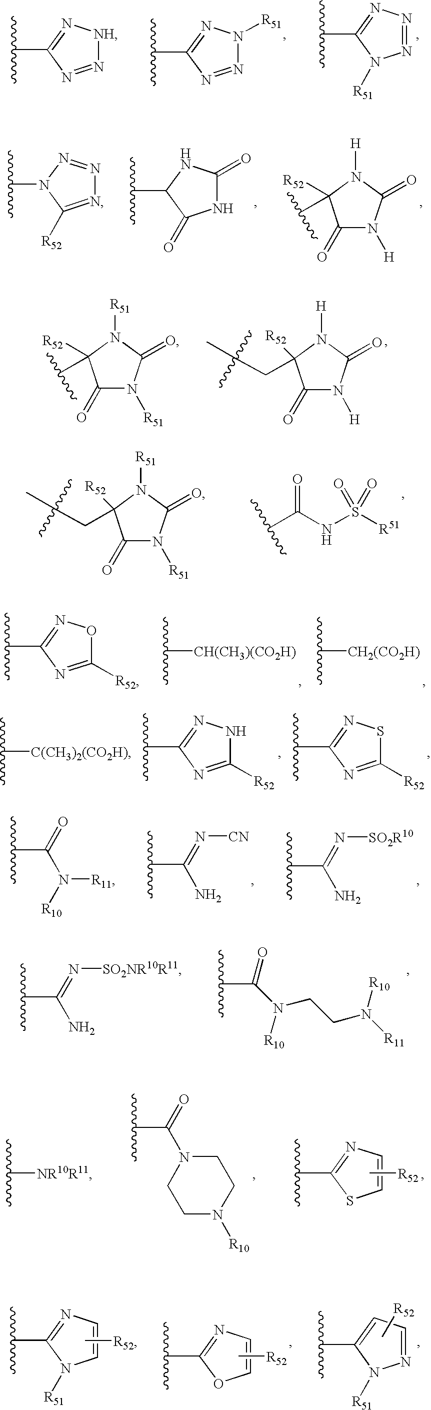

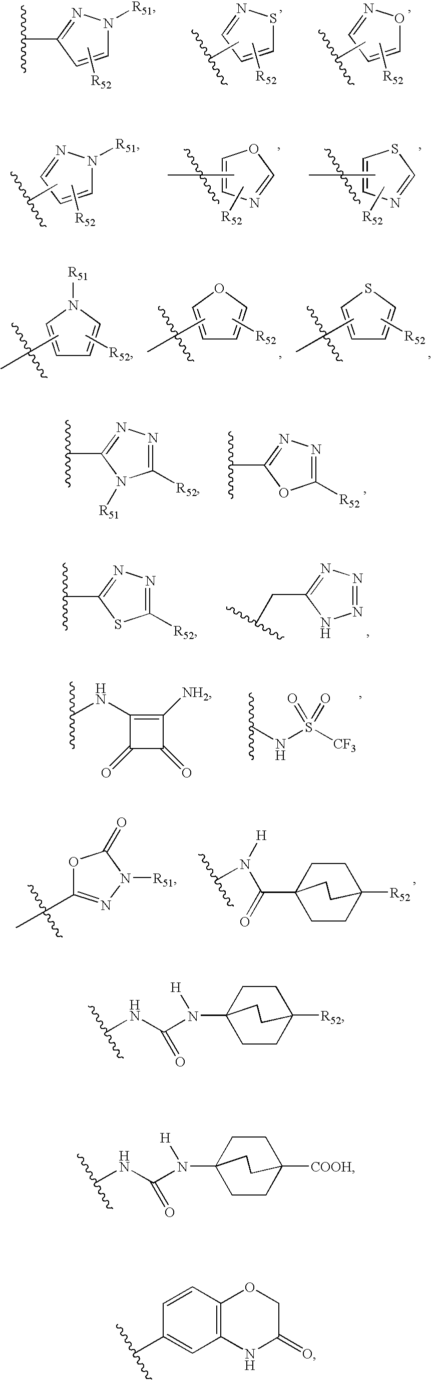

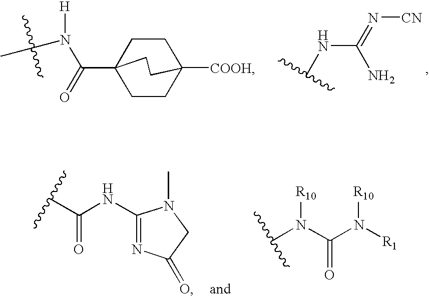

30. The compound according to claim 27, wherein R³ is selected from the group consisting of: R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl.

31. The compound according to claim 27, wherein R³ is selected from the group consisting of: 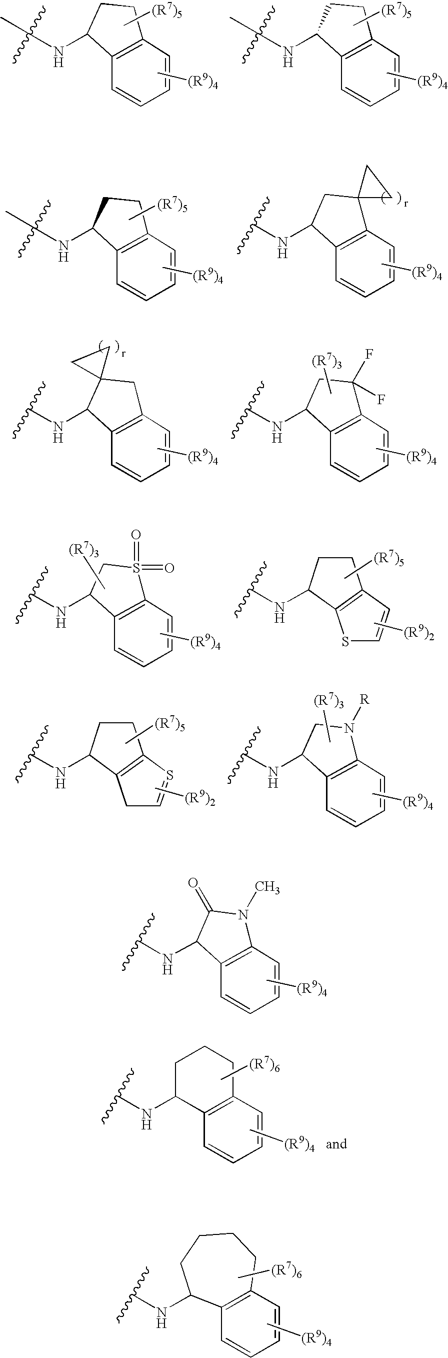
wherein:
R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and
r is selected from 1-4.

32. The compound according to claim 31, wherein R³ selected from the group consisting of:

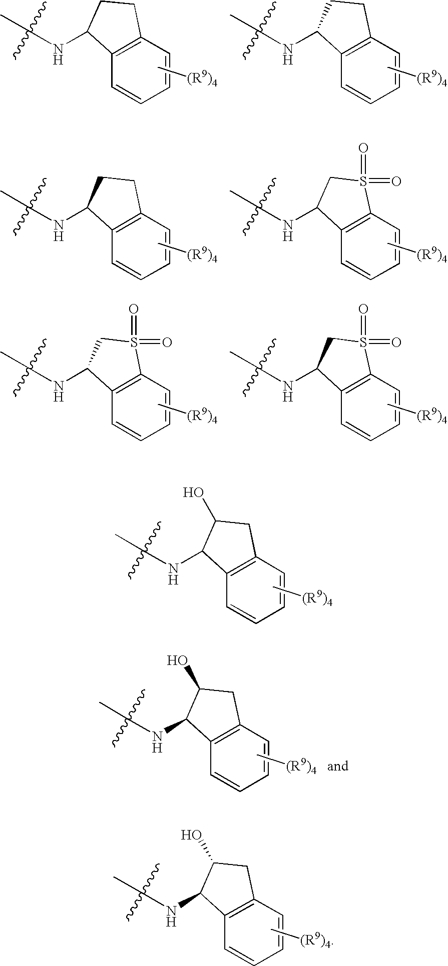

33. The compound according to claim 32, wherein R⁹ is selected from the group consisting of:

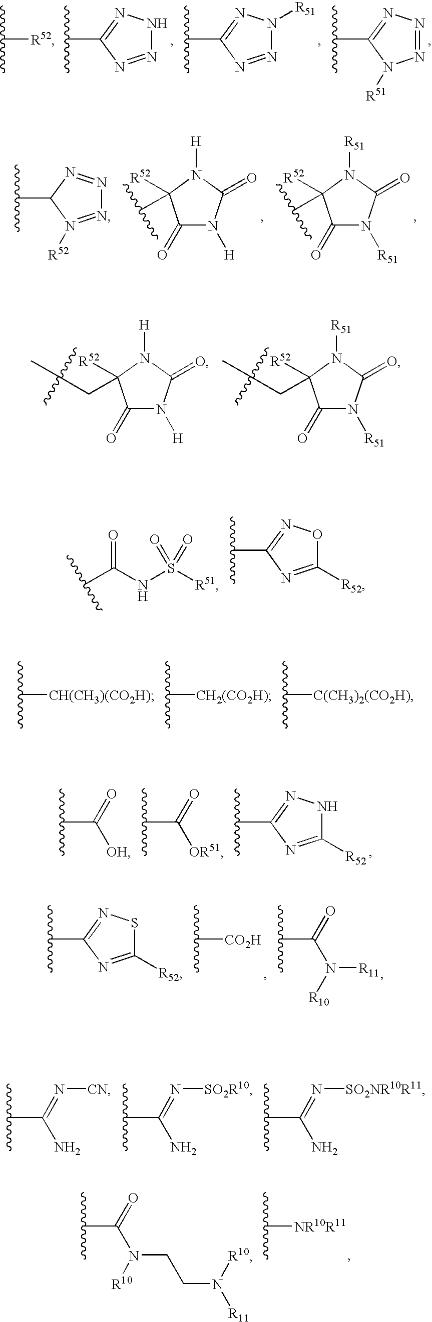

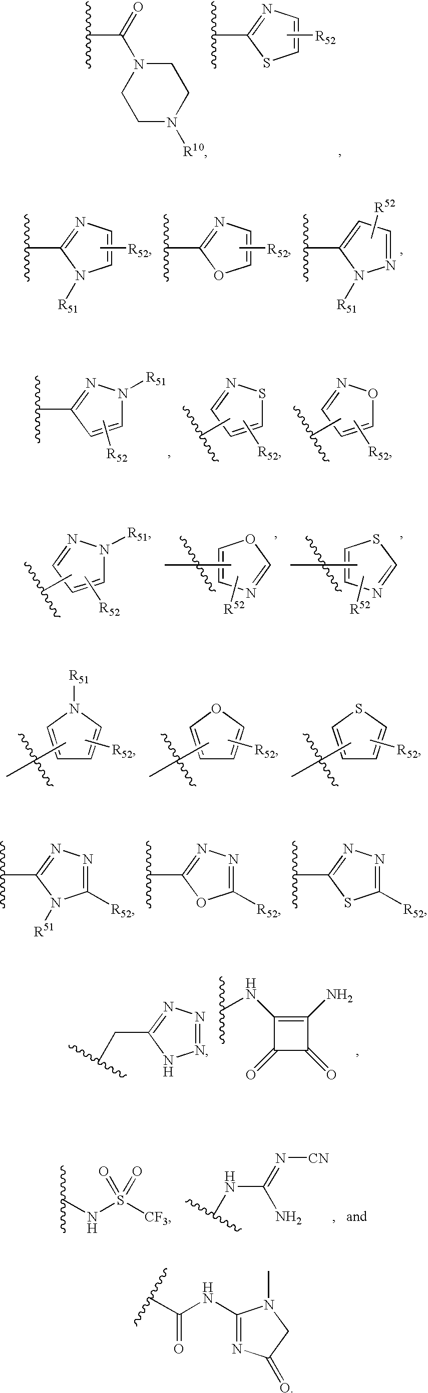

34. The compound according to claim 32, wherein R³ is

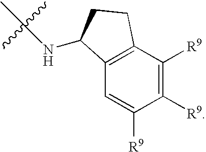

35. The compound according to claim 34, wherein R³ is selected from the group consisting of: 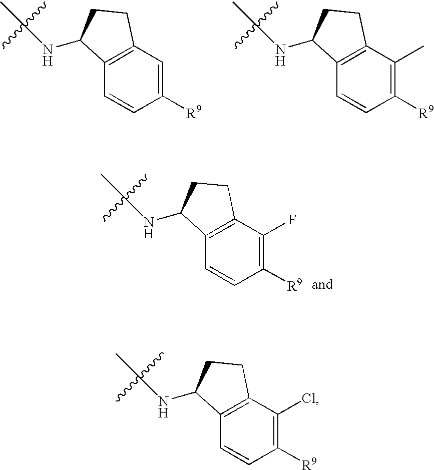
wherein:
R⁹ is selected from the group consisting of hydrogen, fluoro, halo, CN, alkyl, CO₂H,

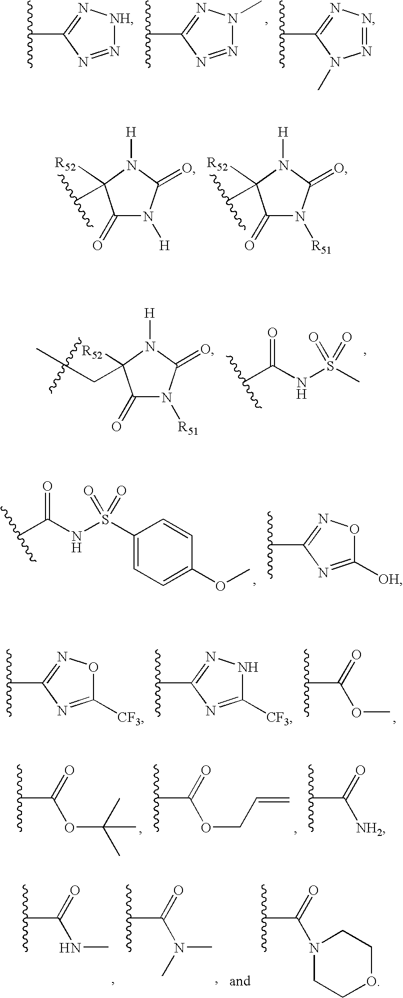

36. The compound according to claim 23, wherein R¹ is selected from the group consisting of: 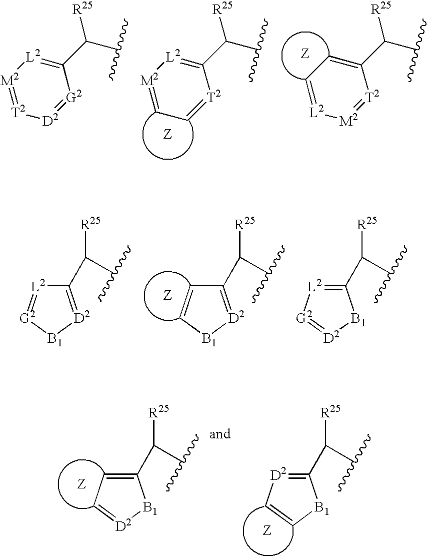
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹¹, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
B₁ is selected from the group consisting of NR¹⁰, O, SO₂, SO and S;
D², G², L², M² and T² are independently selected from the group consisting of CR¹⁸ and N; and
Z is a 5- to 8-membered ring selected from the group consisting of cycloalkyl, heterocycloalkyl, aryl and heteroaryl, wherein cycloalkyl, heterocycloalkyl, aryl and heteroaryl are optionally substituted one or more times.

37. The compound according to claim 36, wherein R¹ is selected from the group consisting of:

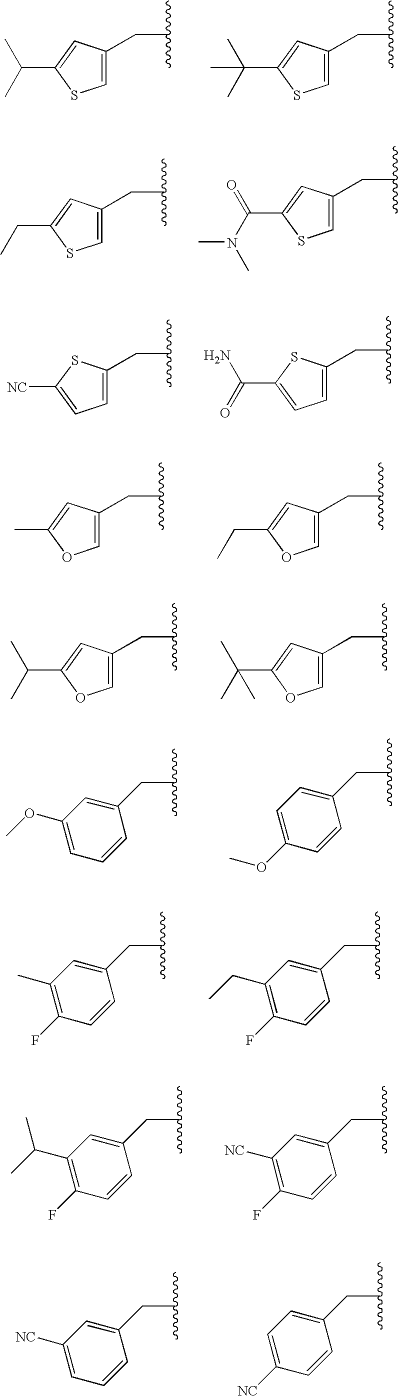

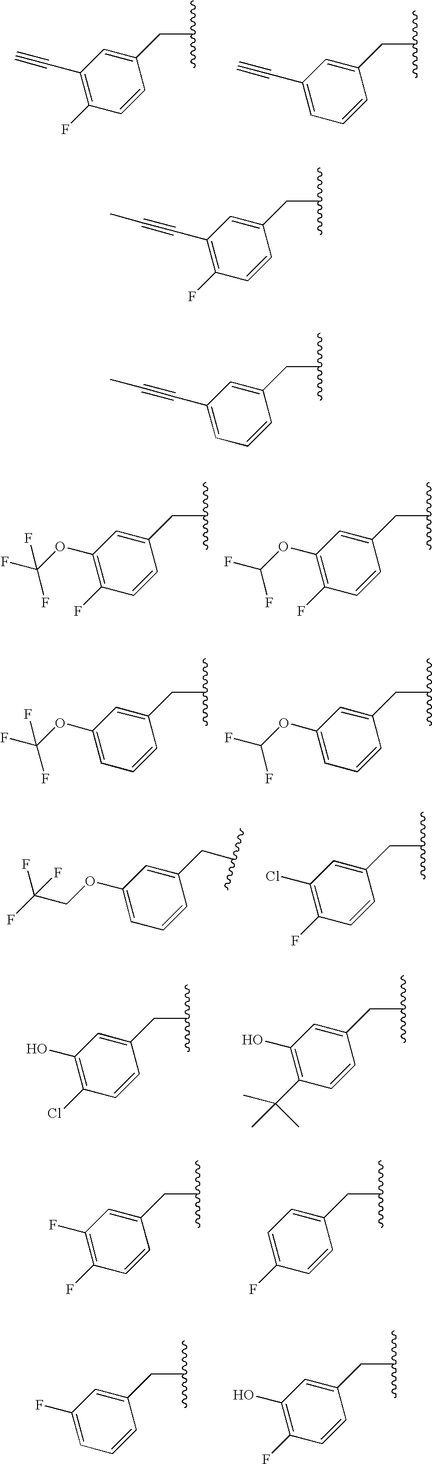

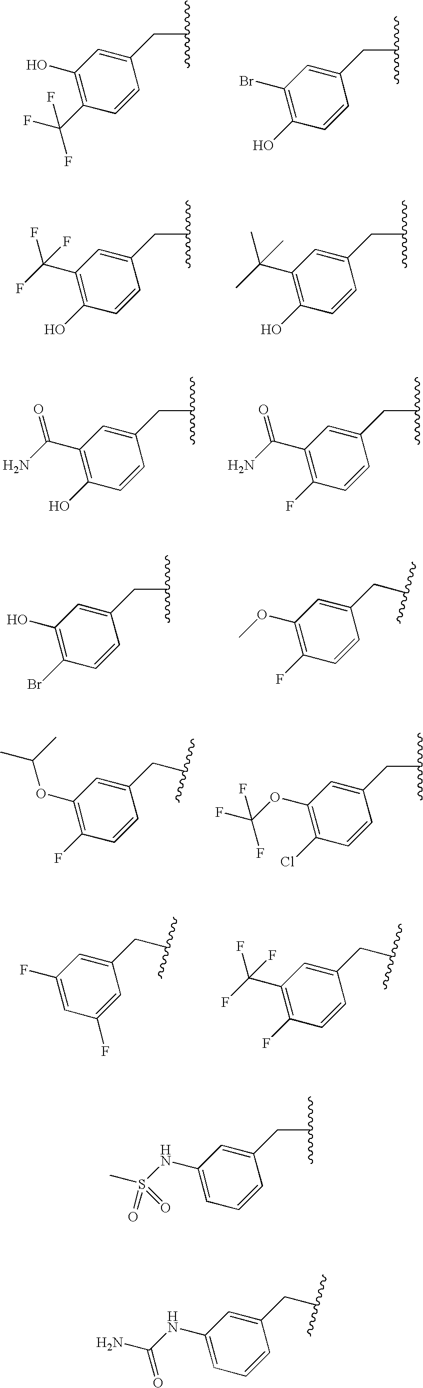

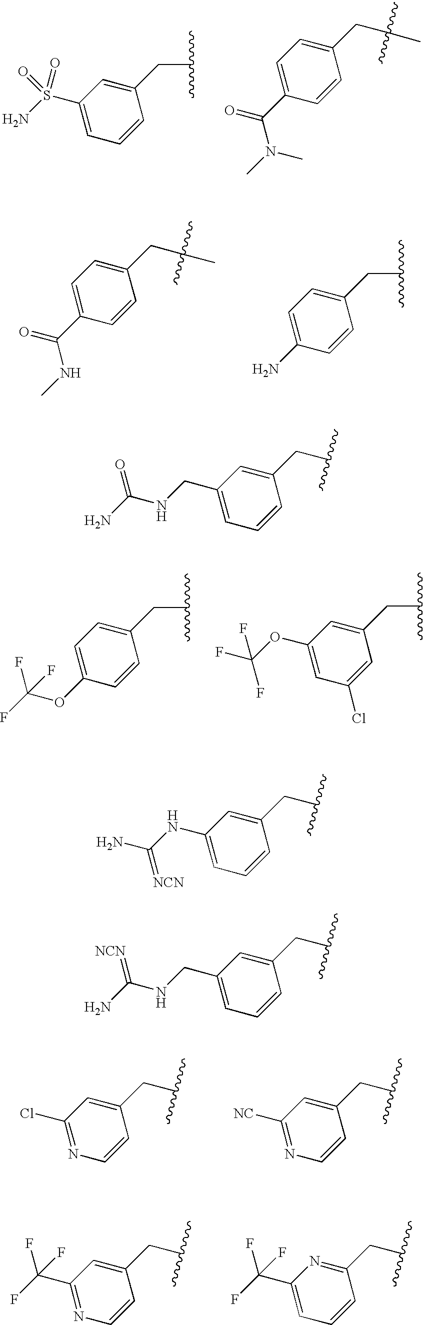

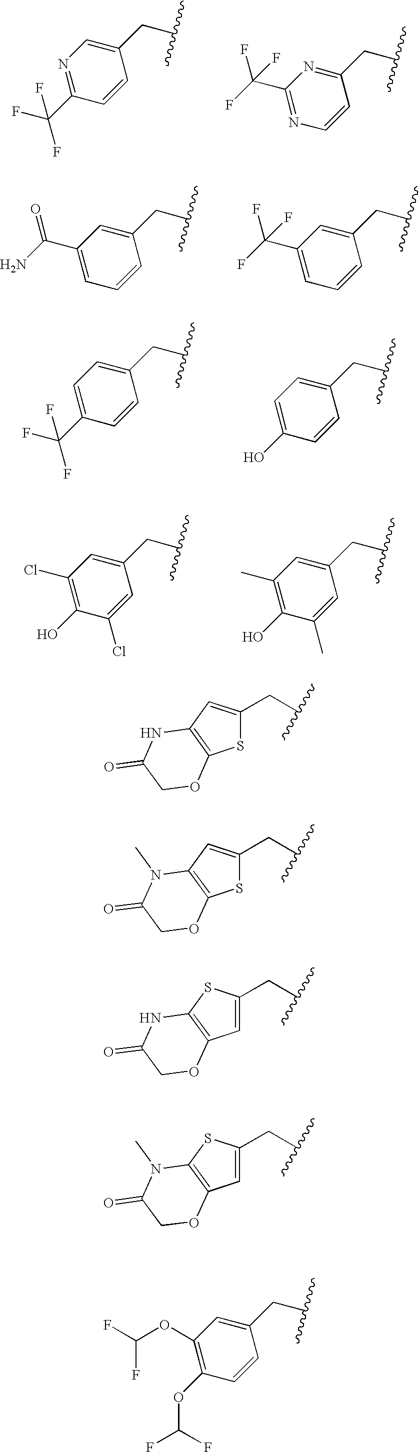

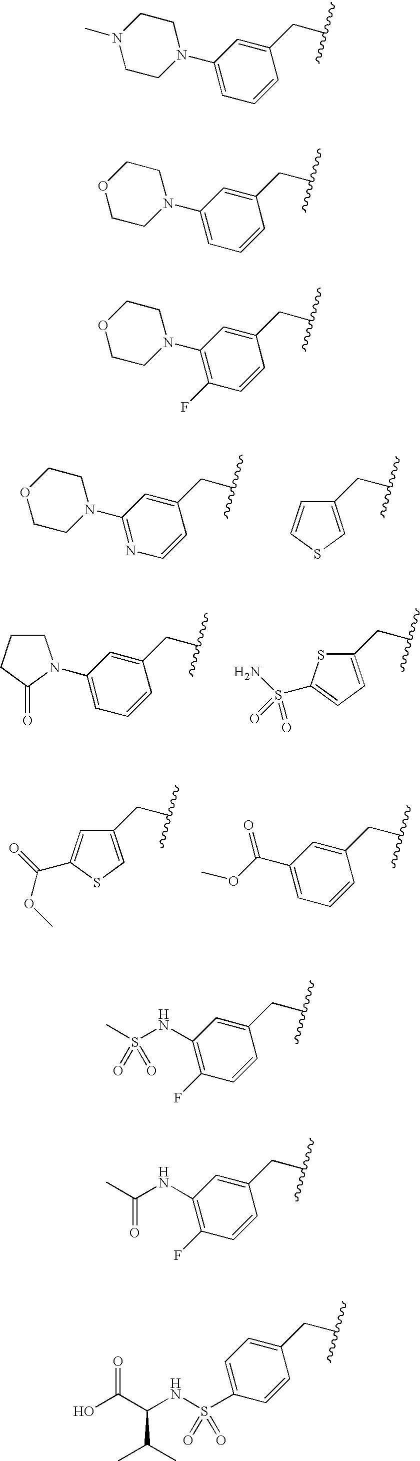

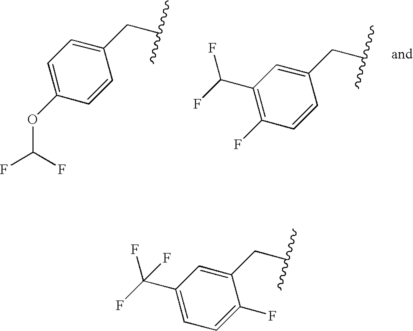

38. The compound according to claim 23, wherein R¹ is selected from the group consisting of: 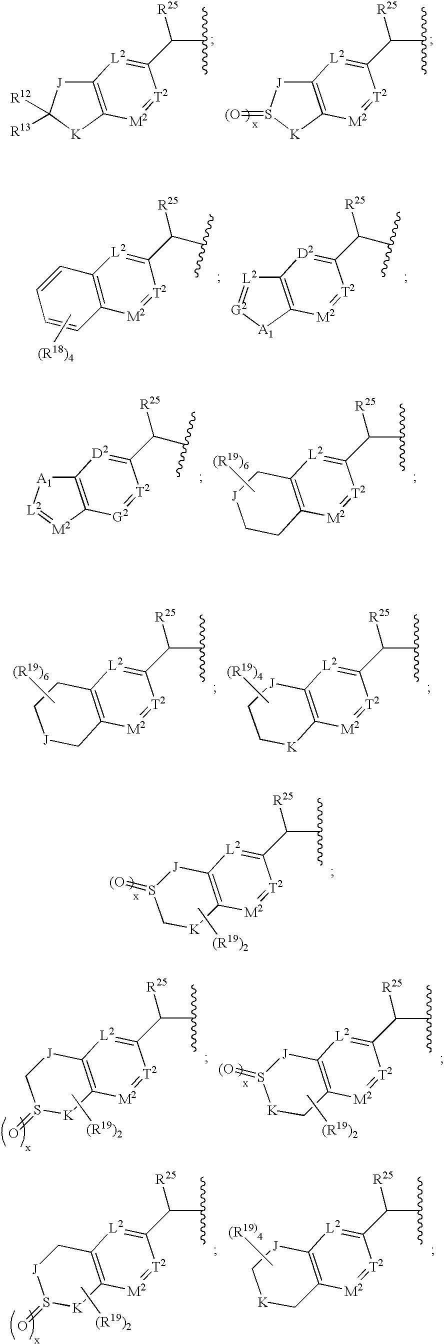 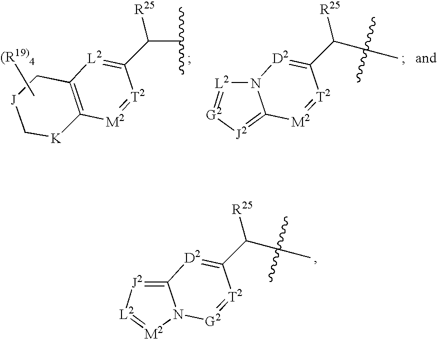
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹¹, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O, SO₂, SO and S; and
D², G², J², L², M² and T² are independently selected from the group consisting of CR¹⁸ and N.

39. The compound according to claim 38, wherein R¹ is selected from the group consisting of:

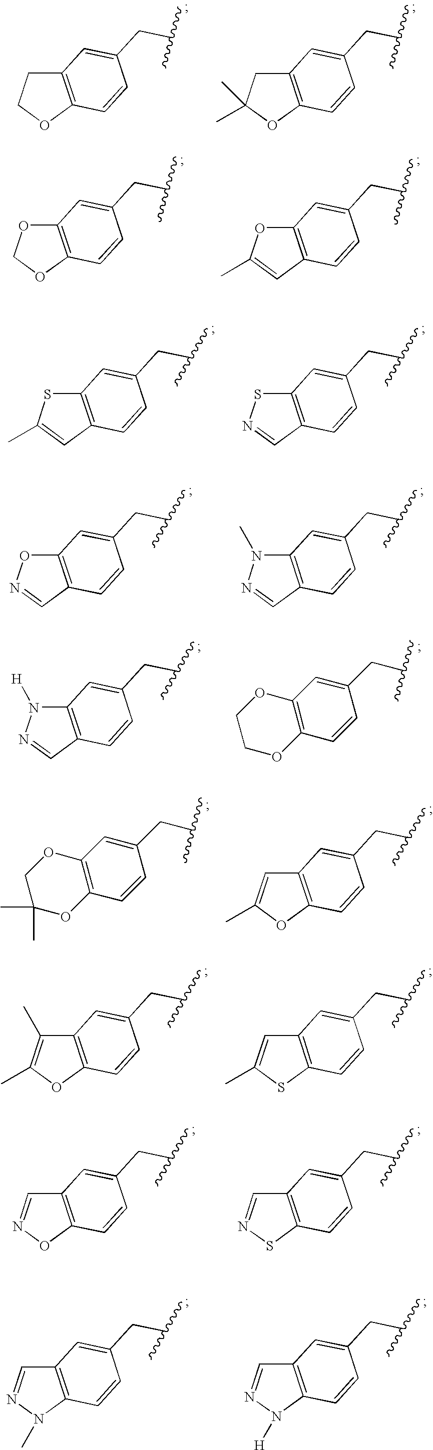

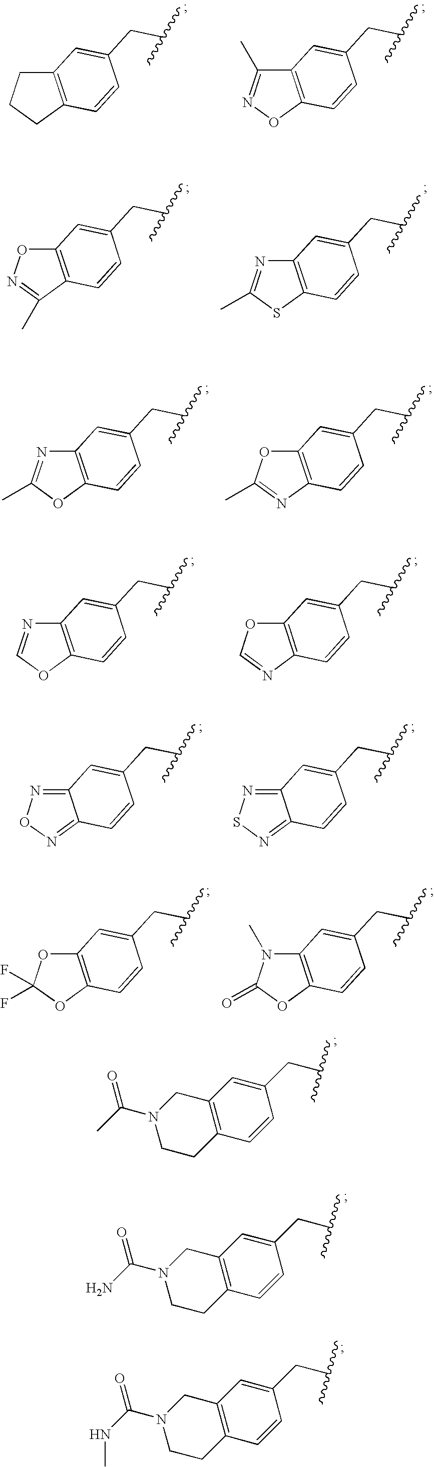

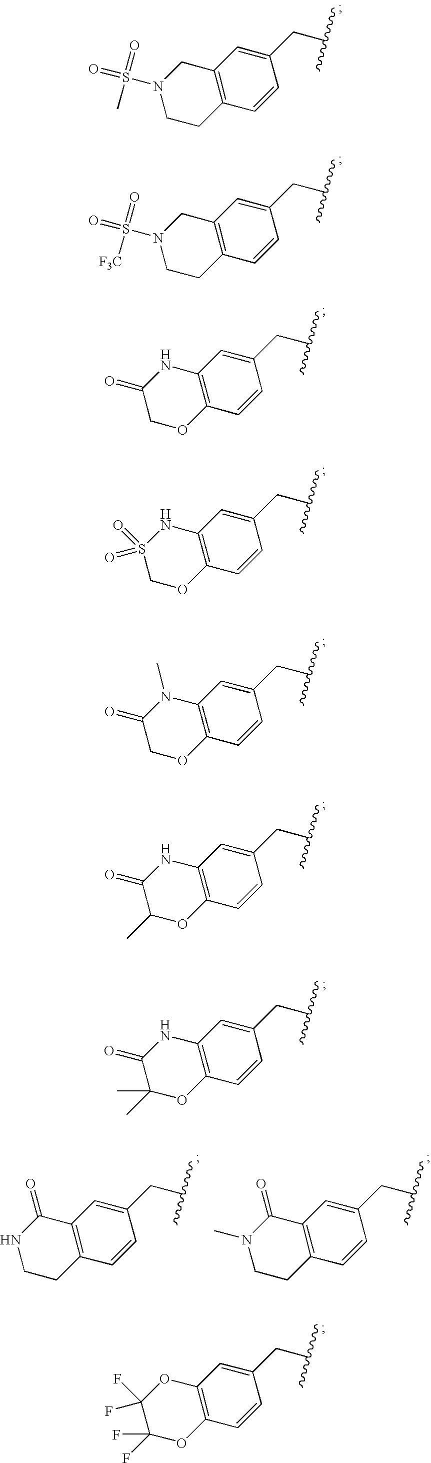

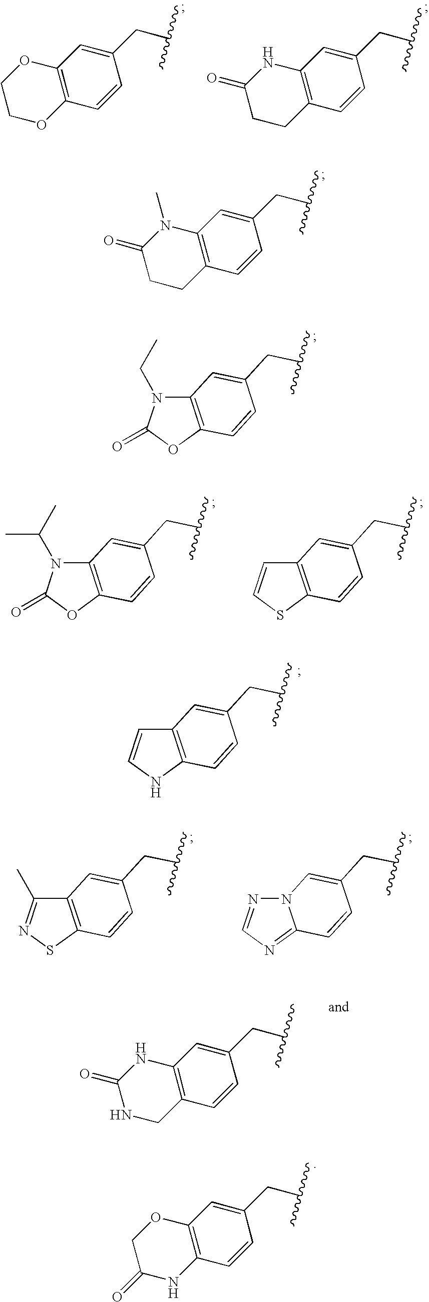

40. The compound according to claim 23, wherein R¹ is selected from the group consisting of: 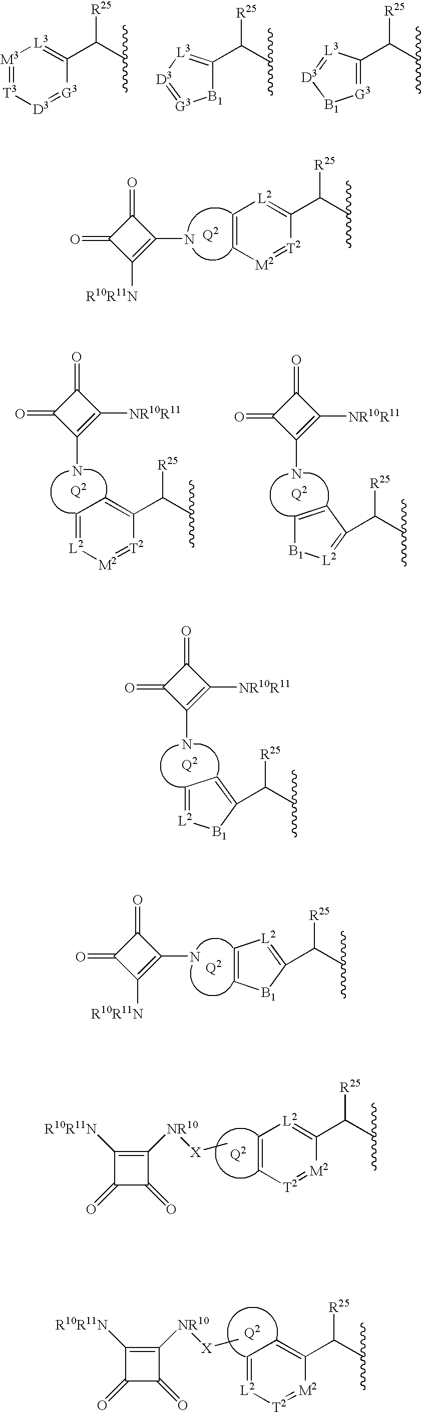 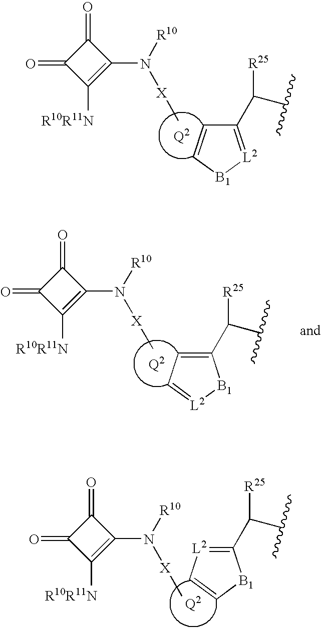
wherein:
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W1— and 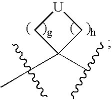
L², M², and T² are independently selected from the group consisting of CR¹⁸ and N;
D³, G³, L³, M³, and T³ are independently selected from N, CR¹⁸, (i) and (ii) 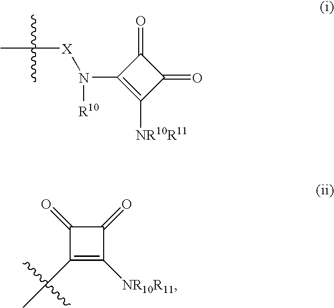
with the proviso that one of L³, M³, T³, D³, and G³ is (i) or (ii)
B, is selected from the group consisting of NR¹⁰, O, SO₂, SO and S; and
Q² is a 5- to 8-membered ring selected from the group consisting of cycloalkyl, heterocycloalkyl, aryl, and heteroaryl, which is optionally substituted one or more times with R¹⁹;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2; and
w is independently selected from 0-4.

41. The compound according to claim 40, wherein R¹ is selected from the group consisting of:

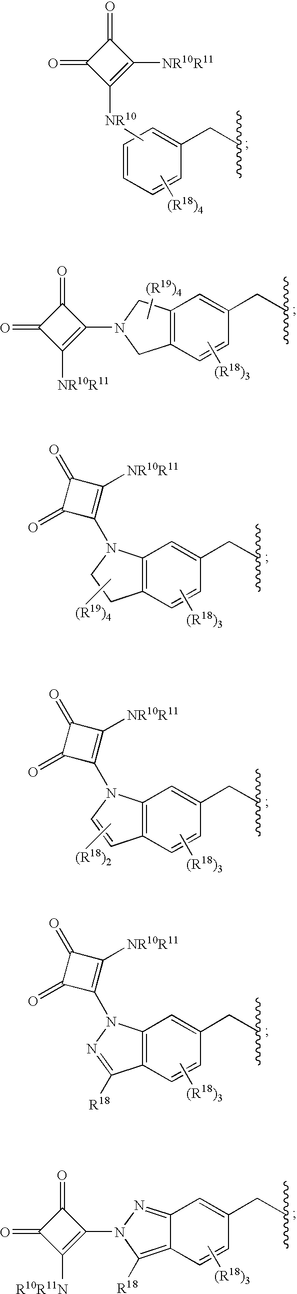

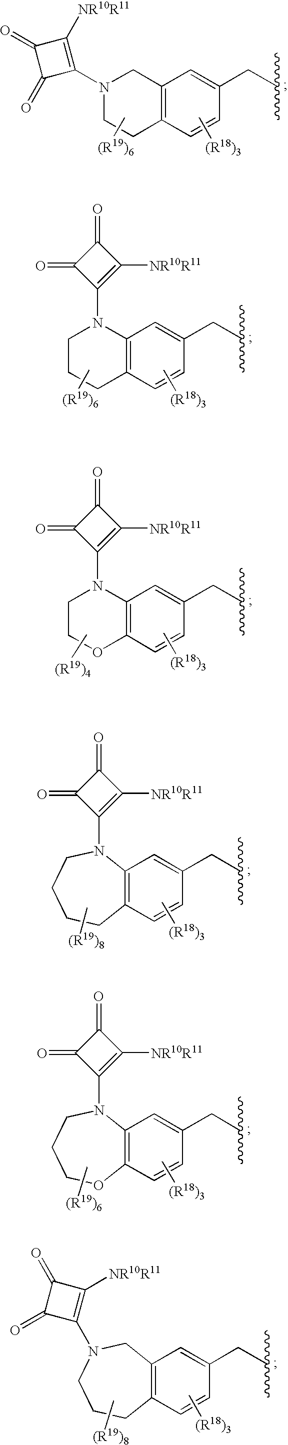

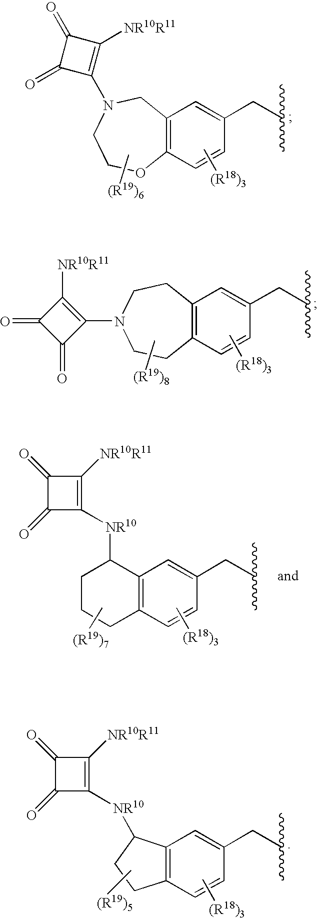

42. The compound according to claim 41, wherein R¹ is selected from the group consisting of:

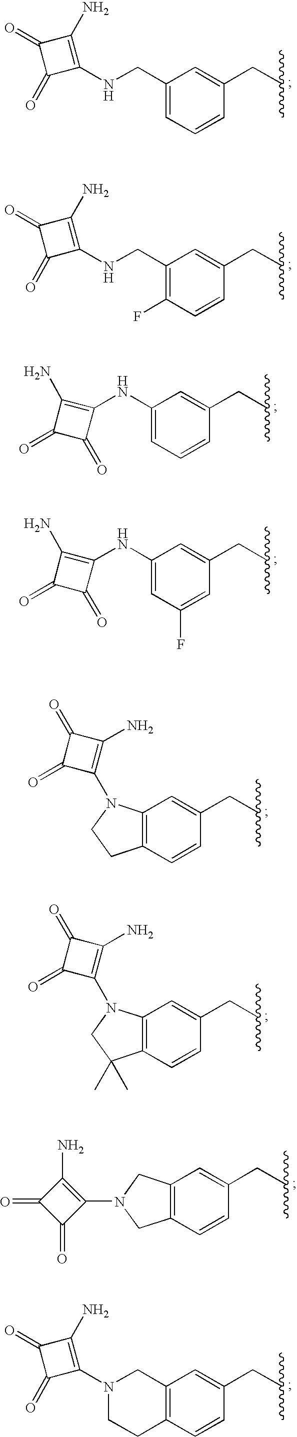

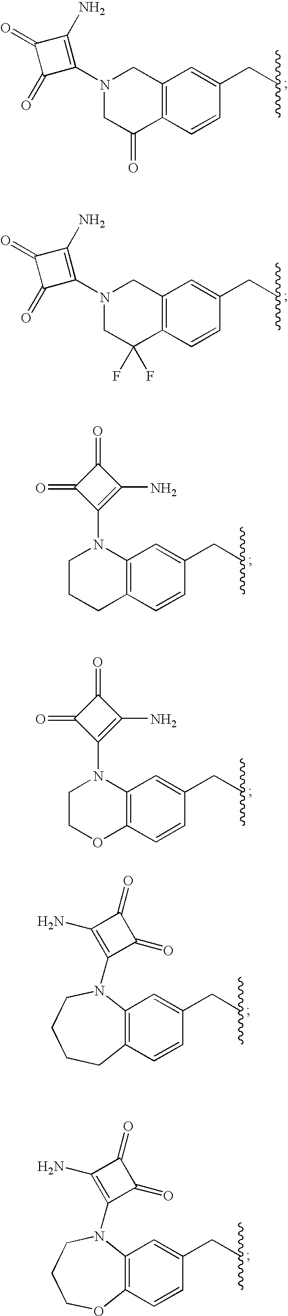

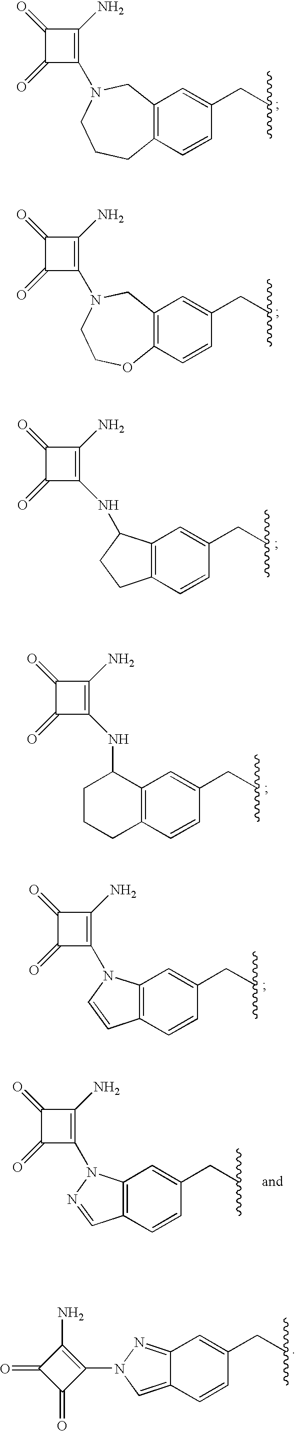

43. The compound of claim 23, wherein
X¹ is a bond, and
R³ is selected from the group consisting of

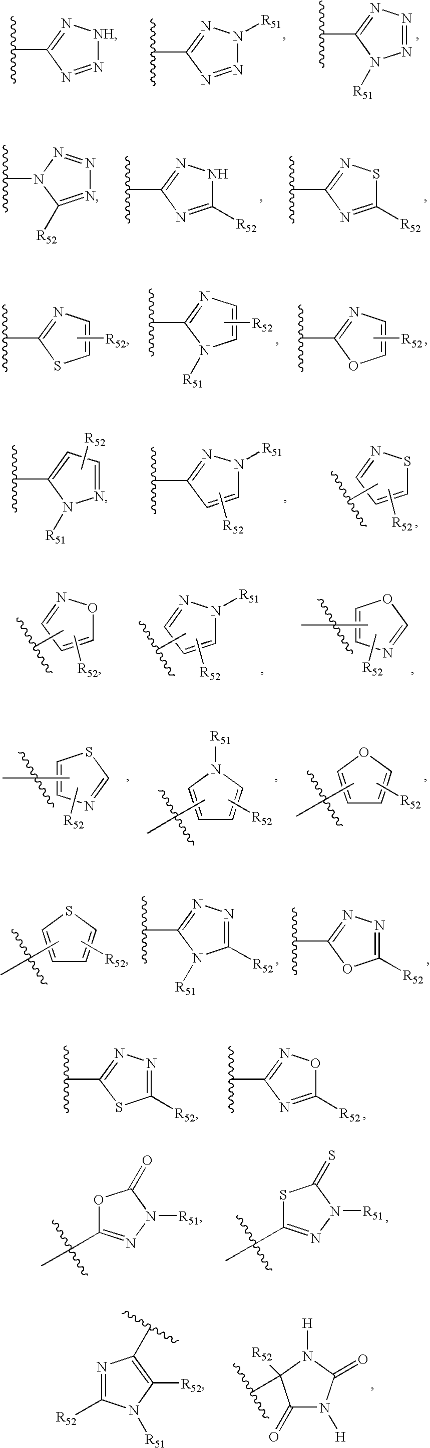

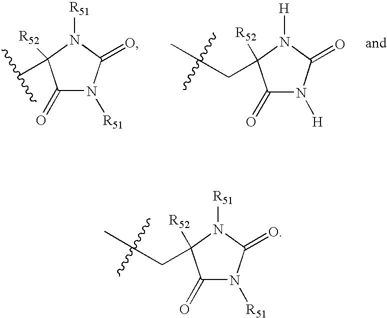

44. The compound of claim 26, wherein said compound is selected from the group consisting of:

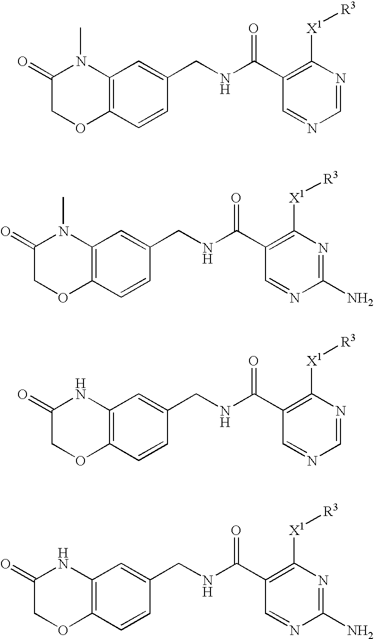

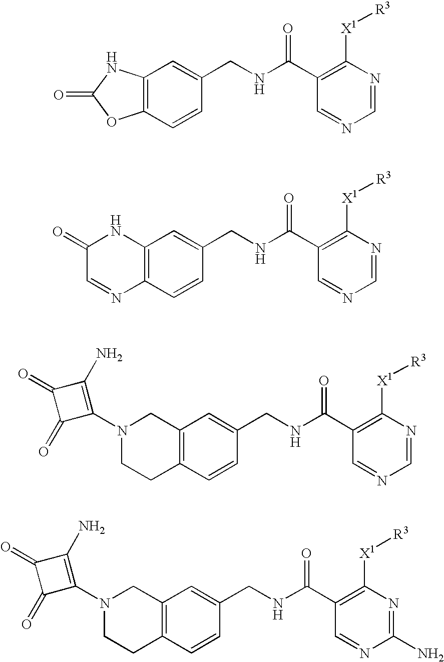

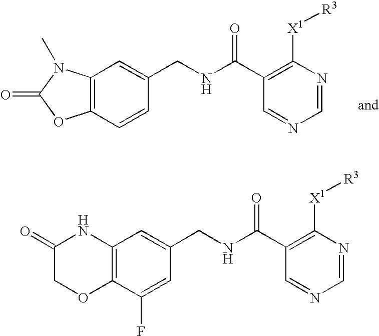

45. The compound selected from the group consisting of: 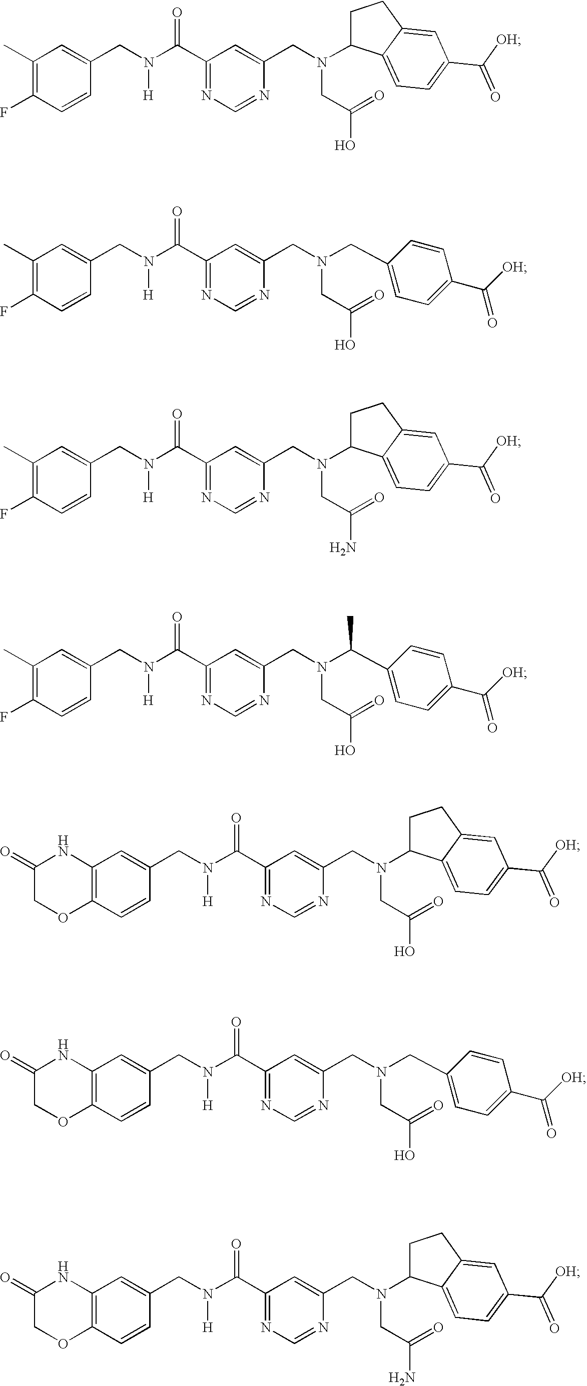 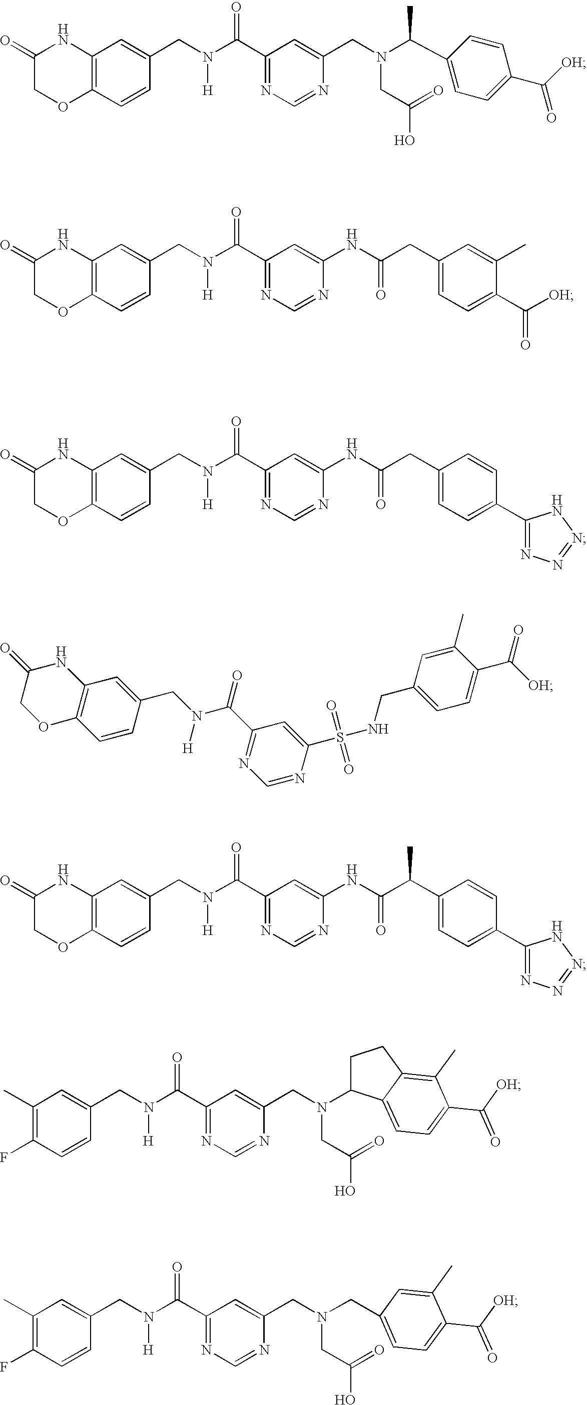 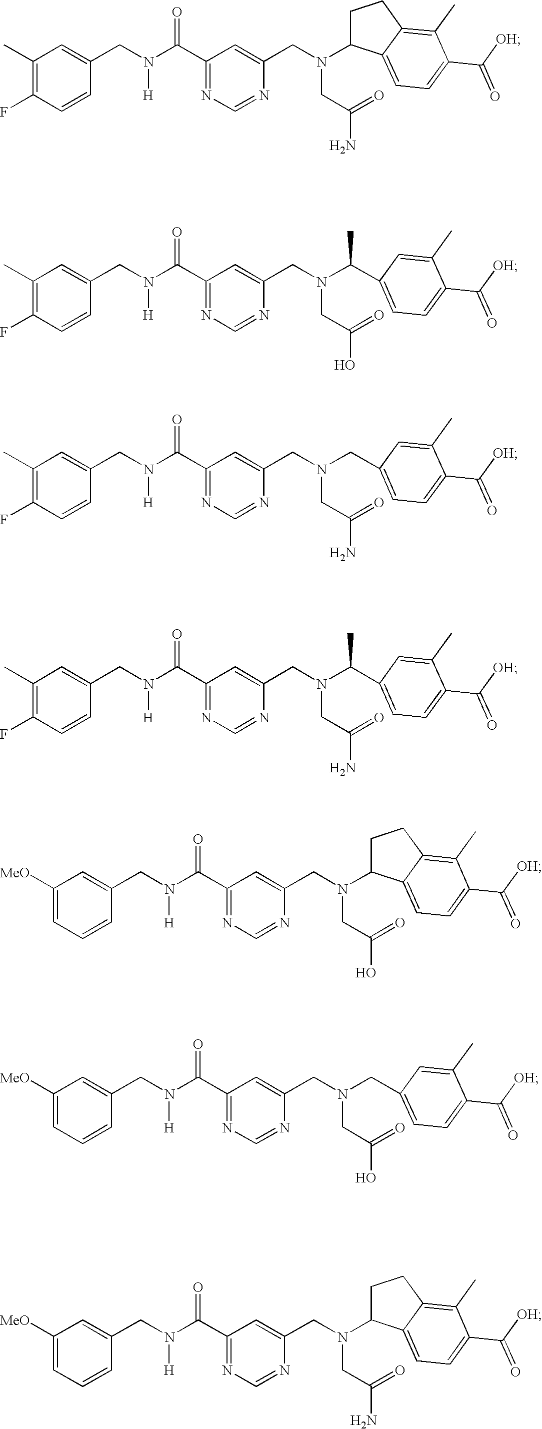 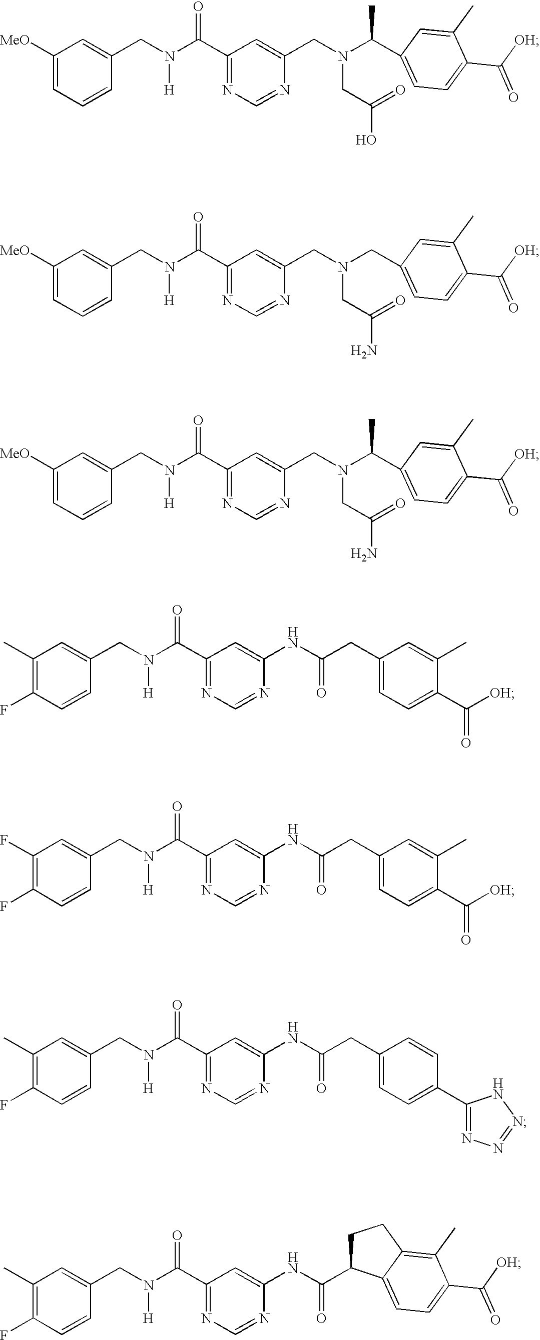 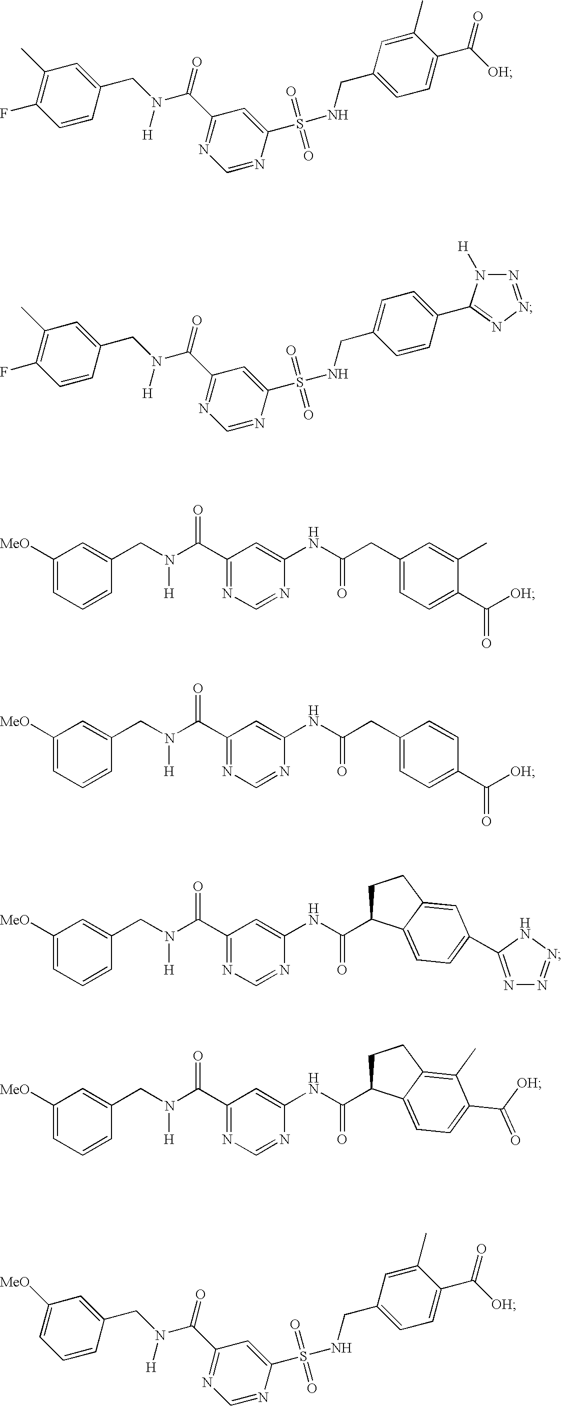 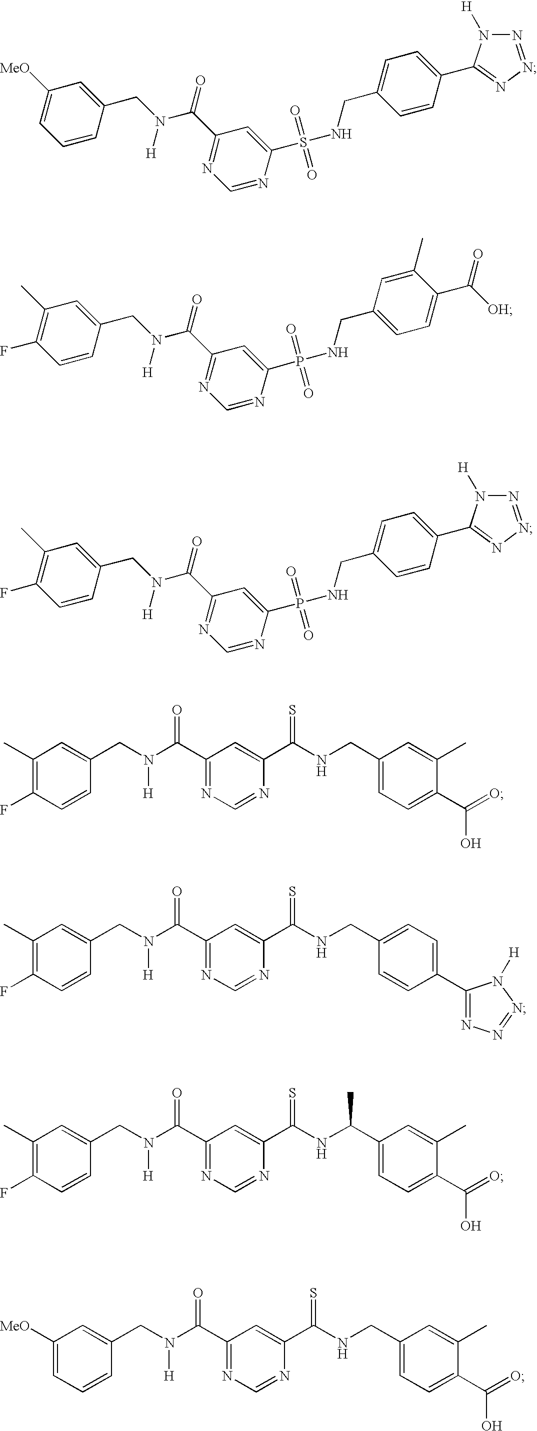 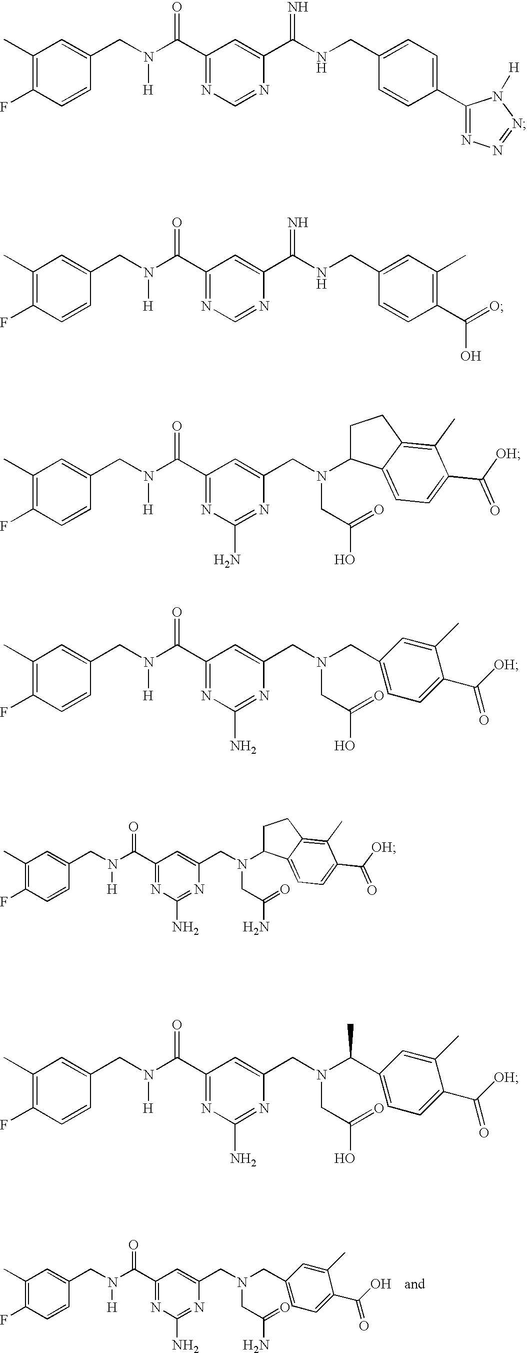 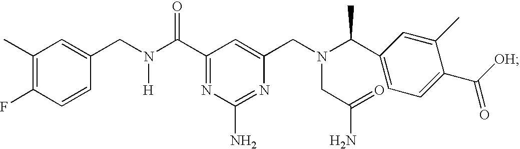
or a pharmaceutically acceptable salt thereof.

46. A compound selected from the group consisting of: 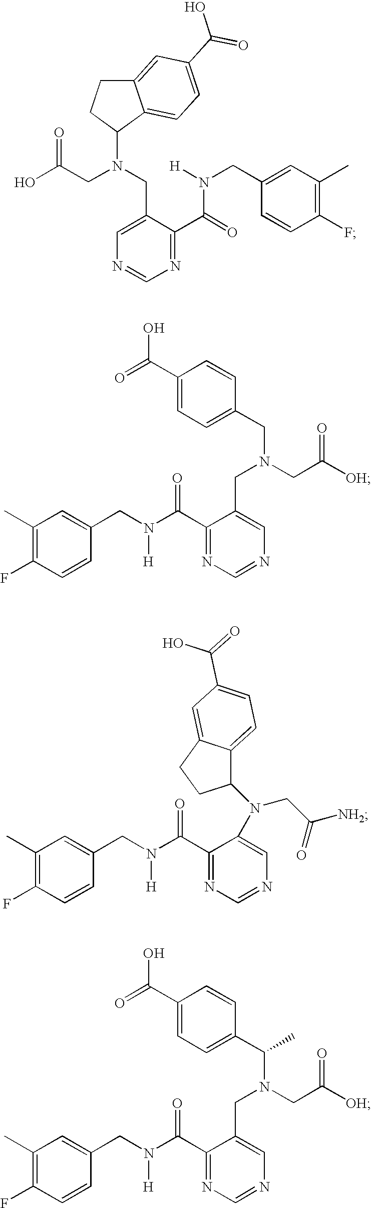 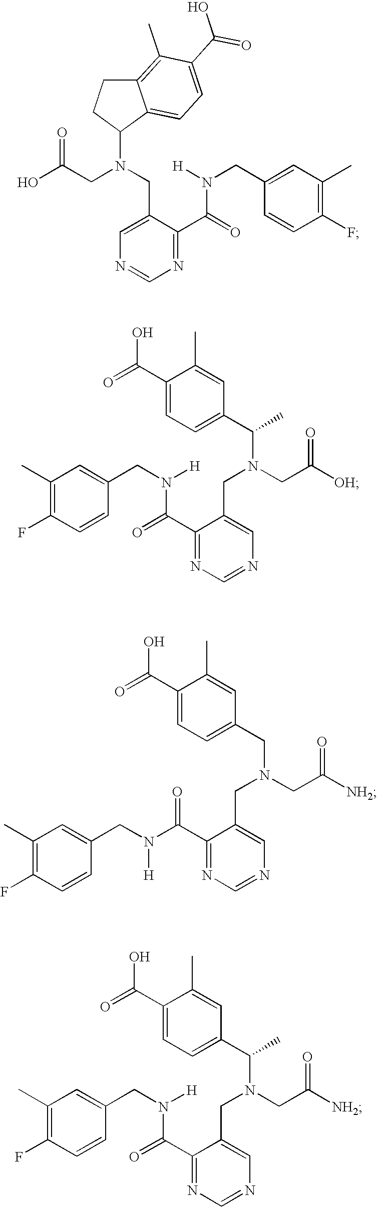 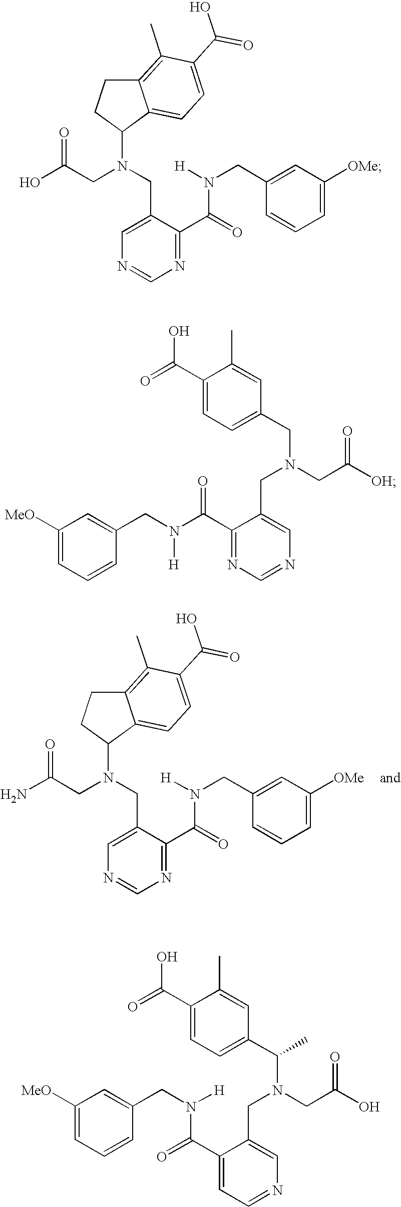
or a pharmaceutically acceptable salt thereof.

47. A compound selected from the group consisting of: 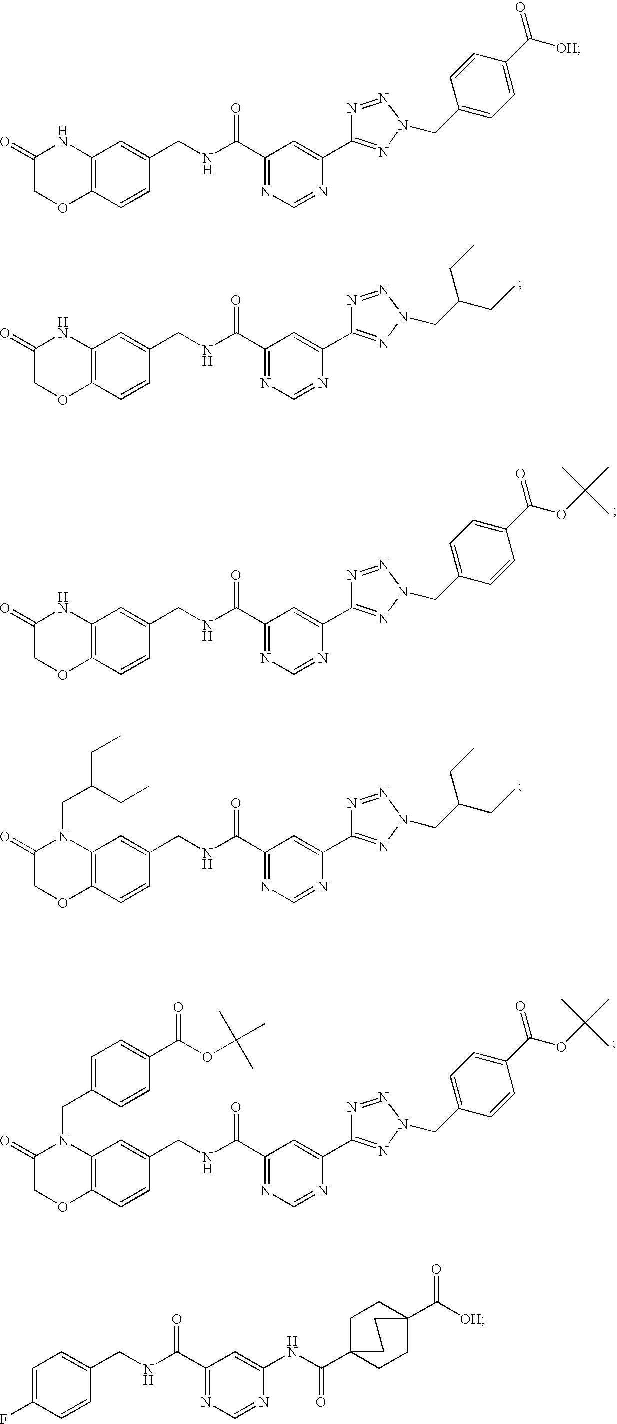 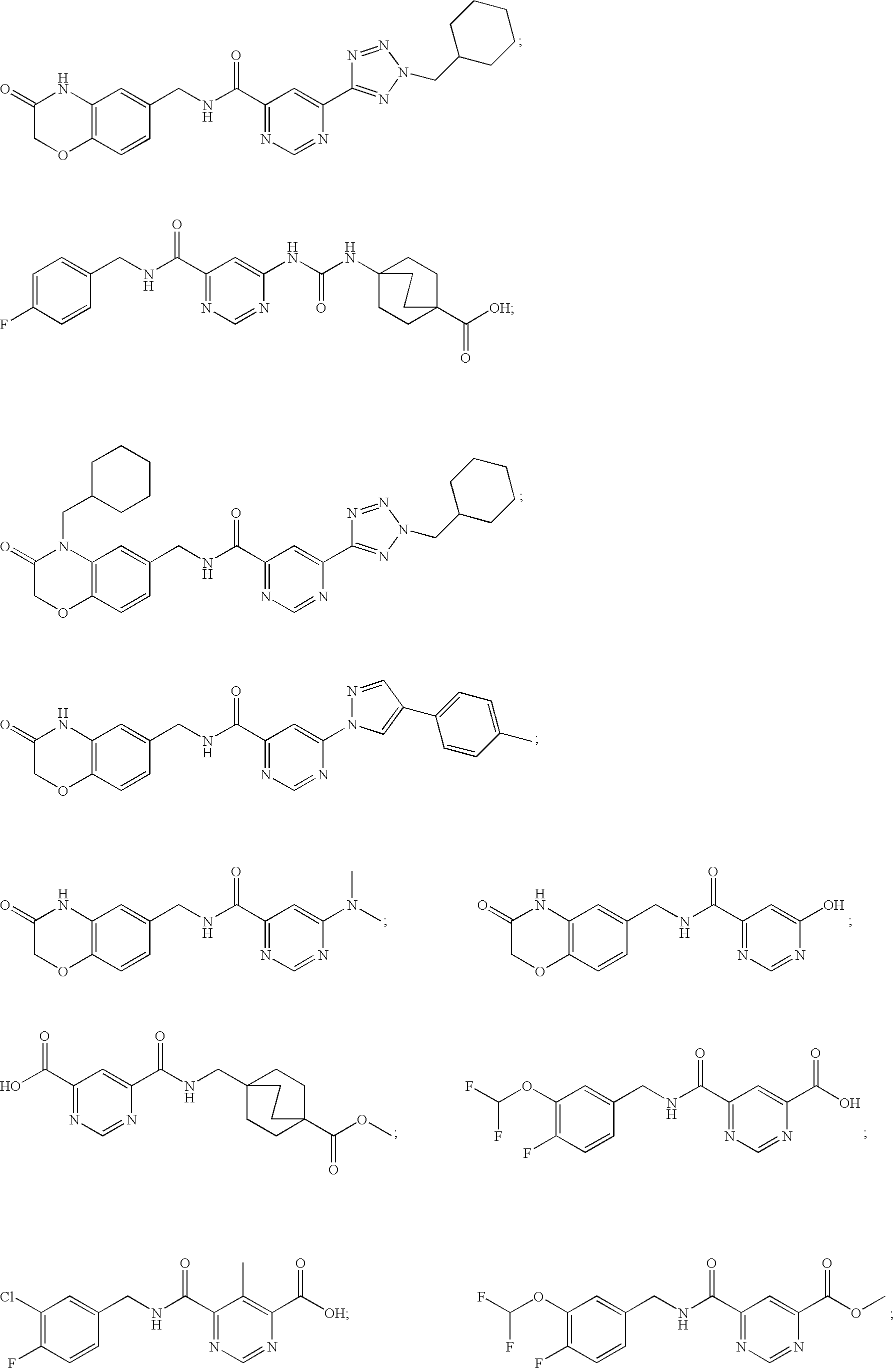 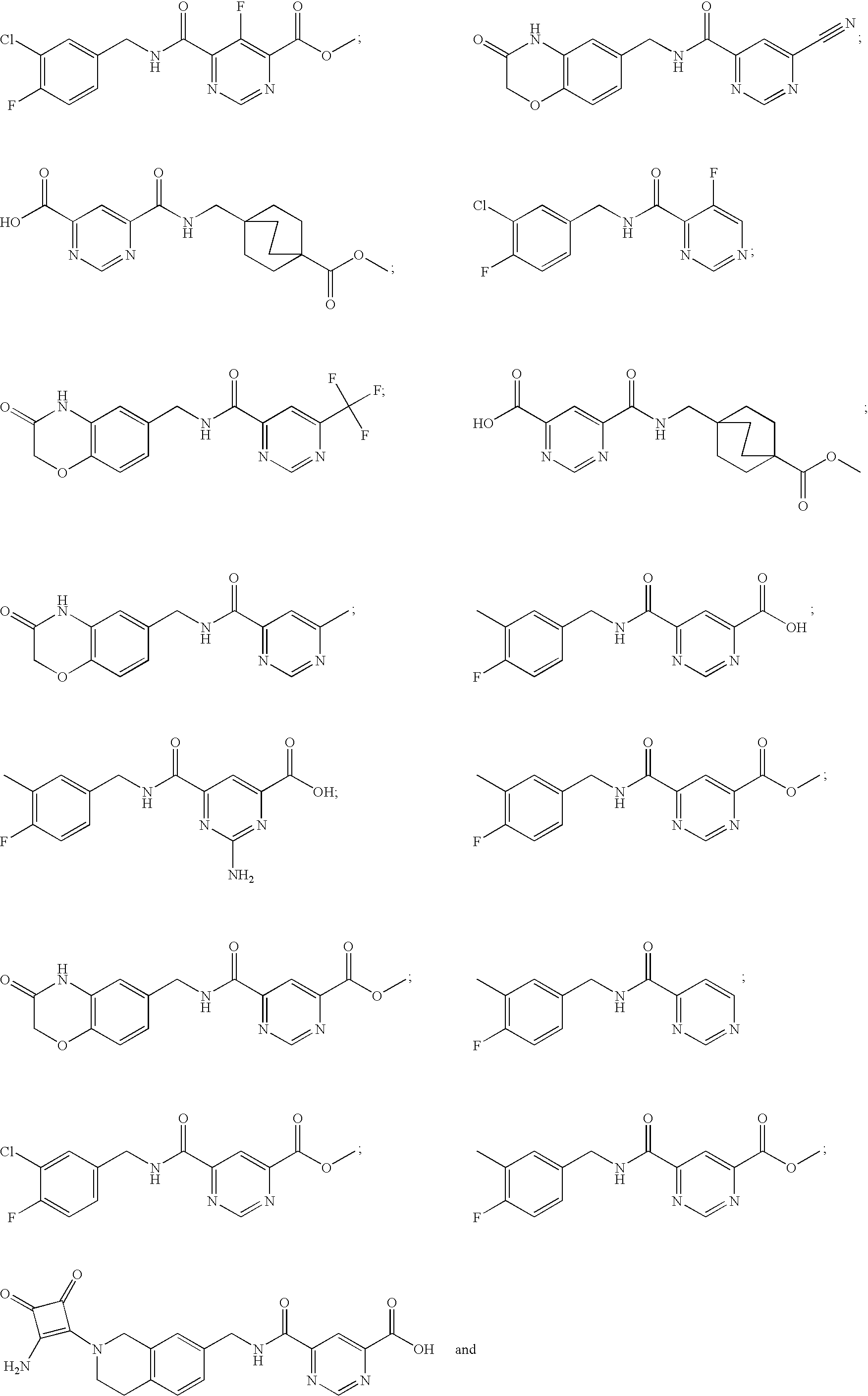 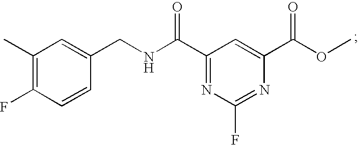
or a pharmaceutically acceptable salt thereof.

48. The compound of claim 1, having the structure: 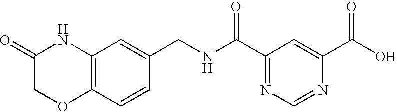
or a pharmaceutically acceptable salt thereof.

49. The compound of claim 1, having the structure:
or a pharmaceutically acceptable salt thereof.

50. The compound of claim 1, having the structure: 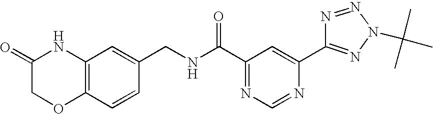 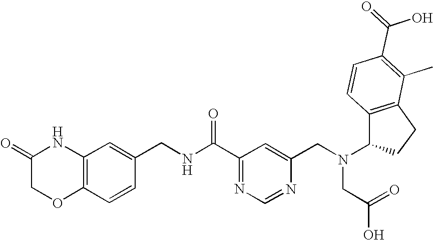
or a pharmaceutically acceptable salt thereof.

51. The compound of claim 1, having the structure: 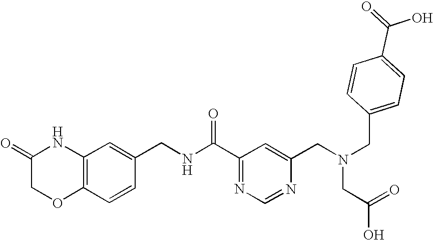
or a pharmaceutically acceptable salt thereof.

52. A pharmaceutical composition comprising an effective amount of the compound of claim 1 and a pharmaceutically acceptable carrier.

53. A pharmaceutical composition comprising an effective amount of the compound of claim 23 and a pharmaceutically acceptable carrier.

54. A method of inhibiting a metalloproteinase enzyme, comprising administering to a subject in need of such treatment a compound of claim 1 and a pharmaceutically acceptable carrier.

55. The method of claim 54, wherein said metalloprotease enzyme is selected one or more times from the group consisting of MMP-13, MMP-8, MMP-3, MMP-12 and ADAMTS-4 enzyme.

56. The method of claim 55, wherein said metalloprotease enzyme is the MMP-13 enzyme.

57. A method of inhibiting a metalloproteinase enzyme, comprising administering to a subject in need of such treatment a compound of claim 23 and a pharmaceutically acceptable carrier.

58. The method of claim 57, wherein said metalloprotease enzyme is selected one or more times from the group consisting of MMP-13, MMP-8, MMP-3, MMP-12 and ADAMTS-4 enzyme.

59. The method of claim 58, wherein said metalloprotease enzyme is the MMP-13 enzyme.

60. A method of treating an MMP-13 mediated disease, comprising administering to a subject in need of such treatment an effective amount of a compound of claim 1 and a pharmaceutically acceptable carrier.

61. A method of treating an MMP-13 mediated disease, comprising administering to a subject in need of such treatment an effective amount of a compound of claim 23 and a pharmaceutically acceptable carrier.

62. The method according to claim 60, wherein the disease is selected from the group consisting of: rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, wound healing, hemorroid, skin beautifying, pain, inflammatory pain, bone pain and joint pain.

63. The method according to claim 61, wherein the disease is selected from the group consisting of: rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, wound healing, hemorroid, skin beautifying, pain, inflammatory pain, bone pain and joint pain.

64. The method according to claim 62, wherein the disease is rheumatoid arthritis.

65. The method according to claim 62, wherein the disease is osteoarthritis.

66. The method according to claim 62, wherein the disease is inflammation.

67. The method according to claim 62, wherein the disease is atherosclerosis.

68. The method according to claim 63, wherein the disease is rheumatoid arthritis.

69. The method according to claim 63, wherein the disease is osteoarthritis.

70. The method according to claim 63, wherein the disease is inflammation.

71. The method according to claim 63, wherein the disease is atherosclerosis.

72. A pharmaceutical composition comprising:
A) an effective amount of a compound of claim 1;
B) a pharmaceutically acceptable carrier; and
C) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

73. A pharmaceutical composition comprising:
A) an effective amount of a compound of claim 23;
B) a pharmaceutically acceptable carrier; and
C) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

74. A pharmaceutical composition comprising at least one compound selected from the group consisting of: 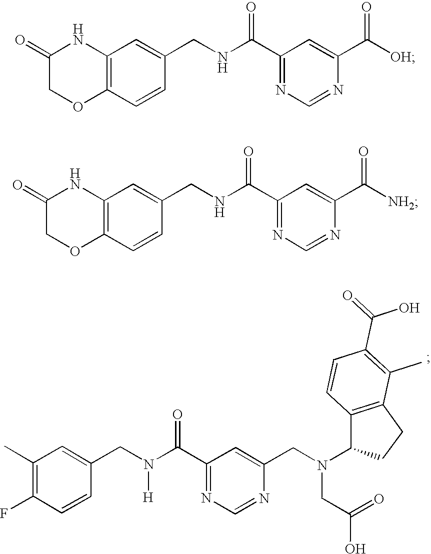 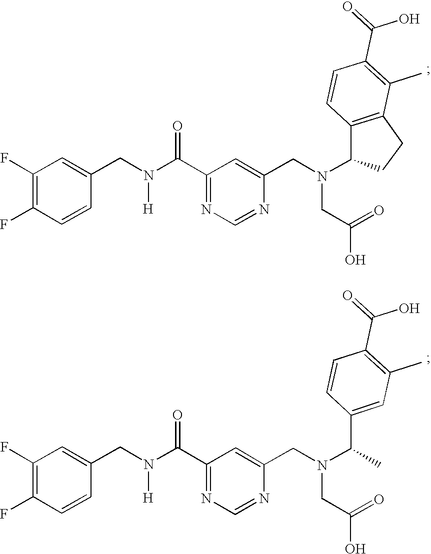 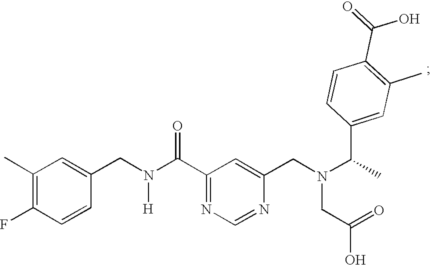 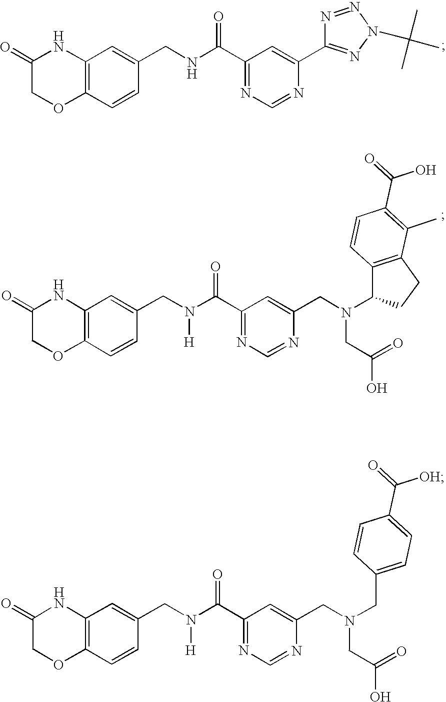 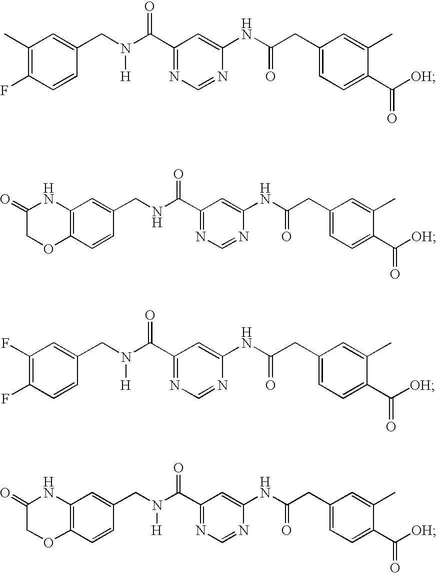 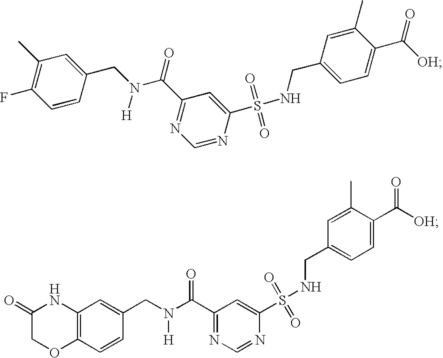 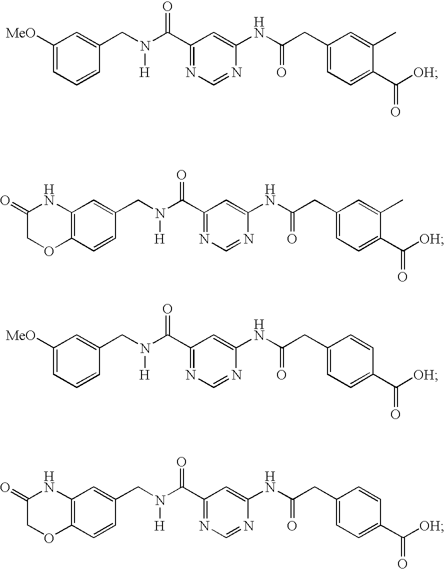 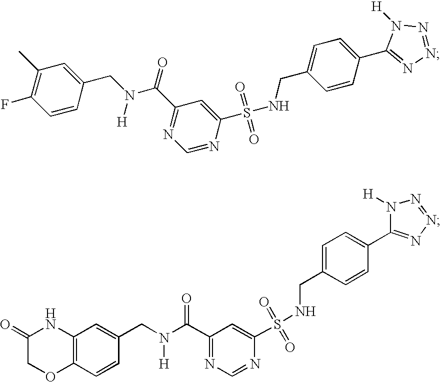 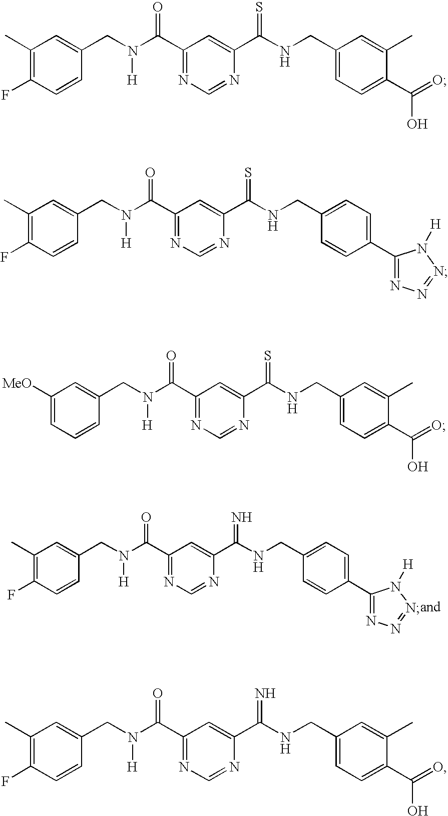
N-oxides, pharmaceutically acceptable salts, prodrugs, formulations, polymorphs, tautomers, racemic mixtures and stereoisomers thereof.
